(19) United States
(12) Patent Application Publication
(10) Pub. No.: US 2025/0250306 A1
(43) Pub. Date: Aug. 7, 2025

(54) IMMUNOGENIC PROTEINS AND NUCLEIC ACIDS ENCODING THE SAME

(71) Applicants: International AIDS Vaccine Initiative, Inc., New York, NY (US); The Scripps Research Institute, La Jolla, CA (US)

(72) Inventors: Jordan WILLIS, New York, NY (US); Manyuan MA, La Jolla, CA (US); Jon STEICHEN, La Jolla, CA (US); William SCHIEF, La Jolla, CA (US)

(21) Appl. No.: 19/061,222

(22) Filed: Feb. 24, 2025

Related U.S. Application Data
(63) Continuation-in-part of application No. PCT/US2023/072816, filed on Aug. 24, 2023.
(60) Provisional application No. 63/373,414, filed on Aug. 24, 2022.

(51) Int. Cl. C07K 14/005; A61K 39/00; A61P 37/04

(52) U.S. Cl. CPC C07K 14/005; A61P 37/04; A61K 39/00; A61K 2039/53; A61K 2039/55555; C12N 2740/16022; C12N 2740/16034

(57) ABSTRACT

The invention relates to proteins and nucleic acids for immunization regimens, modifications thereof, and/or development of nanoparticles, and/or development of membrane-anchored immunogens, and methods of making and using the same. The invention also encompasses cell surface trimers that bind to the broadly neutralizing antibodies and/or nucleic acids encoding the same.

### RELATED APPLICATIONS AND INCORPORATION BY REFERENCE

This application is a continuation-in-part of International Application No. PCT/US2023/072816 filed Aug. 24, 2023, and published as International Publication No. WO 2024/044684 on Feb. 29, 2024 and which claims priority under 35 U.S.C. § 119 (e) to U.S. Provisional Application Ser. No. 63/373,414 filed Aug. 24, 2022. All documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

### FEDERAL FUNDING LEGEND

This invention was made with government support under Grant Nos. UM 1 A 1100663 and UM 1 A 1144462 awarded by the National Institute of Allergy and Infectious Diseases (NIAID). The government has certain rights in the invention.

### SEQUENCE STATEMENT

The instant application contains a Sequence Listing which has been submitted via Patent Center and is hereby incorporated by reference in its entirety. Said.xml copy, created on Sep. 22, 2023 is named Y 7969-02064, and is 229,692 bytes in size.

### FIELD OF THE INVENTION

The invention relates to proteins and nucleic acids for immunization regimens, modifications thereof, and/or development of nanoparticles, and/or development of membrane-anchored immunogens, and methods of making and using the same.

### BACKGROUND OF THE INVENTION

A vaccine for HIV-1 is urgently needed, as there are approximately 1.5 million new infections each year as of 2020 (http://www.unaids.org/en/resources/fact-sheet). The target of HIV neutralizing antibodies, the trimeric envelope (Env) spike, varies substantially in sequence across different HIV-1 isolates, indicating that a vaccine should induce ‘broadly neutralizing antibodies’ (bnAbs), antibodies capable of neutralizing diverse isolates (Burton and Hangartner, 2016, Annu Rev Immunol 34, 635-659. 10.1146/annurev-immunol-041015-055515). Potent HIV bnAbs develop in a small percentage of infected individuals, typically over an extended course of infection (Burton and Mascola, 2015, Nat Immunol 16, 571-576. 10.1038/ni.3158; Kwong and Mascola, 2018, Immunity 48, 855-871. 10.1016/j.immuni.2018.04.029). Passive immunization with HIV-1 bnAbs has been shown to protect against simian/human immunodeficiency virus challenge in non-human primates (Pegu et al., 2019, Immunity 48, 855-871. 10.1016/j.immuni.2018.04.029; Pegu et al., 2017, Immunol Rev 275, 296-312. 10.1111/imr.12511) and to be capable of protecting humans against HIV-1 infection by neutralization-sensitive isolates (Corey et al., 2021, N Engl J Med 384, 1003-1014. 10.1056/NEJMoa2031738). Vaccine induction of bnAbs is regarded as having potential to protect against HIV, but bnAb elicitation in humans has not yet been achieved.

HIV bnAbs target at least five major epitopic regions on the Env trimer: V2-apex, V3-glycan, CD4 binding site, gp120/gp41 interface, and membrane proximal external region (M PER). Here, Applicants focus on V2-apex bnAbs. These include some of the most potent bnAbs and have been isolated from multiple individuals. V2-apex-directed responses are present in the serum of 15-20% of individuals who produce bnAbs (Landais et al., 2016, Plos Pathogens 12, e1005369. 10.1371/journal.ppat.1005369; Walker et al., 2010, PLOS Pathog 6, e1001028. 10.1371/journal.ppat.1001028), thus the human immune system appears to be relatively well-suited to generate responses to this epitope region.

Seven classes of V2-apex bnAbs have been identified, five of which possess long, negatively charged HCDR3s that are often decorated with sulfated tyrosines: PG9/PG16 (Walker et al., 2009, Science 326, 285-289. 10.1126/science.1178746), PGT141-145 and PGDM 1400-1412 (Sok et al., 2014, Proceedings of the National Academy of Sciences of the United States of America 111, 17624-17629. 10.1073/pnas.1415789111; Walker et al., 2011, Nature 477, 466-U 117. 10.1038/nature10373), CH01-CH04 (Bonsignori et al., 2011, Journal of Virology 85, 9998-10009. 10.1128/jvi.05045-11), the CAP256.VRC26 lineage (Doria-Rose et al., 2016, Journal of Virology 90, 76-91. 10.1128/jvi.01791-15; Doria-Rose et al., 2014, Nature 509, 55-62. 10.1038/nature13036), and the PCT64 lineage (Landais et al., 2017, Immunity 47, 990-1003 e1009. 10.1016/j.immuni.2017.11.002). HCDR3s for these bnAbs mediate binding by reaching through the conserved glycan shield to contact a positively charged, semi-conserved protein surface on strand C of the V2 loop (Andrabi et al., 2017, Immunity 47, 524-537 e523. 10.1016/j.immuni.2017.08.006; Andrabi et al., 2015, Immunity 43, 959-973. 10.1016/j.immuni.2015.10.014; Gorman et al., 2016, Nat Struct Mol Biol 23, 81-90. 10.1038/nsmb.3144; Landais et al., 2017, Immunity 47, 990-1003 e1009. 10.1016/j.immuni.2017.11.002; Lee et al., 2017, Immunity 46, 690-702. 10.1016/j.immuni.2017.03.017; Mclellan al., et 2011, Nature 480, 336-343. 10.1038/nature10696; Pancera et al., 2010, J Virol 84, 8098-8110. 10.1128/JVI.00966-10; Pancera et al., 2013, Nature Structural & Molecular Biology 20, 804-+. 10.1038/nsmb.2600; Pejchal et al., 2010, Proc Natl Acad Sci USA 107, 11483-11488. 10.1073/pnas.1004600107; Sok et al., 2014, Proceedings of the National Academy of Sciences of the United States of America 111, 17624-17629. 10.1073/pnas.1415789111). Vaccine elicitation of V2-apex bnAbs may therefore require eliciting antibodies with HCDR3s similar to sequences in the known bnAbs.

The success of any vaccine strategy to induce V2-apex bnAbs with HCDR3s similar to those in the known bnAbs will depend strongly on whether the priming immunogen can activate human naive B cells with appropriate germline-recombined HCDR3s that have potential to mature into bnAb HCDR3s (Steichen et al., 2019, Science 366. 10.1126/science.aax4380). The ability of a priming immunogen to activate appropriate precursors will also depend on the frequency of such precursors in the naive human B cell repertoire—the lower the frequency, the more difficult priming will be. Precursor frequency is a major concern for V2-apex bnAb precursor priming, because the very long HCDR3s of many V2-apex bnAbs suggests that precursor frequencies may be extremely low (Briney et al., 2012, PLOS One 7, e36750. 10.1371/journal.pone.0036750). Multiple candidates have been proposed or investigated as priming immunogens for V2-apex bnAb responses (Alam et al., 2013, Proc Natl Acad Sci USA 110, 18214-18219. 10.1073/pnas.1317855110; Andrabi et al., 2019, Cell Rep 27, 2426-2441 e2426. 10.1016/j.celrep.2019.04.082; Bhiman et al., 2015, Nat Med 21, 1332-1336. 10.1038/nm.3963; Bonsignori et al., 2011, Journal of Virology 85, 9998-10009. 10.1128/jvi.05045-11; Doria-Rose et al., 2014, Nature 509, 55-62. 10.1038/nature13036; Gorman et al., 2016, Nat Struct Mol Biol 23, 81-90. 10.1038/nsmb.3144; Medina-Ramirez et al., 2017, J Exp Med 214, 2573-2590. 10.1084/jem.20161160), and at least one such candidate has entered clinical trials (BG505 SOSIP.GT1.1 gp140; ClinicalTrials.gov Identifier: NCT04224701). However, none of the candidates have been shown to prime bnAb precursors using BCR sequencing of induced responses in an animal model with low precursor frequency approximating the human physiological range, as has been accomplished with germline-targeting immunogens for other bnAb classes (Huang et al., 2020, Proc Natl Acad Sci USA 117, 22920-22931. 10.1073/pnas.2004489117; Jardine et al., 2015, Science 349, 156-161. 10.1126/science.aac5894; Parks et al., 2019, Cell Rep 29, 3060-3072 e3067. 10.1016/j.celrep.2019.10.071; Sok et al., 2016, Science 353, 1557-1560. 10.1126/science.aah3945; Steichen et al., 2019, Science 366. 10.1126/science.aax4380; Tian et al., 2016, Cell 166, 1471-1484 e1418. 10.1016/j.cell.2016.07.029; Wang et al., 2021, EMBO J 40, e105926. 10.15252/embj.2020105926).

Citation or identification of any document in this application is not an admission that such document is available as prior art to the present invention.

### SUMMARY OF THE INVENTION

The present invention relates to non-naturally occurring proteins, which may be involved in forming immunogenic proteins of the present invention.

The invention relates to a non-naturally occurring protein which may comprise any one of the sequences in Table 1.

The protein may have at least 90% or 95% homology or identity with the sequence of the non-naturally occurring protein(s) of the invention.

The invention also encompasses trimers which may comprise any one of the non-naturally occurring protein(s) of the invention.

The invention also encompasses nucleic acids encoding the non-naturally occurring protein(s) of the present invention, including nucleic acids that may have at least 90% or 95% homology or identity with a nucleotide encoding the sequence of the non-naturally occurring protein(s) of the invention.

The invention also encompasses eliciting an immune response which may comprise systemically administering to an animal in need thereof an effective amount of any one of the non-naturally occurring protein(s) or any one of the nucleic acids encoding the non-naturally occurring protein(s) of the present invention, including nucleic acids that may have at least 90% or 95% homology or identity with a nucleotide encoding the sequence of the non-naturally occurring protein(s) of the invention. Advantageously, the nucleic acid is formulated in lipid nanoparticles (LNPs). The animal may be a mammal, advantageously a human.

Accordingly, it is an object of the invention not to encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. § 112, first paragraph) or the EPO (Article 83 of the EPC), such that Applicants reserve the right and hereby disclose a disclaimer of any previously described product, process of making the product, or method of using the product. It may be advantageous in the practice of the invention to be in compliance with Art. 53 (c) EPC and Rule 28 (b) and (c) EPC. All rights to explicitly disclaim any embodiments that are the subject of any granted patent(s) of applicant in the lineage of this application or in any other lineage or in any prior filed application of any third party is explicitly reserved. Nothing herein is to be construed as a promise.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following detailed description, given by way of example, but not intended to limit the invention solely to the specific embodiments described, may best be understood in conjunction with the accompanying drawings.

FIG. 1. Overview of human antibody repertoire-guided immunogen design. Repertoire analysis, immunogen design and structure determination (steps with solid arrows) were carried out in this study. Immunization in a knock-in mouse model (dashed arrows) was carried out Example 2.

FIG. 2. (SEQ ID NO:35-44) V2-apex precursor frequencies. (A) HCDR3 junctions for HCDR3-dominant V2-apex bnAbs aligned with the inferred V_{H}, DH, and JH gene sequences. Amino acids in grey are either located within a junction (non-templated) or mutated from a germline gene. (B) HCDR3 structures for each class of HCDR3-dominant V2-apex antibody: PCT64-35B (PDB: 5FEH), CH04 (PDB: 3TCL), PGDM 1400 (PDB: 4RQQ), PGT145 (PDB: 5V8L), and CAP256-VRC26.25 (PDB: 5DT1). Templated portions of the HCDR3 are colored as in (A). (C) Precursor detection rates among 14 donors, with 95% Wilson confidence intervals. Positive detection was defined as at least one precursor being found in a donor. (D) Precursor frequencies for each donor, and for each class of HCDR3-dominant V2-apex bnAb, considering heavy chains only (H) or heavy and light chains (H+L). The number of unique sequences used in the precursor search for each donor is indicated. Black lines indicate median precursor frequencies. (E) The mean number of mutations to a known bnAb in the class, for each precursor found in (D). Each symbol represents the mean for one donor. Black lines indicate the median over donors.

FIG. 3: (SEQ ID NO:45-57) Rationale for low precursor frequency. (A) HCDR3 length distribution across all 14 donors, in which symbols represent the mean frequency across all donors, and error bars indicate the standard deviations. V2-apex bnAb precursor lengths are highlighted in colored boxes: PCT64, grey; CH04, red; PG9/PG16, magenta; PGT/PGDM, cyan; CAP256, gold. (B) Average D-gene motif start position within HCDR3 for the PCT64 motif in HCDR3s of length 25; the CH01-CH04 motif in HCDR3s of length 26; and the PG9/PG16 motif in HCDR3s of length 30; across the 14 donors. The highlighted colored bar indicates the start position of the D gene motif for each V2-apex bnAb. PGT/PGDM and CAP256 classes had insufficient matches to compute average D motif start positions. (C) DJ-gene usage frequency heat map among all HCDR3s, long HCDR3s (>20 amino acids), and very long HCDR3s (>24 amino acids) in 14 HIV-unexposed donors. The J gene frequency is shown in a single dimension at the bottom of each DJ heatmap. Points are shown for each DJ gene used for the V2-apex bnAbs. (D) PG9 DJ junctional analysis. The frequency of amino acids at position 100p, for all HCDR3s of length 30 with a PG9-like DJ junction. The most common amino acid at 100p was tyrosine, observed in multiple donors and clonotypes. (E) PG9 and PCT64 inferred-germline variant junction alignments used in germline-targeting immunogen design. (F) PG9 and PCT64 inferred-germline variants binding affinities measured by SPR against the native-like trimers BG505 SOSIP.D664 (Sanders et al., 2013, PLOS Pathog 9, e1003618. 10.1371/journal.ppat.1003618) and BG505 SOSIP MD39 (Steichen et al., 2016, Immunity 45, 483-496. 10.1016/j.immuni.2016.08.016). NB, no binding, is the highest concentration tested.

FIG. 4. V2-apex germline targeting immunogen design. (A) The immunogen design pathway starting from BG505 SOSIP.D664. The V1/V2 region is shown with remodeled glycans as cyan spheres. The PG9 binding site is beige and two of the three protomers are shown in shades of grey. The mutations determined from each library are shown in red surface patches. Loop2b (residues 181-191 in HXBC2 numbering) is shown as a red tube representation. The germline variant that bound the immunogen is shown below each step. For clarity, glycans are shown on only a single protomer. (B) Surface plasmon resonance (SPR) K_{D}s for ApexGT trimer analytes binding to PCT64 and PG9 variants as IgG ligands with data fit using a 1:1 binding model. NB, no binding, is the highest concentration tested. (C) ELISA antigenic profiles of MD39 and ApexGT trimers. AUC is the area under the curve of the dilution series of the antibody shown on the x-axis. (D) Glycan composition for ApexGT trimers using two methods, single-site glycan analysis (SSGA) (Allen et al., 2021, bioRxiv. 10.1101/2021.03.08.433764) and DeGlyPHER (Baboo et al., 2021, Anal Chem 93, 13651-13657. 10.1021/acs.analchem.1c03059). High mannose, green; complex, pink; unoccupied, grey; N.D., glycan could not be resolved. (E) DSC melting temperatures of ApexGT trimers.

FIG. 5. Cryo-EM structures of ApexGT2.2M UT bound to PCT64 LMCA Fab, and ApexGT2 bound to PCT64 35S Fab. (A) Refined atomic models of both complexes. (B) Isolated structure and domain organization of both Fabs aligned to their heavy chains (HC), with PCT64 35S shown as partially transparent. (C) Electrostatic potential surfaces of both Fabs and of ApexGT2.2M UT (without glycans). (D) Close-up views of the binding interface showing antibody-gp120 protein interactions for both complexes. The outset shows an additional h-bond between the LMCA HC and gp120 not visible in the close-up. (E) Same as (D) but showing antibody-glycan interactions, with some parts of the LC hidden to enable viewing of specific residue contacts. (F) Table showing the number of h-bonds and total interfacial surface area between the different components of the epitope/paratope of both complexes. (G) Both complexes aligned on gp120A with arrows indicating the change in binding angle from LMCA to 35S. The additional red fragment on the rightmost panel is a model of the native BG505 SOSIP. MD39 loop2B highlighting the potential steric clashes with the LMCA HC.

FIG. 6. Cryo-EM structures of ApexGT3A bound to PG9 iGL Fab, and ApexGT3A.N130 bound to PG9 Fab. (A) Refined atomic models of both complexes. (B) Isolated structure and domain organization of both Fabs aligned on their heavy chains (HC), with PG9 shown as slightly transparent. (C) Electrostatic potential surfaces of both Fabs and of ApexGT3A (without glycans) calculated with APBS. (D) Close-up views of the binding interface showing antibody-gp120 protein interactions for both PCT64 complexes. All gp120 residues within 4 Å of the HCDR3 are shown and h-bonds are indicated with dashed blue lines. (E) Same as (D) but antibody-gp120 glycan interactions. (F) Table showing number of h-bonds and total interfacial surface area between the different components of the epitope/paratope of both complexes. (G) Both complexes aligned on gp120A revealing an identical binding angle. The additional red fragment on the rightmost panel is a model of the native BG505 SOSIP.MD39 loop2B highlighting the potential steric clashes with the HC of both Fabs. (H) NSEM 2-D class averages of ApexGT3 in complex with PG9 showing classes with more than one Fab bound (Fabs false-colored blue) along with a segmented 3-D reconstruction of the 2 Fab bound class (highlighted in red).

FIG. 7. Membrane-bound ApexGT trimers. (A) Cartoon schematic of cell-surfaced displayed ApexGT5 trimer. Link14, shown in pink, bridges gp41 and gp120. Location of GT mutations is indicated in green. (B) Cell surface antigenic profile for DNA-expressed membrane-anchored trimers binding to IgG for control bnAbs (quaternary, PGT151 and PGT145; CD4bs, 12A 12; and V3-glycan, PGT121), non-nAbs (V3, 4025; CD4bs, B6 and F105), V2-apex bnAbs (PG9, PCT64), and V2-apex bnAb precursors (PCT64.LMCA, PCT64.LMCA.JREV, PCT64.iGL). Mean-fluorescence intensity (MFI) via FACS binding was normalized to PGT121 binding. All trimers are based on the BG505 isolate, and all have a c-terminal truncation at residue 709. gp151 contains no other modifications. MD39 contains stabilizing mutations in BG505 SOSIP MD39 (Steichen et al., 2019, Science 366. 10.1126/science.aax4380). ApexGT trimers contain GT mutations described in the text. (C) Similar to (B) but with membrane-anchored trimers expressed from mRNA.

FIG. 8. Overview of Libraries used in this study. ApexGT model shown in grey surface representation. Regions of blue are the library positions. Sequences of sorting probe are shown in FIG. 9A.

FIG. 9A-9C. (SEQ ID NO:58-85,237) ApexGT Trimer Alignment and Extended Binding Analysis. ApexGT model shown in grey surface representation. Regions of blue are the library positions. Sequences of sorting probe are shown in FIG. 9A. (SEQ ID NO:58-75,237) (A) Amino acid sequence alignments for PCT64 and PG9 variants used in this study, aligned to their inferred germline VDJ segments. (B) Amino acid sequences of MD39 and ApexGT variants shown for positions 125-205 (HXBC2 numbering). Deletions are retained relative to HXBC2 to distinguish insertions. Loop2b is shown in grey. (C) Summary table of SPR KD values for ApexGT variants and mature or reverted members of the PCT64 and PG9/PG16 class of antibodies. K_{D}s were measured with trimer analytes and IgG ligands on the sensor chip and were determined by fitting the data with a 1:1 binding model. A dash indicates that the variant was not tested. A “>” or “>*” indicates that binding was not detected at maximal SOSIP concentration of 5 uM or 10 uM, respectively. All KD values are stated in nM.

FIG. 10. Cryo-EM data processing workflow for ApexGT2.2M UT in complex with PCT64 LMCA Fab. (A) A representative raw micrograph and 2-D class averages of picked particles. (B) Flowchart of 3-D data processing steps. (C) Fourier Shell Correlation (FSC) curve and particle angular distribution plots, (D) local resolution estimates, (E) soft mask used during refinement and FSC calculations, and (F) segmentation of the final 3-D reconstruction.

FIG. 11. Cryo-EM data processing workflow for ApexGT2 in complex with PCT64 35S and RM20A 3 Fabs. (A) A representative raw micrograph and 2-D class averages of picked particles. (B) Flowchart of 3-D data processing steps. (C) FSC curve and particle angular distribution plots, (D) local resolution estimates, (E) soft mask used during refinement and FSC calculations, and (F) segmentation of the final 3-D reconstructions both with and without 35S Fab. The base-binding antibody RM20A 3 Fab, included in the PCT64 35S complex to improve angular distribution and resolution, is evident in (B)-(F) but for simplicity is not shown in FIG. 5.

FIG. 12. Extended structural analysis. (A) Crystal structure of the PCT64 LMCA variable region. (B) Ca alignment of the LMCA (dark gray) and LMCASAR (white) variable light (VL) domains. The ‘SAR’ designation refers to the three mutations in the LCDR3. CDR loops of LMCA colored as in (A); HCDR3 of LMCASAR in orange, and LCDR3 in green. (C) Alignment of the HCDR3 in LMCA and LMCASAR upon superposition of the VH regions. The former HCDR3 is colored in magenta, and the latter in orange (top), and electron density map (bottom) of the LMCA HCDR3. Intervening residues between A sp100F and Tyr100J are missing electron density. 2Fo-Fc maps contoured at 1.00. (D) PCT64 LMCA structure from cryo-EM complex and the apo crystal structures of PCT64 LMCA, LMCASAR (PDBID: 6CA9), and PCT64-13C (PDBID: 6CA7) aligned to the heavy chains. (E) PCT64 35S structure from cryo-EM complex and the apo crystal structure of PCT64 35S (PDBID: 6CA6) aligned to the heavy chains. (F) Both PCT64 cryo-EM structures aligned to the HC to highlight changes in glycan conformations. (G) PCT64 structures aligned to the ApexGT2 with unbound trimer apex structure (dark gray) showing the displacement of each apex glycan relative to its unbound trimer apex conformation. (H) Same as in (F) but for PG9 and PG9 iGL. (I) PG9 iGL and PCT64 LMCA structures aligned to gp120 showing their near identical angle of approach. (J) Interprotomer distances as measured between the alpha carbons of residue 167.

FIG. 13: Cryo-EM data processing workflow for ApexGT3A in complex with PG9 iGL Fab. (A) A representative raw micrograph and 2-D class averages of picked particles. (B) Flowchart of 3-D data processing steps. (C) FSC curve and particle angular distribution plots, (D) local resolution estimates, (E) soft mask used during refinement and FSC calculations, and (F) segmentation of the final 3-D reconstruction.

FIG. 14. Cryo-EM data processing workflow for ApexGT3A.N130 in complex with PG9 Fab. (A) A representative raw micrograph and 2-D class averages of picked particles. (B) Flowchart of 3-D data processing steps. (C) FSC curve and particle angular distribution plots, (D) local resolution estimates, (E) soft mask used during refinement and FSC calculations, and (F) segmentation of the final 3-D reconstruction. G) Representative negative stain EM micrograph. H) negative stain 2-D class averages of ApexGT3 in complex with PG9 Fab. I) negative stain 3-D classification and refinement of 2-Fab bound class. J) Fourier shell correlation resolution plot for 2-Fab bound class.

FIG. 15: (SEQ ID NO:86-161) V2-Apex bnAb HCDR3s and Neutralization. Comprehensive V2-apex HCDR3 alignments and neutralization details for all V2 mABs considered in this study. The VDJ junction is shown for each bnAb class from FIG. 2A. Dashes indicate a junction that cannot be assigned to a gene segment. HCDR3 template metrics are shown next to each mA b. The templated portion indicates how many amino acids match the VDJ junction. Templated ratio is the number of templated amino acids divided by the total length of the HCDR3. VH gene mutations are shown in number of amino acids mutated from the parent VH gene. Neutralization metrics are shown from three different sources. The publication column indicates the source of neutralization breath and potency. CATNAP is taken from the CATNAP webservice which is a compiled list of publications where that antibody was tested (Yoon et al., 2015, Nucleic Acids Research, Volume 43, Issue W1, 1 Jul. 2015, Pages doi.org/10.1093/nar/gkv404). Data from the Seaman 109-virus panel are shown for many V2-apex members (Landais et al., 2017, Immunity 47, 990-1003 e1009. 10.1016/j.immuni.2017.11.002).

FIG. 16: (SEQ ID NO:162-165) Precursor Frequency Query Definitions. The query precursor definitions for the each V2-apex series are shown. The HCDR3 lengths are the lengths considered for that class. The D start and match are the regular expression syntax that was used to interrogate Applicants' database (see methods). “.” indicates a wildcard character used in the database search and will return all 20 amino acids at that position. The amino acids in between the brackets indicate the allowed mutations at that position (e.g. [DSG] will return an aspartic acid (D), serine(S) or glycine (G) at that position). V-gene and D-gene are shown for clarity but were not used in the query definition. VK/VL were used to interrogate pairing frequency for a given V gene family to a light chain from Dekosky et al. (DeKosky et al., 2015, Nat Med 21, 86-91. 10.1038/nm.3743).

FIG. 17: Individual Donor Precursor Frequencies. Individual precursor frequencies for each V2-apex bnAb class across donors, shown in counts per million. The columns deconstruct the precursor query by metric. Length, the HCDR3 length searched. Length+D, the HCDR3 length and D gene location described in FIG. 16. Length+D+V, the HCDR3 length, D gene location, and V gene family. Length+D+V+J (H), the HCDR3 length, D gene location, V gene family and J gene. H+L, the frequency computed from Length+D+V+J (H) and accounting for light chain pairing as described in the text. The median and geometric mean is shown for each V2-apex bnAb class. A dash indicates no results were found for that precursor definition.

FIG. 18: Individual Donor Precursor Edit Distances. Individual mean edit distances for the precursors found in each donor from FIG. 17. An edit distance is the number of amino acid differences between a known bnAb from that class and the precursor found in Applicants' dataset. The mean edit distance is the average edit distance for all precursors found, given the precursor frequency definition in that column. The column definitions are described in FIG. 17.

FIG. 19. (SEQ ID NO:166-227) Alignment of example precursors identified by sequence database searching.

HCDR3 amino acid sequence alignment of identified precursors. Inferred germline, mature bnAb and corresponding V/D/J genes are shown for reference. Sequences shown are sampled from the full precursor pool to show diverse junctional residues.

FIG. 20. Cryo-EM data and refined atomic statistics. Data collection and processing statistics for all cryo-EM and NS-EM datasets along with refinement and validation statistics for all atomic models.

FIG. 21. Immunization with GT2 activates PCT64 precursor B cells. A. Human PCT64 LMCA heavy chain (HC) (green), murine HC (dark grey), human PCT64 LMCA light chain (LC) (purple) and murine LC (light grey) sequences amplified from single-cell sorted B220+ naive B cells from two PCT64LMCA mice. Central n=sequence pairs amplified. See also FIGS. 28 and 29A-D. B. Representative plots of epitope-specific GT2-positive and GT2-KO-negative peripheral B cells in naïve PCT64LMCA or C57BL/6J mice. Events were pre-gated on lymphocytes/singlets/CD4-CD8-F4/80-Gr1-/B220+ B cells. C. Quantification of GT2-specific blood peripheral B cells from PCT64LMCA and WT C57BL/6J mice (n=4). D. Human PCT64 LMCA HC (green), human PCT64 LMCA LC (purple) and murine LC (light grey) sequences amplified from single-cell sorted GT2-specific naive B cells in PCT64LMCA mice (n=2). Central n=sequence pairs amplified. E. Schematic of the PCT64LMCA B cell adoptive transfer and immunization protocol used in F-H. F. Representative FACS plots of splenic B cells obtained at 8, 16 and 42 days post immunization (dpi) with GT2 trimers. Events were pre-gated on lymphocytes/singlets/live/CD4-CD8-F4/80-Gr1-/B220+ B cells and represent germinal centers (GC), CD45.1 and CD45.2 cells in GC, and frequency of GT2+CD45.2 cells present in GC. For control groups see FIG. 29E. G. Quantification of B cell subsets responsive to GT2-immunization at 7, 16 and 42 dpi. Left to right: total GCs, CD45.2+ B cells in GCs, GT2-binding CD45.2+ B cells. P values were calculated by Mann-Whitney test, ** P<0.01. See also FIG. 29E. H. ELISA quantification of GT2-binding (left) and GT2-KO-binding (right) serum IgG from PCT64LMCA-HL recipient mice compared to WT C57BL/6J mice. Titers were assessed prior to immunization with GT2 trimers and at 7, 14, 21 and 42 dpi. AUC (area under the curve).

FIG. 22. A single priming immunization with GT2 induces antibodies with mature PCT64-like mutations (A-I). Antigen-specific splenic CD95+CD38low CD45.2+ PCT64 LMCA B cells were sorted at 8 and 42 dpi for single-cell BCR sequencing. A. Phylogenetic clonal lineage trees showing diversification of PCT64LMCA IGH amino acids at 42 dpi. B. Total nucleotide (nt; left) and amino acid (aa; right) mutations acquired in PCT64 LMCA IGHV and IGKV at 8 and 42 dpi. C. HC mutation frequencies per residue observed at 8 (n=93) and 42 (n=57) dpi. HCDRs highlighted in gray. Red=residues present in mature PCT64; blue=on-track mutation present in early PCT64 isolates. AA positions 31, 35, 52B and 100D analyzed in (D). D. Distribution of selected PCT64 LMCA B cell HC aa mutations in positions 31, 35, 52B and 1 over time (8 and 42 dpi). Red=residues present in mature PCT64; blue=on-track mutation present in early PCT64 isolates; black=original LMCA aa; grey=all other mutations. See also FIG. 29F. E. Frequencies of LC mutations observed at each residue 42 dpi (n=39). LCDRs are highlighted in gray. F. SPR affinity measurement against GT2 for 14 antibodies isolated at 42 dpi (white, right) in comparison with the LMCA (green, left). G. Full cryo-EM structure of ApexGT2 in complex with GT2-d42.16 and RM20A 3 Fabs and close-up of the epitope/paratope region with sites of SHM in yellow. See also FIG. 30. H. Structures of GT2-d42.16 and PCT64.LMCA (dark gray) overlayed showing an identical angle of approach relative to their respective ApexGT trimers. I. Close up of the HCDR1 (light blue) and 2 domains (dark blue) with hydrogen-bonds between the N156gp120A glycan and gp120 residues shown as dashed lines.

FIG. 23. PCT64 precursor responses to GT2 are driven by the heavy chain. A. 10× Genomics single-cell BCR sequences from 4703 splenic B cells from a naïve PCT64LMCA-H mouse. Relative bubble size indicates frequency of IGHV genes used. Human PCT64 IGHV gene frequency in green (82.6%). Murine IGHV genes are represented by variable colors. B. Left: Representative FACS plot showing GT2-binding and GT2-K O-negative peripheral B cells in naïve PCT64LMCA-H. Events were pre-gated on lymphocytes/singlets/CD4-CD8-F4/80-Gr1-/B220+B cells. Right: Quantification of GT2-specific blood peripheral B cells from PCT64 LMCA-H model compared to C57BL/6J WT mice. C. Paired human PCT64 LMCA HC (green) and murine LC (variable colors) sequences amplified from single-cell sorted GT2-specific naive B cells from PCT64 LMCA-H mice. Central n=sequence pairs amplified. D. M urine IGK V genes paired with human PCT64 IGH in a naïve PCT64 LMCA-H mouse (n=3888). Relative bubble size indicates frequency of IGKV gene usage. Relevant IGK V genes are highlighted in different colors and analyzed in E and F. See also FIG. 31. E. M urine IGK V genes paired with human PCT64 IGH isolated from GCs at 8 and 42 dpi. Some of the most frequently enriched IGK V genes are highlighted: V2-109 (red), V12-44 (blue), V14-111 (yellow), V4-74 (light green), and V1-135 (teal). See also FIG. 32A. F. SPR affinity measurement against GT2 for 23 antibodies with various murine IGKV pairings isolated from naïve PCT64LMCA-H mice at 42 dpi, compared to human LMCA (black, square). G. Frequencies of HC aa mutations observed per residue at 42 dpi. HCDRs are highlighted in gray. Red=key mutations present in mature PCT64. AA positions 31, 35, 52B and 100D were analyzed in (J). See also FIG. 32B-E. H. Distribution of select PCT64 LMCA B cell HC aa mutations in positions 31, 35, 52B and 100D at 8 and 42 dpi. Red=residues present in mature PCT64; blue=on-track mutation present in early PCT64 isolates; black=original LMCA aa; grey=all other mutations.

FIG. 24. High affinity GT5 immunogen induces PCT64 precursor activation at physiological frequencies. A. Schematic of adoptive transfer model to calibrate PCT64 LMCA B cell frequencies. B. Titration of cell transfer model. Spleens of non-immunized CD45.1 recipient mice were stained for flow cytometry and absolute precursor quantification performed via bead assay. C. Precursor frequencies (y-axis) corresponding to number of B cells transferred (x-axis). D. Analysis of linearity of CD45.2 PCT64LMCA B cells recovered 24 h post transfer. E. Schematic of immunization in an adoptive transfer model with PCT64 precursor B cells at frequencies of 100, 20 and 10 per 106. F. Representative FACS plots at 8 dpi with GT2. G. Quantification of GC B cells and CD45.2+ PCT64LMCA cells present in GC at 8 dpi with GT2. H. SPR of PCT64 LMCA for GT2 and GT5 trimers. I. Representative flow cytometry plots 8 dpi with GT5, showing CD45.2 GC B cell responses at precursor frequency of 100, 20 and 10 per 106 of B cells. J. Quantification of GC cells and CD45.2+ PCT64LMCA cells present in GC at 8 dpi with GT5 at decreasing precursor frequencies. See also FIG. 32F-G. K. Representative FACS plots at 8, 16, and 42 dpi with GT5, showing CD45.2 GC B cell responses at precursor frequency of 100, 10 and 1 per 106 B cells (quantified in L). L. Quantification of CD45.2 present in GC at 8, 16 and 42 dpi in mice with precursors at 100, 10 and 1 per million B cells. M. ELISA quantification of GT2/GT5-binding (top) and GT2/GT5-KO-binding (bottom) serum IgG from PCT64LMCA recipient mice compared to WT C57B L/6J mice. Titers were assessed before and at 7, 14, 21 and 42 dpi with GT2 or GT5. AUC (area under the curve)

FIG. 25. GT5 immunization induces key PCT64-like mutations in IGH A. Quantification of total nt mutations in PCT64 LMCA HC and LCV genes at 42 dpi. See also FIG. 32H. B. Quantification of total aa mutations in PCT64LMCA HC and LCV genes at 42 dpi. C. SPR affinity measurement of antibodies isolated at 42 dpi with GT5 from mice presenting a PCT64 HC (left) or HC+LC (right) (precursor frequency of 10/million B cells). D. PCT64-like aa mutations in PCT64 LMCA HCs isolated at 42 dpi. Numbers inside each square indicate how many sequences that share the total aa mutations (x-axis) and the PCT64-like aa mutations (y-axis). E. Frequencies of observed HC aa mutations per residue at 42 dpi. HCDRs are highlighted in gray. Red=residues present in mature PCT64; blue=residue present in early PCT64 isolates. F. Frequency of selected PCT64LMCA B cell HC aa mutations acquired at 42 dpi in HCDR1 position 28, 31, 35; in HCDR2 position 52, 52B, 52C; in HCDR3 position 92, 97, 100D, 100E. Red=residues present in mature PCT64; blue=on-track mutation present in early PCT64 isolates; black=original LMCA aa; grey=all other mutations. G. Neutralization assay of selected PCT64LMCA 42 dpi mAbs. The five highest affinity mAbs elicited by ApexGT2 or GT5 were evaluated, in addition to PCT64 lineage members and other V2 apex bnAbs. GT5-V2B, BG505.ApexGT5 PSV with loop V2B reverted to BG505 WT; GT5-N167, BG505.ApexGT5 PSV with N167D (BG505 WT has D167); GT5—NR, BG505.ApexGT5 PSV with N167D and R169K (BG505 WT has D167 and K 169). Numbers indicate the percentage of neutralization at 10 μg/mL. H. Cryo-EM structure of ApexGT5 in complex with GT5-d42.16 Fab overlayed with the structure of ApexGT2 in complex with GT2-d42.16 Fab. Sites of SHM are designated in magenta and yellow, respectively. See also FIG. 33. I. Close up of the HCDR1 and 2 domains of both Fabs and their interactions with the N156gp120A glycan and gp120 residues. J. Low pass filtered ApexGT5+GT5-d42.16 cryo-EM map (gray) and (ApexGT2+GT2-d42.16)-(ApexGT5+GT5-d42.16) difference map (purple) showing the density associated with the N187 glycan and the slight shift in angle of approach of the GT2-d42.16 Fab (arrow). K. Close up of ApexGT5 loop2B on protomer C showing the cryo-EM map density (transparent gray) that bridges W188 at the tip of the loop and the N160gp120C glycan along with multiple loop models generated from multi-model refinement. L. All three gp120 protomers of ApexGT5 aligned to one another showing the unique conformation adopted by the protomer C loop.

FIG. 26. GT5 mRNA effectively activates rare precursors. A. Schematic of intramuscular (IM) immunization study. Mice received either GT5 trimers adjuvanted with SIGMA or GT5 mRNA. B. Representative FACS plots of lymph node (LN) B cells at 13, 28, and 42 dpi IM with GT5 trimers and SIGMA adjuvant showing GCs, CD45.2+ B cells in GC, and GT5-binding CD45.2 B cells. C. Quantification of GCs, CD45.2+ B cells in GC, and GT5-binding CD45.2 B cells at 13, 28, and 42 dpi IM with GT5 trimers and SIGMA adjuvant. D. ELISA quantification of GT5-binding (left) and GT5-KO-binding (right) serum IgG from PCT64 LMCA recipient mice immunized with GT5 trimers and SIGMA adjuvant (as in B-C). E. Representative flow cytometry plots of LN B cells at 13, 28, and 42 dpi IM with GT5 mRNA. F. Quantification of LN GCs, CD45.2+ B cells in GC, and GT5-binding CD45.2 B cells at 13, 28, and 42 dpi IM with GT5 mRNA. G. ELISA quantification of GT5-binding (left) and GT5-KO-binding (right) serum IgG from PCT64LMCA recipient mice immunized with GT5 mRNA (as in E-F). H. Representative flow cytometry plots of LN B cells at 13 and 42 dpi IM with GT5 trimers in mice with a starting PCT64 precursor frequency of 10 and 1 per 106 B cells. Plots show the frequency of CD45.2+ B cells in GC. I. Quantification of GCs, CD45.2+ B cells in GC, and GT5-binding CD45.2 B cells at 13 and 42 dpi IM with GT5 trimers in mice with a starting PCT64 precursor frequency of 10 and 1 per 106 B cells. J. Number of total nt and aa mutations acquired in PCT64LMCA HC and LCV genes at 42 dpi with GT5 mRNA. K. Frequencies of observed HC AA mutations per residue at 42 dpi. HCDRs are highlighted in gray. Red=residues present in mature PCT64; blue=on-track mutation present in early PCT64-lineage isolates.

FIG. 27. (SEQ ID NO:228-230) GT5-mRNA immunization activates a J-region-reverted PCT64 germline. A. Sequence alignment of the HCDR3 of PCT64 LMCA.JREV, PCT64 LMCA and germline VH3-15, DH 3-3 and JH6. B. Human PCT64 LMCA.JREVHC (teal), murine HC (dark grey), human PCT64 LMCA.JREV LC (purple) and murine LC (light grey) sequences amplified from single cell sorted B220+ naive B cells from two PCT64 LMCA.JREV mice. Central number=sequence pairs amplified. See also FIG. 34A-B. C. SPR affinity measurement against GT5 of LMCA and LMCA.JREV IGH paired with various murine LC. D. FACS plots with epitope specific GT2-binding (top) and GT5 binding of peripheral B cells in naïve PCT64 LMCA.JREV mouse model. Events were pre-gated on lymphocytes/singlets/CD4-CD8-F4/80-Gr1-/B220+B cells. See also FIG. 34C-F. E. Schematic of immunization study design. Recipient mice received 100, 20 or 10 PCT64 LMCA.JREV per 106 B cells prior to immunization IP with GT5 trimers or IM with GT5 mRNA, responses were analyzed 13 dpi. See also FIG. 34G-H. F. Representative FACS plots of GCs, CD45.2 PCT64LMCA.JREV present in GC, and GT5 specific responses at 13 dpi after immunization with GT5 protein IP (pink) or GT5 mRNA IM (teal). Spleen were analyzed for IP responses and inguinal LN for IM responses. G. Quantification of responses in GCsasin E-F. H. Quantification of GC responses, frequency of CD45.2 LMCA.JREV B cells in GC and GT5 specific responses at 42 dpi.

FIG. 28. Generation of a human Ig least mutated common ancestor (LMCA) PCT64 knock-in mouse, related to FIG. 21. A. Flow cytometry gating strategy for sorting and sequencing single naïve B cell from knock-in mice generated with CRISPR-Cas9. B. Pie chart showing frequency of amplified human PCT64 LMCA heavy chain (green) and murine heavy chain (grey), from single cell BCR sequencing of naïve B cells in PCT64^{LMCA} IGH mice (PCT64^{LMCA-H}) (n=49). C. Pie chart shows frequency of amplified human PCT64 LMCA light chain (purple) and murine heavy chain (grey), from single cell BCR sequencing of naïve B cells in PCT64 LMCA IGK mice (PCT64^{LMCA-L}) (n=22). D. PCT64^{LMCA-H} paired murine light chains V genes (n=26). E. PCT64^{LMCA-L} paired murine heavy chains V genes (n=11). F. Breeding schematic for PCT64^{LMCA-H} and PCT64^{LMCA-L} mouse line. Squares represent males and circles represent females. Upper halves indicate IGK and lower halves IGH, as per key at left. G. Transmission frequency of IGH (H) and IGK (K) to the progeny.

FIG. 29. (SEQ ID NO:231) B lymphocyte development in PCT64^{LMCA-H} and PCT64LMCA-HL mice, related to FIGS. 21 and 22. A. Representative FACS plots of bone marrow progenitor cells isolated from PCT64^{LMCA-H}, PCT64^{LMCA} and WT mice and gating strategy applied for the quantification of early (A, B and C) and late (D, E and F) subfraction of B cell developmental stages accordingly to (Hardy et al. 1991). B. Quantification of early (A, B and C) and late (D, E and F) subfraction of B cell developmental stages in bone marrow from PCT64^{LMCA}-H (green), PCT64^{LMCA} (purple) and wild type (WT) (grey) mice (as in A). C. Representative FACS plots of splenocytes isolated from PCT64^{LMCA-H}, PCT64^{LMCA} and WT mice and gating strategy applied for the quantification of splenic B cells and their differentiation into Follicular, Transitional (T0, T1, T2) and Marginal Zone B cells. D. Quantification of splenic B cells and their differentiation into Follicular, Transitional (T0, T1, T2) and Marginal Zone B cells in spleens from PCT64^{LMCA-H} (green), PCT64^{LMCA} (purple) and Wild Type (grey) mice (as in C). E. Quantification of B cell responses in spleen at 8 days after immunization. CD45.1 WT mice received either 500,000 PCT64^{LMCA} B cells and were immunized with MD39-SOSIP trimers or received 500,000 wild-type CD45.2 B cells and were immunized with GT2 trimers. F. Analysis of the activation-induced deaminase (AID) enzyme binding sites in PCT64^{LMCA} heavy chain. AA position of enriched on-track mutations are highlighted (31, 35, 52b, 100d).

FIG. 30. Cryo-EM data processing workflow for ApexGT2 in complex with GT2-d42.16 Fab, related to FIG. 22. A. Raw EM micrograph and 2-D class averages of selected particles. B. 3-D data processing workflow. C. Fourier shell correlation and angular distribution plots. D. Local resolution estimates. E. Mask used for refinement and sharpening. F. Map segmentation. G. Isolated cryo-EM map density within a 3 Å radius around Fab residues. H. Close up of the epitope/paratope region of ApexGT2+G2-d4.16 showing hydrogen-bonding interactions with gp120 amino acid residues. I. Close up of the epitope/paratope region of ApexGT2+GT2-d42.16 showing hydrogen-bonding interactions with ApexGT2 glycans.

FIG. 31. Light chain repertoire characterization in PCT64LMCA-H mice, related to FIG. 23. A. Bubble graph representing frequency and diversity of murine IGK V genes paired with PCT64 IGH in a naïve PCT64^{LMCA-H} mouse. Bubble size represent relative frequency, colors indicate different V gene. Below, detailed legend of all the isolated IGK V genes and relative frequency. B. Bubble graph representing frequency and diversity of murine IGK V genes paired with murine IGH in a naïve PCT64^{LMCA-H} mouse. Bubble size represent relative frequency, colors indicate different V gene. Below, detailed legend of all the isolated IGK V genes and relative frequency.

FIG. 32. GT2 immunization activates PCT64 heavy chain with different light chains and GC competition after GT2 and GT5 immunization, related to FIGS. 23, 24 and 25. A. FACS quantification of germinal center (GC) responses, CD45.2 cells inside GC, and GT2 binding B cells at 8, 16 and 42 days after immunization with GT2. Recipient mice received 500,000 CD45.2+ PCT64^{LMCA-H} B cells and responses were analyzed in the spleen. B. Phylogenetic clonal lineage trees showing diversification of the PCT64^{LMCA} IGH from day 8 to day 42 after immunization. Branch length is representative of sequence distance. C. Number of total nucleotide and amino acid mutations acquired in PCT64^{LMCA-H} heavy chain V genes (IGHV) at 8 (n=114) and 42 (n=106) DPI. D. Frequencies of heavy chain AA mutations observed per residue at day 8 post-immunization in PCT64^{LMCA} heavy chain. HCDRs are highlighted in gray. E. Distribution of selected PCT64^{LMCA} B cell heavy chain aa mutations in positions 31, 35, 52b and 100d at 8 dpi. Mutations present in mature PCT64 are in red, mutations present in PCT64-lineage early isolates are in blue, original LMCA amino acids are in black, other mutations are in grey. F. Representative FACS plots of antigen-specific germinal center responses after immunization with GT2 (green) or GT5 (purple) (10 ug, adjuvanted with Sigma) in mice with 100, 20 and 10 per 106 PCT64^{LMCA} precursor B cells. G. Quantification of responses in F. H. FACS gating strategy for sorting of class switched antigen specific cells at 42 dpi.

FIG. 33. Cryo-EM data processing workflow for ApexGT5 in complex with GT5-d42.16 Fab, related to FIG. 25. A. Raw EM micrograph and 2-D class averages of selected particles. B. 3-D data processing workflow. C. Global Fourier shell correlation and angular distribution plots. D. Local resolution estimates. E. M ask used for refinement and sharpening. F. Map segmentation. G. Isolated cryo-EM map density within a 3 Å radius around Fab residues. H. Close up of the epitope/paratope region of ApexGT5+GT5-d4.16 showing hydrogen bonding interactions with gp120 amino acid residues. I. Close up of the epitope/paratope region of ApexGT5+GT5-d4.16 showing hydrogen bonding interactions with ApexGT2 glycans. J. Structures of ApexGT2.2M UT+PCT64.LMCA, ApexGT2+GT2-d42.16, and ApexGT5+GT5-d42.16 aligned and overlayed viewed from 3 different angles. K. Close up of the HCDR3 domains and N160gp120A and B glycans. L. N156gp120A glycan. M. And loop2B of protomer C along with N160gp120A glycan.

FIG. 34. (SEQ ID NO:232-234) Generation of a PCT64 LMCA.JREV mouse and immunization strategies, related to FIG. 27. A. Breeding and transmission of the knock-in IGH PCT64^{LMCA.JREV} to the progeny. B. Pie chart showing frequency of amplified human PCT64^{LMCA.JREV} heavy chain (teal) and murine heavy chain (grey), from single cell BCR sequencing of naïve B cells in a PCT64LMCA.JREV IGH (PCT64^{LMCA.JREV-H}) (n=20) mouse (M 10). C. Schematic of immunization study design. Recipient mice received 100 PCT64^{LMCA.JREV} per106 B cells and were immunized IP with either GT2 or GT5 trimers, responses were analyzed 8 dpi. D. FACS plots of germinal centers (GC) responses at 8 dpi after immunization with GT2 or GT5 (left). Quantification of CD45.2 responses in GC (right). E. Localization of sites of mutation enrichment in the IGH of CD45.2 B cell isolated 42 days after GT5 protein immunization. F. HCDR3 alignment of PCT64 LMCA, LMCA.JREV and consensus of mutations acquired 42 days after GT5 immunization. G. FACS analysis of GC responses 13 days after IM immunization with either GT5 mRNA or GT5 soluble protein in PCT64^{LMCA.JREV} recipient mice, quantification is on the right. H. Localization of sites of mutation enrichment in the IGH of CD45.2 B cell isolated 42 days after GT5 mRNA immunization (left). Pie chart with frequency of mutation Y 117D (100% of 50 isolated IGH) (right).

FIG. 35. Cryo-EM data and model refinement statistics.

FIG. 36. BG505-T332N autologous neutralization measured in a TZM-bl assay, for purified serum IgG from week 26 from rabbits immunized at weeks 0, 8, 24 with the indicated constructs in rabbit experiment #1.

FIG. 37. BG505-T332N autologous neutralization measured in a TZM-bl assay, for purified serum IgG from week 10 (left) or week 26 (right) from rabbits immunized at weeks 0, 8, 24 with the indicated constructs in rabbit experiment #2.

FIG. 38. ELISA analysis of serum antibody binding responses from week 10 (post two vaccinations) for rabbit experiments 1 and 2 combined. A: Response to MD39; B: Response to MD39 in presence of base-directed antibody 19R, to reveal responses to epitopes other than the base; C: Fraction of response to MD39 that is directed to the base. Immunogens were mRNA unless indicated otherwise. Boxes indicate data for matched pairs of mRNA-delivered soluble and membrane-bound trimers.

FIG. 39. ELISA analysis of anti-V3 serum antibody binding responses from week 10 (post two vaccinations) for rabbit experiments 1 and 2 combined. A: Response to V3 peptide; B: Response to MD39 in presence of base-directed IgG 19R, to reveal responses to epitopes other than the base; C: Ratio of V3 response to MD39 response other than base. Immunogens were mRNA unless indicated otherwise.

FIG. 40. Cell surface expression and antigenic profile for gp160-dCT and gp151

FIG. 41. Relative expression levels and antigenicity for gp151 constructs assessed by Gag-VLP ELISA. A: Relative Env expression levels as measured by PGT121 reactivity. B: Antigenic profiles normalized by PGT121 reactivity.

FIG. 42. BG505-T332N autologous neutralization measured in a TZM-bl assay, for purified serum IgG from week 10 (left) or week 26 (middle and right, with week 26 repeat at right) from rhesus macaques immunized at weeks 0, 8, 24 with the constructs shown in Table 4.

FIG. 43. ELISA analysis of NHP serum antibody binding responses from week 26 (post three vaccinations). A: MD39 AUC, the response to MD39. B: Delta AUC, the response to MD39 minus the response to MD39 in presence of base-directed antibody 19R. This reveals responses to the base. C: Fraction of response to MD39 that is directed to the base. D: Fraction of the MD39 response that is directed to epitopes other than the base.

### DETAILED DESCRIPTION OF THE INVENTION

The invention relates to improved HIV antigens, including germline-targeting designs, trimer stabilization designs, combinations of those two, trimers designed with modified surfaces helpful for immunization regimens, other types of trimer modifications (see, for example, examples of trimers with combined germline-targeting mutations and stabilization mutations and additional trimer modifications that add functionality and that can be combined with other types of modifications as described herein) and on development of trimer nanoparticles and membrane-bound trimers. The invention also encompasses combinations of any of the herein described modifications, such as but not limited to, combinations of stabilization and modified surfaces with nanoparticles or membrane-bound trimers.

The HIV envelope protein trimer is the target of broadly neutralizing antibodies (bNAbs). The high mannose patch, including the N332-linked glycan at the base of the V3 loop of gp120, is frequently targeted by bnAbs during natural infection and hence is an appealing vaccine epitope. Germline targeting has potential to initiate the elicitation of N332-dependent bnAbs by vaccination, but no immunogen has been reported to bind germline-reverted precursors of N332-dependent bnAbs.

VRC01-class antibodies are defined as those with a VH1-2 gene in the heavy chain and a five amino acid CDR3 in the light chain. The VH1-2 mouse employed here was originally developed by Ming Tian in the Fred Alt lab at Harvard and was first reported in Tian et al. Cell 2016. It is a stringent model system for inducing VRC01-class responses, in which Applicants have measured a VRC01-class precursor frequency of approximately 1 in 1 million naive B cells, which is similar to the frequency measured in humans as reported in Jardine et al. Science 2016 and Havenar-Daughton et al Science Translational Medicine 2018. eOD-GT8 60mer and derivatives are the only immunogens reported to be capable of priming VRC01-class responses in this model (Tian et al. Cell 2016; Duan et al Immunity 2018).

Some sequences contain a sequence leader (MGILPSPGMPALLSLVSLLSVLLMGCVAETG) (SEQ ID NO: 1) are cleaved during expression/secretion and is not present in the final expressed protein product. The embodiments contained herein are not limited to this particular leader sequence as different leader sequences could be used to serve the same purpose.

The invention also encompasses a protein having at least 90% homology or identity with the sequence of the protein of any one of the trimers disclosed herein. The invention also encompasses a protein having at least 95% homology or identity with the sequence of the protein of any one of trimers disclosed herein.

The invention also encompasses any nucleic acid encoding the protein of any one of the immunogens disclosed herein. The invention also encompasses a nucleic acid having at least 90% or 95% homology or identity with the sequence of said nucleic acid.

The invention also encompasses eliciting an immune response which may comprise systemically administering to an animal in need thereof an effective amount of any one of the non-naturally occurring protein(s) or any one of the nucleic acids encoding the non-naturally occurring protein(s) of the present invention, including nucleic acids that may have at least 90% or 95% homology or identity with a nucleotide encoding the sequence of the non-naturally occurring protein(s) of the invention. In one embodiment, the nucleic acid may be a RNA, advantageously a mRNA. Advantageously, the nucleic acid is formulated in lipid nanoparticles (LNPs). The animal may be a mammal, advantageously a human.

The invention pertains to the identification, design, synthesis and isolation of mutant trimers disclosed herein as well as nucleic acids encoding the same. The present invention also relates to homologues, derivatives and variants of the sequences of the mutant trimers and nucleic acids encoding the same, wherein it is preferred that the homologue, derivative or variant have at least 50%, at least 60%, at least 70%, at least 75%, at least 80%, at least 85%, at least 90%, at least 93%, at least 95%, at least 97%, at least 98% or at least 99% homology or identity with the sequence of the mutant trimers and nucleic acids encoding the same. It is noted that within this specification, homology to sequences of the mutant proteins and nucleic acids encoding the same refers to the homology of the homologue, derivative or variant to the binding site of the mutant proteins and nucleic acids encoding the same.

The invention still further relates to nucleic acid sequences expressing the mutant immunogens disclosed herein, or homologues, variants or derivatives thereof. One of skill in the art will know, recognize and understand techniques used to create such. Additionally, one of skill in the art will be able to incorporate such a nucleic acid sequence into an appropriate vector, allowing for production of the amino acid sequence of mutant proteins and nucleic acids encoding the same or a homologue, variant or derivative thereof.

Where used herein and unless specifically indicated otherwise, the following terms are intended to have the following meanings in addition to any broader (or narrower) meanings the terms might enjoy in the art:

The term “isolated” or “non-naturally occurring” is used herein to indicate that the isolated moiety (e.g. peptide or compound) exists in a physical milieu distinct from that in which it occurs in nature. For example, the isolated peptide may be substantially isolated with respect to the complex cellular milieu in which it naturally occurs. The absolute level of purity is not critical, and those skilled in the art may readily determine appropriate levels of purity according to the use to which the peptide is to be put. The term “isolating” when used a step in a process is to be interpreted accordingly.

In many circumstances, the isolated moiety will form part of a composition (for example a more or less crude extract containing many other molecules and substances), buffer system, matrix or excipient, which may for example contain other components (including proteins, such as albumin).

In other circumstances, the isolated moiety may be purified to essential homogeneity, for example as determined by PAGE or column chromatography (for example HPLC or mass spectrometry). In preferred embodiments, the isolated peptide or nucleic acid of the invention is essentially the sole peptide or nucleic acid in a given composition.

In an advantageous embodiment, a tag may be utilized for purification or biotinylation. The tag for purification may be a his tag. In another embodiment, the tag for biotinylation may be an avi-tag. Other tags are contemplated for purification, however, purification may be accomplished without a tag. In another embodiment, antibody (such as, not limited to, a broadly neutralizing antibody) affinity columns are contemplated. In another embodiment, lectin columns are contemplated.

Native-like soluble trimers can be made by several methods that all involve stabilizing associations between envelope protein subunits. See, e.g., Steichen et al., Immunity. 2016 Sep. 20; 45 (3): 483-496. doi: 10.1016/j.immuni.2016.08.016. Epub 2016 Sep. 8.PMID: 27617678, Kulp et al., Nat Commun. 2017 Nov. 21; 8 (1): 1655. doi: 10.1038/s41467-017-01549-6.PMID: 29162799 and R. W. Sanders et al., “HIV-1 neutralizing antibodies induced by native-like envelope trimers,” Science, doi: 10.1126/science.aac4223, 2015.

The proteins and compounds of the invention need not be isolated in the sense defined above, however.

The term “pharmaceutical composition” is used herein to define a solid or liquid composition in a form, concentration and level of purity suitable for administration to a patient (e.g. a human patient) upon which administration it may elicit the desired physiological changes. The terms “immunogenic composition” and “immunological composition” and “immunogenic or immunological composition” cover any composition that elicits an immune response against the targeted pathogen, HIV. Terms such as “vaccinal composition” and “vaccine” and “vaccine composition” cover any composition that induces a protective immune response against the targeted pathogen or which efficaciously protects against the pathogen; for instance, after administration or injection, elicits a protective immune response against the targeted pathogen or provides efficacious protection against the pathogen. Accordingly, an immunogenic or immunological composition induces an immune response, which may, but need not, be a protective immune response. A n immunogenic or immunological composition may be used in the treatment of individuals infected with the pathogen, e.g., to stimulate an immune response against the pathogen, such as by stimulating antibodies against the pathogen. Thus, an immunogenic or immunological composition may be a pharmaceutical composition. Furthermore, when the text speaks of “immunogen, antigen or epitope”, an immunogen may be an antigen or an epitope of an antigen. A diagnostic composition is a composition containing a compound or antibody, e.g., a labeled compound or antibody, that is used for detecting the presence in a sample, such as a biological sample, e.g., blood, semen, vaginal fluid, etc., of an antibody that binds to the compound or an immunogen, antigen or epitope that binds to the antibody; for instance, an anti-HIV antibody or an HIV immunogen, antigen or epitope.

A “conservative amino acid change” is one in which the amino acid residue is replaced with an amino acid residue having a similar side chain. Families of amino acid residues having similar side chains have been defined in the art. These families include amino acids with basic side chains (e.g. lysine, arginine and histidine), acidic side chains (e.g. aspartic acid and glutamic acid), non-charged amino acids or polar side chains (e.g. glycine, asparagine, glutamine, serine, threonine, tyrosine and cysteine), non-polar side chains (e.g. alanine, valine, leucine, isoleucine, proline, phenylalanine, methionine and tryptophan), beta-branched side chains (e.g. threonine, valine and isoleucine), and aromatic side chains (e.g. tyrosine, phenylalanine, tryptophan and histidine).

The terms “protein”, “peptide”, “polypeptide”, and “amino acid sequence” are used interchangeably herein to refer to polymers of amino acid residues of any length. The polymer may be linear or branched, it may comprise modified amino acids or amino acid analogs, and it may be interrupted by chemical moieties other than amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling or bioactive component.

As used herein, the terms “antigen” or “immunogen” are used interchangeably to refer to a substance, typically a protein, which is capable of inducing an immune response in a subject. The term also refers to proteins that are immunologically active in the sense that once administered to a subject (either directly or by administering to the subject a nucleotide sequence or vector that encodes the protein) is able to evoke an immune response of the humoral and/or cellular type directed against that protein.

The term “antibody” includes intact molecules as well as fragments thereof, such as Fab, F(ab′)₂, Fv and scFv which are capable of binding the epitope determinant. These antibody fragments retain some ability to selectively bind with its antigen or receptor and include, for example:
(a) Fab, the fragment which contains a monovalent antigen-binding fragment of an antibody molecule may be produced by digestion of whole antibody with the enzyme papain to yield an intact light chain and a portion of one heavy chain;
(b) Fab′, the fragment of an antibody molecule may be obtained by treating whole antibody with pepsin, followed by reduction, to yield an intact light chain and a portion of the heavy chain; two Fab′ fragments are obtained per antibody molecule;
(c) F (ab′)₂, the fragment of the antibody that may be obtained by treating whole antibody with the enzyme pepsin without subsequent reduction; F (ab′)₂ is a dimer of two Fab′ fragments held together by two disulfide bonds;
(d) scFv, including a genetically engineered fragment containing the variable region of a heavy and a light chain as a fused single chain molecule.

General methods of making these fragments are known in the art. (See for example, Harlow and Lane, Antibodies: A Laboratory Manual, Cold Spring Harbor Laboratory, New York (1988), which is incorporated herein by reference). Fabs, Fv and scFV may also be made recombinantly, i.e. expressed as Fab, Fv or scFV rather than cleaving an intact IgG.

A “neutralizing antibody” may inhibit the entry of HIV-1 virus for example SF162 and/or JR-CSF with a neutralization index>1.5 or >2.0. Broad and potent neutralizing antibodies may neutralize greater than about 50% of HIV-1 viruses (from diverse clades and different strains within a clade) in a neutralization assay. The inhibitory concentration of the monoclonal antibody may be less than about 25 mg/ml to neutralize about 50% of the input virus in the neutralization assay.

An “isolated antibody” or “non-naturally occurring antibody” is one that has been separated and/or recovered from a component of its natural environment. Contaminant components of its natural environment are materials that would interfere with diagnostic or therapeutic uses for the antibody, and may include enzymes, hormones, and other proteinaceous or nonproteinaceous solutes. In preferred embodiments, the antibody is purified: (1) to greater than 95% by weight of antibody as determined by the Lowry method, and most preferably more than 99% by weight; (2) to a degree sufficient to obtain at least 15 residues of N-terminal or internal amino acid sequence by use of a spinning cup sequenator; or (3) to homogeneity by SDS-PAGE under reducing or non-reducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

The term “monoclonal antibody” as used herein refers to an antibody obtained from a population of substantially homogeneous antibodies, i.e., the individual antibodies which may comprise the population are identical except for possible naturally occurring mutations that may be present in minor amounts. Monoclonal antibodies are highly specific, being directed against a single antigenic site. Furthermore, in contrast to polyclonal antibody preparations that include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” is not to be construed as requiring production of the antibody by any particular method. For example, the monoclonal antibodies useful in the present invention may be prepared by the hybridoma methodology first described by Kohler et al., Nature, 256:495 (1975), or may be made using recombinant DNA methods in bacterial, eukaryotic animal or plant cells (see, e.g., U.S. Pat. No. 4,816,567). The “monoclonal antibodies” may also be isolated from phage antibody libraries using the techniques described in Clackson et al., Nature, 352: 624-628 (1991) and Marks et al., J. Mol. Biol., 222: 581-597 (1991), for example.

An “antibody fragment” may comprise a portion of an intact antibody, preferably the antigen binding or variable region of the intact antibody. Examples of antibody fragments include Fab, Fab′, F(ab′)₂, scFV and Fv fragments; diabodies; linear antibodies (see U.S. Pat. No. 5,641,870; Zapata et al., Protein Eng. 8 (10): 1057-1062 [1995]); single-chain antibody molecules; and multispecific antibodies formed from antibody fragments.

It should be understood that the proteins of the invention may differ from the exact sequences illustrated and described herein. Thus, the invention contemplates deletions, additions and substitutions to the sequences shown, so long as the sequences function in accordance with the methods of the invention. In this regard, particularly preferred substitutions will generally be conservative in nature, i.e., those substitutions that take place within a family of amino acids. For example, amino acids are generally divided into four families: (1) acidic-aspartate and glutamate; (2) basic-lysine, arginine, histidine; (3) non-polar-alanine, valine, leucine, isoleucine, proline, phenylalanine, methionine, tryptophan; and (4) uncharged polar—glycine, asparagine, glutamine, cysteine, serine threonine, tyrosine. Phenylalanine, tryptophan, and tyrosine are sometimes classified as aromatic amino acids. It is reasonably predictable that an isolated or non-naturally occurring replacement of leucine with isoleucine or valine, or vice versa; an aspartate with a glutamate or vice versa; a threonine with a serine or vice versa; or a similar conservative replacement of an amino acid with a structurally related amino acid, will not have a major effect on the biological activity. Proteins having substantially the same amino acid sequence as the sequences illustrated and described but possessing minor amino acid substitutions that do not substantially affect the immunogenicity of the protein are, therefore, within the scope of the invention.

As used herein the terms “nucleotide sequences” and “nucleic acid sequences” refer to deoxyribonucleic acid (DNA) or ribonucleic acid (RNA) sequences, including, without limitation, messenger RNA (mRNA), DNA/RNA hybrids, or synthetic nucleic acids. The nucleic acid may be single-stranded, or partially or completely double-stranded (duplex). Duplex nucleic acids may be homoduplex or heteroduplex.

As used herein the term “transgene” may used to refer to “recombinant” nucleotide sequences that may be derived from any of the nucleotide sequences encoding the proteins of the present invention. The term “recombinant” means a nucleotide sequence that has been manipulated “by man” and which does not occur in nature, or is linked to another nucleotide sequence or found in a different arrangement in nature. It is understood that manipulated “by man” means manipulated by some artificial means, including by use of machines, codon optimization, restriction enzymes, etc.

For example, in one embodiment the nucleotide sequences may be mutated such that the activity of the encoded proteins in vivo is abrogated. In another embodiment the nucleotide sequences may be codon optimized, for example the codons may be optimized for human use. In preferred embodiments the nucleotide sequences of the invention are both mutated to abrogate the normal in vivo function of the encoded proteins, and codon optimized for human use. For example, each of the sequences of the invention, such as the mutant trimers, may be altered in these ways.

As regards codon optimization, the nucleic acid molecules of the invention have a nucleotide sequence that encodes the antigens of the invention and may be designed to employ codons that are used in the genes of the subject in which the antigen is to be produced. Many viruses, including HIV and other lentiviruses, use a large number of rare codons and, by altering these codons to correspond to codons commonly used in the desired subject, enhanced expression of the antigens may be achieved. In a preferred embodiment, the codons used are “humanized” codons, i.e., the codons are those that appear frequently in highly expressed human genes (Andre et al., J. Virol. 72:1497-1503, 1998) instead of those codons that are frequently used by HIV. Such codon usage provides for efficient expression of the transgenic HIV proteins in human cells. A ny suitable method of codon optimization may be used. Such methods, and the selection of such methods, are well known to those of skill in the art. In addition, there are several companies that will optimize codons of sequences, such as Geneart (geneart.com). Thus, the nucleotide sequences of the invention may readily be codon optimized.

The invention further encompasses nucleotide sequences encoding functionally and/or antigenically equivalent variants and derivatives of the antigens of the invention and functionally equivalent fragments thereof. These functionally equivalent variants, derivatives, and fragments display the ability to retain antigenic activity. For instance, changes in a DNA sequence that do not change the encoded amino acid sequence, as well as those that result in conservative substitutions of amino acid residues, one or a few amino acid deletions or additions, and substitution of amino acid residues by amino acid analogs are those which will not significantly affect properties of the encoded polypeptide. Conservative amino acid substitutions are glycine/alanine; valine/isoleucine/leucine; asparagine/glutamine; aspartic acid/glutamic acid; serine/threonine/methionine; lysine/arginine; and phenylalanine/tyrosine/tryptophan. In one embodiment, the variants have at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% homology or identity to the antigen, epitope, immunogen, peptide or polypeptide of interest.

For the purposes of the present invention, sequence identity or homology is determined by comparing the sequences when aligned so as to maximize overlap and identity while minimizing sequence gaps. In particular, sequence identity may be determined using any of a number of mathematical algorithms. A nonlimiting example of a mathematical algorithm used for comparison of two sequences is the algorithm of Karlin & Altschul, Proc. Natl. Acad. Sci. USA 1990; 87:2264-2268, modified as in Karlin & Altschul, Proc. Natl. Acad. Sci. USA 1993; 90:5873-5877.

Another example of a mathematical algorithm used for comparison of sequences is the algorithm of Myers & Miller, CABIOS 1988; 4:11-17. Such an algorithm is incorporated into the ALIGN program (version 2.0) which is part of the GCG sequence alignment software package. When utilizing the ALIGN program for comparing amino acid sequences, a PAM 120 weight residue table, a gap length penalty of 12, and a gap penalty of 4 may be used. Y et another useful algorithm for identifying regions of local sequence similarity and alignment is the FASTA algorithm as described in Pearson & Lipman, Proc. Natl. Acad. Sci. USA 1988; 85:2444-2448.

Advantageous for use according to the present invention is the WU-BLAST (Washington University BLAST) version 2.0 software. WU-BLAST version 2.0 executable programs for several UNIX platforms may be downloaded from ftp://blast.wustl.edu/blast/executables. This program is based on WU-BLAST version 1.4, which in turn is based on the public domain NCBI-BLAST version 1.4 (Altschul & Gish, 1996, Local alignment statistics, Doolittle ed., Methods in Enzymology 266: 460-480; Altschul et al., Journal of Molecular Biology 1990; 215:403-410; Gish & States, 1993; Nature Genetics 3:266-272; Karlin & Altschul, 1993; Proc. Natl. Acad. Sci. USA 90:5873-5877; all of which are incorporated by reference herein).

The various recombinant nucleotide sequences and proteins of the invention are made using standard recombinant DNA and cloning techniques. Such techniques are well known to those of skill in the art. See for example, “Molecular Cloning: A Laboratory Manual”, second edition (Sambrook et al. 1989).

The nucleotide sequences of the present invention may be inserted into “vectors.” The term “vector” is widely used and understood by those of skill in the art, and as used herein the term “vector” is used consistent with its meaning to those of skill in the art. For example, the term “vector” is commonly used by those skilled in the art to refer to a vehicle that allows or facilitates the transfer of nucleic acid molecules from one environment to another or that allows or facilitates the manipulation of a nucleic acid molecule.

Any vector that allows expression of the proteins of the present invention may be used in accordance with the present invention. In certain embodiments, the proteins of the present invention may be used in vitro (such as using cell-free expression systems) and/or in cultured cells grown in vitro in order to produce the encoded HIV-proteins, which may then be used for various applications such as in the production of proteinaceous vaccines. For such applications, any vector that allows expression of the proteins in vitro and/or in cultured cells may be used.

For applications where it is desired that the proteins be expressed in vivo, for example when the transgenes of the invention are used in DNA or DNA-containing vaccines, any vector that allows for the expression of the proteins of the present invention and is safe for use in vivo may be used. In preferred embodiments the vectors used are safe for use in humans, mammals and/or laboratory animals.

For the proteins of the present invention to be expressed, the protein coding sequence should be “operably linked” to regulatory or nucleic acid control sequences that direct transcription and translation of the protein. As used herein, a coding sequence and a nucleic acid control sequence or promoter are said to be “operably linked” when they are covalently linked in such a way as to place the expression or transcription and/or translation of the coding sequence under the influence or control of the nucleic acid control sequence. The “nucleic acid control sequence” may be any nucleic acid element, such as, but not limited to promoters, enhancers, IRES, introns, and other elements described herein that direct the expression of a nucleic acid sequence or coding sequence that is operably linked thereto. The term “promoter” will be used herein to refer to a group of transcriptional control modules that are clustered around the initiation site for RNA polymerase II and that when operationally linked to the protein coding sequences of the invention lead to the expression of the encoded protein. The expression of the transgenes of the present invention may be under the control of a constitutive promoter or of an inducible promoter, which initiates transcription only when exposed to some particular external stimulus, such as, without limitation, antibiotics such as tetracycline, hormones such as ecdysone, or heavy metals. The promoter may also be specific to a particular cell-type, tissue or organ. Many suitable promoters and enhancers are known in the art, and any such suitable promoter or enhancer may be used for expression of the transgenes of the invention. For example, suitable promoters and/or enhancers may be selected from the Eukaryotic Promoter Database (EPDB).

The vectors used in accordance with the present invention should typically be chosen such that they contain a suitable gene regulatory region, such as a promoter or enhancer, such that the proteins of the invention may be expressed.

Any suitable vector may be used depending on the application. For example, plasmids, viral vectors, bacterial vectors, protozoal vectors, insect vectors, baculovirus expression vectors, yeast vectors, mammalian cell vectors, and the like, may be used. Suitable vectors may be selected by the skilled artisan taking into consideration the characteristics of the vector and the requirements for expressing the proteins under the identified circumstances.

In an advantageous embodiment, IgG1 and Fab expression vectors may be utilized to reconstitute heavy and light chain constant regions if heavy and light chain genes of the proteins of the present invention are cloned.

When the aim is to express the proteins of the invention in vivo in a subject, for example in order to generate an immune response against an HIV-1 antigen and/or protective immunity against HIV-1, expression vectors that are suitable for expression on that subject, and that are safe for use in vivo, should be chosen. For example, in some embodiments it may be desired to express the proteins of the invention in a laboratory animal, such as for pre-clinical testing of the HIV-1 immunogenic compositions and vaccines of the invention. In other embodiments, it will be desirable to express the proteins of the invention in human subjects, such as in clinical trials and for actual clinical use of the immunogenic compositions and vaccine of the invention. A ny vectors that are suitable for such uses may be employed, and it is well within the capabilities of the skilled artisan to select a suitable vector. In some embodiments it may be preferred that the vectors used for these in vivo applications are attenuated to vector from amplifying in the subject. For example, if plasmid vectors are used, preferably they will lack an origin of replication that functions in the subject so as to enhance safety for in vivo use in the subject. If viral vectors are used, preferably they are attenuated or replication-defective in the subject, again, so as to enhance safety for in vivo use in the subject.

In preferred embodiments of the present invention viral vectors are used. Viral expression vectors are well known to those skilled in the art and include, for example, viruses such as adenoviruses, adeno-associated viruses (AAV), alphaviruses, herpesviruses, retroviruses and poxviruses, including avipox viruses, attenuated poxviruses, vaccinia viruses, and particularly, the modified vaccinia Ankara virus (MVA; ATCC Accession No. VR-1566). Such viruses, when used as expression vectors are innately non-pathogenic in the selected subjects such as humans or have been modified to render them non-pathogenic in the selected subjects. For example, replication-defective adenoviruses and alphaviruses are well known and may be used as gene delivery vectors.

The nucleotide sequences and vectors of the invention may be delivered to cells, for example if the aim is to express the HIV-1 antigens in cells in order to produce and isolate the expressed proteins, such as from cells grown in culture. For expressing the proteins in cells any suitable transfection, transformation, or gene delivery methods may be used. Such methods are well known by those skilled in the art, and one of skill in the art would readily be able to select a suitable method depending on the nature of the nucleotide sequences, vectors, and cell types used. For example, transfection, transformation, microinjection, infection, electroporation, lipofection, or liposome-mediated delivery could be used. Expression of the proteins may be carried out in any suitable type of host cells, such as bacterial cells, yeast, insect cells, and mammalian cells. The proteins of the invention may also be expressed using including in vitro transcription/translation systems. All of such methods are well known by those skilled in the art, and one of skill in the art would readily be able to select a suitable method depending on the nature of the nucleotide sequences, vectors, and cell types used.

A synthetic mutant trimer may be chemically synthesized in whole or part using techniques that are well-known in the art (see, e.g., Kochendoerfer, G. G., 2001). Additionally, homologs and derivatives of the polypeptide may be also be synthesized.

Alternatively, methods which are well known to those skilled in the art may be used to construct expression vectors containing nucleic acid molecules that encode the polypeptide or homologs or derivatives thereof under appropriate transcriptional/translational control signals, for expression. These methods include in vitro recombinant DNA techniques, synthetic techniques and in vivo recombination/genetic recombination. See, for example, the techniques described in Maniatis et al., 1989.

The HIV envelope protein (Env) is the target of broadly neutralizing antibodies (bnAbs) in natural infection. Env is a membrane protein composed of a trimer of gp120 and gp41 subunits that contains a high degree of sequence diversity and a surface that is shielded by N-linked glycans. The bnAbs that target Env often have unusual features such as a long complementarity-determining region (CDR) H3, high levels of somatic hypermutation (SHM), and insertions and deletions (INDELS). Furthermore, most of the bnAbs recognize complex epitopes that are typically non-linear and have both protein and glycan components.

Applicants claim sequences of different types of immunogen sequences. The sequences provided below are exemplary examples, the stabilizing mutations, modifications, (such as, but not limited to, cleavage-independent modifications), and/or a membrane anchoring strategy (such as, but not limited to, linker plus platelet-derived growth factor receptor (PDGFR)) described herein are applicable to any HIV strain or clade, such as but not limited to, those described below.

The present invention relates to non-naturally occurring proteins, which may be involved in forming immunogenic proteins of the present invention.

The invention relates to a non-naturally occurring protein which may comprise any one of the following sequences in Table 1.

TABLE 1
immunogenic proteins

ApexGT5.2_link14
AENLWVTVYYGVPVWKDAETTLFCASDAKAYETEKHNVWATHACVPTDPN
PQEIHLENVTEEFNMWKNNMVEQMHEDIISLWDQSLKPCVKLTPLCVTLQ
CTNVTNNITDDMRGELKNCSFNATTELRNKROKVYSLFYRLDIVPMVDLW
TNYRLINCNTSAITQACPKVSFEPIPIHYCAPAGFAILKCKDKKFNGTGP
CPSVSTVQCTHGIKPVVSTQLLLNGSLAEEEVIIRSENITNNAKNILVQL
NTPVQINCTRPNNNTVKSIRIGPGQAFYYTGDIIGDIRQAHCNVSKATWN
ETLGKVVKQLRKHFGNNTIIRFAQSSGGDLEVTTHSFNCGGEFFYCNTSG
LFNSTWISNTSVQGSNSTGSNDSITLPCRIKQIINMWQRIGQAMYAPPIQ
GVIRCVSNITGLILTRDGGSTNSTTETFRPGGGDMRDNWRSELYKYKVVK
IEPLGVAPTRCKRRVVGSHSGSGGSGSGGHAAVGIGAVSLGFLGAAGSTM
GAASMTLTVQARNLLSGIVQQQSNLLRAPEPQQHLLKDTHWGIKQLQARV
LAVEHYLRDQQLLGIWGCSGKLICCTNVPWNSSWSNRNLSEIWDNMTWLQ
WDKEISNYTQIIYGLLEESQNQQEKNEQDLLALD** (SEQ ID NO:
2)
ApexGT6.2_link14
AENLWVTVYYGVPVWKDAETTLFCASDAKAYETEKHNVWATHACVSTDPN
PQEIHLENVTEEFNMWKNNMVEQMHEDIISLWDQSLKPCVKLTPLCVGLQ
CTNVTNNITDDMRGELKNCSFNATTELRNKROKVYSLFYRLDIVPMVDLW
TNYRLISCNTSAITQACPKVSFEPIPIHYCAPAGFAILKCKDKKFNGTGP
CPSVSTVQCTHGIKPVVSTQLLLNGSLAEEEVIIRSENITNNAKNILVQL
NTPVQINCTRPNNNTVKSIRIGPGQAFYYTGDIIGDIRQAHCNVSKATWN
ETLGKVVKQLRKHFGNNTIIRFAQSSGGDLEVTTHSFNCGGEFFYCNTSG
LFNSTWISNTSVQGSNSTGSNDSITLPCRIKQIINMWQRIGQAMYAPPIQ
GVIRCVSNITGLILTRDGGSTNSTTETFRPGGGDMRDNWRSELYKYKVVK
IEPLGVAPTRCKRRVVGSHSGSGGSGSGGHAAVGIGAVSLGFLGAAGSTM
GAASMTLTVQARNLLSGIVQQQSNLLRAPEPQQHLLKDTHWGIKQLQARV
LAVEHYLRDQQLLGIWGCSGKLICCTNVPWNSSWSNRNLSEIWDNMTWLQ
WDKEISNYTQIIYGLLEESQNQQEKNEQDLLALD** (SEQ ID NO:
3)
BG505.ApexGT2
AENLWVTVYYGVPVWKDAETTLFCASDAKAYETEKHNVWATHACVPTDPN
PQEIHLENVTEEFNMWKNNMVEQMHEDIISLWDQSLKPCVKLTPLCVTLQ
CTNVTNNITDDMRGELKNCSFNATTELRNKROKVYSLFYRLDIVPMGENS
TNYRLINCNTSAITQACPKVSFEPIPIHYCAPAGFAILKCKDKKFNGTGP
CPSVSTVQCTHGIKPVVSTQLLLNGSLAEEEVIIRSENITNNAKNILVQL
NTPVQINCTRPNNNTVKSIRIGPGQAFYYTGDIIGDIRQAHCNVSKATWN
ETLGKVVKQLRKHFGNNTIIRFAQSSGGDLEVTTHSFNCGGEFFYCNTSG
LFNSTWISNTSVQGSNSTGSNDSITLPCRIKQIINMWQRIGQAMYAPPIQ
GVIRCVSNITGLILTRDGGSTNSTTETFRPGGGDMRDNWRSELYKYKVVK
IEPLGVAPTRCKRRVVGRRRRRRAVGIGAVSLGFLGAAGSTMGAASMTLT
VQARNLLSGIVQQQSNLLRAPEPQQHLLKDTHWGIKQLQARVLAVEHYLR
DQQLLGIWGCSGKLICCTNVPWNSSWSNRNLSEIWDNMTWLQWDKEISNY
TQIIYGLLEESQNQQEKNEQDLLALD (SEQ ID NO: 4)
BG505_ApexGT5.2_congly_CD4KO_gp151_link14
AENLWVTVYYGVPVWKDAETTLFCASDAKAYETEKHNVWATHACVPTDPN
PQEIHLENVTEEFNMWKNNMVEQMHEDIISLWDQSLKPCVKLTPLCVTLQ
CTNVTNNITDDMRGELKNCSFNATTELRNKROKVYSLFYRLDIVPMVDLW
TNYRLINCNTSAITQACPKVSFEPIPIHYCAPAGFAILKCKDKKFNGTGP
CQNVSTVQCTHGIKPVVSTQLLLNGSLAEEEVIIRSENITNNAKNILVQL
NTSVQINCTRPNNNTVKSIRIGPGQAFYYTGDIIGDIRQAHCNVSKATWN
ETLGKVVKQLRKHFGNNTIIRFAQSSGGDLEVTTHSFNCGGEFFYCNTSG
LFNSTWISNTSVQGSNSTGSNDSITLPCRIKQIINMWQRIGQAMYAPPIQ
GVIRCVSNITGLILTRDGGSTNSTTETFRPGGTDMRDNWRSELYKYKVVK
IEPLGVAPTRCKRRVVGSHSGSGGSGSGGHAAVGIGAVSLGFLGAAGSTM
GAASMTLTVQARNLLSGIVQQQSNLLRAPEPQQHLLKDTHWGIKQLQARV
LAVEHYLRDQQLLGIWGCSGKLICCTNVPWNSSWSNRNLSEIWDNMTWLQ
WDKEISNYTQIIYGLLEESQNQQEKNEQDLLALDKWASLWNWFDISNWLW
YIKIFIMIVGGLIGLRIVFAVLSVIHRVR (SEQ ID NO: 5)
BG505_ApexGT5.2_congly_CD4KO_link14
AENLWVTVYYGVPVWKDAETTLFCASDAKAYETEKHNVWATHACVPTDPN
PQEIHLENVTEEFNMWKNNMVEQMHEDIISLWDQSLKPCVKLTPLCVTLQ
CTNVTNNITDDMRGELKNCSFNATTELRNKRQKVYSLFYRLDIVPMVDLW
TNYRLINCNTSAITQACPKVSFEPIPIHYCAPAGFAILKCKDKKFNGTGP
CQNVSTVQCTHGIKPVVSTQLLLNGSLAEEEVIIRSENITNNAKNILVQL
NTSVQINCTRPNNNTVKSIRIGPGQAFYYTGDIIGDIRQAHCNVSKATWN
ETLGKVVKQLRKHFGNNTIIRFAQSSGGDLEVTTHSFNCGGEFFYCNTSG
LFNSTWISNTSVQGSNSTGSNDSITLPCRIKQIINMWQRIGQAMYAPPIQ
GVIRCVSNITGLILTRDGGSTNSTTETFRPGGTDMRDNWRSELYKYKVVK
IEPLGVAPTRCKRRVVGSHSGSGGSGSGGHAAVGIGAVSLGFLGAAGSTM
GAASMTLTVQARNLLSGIVQQQSNLLRAPEPQQHLLKDTHWGIKQLQARV
LAVEHYLRDQQLLGIWGCSGKLICCTNVPWNSSWSNRNLSEIWDNMTWLQ
WDKEISNYTQIIYGLLEESQNQQEKNEQDLLALD (SEQ ID NO: 6)
BG505_ApexGT5.2_congly_gp151_link14
AENLWVTVYYGVPVWKDAETTLFCASDAKAYETEKHNVWATHACVPTDPN
PQEIHLENVTEEFNMWKNNMVEQMHEDIISLWDQSLKPCVKLTPLCVTLQ
CTNVTNNITDDMRGELKNCSFNATTELRNKRQKVYSLFYRLDIVPMVDLW
TNYRLINCNTSAITQACPKVSFEPIPIHYCAPAGFAILKCKDKKFNGTGP
CQNVSTVQCTHGIKPVVSTQLLLNGSLAEEEVIIRSENITNNAKNILVQL
NTSVQINCTRPNNNTVKSIRIGPGQAFYYTGDIIGDIRQAHCNVSKATWN
ETLGKVVKQLRKHFGNNTIIRFAQSSGGDLEVTTHSFNCGGEFFYCNTSG
LFNSTWISNTSVQGSNSTGSNDSITLPCRIKQIINMWQRIGQAMYAPPIQ
GVIRCVSNITGLILTRDGGSTNSTTETFRPGGGDMRDNWRSELYKYKVVK
IEPLGVAPTRCKRRVVGSHSGSGGSGSGGHAAVGIGAVSLGFLGAAGSTM
GAASMTLTVQARNLLSGIVQQQSNLLRAPEPQQHLLKDTHWGIKQLQARV
LAVEHYLRDQQLLGIWGCSGKLICCTNVPWNSSWSNRNLSEIWDNMTWLQ
WDKEISNYTQIIYGLLEESQNQQEKNEQDLLALDKWASLWNWFDISNWLW
YIKIFIMIVGGLIGLRIVFAVLSVIHRVR (SEQ ID NO: 7)
BG505_ApexGT5.2_congly_link14
AENLWVTVYYGVPVWKDAETTLFCASDAKAYETEKHNVWATHACVPTDPN
PQEIHLENVTEEFNMWKNNMVEQMHEDIISLWDQSLKPCVKLTPLCVTLQ
CTNVTNNITDDMRGELKNCSFNATTELRNKRQKVYSLFYRLDIVPMVDLW
TNYRLINCNTSAITQACPKVSFEPIPIHYCAPAGFAILKCKDKKFNGTGP
CQNVSTVQCTHGIKPVVSTQLLLNGSLAEEEVIIRSENITNNAKNILVQL
NTSVQINCTRPNNNTVKSIRIGPGQAFYYTGDIIGDIRQAHCNVSKATWN
ETLGKVVKQLRKHFGNNTIIRFAQSSGGDLEVTTHSFNCGGEFFYCNTSG
LFNSTWISNTSVQGSNSTGSNDSITLPCRIKQIINMWQRIGQAMYAPPIQ
GVIRCVSNITGLILTRDGGSTNSTTETFRPGGGDMRDNWRSELYKYKVVK
IEPLGVAPTRCKRRVVGSHSGSGGSGSGGHAAVGIGAVSLGFLGAAGSTM
GAASMTLTVQARNLLSGIVQQQSNLLRAPEPQQHLLKDTHWGIKQLQARV
LAVEHYLRDQQLLGIWGCSGKLICCTNVPWNSSWSNRNLSEIWDNMTWLQ
WDKEISNYTQIIYGLLEESQNQQEKNEQDLLALD (SEQ ID NO: 8)
BG505_ApexGT5.2_gp151_link14
AENLWVTVYYGVPVWKDAETTLFCASDAKAYETEKHNVWATHACVPTDPN
PQEIHLENVTEEFNMWKNNMVEQMHEDIISLWDQSLKPCVKLTPLCVTLQ
CTNVTNNITDDMRGELKNCSFNATTELRNKROKVYSLFYRLDIVPMVDLW
TNYRLINCNTSAITQACPKVSFEPIPIHYCAPAGFAILKCKDKKFNGTGP
CPSVSTVQCTHGIKPVVSTQLLLNGSLAEEEVIIRSENITNNAKNILVQL
NTPVQINCTRPNNNTVKSIRIGPGQAFYYTGDIIGDIRQAHCNVSKATWN
ETLGKVVKQLRKHFGNNTIIRFAQSSGGDLEVTTHSFNCGGEFFYCNTSG
LFNSTWISNTSVQGSNSTGSNDSITLPCRIKQIINMWQRIGQAMYAPPIQ
GVIRCVSNITGLILTRDGGSTNSTTETFRPGGGDMRDNWRSELYKYKVVK
IEPLGVAPTRCKRRVVGSHSGSGGSGSGGHAAVGIGAVSLGFLGAAGSTM
GAASMTLTVQARNLLSGIVQQQSNLLRAPEPQQHLLKDTHWGIKQLQARV
LAVEHYLRDQQLLGIWGCSGKLICCTNVPWNSSWSNRNLSEIWDNMTWLQ
WDKEISNYTQIIYGLLEESQNQQEKNEQDLLALDKWASLWNWFDISNWLW
YIKIFIMIVGGLIGLRIVFAVLSVIHRVR** (SEQ ID NO: 9)
BG505_ApexGT6.2_gp151_link14
AENLWVTVYYGVPVWKDAETTLFCASDAKAYETEKHNVWATHACVSTDPN
PQEIHLENVTEEFNMWKNNMVEQMHEDIISLWDQSLKPCVKLTPLCVGLQ
CTNVTNNITDDMRGELKNCSFNATTELRNKRQKVYSLFYRLDIVPMVDLW
TNYRLISQNTSAITQACPKVSFEPIPIHYCAPAGFAILKCKDKKFNGTGP
CPSVSTVQCTHGIKPVVSTQLLLNGSLAEEEVIIRSENITNNAKNILVQL
NTPVQINCTRPNNNTVKSIRIGPGQAFYYTGDIIGDIRQAHCNVSKATWN
ETLGKVVKQLRKHFGNNTIIRFAQSSGGDLEVTTHSFNCGGEFFYCNTSG
LFNSTWISNTSVQGSNSTGSNDSITLPCRIKQIINMWQRIGQAMYAPPIQ
GVIRCVSNITGLILTRDGGSTNSTTETFRPGGGDMRDNWRSELYKYKVVK
IEPLGVAPTRCKRRVVGSHSGSGGSGSGGHAAVGIGAVSLGFLGAAGSTM
GAASMTLTVQARNLLSGIVQQQSNLLRAPEPQQHLLKDTHWGIKQLQARV
LAVEHYLRDQQLLGIWGCSGKLICCTNVPWNSSWSNRNLSEIWDNMTWLQ
WDKEISNYTQIIYGLLEESQNQQEKNEQDLLALDKWASLWNWFDISNWLW
YIKIFIMIVGGLIGLRIVFAVLSVIHRVR** (SEQ ID NO: 10)
BG505_SOSIP_D664_m
AENLWVTVYYGVPVWKDAETTLFCASDAKAYETEKHNVWATHACVPTDPN
PQEIHLENVTEEFNMWKNNMVEQMHTDIISLWDQSLKPCVKLTPLCVTLQ
CTNVTNNITDDMRGELKNCSFNMTTELRDKKQKVYSLFYRLDVVQINENQ
GNRSNNSNKEYRLINCNTSAITQACPKVSFEPIPIHYCAPAGFAILKCKD
KKFNGTGPCPSVSTVQCTHGIKPVVSTQLLLNGSLAEEEVMIRSENITNN
AKNILVQFNTPVQINCTRPNNNTRKSIRIGPGQAFYATGDIIGDIRQAHC
NVSKATWNETLGKVVKQLRKHFGNNTIIRFANSSGGDLEVTTHSFNCGGE
FFYCNTSGLFNSTWISNTSVQGSNSTGSNDSITLPCRIKQIINMWQRIGQ
AMYAPPIQGVIRCVSNITGLILTRDGGSTNSTTETFRPGGGDMRDNWRSE
LYKYKVVKIEPLGVAPTRCKRRVVGRRRRRRAVGIGAVFLGFLGAAGSTM
GAASMTLTVQARNLLSGIVQQQSNLLRAPEAQQHLLKLTVWGIKQLQARV
LAVERYLRDQQLLGIWGCSGKLICCTNVPWNSSWSNRNLSEIWDNMTWLQ
WDKEISNYTQIIYGLLEESQNQQEKNEQDLLALD** (SEQ ID NO:
11)
BG505_MD39_gp140-PDGFR_link14_m
AENLWVTVYYGVPVWKDAETTLFCASDAKAYETEKHNVWATHACVPTDPN
PQEIHLENVTEEFNMWKNNMVEQMHEDIISLWDQSLKPCVKLTPLCVTLQ
CTNVTNNITDDMRGELKNCSFNMTTELRDKKQKVYSLFYRLDVVQINENQ
GNRSNNSNKEYRLINCNTSAITQACPKVSFEPIPIHYCAPAGFAILKCKD
KKFNGTGPCPSVSTVQCTHGIKPVVSTQLLLNGSLAEEEVIIRSENITNN
AKNILVQLNTPVQINCTRPNNNTVKSIRIGPGQAFYYTGDIIGDIRQAHC
NVSKATWNETLGKVVKQLRKHFGNNTIIRFAQSSGGDLEVTTHSFNCGGE
FFYCNTSGLFNSTWISNTSVQGSNSTGSNDSITLPCRIKQIINMWQRIGQ
AMYAPPIQGVIRCVSNITGLILTRDGGSTNSTTETFRPGGGDMRDNWRSE
LYKYKVVKIEPLGVAPTRCKRRVVGSHSGSGGSGSGGHAAVGIGAVSLGF
LGAAGSTMGAASMTLTVQARNLLSGIVQQQSNLLRAPEPQQHLLKDTHWG
IKQLQARVLAVEHYLRDQQLLGIWGCSGKLICCTNVPWNSSWSNRNLSEI
WDNMTWLQWDKEISNYTQIIYGLLEESQNQQEKNEQDLLALDGGGSGGSG
GSEQKLISEEDLGGSGGSGGSNAVGQDTQEVIVVPHSLPFKVVVISAILA
LVVLTIISLIILIMLWQKKPR** (SEQ ID NO: 12)
BG505_MD39_gp160-dCT_link14_congly
(MD39.3 gp160-dCT)
AENLWVTVYYGVPVWKDAETTLFCASDAKAYETEKHNVWATHACVPTDPN
PQEIHLENVTEEFNMWKNNMVEQMHEDIISLWDQSLKPCVKLTPLCVTLQ
CTNVTNNITDDMRGELKNCSFNMTTELRDKKQKVYSLFYRLDVVQINENQ
GNRSNNSNKEYRLINCNTSAITQACPKVSFEPIPIHYCAPAGFAILKCKD
KKFNGTGPCQNVSTVQCTHGIKPVVSTQLLLNGSLAEEEVIIRSENITNN
AKNILVQLNTSVQINCTRPNNNTVKSIRIGPGQAFYYTGDIIGDIRQAHC
NVSKATWNETLGKVVKQLRKHFGNNTIIRFAQSSGGDLEVTTHSFNCGGE
FFYCNTSGLFNSTWISNTSVQGSNSTGSNDSITLPCRIKQIINMWQRIGQ
AMYAPPIQGVIRCVSNITGLILTRDGGSTNSTTETFRPGGGDMRDNWRSE
LYKYKVVKIEPLGVAPTRCKRRVVGSHSGSGGSGSGGHAAVGIGAVSLGF
LGAAGSTMGAASMTLTVQARNLLSGIVQQQSNLLRAPEPQQHLLKDTHWG
IKQLQARVLAVEHYLRDQQLLGIWGCSGKLICCTNVPWNSSWSNRNLSEI
WDNMTWLQWDKEISNYTQIIYGLLEESQNQQEKNEQDLLALDKWASLWNW
FDISNWLWYIKIFIMIVGGLIGLRIVFAVLSVIHRVRQGYSPLS**
(SEQ ID NO: 13)
BG505_MD39_gp160-dCT_link14_m (MD39.2
gp160-dCT)
AENLWVTVYYGVPVWKDAETTLFCASDAKAYETEKHNVWATHACVPTDPN
PQEIHLENVTEEFNMWKNNMVEQMHEDIISLWDQSLKPCVKLTPLCVTLQ
CTNVTNNITDDMRGELKNCSFNMTTELRDKKQKVYSLFYRLDVVQINENQ
GNRSNNSNKEYRLINCNTSAITQACPKVSFEPIPIHYCAPAGFAILKCKD
KKFNGTGPCPSVSTVQCTHGIKPVVSTQLLLNGSLAEEEVIIRSENITNN
AKNILVQLNTPVQINCTRPNNNTVKSIRIGPGQAFYYTGDIIGDIRQAHC
NVSKATWNETLGKVVKQLRKHFGNNTIIRFAQSSGGDLEVTTHSFNCGGE
FFYCNTSGLFNSTWISNTSVQGSNSTGSNDSITLPCRIKQIINMWQRIGQ
AMYAPPIQGVIRCVSNITGLILTRDGGSTNSTTETFRPGGGDMRDNWRSE
LYKYKVVKIEPLGVAPTRCKRRVVGSHSGSGGSGSGGHAAVGIGAVSLGF
LGAAGSTMGAASMTLTVQARNLLSGIVQQQSNLLRAPEPQQHLLKDTHWG
IKQLQARVLAVEHYLRDQQLLGIWGCSGKLICCTNVPWNSSWSNRNLSEI
WDNMTWLQWDKEISNYTQIIYGLLEESQNQQEKNEQDLLALDKWASLWNW
FDISNWLWYIKIFIMIVGGLIGLRIVFAVLSVIHRVRQGYSPLS**
(SEQ ID NO: 14)
BG505_MD39_link14_2JD6_m (MD39.2
ferritin)
AENLWVTVYYGVPVWKDAETTLFCASDAKAYETEKHNVWATHACVPTDPN
PQEIHLENVTEEFNMWKNNMVEQMHEDIISLWDQSLKPCVKLTPLCVTLQ
CTNVTNNITDDMRGELKNCSFNMTTELRDKKQKVYSLFYRLDVVQINENQ
GNRSNNSNKEYRLINCNTSAITQACPKVSFEPIPIHYCAPAGFAILKCKD
KKFNGTGPCPSVSTVQCTHGIKPVVSTQLLLNGSLAEEEVIIRSENITNN
AKNILVQLNTPVQINCTRPNNNTVKSIRIGPGQAFYYTGDIIGDIRQAHC
NVSKATWNETLGKVVKQLRKHFGNNTIIRFAQSSGGDLEVTTHSFNCGGE
FFYCNTSGLFNSTWISNTSVQGSNSTGSNDSITLPCRIKQIINMWQRIGQ
AMYAPPIQGVIRCVSNITGLILTRDGGSTNSTTETFRPGGGDMRDNWRSE
LYKYKVVKIEPLGVAPTRCKRRVVGSHSGSGGSGSGGHAAVGIGAVSLGF
LGAAGSTMGAASMTLTVQARNLLSGIVQQQSNLLRAPEPQQHLLKDTHWG
IKQLQARVLAVEHYLRDQQLLGIWGCSGKLICCTNVPWNSSWSNRNLSEI
WDNMTWLQWDKEISNYTQIIYGLLEESQNQQEKNEQDLLALDGSGGLSER
MLKALNDQLNRELYSAYLYFAMAAYFEDLGLEGFANWMKAQAEEEIGHAL
RFYNYIYDKNGRVELDEIPKPPKEWESPLKAFEAAYEHEKFISKSIYELA
ALAEEEKDYSTRAFLEWFINEQVEEEASVKKILDKLKFAKDSPQILFMLD
KELSARAPKLPGLLMQGGE (SEQ ID NO: 15)
BG505MD39.3(aka MD39_link14_congly aka
BG505_SOSIP_MD39_link14_congly_m)
AENLWVTVYYGVPVWKDAETTLFCASDAKAYETEKHNVWATHACVPTDPN
PQEIHLENVTEEFNMWKNNMVEQMHEDIISLWDQSLKPCVKLTPLCVTLQ
CTNVTNNITDDMRGELKNCSFNMTTELRDKKQKVYSLFYRLDVVQINENQ
GNRSNNSNKEYRLINCNTSAITQACPKVSFEPIPIHYCAPAGFAILKCKD
KKFNGTGPCONVSTVQCTHGIKPVVSTQLLLNGSLAEEEVIIRSENITNN
AKNILVQLNTSVQINCTRPNNNTVKSIRIGPGQAFYYTGDIIGDIRQAHC
NVSKATWNETLGKVVKQLRKHFGNNTIIRFAQSSGGDLEVTTHSFNCGGE
FFYCNTSGLFNSTWISNTSVQGSNSTGSNDSITLPCRIKQIINMWQRIGQ
AMYAPPIQGVIRCVSNITGLILTRDGGSTNSTTETFRPGGGDMRDNWRSE
LYKYKVVKIEPLGVAPTRCKRRVVGSHSGSGGSGSGGHAAVGIGAVSLGF
LGAAGSTMGAASMTLTVQARNLLSGIVQQQSNLLRAPEPQQHLLKDTHWG
IKQLQARVLAVEHYLRDQQLLGIWGCSGKLICCTNVPWNSSWSNRNLSEI
WDNMTWLQWDKEISNYTQIIYGLLEESQNQQEKNEQDLLALD** (SEQ
ID NO: 16)
BG505 MD39.3CD4KO gp151 (aka BG505
MD39.3CD4KO gp151)
AENLWVTVYYGVPVWKDAETTLFCASDAKAYETEKHNVWATHACVPTDPN
PQEIHLENVTEEFNMWKNNMVEQMHEDIISLWDQSLKPCVKLTPLCVTLQ
CTNVTNNITDDMRGELKNCSFNMTTELRDKKQKVYSLFYRLDVVQINENQ
GNRSNNSNKEYRLINCNTSAITQACPKVSFEPIPIHYCAPAGFAILKCKD
KKFNGTGPCQNVSTVQCTHGIKPVVSTQLLLNGSLAEEEVIIRSENITNN
AKNILVQLNTSVQINCTRPNNNTVKSIRIGPGQAFYYTGDIIGDIRQAHC
NVSKATWNETLGKVVKQLRKHFGNNTIIRFAQSSGGDLEVTTHSFNCGGE
FFYCNTSGLFNSTWISNTSVQGSNSTGSNDSITLPCRIKQIINMWQRIGQ
AMYAPPIQGVIRCVSNITGLILTRDGGSTNSTTETFRPGGTDMRDNWRSE
LYKYKVVKIEPLGVAPTRCKRRVVGSHSGSGGSGSGGHAAVGIGAVSLGF
LGAAGSTMGAASMTLTVQARNLLSGIVQQQSNLLRAPEPQQHLLKDTHWG
IKQLQARVLAVEHYLRDQQLLGIWGCSGKLICCTNVPWNSSWSNRNLSEI
WDNMTWLQWDKEISNYTQIIYGLLEESQNQQEKNEQDLLALDKWASLWNW
FDISNWLWYIKIFIMIVGGLIGLRIVFAVLSVIHRVR** (SEQ ID
NO: 17)
BG505 MD39.3 gp151 (aka
BG505_MD39_gp151_link14_congly)
AENLWVTVYYGVPVWKDAETTLFCASDAKAYETEKHNVWATHACVPTDPN
PQEIHLENVTEEFNMWKNNMVEQMHEDIISLWDQSLKPCVKLTPLCVTLQ
CTNVTNNITDDMRGELKNCSFNMTTELRDKKQKVYSLFYRLDVVQINENQ
GNRSNNSNKEYRLINCNTSAITQACPKVSFEPIPIHYCAPAGFAILKCKD
KKFNGTGPCQNVSTVQCTHGIKPVVSTQLLLNGSLAEEEVIIRSENITNN
AKNILVQLNTSVQINCTRPNNNTVKSIRIGPGQAFYYTGDIIGDIRQAHC
NVSKATWNETLGKVVKQLRKHFGNNTIIRFAQSSGGDLEVTTHSFNCGGE
FFYCNTSGLFNSTWISNTSVQGSNSTGSNDSITLPCRIKQIINMWQRIGQ
AMYAPPIQGVIRCVSNITGLILTRDGGSTNSTTETFRPGGGDMRDNWRSE
LYKYKVVKIEPLGVAPTRCKRRVVGSHSGSGGSGSGGHAAVGIGAVSLGF
LGAAGSTMGAASMTLTVQARNLLSGIVQQQSNLLRAPEPQQHLLKDTHWG
IKQLQARVLAVEHYLRDQQLLGIWGCSGKLICCTNVPWNSSWSNRNLSEI
WDNMTWLQWDKEISNYTQIIYGLLEESQNQQEKNEQDLLALDKWASLWNW
FDISNWLWYIKIFIMIVGGLIGLRIVFAVLSVIHRVR** (SEQ ID
NO: 18)
BG505_SOSIP_MD39_link14_m (MD39.2)
AENLWVTVYYGVPVWKDAETTLFCASDAKAYETEKHNVWATHACVPTDPN
PQEIHLENVTEEFNMWKNNMVEQMHEDIISLWDQSLKPCVKLTPLCVTLQ
CTNVTNNITDDMRGELKNCSFNMTTELRDKKQKVYSLFYRLDVVQINENQ
GNRSNNSNKEYRLINCNTSAITQACPKVSFEPIPIHYCAPAGFAILKCKD
KKFNGTGPCPSVSTVQCTHGIKPVVSTQLLLNGSLAEEEVIIRSENITNN
AKNILVQLNTPVQINCTRPNNNTVKSIRIGPGQAFYYTGDIIGDIRQAHC
NVSKATWNETLGKVVKQLRKHFGNNTIIRFAQSSGGDLEVTTHSFNCGGE
FFYCNTSGLFNSTWISNTSVQGSNSTGSNDSITLPCRIKQIINMWQRIGQ
AMYAPPIQGVIRCVSNITGLILTRDGGSTNSTTETFRPGGGDMRDNWRSE
LYKYKVVKIEPLGVAPTRCKRRVVGSHSGSGGSGSGGHAAVGIGAVSLGF
LGAAGSTMGAASMTLTVQARNLLSGIVQQQSNLLRAPEPQQHLLKDTHWG
IKQLQARVLAVEHYLRDQQLLGIWGCSGKLICCTNVPWNSSWSNRNLSEI
WDNMTWLQWDKEISNYTQIIYGLLEESQNQQEKNEQDLLALD** (SEQ
ID NO: 19)
BG505.ApexGT2.Gmax
AENLWVTVYYGVPVWKDAETTLFCASDAKAYETEKHNVWATHACVPTDPN
PQEIHLENVTEEFNMWKNNMVEQMHEDIISLWDQSLKPCVKLTPLCVTLN
CTNVTNNITDDMRGELKNCSFNATTELRNKROKVYSLFYRLDIVPMGENS
TNYRLINCNTSAITQACPKVSFEPIPIHYCAPAGFAILKCKDKKFNGTGP
CQNVSTVQCTHGIKPVVSTQLLLNGSLAEEEVIIRSENITNNAKNILVQL
NTSVQINCTRPNNNTVKSIRIGPGQAFYYTGDIIGDIRQAHCNVSKATWN
ETLGKVVKQLRKHFGNNTIIRFAQSSGGDLEVTTHSFNCGGEFFYCNTSG
LFNSTWISNTSVQGSNSTGSNDSITLPCRIKQIINMWQRIGQAMYAPPIQ
GVIRCVSNITGLILTRDGGSTNSTTETFRPGGGDMRDNWRSELYKYKVVK
IEPLGVAPTRCKRRVVGRRRRRRAVGIGAVSLGFLGAAGSTMGAASMTLT
VQARNLLSGIVQQQSNLLRAPEPQQHLLKDTHWGIKQLQARVLAVEHYLR
DQQLLGIWGCSGKLICCTNVPWNSSWSNRNLSEIWDNMTWLQWDKEISNY
TQIIYGLLEESQNQQEKNEQDLLALD (SEQ ID NO: 20)
BG505.ApexGT2.2M UT
AENLWVTVYYGVPVWKDAETTLFCASDAKAYETEKHNVWATHACVPTDPN
PQEIHLENVTEEFNMWKNNMVEQMHEDIISLWDQSLKPCVKLTPLCVTLQ
CTNVTNNITDDMRGELKNCSFNATTELRNKROKVYSLFYRLDIVPMGENS
ANYRLIDCNTSAITQACPKVSFEPIPIHYCAPAGFAILKCKDKKFNGTGP
CPSVSTVQCTHGIKPVVSTQLLLNGSLAEEEVIIRSENITNNAKNILVQL
NTPVQINCTRPNNNTVKSIRIGPGQAFYYTGDIIGDIRQAHCNVSKATWN
ETLGKVVKQLRKHFGNNTIIRFAQSSGGDLEVTTHSFNCGGEFFYCNTSG
LFNSTWISNTSVQGSNSTGSNDSITLPCRIKQIINMWQRIGQAMYAPPIQ
GVIRCVSNITGLILTRDGGSTNSTTETFRPGGGDMRDNWRSELYKYKVVK
IEPLGVAPTRCKRRVVGRRRRRRAVGIGAVSLGFLGAAGSTMGAASMTLT
VQARNLLSGIVQQQSNLLRAPEPQQHLLKDTHWGIKQLQARVLAVEHYLR
DQQLLGIWGCSGKLICCTNVPWNSSWSNRNLSEIWDNMTWLQWDKEISNY
TQIIYGLLEESQNQQEKNEQDLLALD (SEQ ID NO: 21
BG505.ApexGT3A
AENLWVTVYYGVPVWKDAETTLFCASDAKAYETEKHNVWATHACVPTDPN
PQEIHLENVTEEFNMWKNNMVEQMHEDIISLWDQSLKPCVKLTPLCVTLQ
CTNVTNNITDDMRGELKNCSFNATTELRNKRVKRYSLFYRLDIVQIDSNR
AKSHYRLINCNTSAITQACPKVSFEPIPIHYCAPAGFAILKCKDKKFNGT
GPCPSVSTVQCTHGIKPVVSTQLLLNGSLAEEEVIIRSENITNNAKNILV
QLNTPVQINCTRPNNNTVKSIRIGPGQAFYYTGDIIGDIRQAHCNVSKAT
WNETLGKVVKQLRKHFGNNTIIRFAQSSGGDLEVTTHSFNCGGEFFYCNT
SGLFNSTWISNTSVQGSNSTGSNDSITLPCRIKQIINMWQRIGQAMYAPP
IQGVIRCVSNITGLILTRDGGSTNSTTETFRPGGGDMRDNWRSELYKYKV
VKIEPLGVAPTRCKRRVVGRRRRRRAVGIGAVSLGFLGAAGSTMGAASMT
LTVQARNLLSGIVQQQSNLLRAPEPQQHLLKDTHWGIKQLQARVLAVEHY
LRDQQLLGIWGCSGKLICCTNVPWNSSWSNRNLSEIWDNMTWLQWDKEIS
NYTQIIYGLLEESQNQQEKNEQDLLALD (SEQ ID NO: 22
BG505.ApexGT3A.N130
AENLWVTVYYGVPVWKDAETTLFCASDAKAYETEKHNVWATHACVPTDPN
PQEIHLENVTEEFNMWKNNMVEQMHEDIISLWDQSLKPCVKLTPLCVTLN
CTNVTNNITDDMRGELKNCSFNATTELRNKRVKRYSLFYRLDIVQIDSNR
TKSHYRLINCNTSAITQACPKVSFEPIPIHYCAPAGFAILKCKDKKFNGT
GPCPSVSTVQCTHGIKPVVSTQLLLNGSLAEEEVIIRSENITNNAKNILV
QLNTPVQINCTRPNNNTVKSIRIGPGQAFYYTGDIIGDIRQAHCNVSKAT
WNETLGKVVKQLRKHFGNNTIIRFAQSSGGDLEVTTHSFNCGGEFFYCNT
SGLFNSTWISNTSVQGSNSTGSNDSITLPCRIKQIINMWQRIGQAMYAPP
IQGVIRCVSNITGLILTRDGGSTNSTTETFRPGGGDMRDNWRSELYKYKV
VKIEPLGVAPTRCKRRVVGRRRRRRAVGIGAVSLGFLGAAGSTMGAASMT
LTVQARNLLSGIVQQQSNLLRAPEPQQHLLKDTHWGIKQLQARVLAVEHY
LRDQQLLGIWGCSGKLICCTNVPWNSSWSNRNLSEIWDNMTWLQWDKEIS
NYTQIIYGLLEESQNQQEKNEQDLLALD** (SEQ ID NO: 23)
BG505.ApexGT3A.Gmax
AENLWVTVYYGVPVWKDAETTLFCASDAKAYETEKHNVWATHACVPTDPN
PQEIHLENVTEEFNMWKNNMVEQMHEDIISLWDQSLKPCVKLTPLCVTLN
CTNVTNNITDDMRGELKNCSFNATTELRNKRVKRYSLFYRLDIVQIDSNR
TKSHYRLINCNTSAITQACPKVSFEPIPIHYCAPAGFAILKCKDKKFNGT
GPCQNVSTVQCTHGIKPVVSTQLLLNGSLAEEEVIIRSENITNNAKNILV
QLNTSVQINCTRPNNNTVKSIRIGPGQAFYYTGDIIGDIRQAHCNVSKAT
WNETLGKVVKQLRKHFGNNTIIRFAQSSGGDLEVTTHSFNCGGEFFYCNT
SGLFNSTWISNTSVQGSNSTGSNDSITLPCRIKQIINMWQRIGQAMYAPP
IQGVIRCVSNITGLILTRDGGSTNSTTETFRPGGGDMRDNWRSELYKYKV
VKIEPLGVAPTRCKRRVVGRRRRRRAVGIGAVSLGFLGAAGSTMGAASMT
LTVQARNLLSGIVQQQSNLLRAPEPQQHLLKDTHWGIKQLQARVLAVEHY
LRDQQLLGIWGCSGKLICCTNVPWNSSWSNRNLSEIWDNMTWLQWDKEIS
NYTQIIYGLLEESQNQQEKNEQDLLALD (SEQ ID NO: 24)
BG505.ApexGT5
AENLWVTVYYGVPVWKDAETTLFCASDAKAYETEKHNVWATHACVPTDPN
PQEIHLENVTEEFNMWKNNMVEQMHEDIISLWDQSLKPCVKLTPLCVTLQ
CTNVTNNITDDMRGELKNCSFNATTELRNKRQKVYSLFYRLDIVPMVDLW
TNYRLINCNTSAITQACPKVSFEPIPIHYCAPAGFAILKCKDKKFNGTGP
CPSVSTVQCTHGIKPVVSTQLLLNGSLAEEEVIIRSENITNNAKNILVQL
NTPVQINCTRPNNNTVKSIRIGPGQAFYYTGDIIGDIRQAHCNVSKATWN
ETLGKVVKQLRKHFGNNTIIRFAQSSGGDLEVTTHSFNCGGEFFYCNTSG
LFNSTWISNTSVQGSNSTGSNDSITLPCRIKQIINMWQRIGQAMYAPPIQ
GVIRCVSNITGLILTRDGGSTNSTTETFRPGGGDMRDNWRSELYKYKVVK
IEPLGVAPTRCKRRVVGRRRRRRAVGIGAVSLGFLGAAGSTMGAASMTLT
VQARNLLSGIVQQQSNLLRAPEPQQHLLKDTHWGIKQLQARVLAVEHYLR
DQQLLGIWGCSGKLICCTNVPWNSSWSNRNLSEIWDNMTWLQWDKEISNY
TQIIYGLLEESQNQQEKNEQDLLALDGTKHHHHHH (SEQ ID NO:
25)
BG505.ApexGT5.Gmax
AENLWVTVYYGVPVWKDAETTLFCASDAKAYETEKHNVWATHACVPTDPN
PQEIHLENVTEEFNMWKNNMVEQMHEDIISLWDQSLKPCVKLTPLCVTLN
CTNVTNNITDDMRGELKNCSFNATTELRNKROKVYSLFYRLDIVPMVDLW
TNYRLINCNTSAITQACPKVSFEPIPIHYCAPAGFAILKCKDKKFNGTGP
CQNVSTVQCTHGIKPVVSTQLLLNGSLAEEEVIIRSENITNNAKNILVQL
NTSVQINCTRPNNNTVKSIRIGPGQAFYYTGDIIGDIRQAHCNVSKATWN
ETLGKVVKQLRKHFGNNTIIRFAQSSGGDLEVTTHSFNCGGEFFYCNTSG
LFNSTWISNTSVQGSNSTGSNDSITLPCRIKQIINMWQRIGQAMYAPPIQ
GVIRCVSNITGLILTRDGGSTNSTTETFRPGGGDMRDNWRSELYKYKVVK
IEPLGVAPTRCKRRVVGRRRRRRAVGIGAVSLGFLGAAGSTMGAASMTLT
VQARNLLSGIVQQQSNLLRAPEPQQHLLKDTHWGIKQLQARVLAVEHYLR
DQQLLGIWGCSGKLICCTNVPWNSSWSNRNLSEIWDNMTWLQWDKEISNY
TQIIYGLLEESQNQQEKNEQDLLALDGT (SEQ ID NO: 26)
MD64_CPG9 (aka BG505_SOSIP_MD39C_CPG9_m)
GGNSSGSLGFLGAAGSTMGAASMTLTVQARNLLSGIVQQQSNLLRAPEPQ
QHLLKLTVWGIKQLQARVLAVEHYLRDQQLLGIWGCSGKLICCTNVPWNS
SWSNRNLSEIWDNMTWLNWSKEISNYTQIIYGLLEESQNQNESNEQDLGG
NGSGGGSGSGGNGSSGLWVTVYYGVPVWKDAETTLFCASDAKAYETEKHN
VWATHECVPTDPNSSEIHLENVTEEFNMWKNNMVEQMHTDIIELWDQSLK
PCVKLTPLCVTLQCTNVTNNITDDMRGELKNCSFNMTTELRDKKQKVYSL
FYRLDVVQINENQGNRSNNSNKEYRLINCNTSAITQACPKVSFEPIPIHY
CAPAGFAILKCKDKKFNGTGPCQNVSTVQCTHGIKPVVSTQLLLNGSLAE
EEVIIRSENITNNAKNILVQLNTSVQINCTRPNNNTVKSIRIGPGQAFYY
TGDIIGDIRQAHCNVSKATWNETLGKVVKQLRKHFGNNTIIRFAQSSGGD
LEVTTHSFNCGGEFFYCNTSGLFNSTWISNTSVQGSNSTGSNDSITLPCR
IKQIINMWQRIGQAMYAPPIQGVIRCVSNITGLILTRDGGSTNSTTETFR
PGGGDMRDNWRSELYKYKVVKIEPLGVAPTRCNRS** (SEQ ID NO:
27)
MD39_link14_congly (aka BG505_SOSIP_
MD39_link14_congly_m) (MD39.3)
AENLWVTVYYGVPVWKDAETTLFCASDAKAYETEKHNVWATHACVPTDPN
PQEIHLENVTEEFNMWKNNMVEQMHEDIISLWDQSLKPCVKLTPLCVTLQ
CTNVTNNITDDMRGELKNCSFNMTTELRDKKQKVYSLFYRLDVVQINENQ
GNRSNNSNKEYRLINCNTSAITQACPKVSFEPIPIHYCAPAGFAILKCKD
KKFNGTGPCQNVSTVQCTHGIKPVVSTQLLLNGSLAEEEVIIRSENITNN
AKNILVQLNTSVQINCTRPNNNTVKSIRIGPGQAFYYTGDIIGDIRQAHC
NVSKATWNETLGKVVKQLRKHFGNNTIIRFAQSSGGDLEVTTHSFNCGGE
FFYCNTSGLFNSTWISNTSVQGSNSTGSNDSITLPCRIKQIINMWQRIGQ
AMYAPPIQGVIRCVSNITGLILTRDGGSTNSTTETFRPGGGDMRDNWRSE
LYKYKVVKIEPLGVAPTRCKRRVVGSHSGSGGSGSGGHAAVGIGAVSLGF
LGAAGSTMGAASMTLTVQARNLLSGIVQQQSNLLRAPEPQQHLLKDTHWG
IKQLQARVLAVEHYLRDQQLLGIWGCSGKLICCTNVPWNSSWSNRNLSEI
WDNMTWLQWDKEISNYTQIIYGLLEESQNQQEKNEQDLLALD** (SEQ
ID NO: 16)

The protein may have at least 90% or 95% homology or identity with the sequence of the non-naturally occurring protein(s) of the invention.

Each of the protein sequences of Table 1 optionally has a leader sequence of MGILPSPGMPALLSLVSLLSVLLMGCVAETG (SEQ ID NO: 1).

Some of the protein sequences of Table 1 may comprise a link14 sequence of SHSGSGGSGSGGHA (SEQ ID NO: 28). The link14 sequence is optional in each of the sequences comprising a link14 sequence.

The invention also encompasses trimers which may comprise any one of the non-naturally occurring protein(s) of the invention.

The proteins of the invention may comprise an additional cysteine and/or be fused to be a multimerization motif. The proteins of the invention may also comprise a tag for purification or biotinylation, such as a his tag or a avi-tag.

The invention also encompasses nucleic acids encoding the non-naturally occurring protein(s) of the present invention, including nucleic acids that may have at least 90% or 95% homology or identity with a nucleotide encoding the sequence of the non-naturally occurring protein(s) of the invention. In one embodiment, the nucleic acid may be a mRNA.

In one embodiment, the nucleic acids of the present invention may be delivered as a therapeutic mRNA.

The present invention contemplates expressing the herein disclosed immunogens as mRNAs, advantageously as mRNA vaccines. The disclosures of U.S. Pat. Nos. 9,675,668; 9,464,124; 9,447,164; 9,428,535; 9,334,328; 9,303,079; 9,301,993; 9,295,689; 9,283,287; 9,271,996; 9,255,129; 9,254,311; 9,233,141; 9,221,891; 9,220,792; 9,220,755; 9,216,205; 9,192,651; 9,186,372; 9,181,319; 9,149,506; 9,114,113; 9,107,886; 9,095,552; 9,089,604; 9,061,059; 9,050,297; 8,999,380; 8,980,864; 8,822,663; 8,754,062; 8,710,200; 8,680,069 and 8,664,194 and US Patent Publication Nos. 20220047518, 20200254086, 20200206362, 20180311336, 20180303929, 20170204152, 20160331828, 20160317647 and 20160194368 are herein incorporated by reference.

Exemplary aspects of the invention feature efficacious mRNA vaccines. Described herein are mRNA vaccines designed to achieve particular biologic effects. Exemplary vaccines of the invention feature mRNAs encoding a particular antigen of interest (or and mRNA or mRNAs encoding antigens of interest), optionally formulated with additional components designed to facilitate efficacious delivery of mRNAs in vivo. In exemplary aspects, the vaccines of the invention feature and mRNA or mRNAs encoding antigen(s) of interest, complexed with polymeric or lipid components, or in certain aspects, encapsulated in liposomes, or alternatively, in lipid nanoparticles (LNPs). Chemical modification of mRNAs can facilitate certain desirable properties of vaccines on the invention, for example, influencing the type of immune response to the vaccine. For example, appropriate chemical modification of mRNAs can reduce unwanted innate immune responses against mRNA components and/or can facilitate desirable levels of protein expression of the antigen or antigens of interest. Further description of such features of the invention is provided infra.

Provided herein are isolated nucleic acids (e.g., modified mRNAs encoding a peptide described herein) which may comprise a translatable region and at least two different nucleoside modifications, wherein the nucleic acid exhibits reduced degradation in a cell into which the nucleic acid is introduced, relative to a corresponding unmodified nucleic acid. For example, the degradation rate of the nucleic acid is reduced by at least 10%, 20%, 30%, 40%, 50%, 60%, 70%, 80%, or 90%, compared to the degradation rate of the corresponding unmodified nucleic acid. In certain embodiments, the nucleic acid may comprise RNA, DNA, TNA, GNA, or a hybrid thereof. In certain embodiments, the nucleic acid comprises messenger RNA (mRNA). In certain embodiments, the mRNA does not substantially induce an innate immune response of the cell into which the mRNA is introduced. In certain embodiments, the mRNA may comprise at least one nucleoside selected from the group consisting of pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, and 4-methoxy-2-thio-pseudouridine. In certain embodiments, the mRNA may comprise at least one nucleoside selected from the group consisting of 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine. In other embodiments, the mRNA may comprise at least one nucleoside selected from the group consisting of 2-aminopurine, 2,6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl) adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine. In yet other embodiments, the mRNA may comprise at least one nucleoside selected from the group consisting of inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2, N2-dimethyl-6-thio-guanosine.

In some embodiments, the nucleic acids provided herein comprise a 5′ untranslated region (UTR) and/or a 3′UTR, wherein each of the two different nucleoside modifications are independently present in the 5′UTR and/or 3′UTR. In some embodiments, nucleic acids are provided herein, wherein at least one of the two different nucleoside modifications are present in the translatable region. In some embodiments, nucleic acids provided herein are capable of binding to at least one polypeptide that prevents or reduces an innate immune response of a cell into which the nucleic acid is introduced.

Further provided herein are isolated nucleic acids (e.g., modified mRNAs described herein) which may comprise (i) a translatable region encoding a peptide described herein, (ii) at least one nucleoside modification, and (iii) at least one intronic nucleotide sequence capable of being excised from the nucleic acid.

Further provided herein are isolated nucleic acids (e.g., modified mRNAs described herein) which may comprise (i) a translatable region encoding a peptide described herein, (ii) at least two different nucleoside modifications, and (iii) a degradation domain.

Further provided herein are non-enzymatically synthesized nucleic acids (e.g., modified mRNAs described herein) which may comprise at least one nucleoside modification, and which may comprise a translatable region encoding a peptide described herein. In certain embodiments, the non-enzymatically synthesized mRNA may comprise at least two different nucleoside modifications.

Further provided herein are isolated nucleic acids (e.g., modified mRNAs described herein) which may comprise a noncoding region and at least one nucleoside modification that reduces an innate immune response of a cell into which the nucleic acid is introduced, wherein the nucleic acid sequesters one or more translational machinery components. In certain embodiments, the isolated nucleic acids which may comprise a noncoding region and at least one nucleoside modification described herein are provided in an amount effective to reduce protein expression in the cell. In certain embodiments, the translational machinery component is a ribosomal protein or a transfer RNA (tRNA). In certain embodiments, the nucleic acid may comprise a small nucleolar RNA (sno-RNA), microRNA (miRNA), small interfering RNA (siRNA) or Piwi-interacting RNA (piRNA).

Further provided herein are isolated nucleic acids (e.g., modified mRNAs described herein) which may comprise (i) a first translatable region, (ii) at least one nucleoside modification, and (iii) an internal ribosome entry site (IRES). In certain embodiments, the IRES is obtained from a picornavirus, a pest virus, a polio virus, an encephalomyocarditis virus, a foot-and-mouth disease virus, a hepatitis C virus, a classical swine fever virus, a murine leukemia virus, a simian immune deficiency virus or a cricket paralysis virus. In certain embodiments, the isolated nucleic acid further may comprise a second translatable region. In certain embodiments, the isolated nucleic acid further may comprise a Kozak sequence. In some embodiments, the first translatable region encodes a peptide described herein. In some embodiments, the second translatable region encodes peptide described herein. In some embodiments, the first and the second translatable regions encode peptides described herein.

Provided herein are pharmaceutical compositions which may comprise: (i) an effective amount of a synthetic messenger ribonucleic acid (mRNA) encoding peptide described herein; and (ii) a pharmaceutically acceptable carrier, wherein i) the mRNA may comprise pseudouridine, 5′methyl-cytidine, or a combination thereof, or ii) the mRNA does not comprise a substantial amount of a nucleotide or nucleotides selected from the group consisting of uridine, cytidine, and a combination of uridine and cytidine, and wherein the composition is suitable for repeated administration (e.g., intravenous administration) to a mammalian subject in need thereof. In some embodiments,

Further provided herein are pharmaceutical compositions which may comprise and/or consisting essentially of: (i) an effective amount of a synthetic messenger ribonucleic acid (mRNA) encoding peptide described herein; (ii) a cell penetration agent; and (iii) a pharmaceutically acceptable carrier, wherein i) the mRNA may comprise pseudouridine, 5′methyl-cytidine or a combination thereof, or ii) the mRNA does not comprise a substantial amount of a nucleotide or nucleotides selected from the group consisting of uridine, cytidine, and a combination of uridine and cytidine, and wherein the composition is suitable for repeated administration (e.g., intravenous administration) to an animal (e.g., mammalian) subject in need thereof.

This invention provides nucleic acids, including RNAs such as mRNAs that contain one or more modified nucleosides (termed “modified nucleic acids”), which have useful properties including the lack of a substantial induction of the innate immune response of a cell into which the mRNA is introduced. Because these modified nucleic acids enhance the efficiency of protein production, intracellular retention of nucleic acids, and viability of contacted cells, as well as possess reduced immunogenicity, these nucleic acids having these properties are termed “enhanced nucleic acids” herein.

The term “nucleic acid,” in its broadest sense, includes any compound and/or substance that is or can be incorporated into an oligonucleotide chain. Exemplary nucleic acids for use in accordance with the present invention include, but are not limited to, one or more of DNA, RNA, hybrids thereof, RNAi-inducing agents, RNAi agents, siRNAs, shRNAs, miRNAs, antisense RNAs, ribozymes, catalytic DNA, RNA s that induce triple helix formation, aptamers, vectors, etc., described in detail herein.

Provided are modified nucleic acids containing a translatable region encoding a peptide described herein, and one, two, or more than two different nucleoside modifications. In some embodiments, the modified nucleic acid exhibits reduced degradation in a cell into which the nucleic acid is introduced, relative to a corresponding unmodified nucleic acid. For example, the degradation rate of the nucleic acid is reduced by at least 10%, 20%, 30%, 40%, 50%, 60%, 70%, 80%, or 90%, compared to the degradation rate of the corresponding unmodified nucleic acid. Exemplary nucleic acids include ribonucleic acids (RNAs), deoxyribonucleic acids (DNAs), threose nucleic acids (TNAs), glycol nucleic acids (GNA s), peptide nucleic acids (PNAs), locked nucleic acids (LNAs) or a hybrid thereof. In preferred embodiments, the modified nucleic acid includes messenger RNAs (mRNAs). As described herein, the nucleic acids of the invention do not substantially induce an innate immune response of a cell into which the mRNA is introduced.

In some embodiments, modified nucleosides include pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, and 4-methoxy-2-thio-pseudouridine.

In some embodiments, modified nucleosides include 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine.

In other embodiments, modified nucleosides include 2-aminopurine, 2,6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl) adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine.

In certain embodiments it is desirable to intracellularly degrade a modified nucleic acid introduced into the cell, for example if precise timing of protein production is desired. Thus, the invention provides a modified nucleic acid containing a degradation domain, which is capable of being acted on in a directed manner within a cell.

In other embodiments, modified nucleosides include inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2, N2-dimethyl-6-thio-guanosine.

Other components of nucleic acid are optional, and are beneficial in some embodiments. For example, a 5′ untranslated region (UTR) and/or a 3′UTR are provided, wherein either or both may independently contain one or more different nucleoside modifications. In such embodiments, nucleoside modifications may also be present in the translatable region. Also provided are nucleic acids containing a Kozak sequence.

Further, nucleic acids encoding a peptide described herein, and containing an internal ribosome entry site (IRES) are provided herein. An IRES may act as the sole ribosome binding site, or may serve as one of multiple ribosome binding sites of an mRNA. An mRNA containing more than one functional ribosome binding site may encode several peptides or polypeptides that are translated independently by the ribosomes (“multicistronic mRNA”). When nucleic acids are provided with an IRES, further optionally provided is a second translatable region. Examples of IRES sequences that can be used according to the invention include without limitation, those from picornaviruses (e.g., FMDV), pest viruses (CFFV), polio viruses (PV), encephalomyocarditis viruses (ECMV), foot-and-mouth disease viruses (FMDV), hepatitis C viruses (HCV), classical swine fever viruses (CSFV), murine leukemia virus (MLV), simian immune deficiency viruses (SIV) or cricket paralysis viruses (CrPV).

The therapeutic mRNAs as described, for example, in U.S. Pat. Nos. 9,464,124; 9,447,164; 9,428,535; 9,334,328; 9,303,079; 9,301,993; 9,295,689; 9,283,287; 9,271,996; 9,255,129; 9,254,311; 9,233,141; 9,221,891; 9,220,792; 9,220,755; 9,216,205; 9,192,651; 9,186,372; 9,181,319; 9,149,506; 9,114,113; 9,107,886; 9,095,552; 9,089,604; 9,061,059; 9,050,297; 8,999,380; 8,980,864; 8,822,663; 8,754,062; 8,710,200; 8,680,069 and 8,664,194 may be utilized for the present invention.

Advantageously, the mRNA of the present invention is formulated as a lipid nanoparticle (LNP) formulation, such as a PEG lipid, which are useful in pharmaceutical compositions, cosmetic compositions, and drug delivery systems, e.g., for use in LNP formulations. The LNPs described in US Patent Publication Nos. 20220047518 and 20200254086 are useful for the delivery of an agent (e.g., therapeutic agent such as a nucleic acid) to a subject.

In one set of embodiments, lipid nanoparticles (LNPs) are provided. In one embodiment, a lipid nanoparticle comprises lipids including an ionizable lipid (such as an ionizable cationic lipid), a structural lipid, a phospholipid, and mRNA. Each of the LN Ps described herein may be used as a formulation for the mRNA described herein. In one embodiment, a lipid nanoparticle comprises an ionizable lipid, a structural lipid, a phospholipid, and mRNA. In some embodiments, the LNP comprises an ionizable lipid, a PEG-modified lipid, a phospholipid and a structural lipid. In some embodiments, the LNP has a molar ratio of about 20-60% ionizable lipid: about 5-25% phospholipid: about 25-55% structural lipid; and about 0.5-15% PEG-modified lipid. In some embodiments, the LNP comprises a molar ratio of about 50% ionizable lipid, about 1.5% PEG-modified lipid, about 38.5% structural lipid and about 10% phospholipid. In some embodiments, the LNP comprises a molar ratio of about 55% ionizable lipid, about 2.5% PEG lipid, about 32.5% structural lipid and about 10% phospholipid. In some embodiments, the ionizable lipid is an ionizable amino or cationic lipid and the phospholipid is a neutral lipid, and the structural lipid is a cholesterol. In some embodiments, the LNP has a molar ratio of 50:38.5:10:1.5 of ionizable lipid: cholesterol: DSPC: PEG2000-DM G.

In other aspects the invention is a composition for or method of vaccinating a subject comprising administering to the subject a nucleic acid vaccine comprising one or more RNA polynucleotides having an open reading frame encoding a first antigenic polypeptide wherein a dosage of between 10 μg/kg and 400 μg/kg of the nucleic acid vaccine is administered to the subject. In some embodiments the dosage of the RNA polynucleotide is 1-5 μg, 5-10 μg, 10-15 μg, 15-20 μg, 10-25 μg, 20-25 μg, 20-50 μg, 30-50 μg, 40-50 μg, 40-60 μg, 60-80 μg, 60-100 μg, 50-100 μg, 80-120 μg, 40-120 μg, 40-150 μg, 50-150 μg, 50-200 μg, 80-200 μg, 100-200 μg, 120-250 μg, 150-250 μg, 180-280 μg, 200-300 μg, 50-300 μg, 80-300 μg, 100-300 μg, 40-300 μg, 50-350 μg, 100-350 μg, 200-350 μg, 300-350 μg, 320-400 μg, 40-380 μg, 40-100 μg, 100-400 μg, 200-400 μg, or 300-400 μg per dose. In some embodiments, the nucleic acid vaccine is administered to the subject by intradermal, intraperitoneal or intramuscular injection. Advantageously, the administration is an intramuscular injection. In some embodiments, the nucleic acid vaccine is administered to the subject on day zero. In some embodiments, a second dose of the nucleic acid vaccine is administered to the subject on day twenty one.

In some embodiments, a dosage of 25 micrograms of the RNA polynucleotide is included in the nucleic acid vaccine administered to the subject. In some embodiments, a dosage of 100 micrograms of the RNA polynucleotide is included in the nucleic acid vaccine administered to the subject. In some embodiments, a dosage of 50 micrograms of the RNA polynucleotide is included in the nucleic acid vaccine administered to the subject. In some embodiments, a dosage of 75 micrograms of the RNA polynucleotide is included in the nucleic acid vaccine administered to the subject. In some embodiments, a dosage of 150 micrograms of the RNA polynucleotide is included in the nucleic acid vaccine administered to the subject. In some embodiments, a dosage of 400 micrograms of the RNA polynucleotide is included in the nucleic acid vaccine administered to the subject. In some embodiments, a dosage of 200 micrograms of the RNA polynucleotide is included in the nucleic acid vaccine administered to the subject. In some embodiments, the RNA polynucleotide accumulates at a 100 fold higher level in the local lymph node in comparison with the distal lymph node. In other embodiments the nucleic acid vaccine is chemically modified and in other embodiments the nucleic acid vaccine is not chemically modified.

In exemplary embodiments of the invention, an efficacious vaccine produces an antibody titer of greater than 1:40, greater that 1:100, greater than 1:400, greater than 1:1000, greater than 1:2000, greater than 1:3000, greater than 1:4000, greater than 1:500, greater than 1:6000, greater than 1:7500, greater than 1:10000. In exemplary embodiments, the antibody titer is produced or reached by 10 days following vaccination, by 20 days following vaccination, by 30 days following vaccination, by 40 days following vaccination, or by 50 or more days following vaccination. In exemplary embodiments, the titer is produced or reached following a single dose of vaccine administered to the subject. In other embodiments, the titer is produced or reached following multiple doses, e.g., following a first and a second dose (e.g., a booster dose.) In exemplary aspects of the invention, antigen-specific antibodies are measured in units of ug/ml or are measured in units of IU/L (International Units per liter) or mIU/ml (milli International Units per ml). In exemplary embodiments of the invention, an efficacious vaccine produces >0.5 mg/ml, >0.1 μg/ml, >0.2 μg/ml, >0.35 μg/ml, >0.5 μg/ml, >1 μg/ml, >2 μg/ml, >5 μg/ml or >10 μg/ml. In exemplary embodiments of the invention, an efficacious vaccine produces >10 mIU/ml, >20 mIU/ml, >50 mIU/ml, >100 mIU/ml, >200 mIU/ml, >500 mIU/ml or >1000 mIU/ml. In exemplary embodiments, the antibody level or concentration is produced or reached by 10 days following vaccination, by 20 days following vaccination, by 30 days following vaccination, by 40 days following vaccination, or by 50 or more days following vaccination. In exemplary embodiments, the level or concentration is produced or reached following a single dose of vaccine administered to the subject. In other embodiments, the level or concentration is produced or reached following multiple doses, e.g., following a first and a second dose (e.g., a booster dose.) In exemplary embodiments, antibody level or concentration is determined or measured by enzyme-linked immunosorbent assay (ELISA). In exemplary embodiments, antibody level or concentration is determined or measured by neutralization assay, e.g., by microneutralization assay.

Methods for the chemical conjugation of polypeptides, carbohydrates, and/or lipids are well known in the art (see, for example, Hermanson. Bioconjugate Techniques (Academic Press; 1992); Aslam and Dent, eds. Bioconjugation: Protein coupling Techniques for the Biomedical Sciences (MacMillan: 1998); and Wong Chemistry of Protein Conjugation and Cross-linking (CRC Press: 1991)). For instance, primary amino groups may be incorporated by reaction with ethylenediamine in the presence of sodium cyanoborohydride and sulfhydryls may be introduced by reaction of cysteamin dihydrochloride followed by reduction with a standard disulfide reducing agent. Heterobifunctional crosslinkers, such as, for example, sulfosuccinimidyl (4-iodoacetyl) aminobenzoate, which link the epsilon amino group on the D-lysine residues of copolymers of D-lysine and D-glutamate to a sulfhydryl side chain from an amino terminal cysteine residue on the peptide to be coupled, may be used as well. Chemical conjugation also includes anything covalently bonded directly via side chain bonds or via a linker or spacer group.

The nanoparticle formulations may be a carbohydrate nanoparticle which may comprise a carbohydrate carrier and a modified nucleic acid molecule (e.g., mmRNA). As a non-limiting example, the carbohydrate carrier may include, but is not limited to, an anhydride-modified phytoglycogen or glycogen-type material, phtoglycogen octenyl succinate, phytoglycogen beta-dextrin, anhydride-modified phytoglycogen beta-dextrin. (See e.g., International Publication No. WO2012109121; herein incorporated by reference in its entirety).

Lipid nanoparticle formulations may be improved by replacing the cationic lipid with a biodegradable cationic lipid which is known as a rapidly eliminated lipid nanoparticle (reLNP). Ionizable cationic lipids, such as, but not limited to, DLinDMA, DLin-KC2-DMA, and DLin-MC3-DMA, have been shown to accumulate in plasma and tissues over time and may be a potential source of toxicity. The rapid metabolism of the rapidly eliminated lipids can improve the tolerability and therapeutic index of the lipid nanoparticles by an order of magnitude from a 1 mg/kg dose to a 10 mg/kg dose in rat. Inclusion of an enzymatically degraded ester linkage can improve the degradation and metabolism profile of the cationic component, while still maintaining the activity of the reLNP formulation. The ester linkage can be internally located within the lipid chain or it may be terminally located at the terminal end of the lipid chain. The internal ester linkage may replace any carbon in the lipid chain.

The average diameter of the nanoparticle employed in the compositions of the invention can be at least one member selected from the group consisting of about 20 nanometers, about 25 nanometers, about 30 nanometers, about 40 nanometers, about 50 nanometers, about 75 nanometers, about 100 nanometers, about 125 nanometers, about 150 nanometers, about 175 nanometers and about 200 nanometers. In another embodiment, the average diameter of the particle is at least one member selected from the group consisting of between about 10 to about 200 nanometers, between about 0.5 to about 5 microns and between about 5 to about 10 microns. In another embodiment, the average diameter of the microparticle is selected from the group consisting of about 0.1 μm, about 0.2 μm, about 0.4 μm, about 0.5 μm, about 1 μm and about 2 μm.

Nanoparticles for use in the compositions of the invention can be made from lipids or other fatty acids (see, for example, U.S. Pat. Nos. 5,709,879; 6,342,226; 6,090,406; Lian, et al., J. of Pharma. Sci. 90:667-680 (2001) and van Slooten, et al., Pharm Res. 17:42-48 (2000)) and non-lipid compositions (see, for example, K reuter, J. A nat. 189:503-505 (1996), the teachings of all of which are hereby incorporated by reference in their entirety). The compositions can be bilayer or multilamellar liposomes and phospholipid based. Polymerized nanoparticles, as described, for example, in U.S. Pat. No. 7,285,289, the teachings of which are incorporated by reference in their entirety.

Metallic oxide nanoparticles for use in the compositions of the invention can be chemically substituted with at least one reactive moiety capable of forming a thioether bond employing conventionally techniques as described herein and in U.S. Pat. No. 6,086,881, the teachings of which are hereby incorporated by reference in their entirety. The antigen described herein can be coupled in a single step onto the metallic oxide particles by the formation of at least one thioether bond or it may be synthesized or assembled stepwise onto the metallic oxide particles after the initial thioether bond formation. The chemical derivatization reagents for the metallic oxide particles can include organosilane reagents that provide thioalkane functionality or other groups that may readily be converted into thiols or thiol-reactive moieties. Organosilane reagents which may be utilized for this purpose may be, but are not limited to, 3-mercaptopropyltrimethoxysilane, 3-aminopropyltriethoxysilane, 3-iodopropyltrimethoxysilane, 2-chloroethyltrichlorosilane, 3-glycidoxypropyltrimethoxysilane, vinyltrichlorosilane and 3-acryloxypropyltrimethoxysilane. M oieties that include one or more disulfide components may also be joined to the metallic oxide particle surface and thereby provide the corresponding reactive moiety able to enter into and form a thioether bond and juncture. Exemplary nanoparticles for use in the compositions of the invention include at least one member selected from the group consisting of poly (D,L-lactide-co-glycolide, also referred to as “poly (lactic-co-glycolic acid) and bisacyloxypropylcysteine.

Nanoparticles for use in the compositions of the invention can be made of inorganic material. Nanoparticles for use in the compositions of the invention can be made of a polymer material, such as at least one member selected from the group consisting of polystyrene, brominated polystyrene, polyacrylic acid, polyacrylonitrile, polyamide, polyacrylamide, polyacrolein, polybutadiene, polycaprolactone, polycarbonate, polyester, polyethylene, polyethylene terephthalate, polydimethylsiloxane, polyisoprene, polyurethane, polyvinylacetate, polyvinylchloride, polyvinylpyridine, polyvinylbenzylchloride, polyvinyltoluene, polyvinylidene chloride, polydivinylbenzene, polymethylmethacrylate, polylactide, polyglycolide, poly (lactide-co-glycolide), polyanhydride, polyorthoester, polyphosphazene, polyphosophaze, a carbohydrate, carboxymethyl cellulose, hydroxyethyl cellulose, agar, gel, proteinaceous polymer, polypeptide, eukaryotic and prokaryotic cells, viruses, lipid, metal, resin, latex, rubber, silicone (e.g., polydimethyldiphenyl siloxane), glass, ceramic, charcoal, kaolinite and bentonite.

It is noted that these therapeutics may be a chemical compound, a composition which may comprise a polypeptide of the present invention and/or antibody elicited by such a chemical compound and/or portion thereof or a pharmaceutically acceptable salt or a composition which may comprise a polypeptide of the invention, and may be administered alone or as an active ingredient in combination with pharmaceutically acceptable carriers, diluents, and vehicles, as well as other active ingredients.

The compounds or compositions may be administered orally, subcutaneously or parenterally including intravenous, intraarterial, intramuscular, intraperitoneally, and intranasal administration as well as intrathecal and infusion techniques.

It is noted that humans are treated generally longer than the mice or other experimental animals which treatment has a length proportional to the length of the disease process and drug effectiveness. The doses may be single doses or multiple doses over a period of several days, but single doses are preferred. Thus, one may scale up from animal experiments, e.g., rats, mice, and the like, to humans, by techniques from this disclosure and documents cited herein and the knowledge in the art, without undue experimentation.

In a particularly advantageous embodiment, the mRNAs of the present invention are administered in combinations of a prime dose followed by one or more boost doses over time. mRNA doses of about 100 μg are advantageous, however, dosages of about 10 μg to about 1000 μg, about 20 μg to about 900 μg, about 30 μg to about 800 μg, about 40 μg to about 700 μg, about 50 μg to about 600 μg, about 60 μg to about 500 μg, about about 70 μg to about 400 μg, about 80 μg to about 300 μg, or about 900 μg to about 200 μg, are contemplated. Varying combinations are presented below as non-limiting examples.

The treatment generally has a length proportional to the length of the disease process and drug effectiveness and the patient being treated.

When administering a therapeutic of the present invention parenterally, it will generally be formulated in a unit dosage injectable form (solution, suspension, emulsion). The pharmaceutical formulations suitable for injection include sterile aqueous solutions or dispersions and sterile powders for reconstitution into sterile injectable solutions or dispersions. The carrier may be a solvent or dispersing medium containing, for example, water, ethanol, polyol (for example, glycerol, propylene glycol, liquid polyethylene glycol, and the like), suitable mixtures thereof, and vegetable oils.

Proper fluidity may be maintained, for example, by the use of a coating such as lecithin, by the maintenance of the required particle size in the case of dispersion and by the use of surfactants. Nonaqueous vehicles such a cottonseed oil, sesame oil, olive oil, soybean oil, corn oil, sunflower oil, or peanut oil and esters, such as isopropyl myristate, may also be used as solvent systems for compound compositions.

Additionally, various additives which enhance the stability, sterility, and isotonicity of the compositions, including antimicrobial preservatives, antioxidants, chelating agents, and buffers, may be added. Prevention of the action of microorganisms may be ensured by various antibacterial and antifungal agents, for example, parabens, chlorobutanol, phenol, sorbic acid, and the like. In many cases, it will be desirable to include isotonic agents, for example, sugars, sodium chloride, and the like. Prolonged absorption of the injectable pharmaceutical form may be brought about by the use of agents delaying absorption, for example, aluminum monostearate and gelatin. According to the present invention, however, any vehicle, diluent, or additive used would have to be compatible with the compounds.

Sterile injectable solutions may be prepared by incorporating the compounds utilized in practicing the present invention in the required amount of the appropriate solvent with various amounts of the other ingredients, as desired.

A pharmacological formulation of the present invention, e.g., which may comprise a therapeutic compound or polypeptide of the present invention, may be administered to the patient in an injectable formulation containing any compatible carrier, such as various vehicles, adjuvants, additives, and diluents; or the compounds utilized in the present invention may be administered parenterally to the patient in the form of slow-release subcutaneous implants or targeted delivery systems such as monoclonal antibodies, iontophoretic, polymer matrices, liposomes, and microspheres.

A pharmacological formulation of the compound and composition which may comprise a polypeptide utilized in the present invention may be administered orally to the patient. Conventional methods such as administering the compounds in tablets, suspensions, solutions, emulsions, capsules, powders, syrups and the like are usable. Known techniques, which deliver the compound orally or intravenously and retain the biological activity, are preferred.

In one embodiment, a formulation of the present invention may be administered initially, and thereafter maintained by further administration. For instance, a formulation of the invention may be administered in one type of composition and thereafter further administered in a different or the same type of composition. For example, a formulation of the invention may be administered by intravenous injection to bring blood levels to a suitable level. The patient's levels are then maintained by an oral dosage form, although other forms of administration, dependent upon the patient's condition, may be used. In the instance of a vaccine composition, the vaccine may be administered as a single dose, or the vaccine may incorporate set booster doses. For example, booster doses may comprise variants in order to provide protection against multiple clades of HIV. For example, one or more boost immunogens may be from HIV pseudo viruses (PSV s) or derivatives or mutations or a portion thereof.

The quantity to be administered will vary for the patient being treated and whether the administration is for treatment or prevention and will vary from a few micrograms to a few milligrams for an average 70 kg patient, e.g., 5 micrograms to 5 milligrams such as 500 micrograms, or about 100 ng/kg of body weight to 100 mg/kg of body weight per administration and preferably will be from 10 pg/kg to 10 mg/kg per administration. Typically, however, the antigen is present in an amount on, the order of micrograms to milligrams, or, about 0.001 to about 20 wt %, preferably about 0.01 to about 10 wt %, and most preferably about 0.05 to about 5 wt %.

Of course, for any composition to be administered to an animal or human, including the components thereof, and for any particular method of administration, it is preferred to determine therefor: toxicity, such as by determining the lethal dose (LD) and LD₅₀ in a suitable animal model e.g., rodent such as mouse; and, the dosage of the composition(s), concentration of components therein and timing of administering the composition(s), which elicit a suitable immunological response, such as by titrations of sera and analysis thereof for antibodies or antigens, e.g., by ELISA and/or RFFIT analysis. Such determinations do not require undue experimentation from the knowledge of the skilled artisan, this disclosure and the documents cited herein. And, the time for sequential administrations may be ascertained without undue experimentation. For instance, dosages may be readily ascertained by those skilled in the art from this disclosure and the knowledge in the art. Thus, the skilled artisan may readily determine the amount of compound and optional additives, vehicles, and/or carrier in compositions and to be administered in methods of the invention. Typically, an adjuvant or additive is commonly used as 0.001 to 50 wt % solution in phosphate buffered saline, and the active ingredient is present in the order of micrograms to milligrams, such as about 0.0001 to about 5 wt %, preferably about 0.0001 to about 1 wt %, most preferably about 0.0001 to about 0.05 wt % or about 0.001 to about 20 wt %, preferably about 0.01 to about 10 wt %, and most preferably about 0.05 to about 5 wt %. Such determinations do not require undue experimentation from the knowledge of the skilled artisan, this disclosure and the documents cited herein. And, the time for sequential administrations may be ascertained without undue experimentation.

Examples of compositions which may comprise a therapeutic of the invention include liquid preparations for orifice, e.g., oral, nasal, anal, vaginal, peroral, intragastric, mucosal (e.g., perlingual, alveolar, gingival, olfactory or respiratory mucosa) etc., administration such as suspensions, syrups or elixirs; and, preparations for parenteral, subcutaneous, intradermal, intramuscular or intravenous administration (e.g., injectable administration), such as sterile suspensions or emulsions. Such compositions may be in admixture with a suitable carrier, diluent, or excipient such as sterile water, physiological saline, glucose or the like. The compositions may also be lyophilized. The compositions may contain auxiliary substances such as wetting or emulsifying agents, pH buffering agents, gelling or viscosity enhancing additives, preservatives, flavoring agents, colors, and the like, depending upon the route of administration and the preparation desired. Standard texts, such as “REMINGTON'S PHARMACEUTICAL SCIENCE”, 17th edition, 1985, incorporated herein by reference, may be consulted to prepare suitable preparations, without undue experimentation.

Compositions of the invention, are conveniently provided as liquid preparations, e.g., isotonic aqueous solutions, suspensions, emulsions or viscous compositions which may be buffered to a selected pH. If digestive tract absorption is preferred, compositions of the invention may be in the “solid” form of pills, tablets, capsules, caplets and the like, including “solid” preparations which are time-released or which have a liquid filling, e.g., gelatin covered liquid, whereby the gelatin is dissolved in the stomach for delivery to the gut. If nasal or respiratory (mucosal) administration is desired, compositions may be in a form and dispensed by a squeeze spray dispenser, pump dispenser or aerosol dispenser. Aerosols are usually under pressure by means of a hydrocarbon. Pump dispensers may preferably dispense a metered dose or, a dose having a particular particle size.

Compositions of the invention may contain pharmaceutically acceptable flavors and/or colors for rendering them more appealing, especially if they are administered orally. The viscous compositions may be in the form of gels, lotions, ointments, creams and the like (e.g., for transdermal administration) and will typically contain a sufficient amount of a thickening agent so that the viscosity is from about 2500 to 6500 cps, although more viscous compositions, even up to 10,000 cps may be employed. Viscous compositions have a viscosity preferably of 2500 to 5000 cps, since above that range they become more difficult to administer. However, above that range, the compositions may approach solid or gelatin forms, which are then easily administered as a swallowed pill for oral ingestion.

Liquid preparations are normally easier to prepare than gels, other viscous compositions, and solid compositions. Additionally, liquid compositions are somewhat more convenient to administer, especially by injection or orally. Viscous compositions, on the other hand, may be formulated within the appropriate viscosity range to provide longer contact periods with mucosa, such as the lining of the stomach or nasal mucosa.

Obviously, the choice of suitable carriers and other additives will depend on the exact route of administration and the nature of the particular dosage form, e.g., liquid dosage form (e.g., whether the composition is to be formulated into a solution, a suspension, gel or another liquid form), or solid dosage form (e.g., whether the composition is to be formulated into a pill, tablet, capsule, caplet, time release form or liquid-filled form).

Solutions, suspensions and gels, normally contain a major amount of water (preferably purified water) in addition to the active compound. Minor amounts of other ingredients such as pH adjusters (e.g., a base such as NaOH), emulsifiers or dispersing agents, buffering agents, preservatives, wetting agents, jelling agents, (e.g., methylcellulose), colors and/or flavors may also be present. The compositions may be isotonic, i.e., it may have the same osmotic pressure as blood and lacrimal fluid.

The desired isotonicity of the compositions of this invention may be accomplished using sodium chloride, or other pharmaceutically acceptable agents such as dextrose, boric acid, sodium tartrate, propylene glycol or other inorganic or organic solutes. Sodium chloride is preferred particularly for buffers containing sodium ions.

Viscosity of the compositions may be maintained at the selected level using a pharmaceutically acceptable thickening agent. Methylcellulose is preferred because it is readily and economically available and is easy to work with. Other suitable thickening agents include, for example, xanthan gum, carboxymethyl cellulose, hydroxypropyl cellulose, carbomer, and the like. The preferred concentration of the thickener will depend upon the agent selected. The important point is to use an amount that will achieve the selected viscosity. Viscous compositions are normally prepared from solutions by the addition of such thickening agents.

A pharmaceutically acceptable preservative may be employed to increase the shelf-life of the compositions. Benzyl alcohol may be suitable, although a variety of preservatives including, for example, parabens, thimerosal, chlorobutanol, or benzalkonium chloride may also be employed. A suitable concentration of the preservative will be from 0.02% to 2% based on the total weight although there may be appreciable variation depending upon the agent selected.

Those skilled in the art will recognize that the components of the compositions should be selected to be chemically inert with respect to the active compound. This will present no problem to those skilled in chemical and pharmaceutical principles, or problems may be readily avoided by reference to standard texts or by simple experiments (not involving undue experimentation), from this disclosure and the documents cited herein.

It is generally envisaged that compounds and compositions of the invention will be administered by injection, as such compounds are to elicit anti-HIV antibodies, and the skilled artisan may, from this disclosure and the knowledge in the art, formulate compounds and compositions identified by herein methods for administration by injection and administer such compounds and compositions by injection.

The inventive compositions of this invention are prepared by mixing the ingredients following generally accepted procedures. For example the selected components may be simply mixed in a blender, or other standard device to produce a concentrated mixture which may then be adjusted to the final concentration and viscosity by the addition of water or thickening agent and possibly a buffer to control pH or an additional solute to control tonicity. Generally the pH may be from about 3 to 7.5. Compositions may be administered in dosages and by techniques well known to those skilled in the medical arts taking into consideration such factors as the age, sex, weight, and condition of the particular patient, and the composition form used for administration (e.g., solid vs. liquid). Dosages for humans or other mammals may be determined without undue experimentation by the skilled artisan, from this disclosure, the documents cited herein, and the knowledge in the art.

Suitable regimes for initial administration and further doses or for sequential administrations also are variable, may include an initial administration followed by subsequent administrations; but nonetheless, may be ascertained by the skilled artisan, from this disclosure, the documents cited herein, and the knowledge in the art.

Although the present invention and its advantages have been described in detail, it should be understood that various changes, substitutions and alterations can be made herein without departing from the spirit and scope of the invention as defined in the appended claims.

The present invention will be further illustrated in the following Examples which are given for illustration purposes only and are not intended to limit the invention in any way.

### EXAMPLES

### Example 1: Human Immunoglobulin Repertoire-Guided Immunogen Design Targeting HIV V2-Apex Broadly Neutralizing Antibody Precursors

Broadly neutralizing antibodies (bnAbs) to the HIV envelope (Env) V2-apex region are important leads for HIV vaccine design. Most V2-apex bnAbs engage Env with an uncommonly long heavy chain complementarity-determining region 3 (HCDR3), suggesting that rarity of bnAb precursors poses a challenge for vaccine priming. Applicants created precursor sequence definitions for V2-apex HCDR3-dependent bnAbs and searched for related precursors in human antibody heavy chain ultradeep sequencing data from 14 HIV-unexposed donors. Applicants found potential precursors in a majority of donors for only two long-HCDR3 V2-apex bnAbs, PCT64 and PG9, identifying these bnAbs as priority vaccine targets. Applicants then engineered ApexGT Env trimers that bind inferred germlines for PCT64 and PG9 and have higher affinities for bnAbs; determined cryo-EM structures of ApexGT trimers bound to inferred-germline and bnAb forms of PCT64 and PG9; and developed an mRNA-encoded cell-surface ApexGT trimer. These methods and immunogens have promise to assist HIV vaccine development.

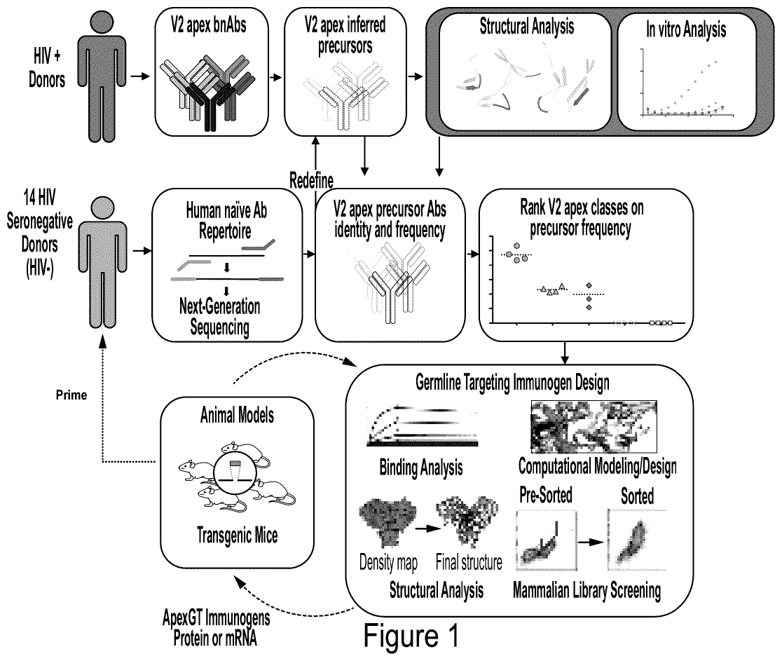

In this study (summarized in FIG. 1), Applicants used structural modeling, analysis of VDJ recombination of known V2-apex bnAbs, and bioinformatic analysis of ultra-deep next-generation sequencing (NGS) of human BCR heavy chains from 14 HIV-unexposed donors to assess the relative frequencies of potential precursors. Applicants' analysis revealed major differences in potential precursor frequencies, with two classes, PCT64 and PG9, having the highest and most consistent frequencies among donors. Potential precursors for these two bnAb classes, whether inferred-germline (iGL) or least mutated common ancestor (LMCA) precursors, had no detectable binding to native-like trimers from HIV isolate BG505. Applicants then engineered BG505-based trimer immunogens (ApexGT immunogens) that bind to inferred germlines for PCT64 and PG9, and Applicants characterized these new immunogens by cryo-electron microscopy. Finally, Applicants developed mRNA-encoded membrane-bound ApexGT trimer vaccine candidates. In a companion manuscript, immunogenicity studies are reported for protein and mRNA-encoded ApexGT immunogens in PCT64 precursor knock-in mouse models under conditions of very low precursor frequencies approximating the human physiological range and with BCR sequencing to prove whether or not targeted responses have been primed (Melzi et al., 2022). These results suggest that a germline-targeting strategy may be possible for two classes of HCDR3-dominant V2-apex bnAbs.

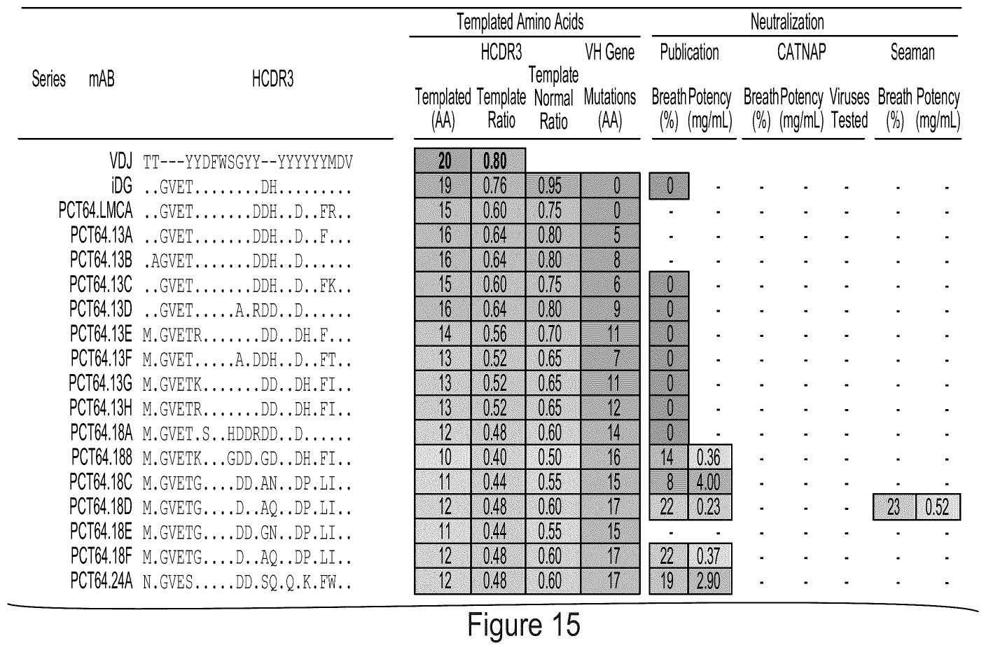

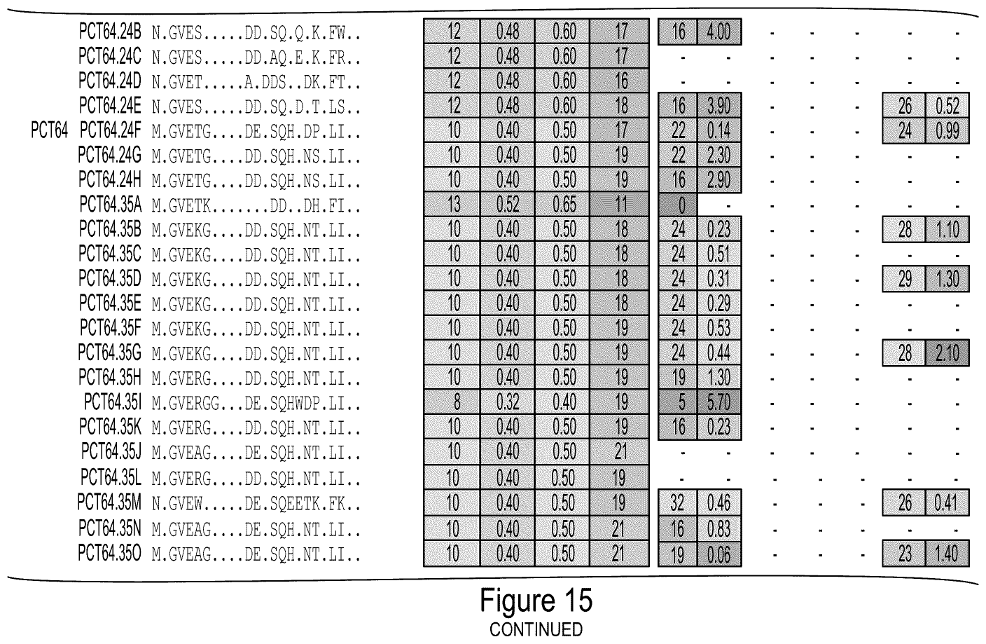

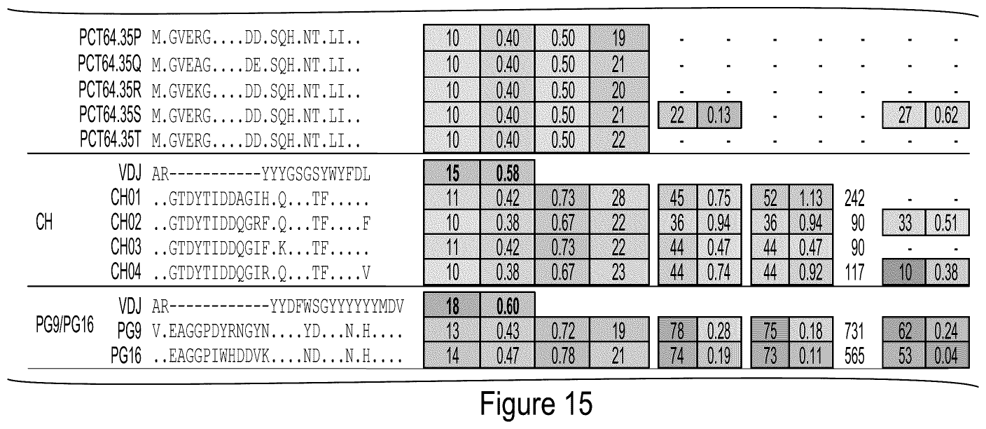

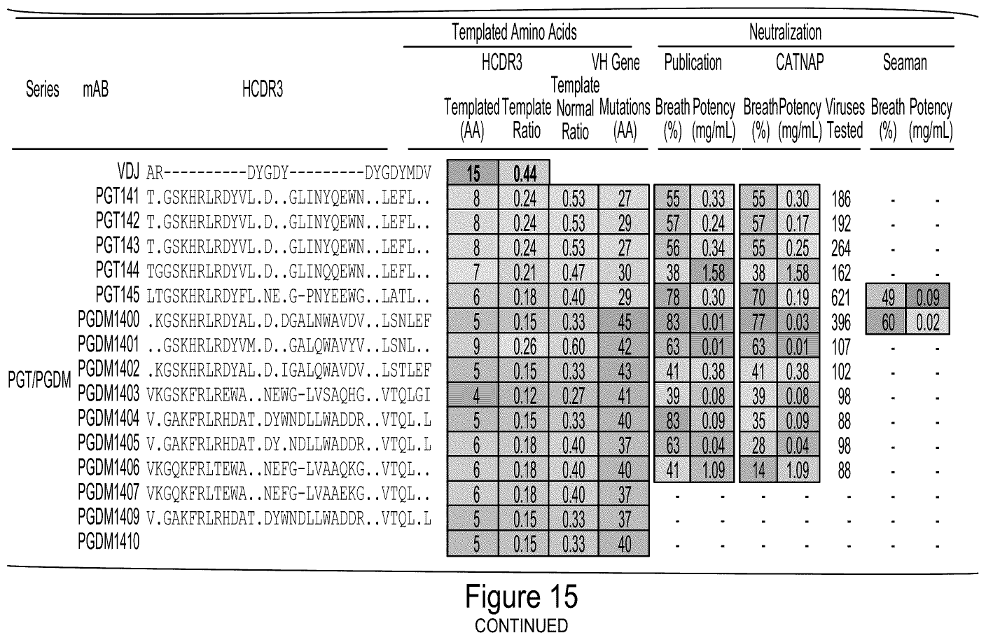

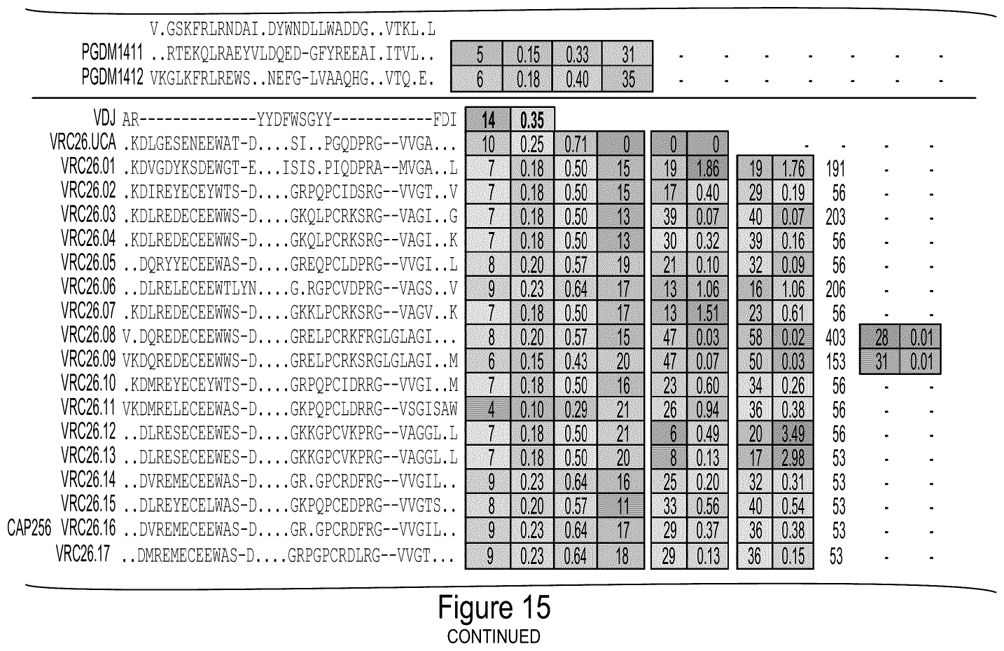

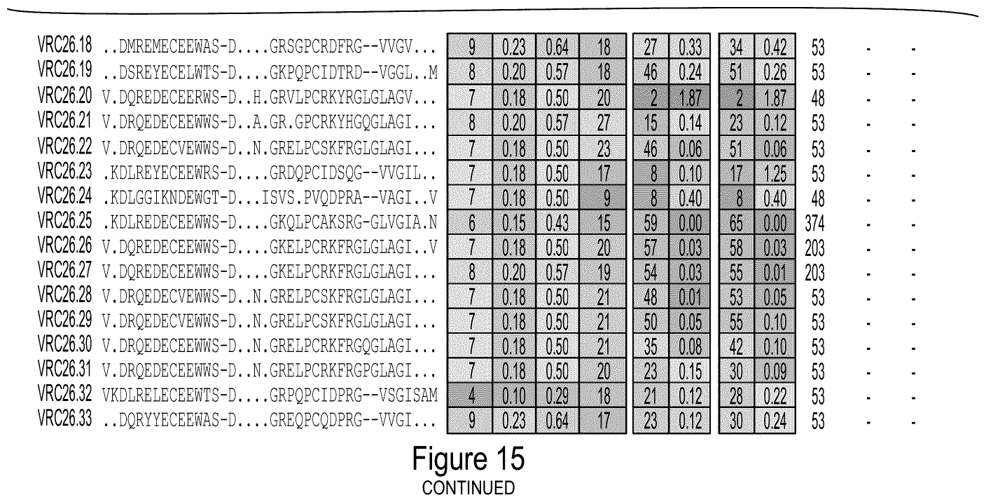

V2-Apex bnAb Precursor Frequencies. Applicants developed precursor definitions for five classes of V2-apex HCDR3-dominant bnAbs. These include the PCT64 lineage (PCT64 donor), CH01-CH04 bnAbs (CH0219 donor), PG9/PG16 bnAbs (IAVI 24 donor), PGT141-145 and PGDM 1400-1414 bnAbs (IAVI 84 donor), and the CAP256-VRC26 lineage (CAP256 donor) (Bonsignori et al., 2011; Doria-Rose et al., 2016; Doria-Rose et al., 2014; Landais et al., 2017; Walker et al., 2011; Walker et al., 2009). These classes range in neutralization breadth at IC 50<50 μg/mL from 27% (PCT64 35S) to 62% (PG9) and in median IC 50 potency from 0.62 μg/mL (PCT64 35S) to 0.005 μg/mL (CAP256-VRC26.09) (FIG. 15). Applicants did not address two V2-apex bnAb classes with Env binding modes not dominated by HCDR3, VRC38 and BG1 (Cale et al., 2017; Freund et al., 2017; Kwong and Mascola, 2018; Wang et al., 2017).

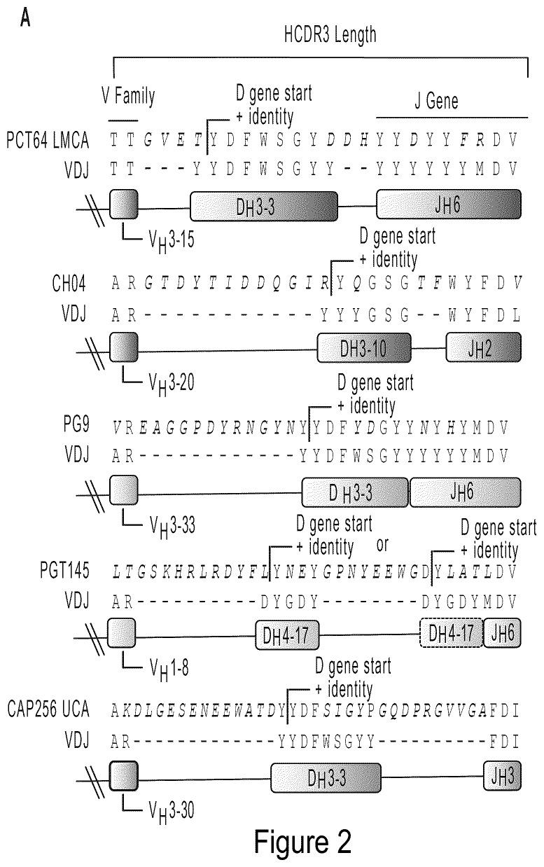

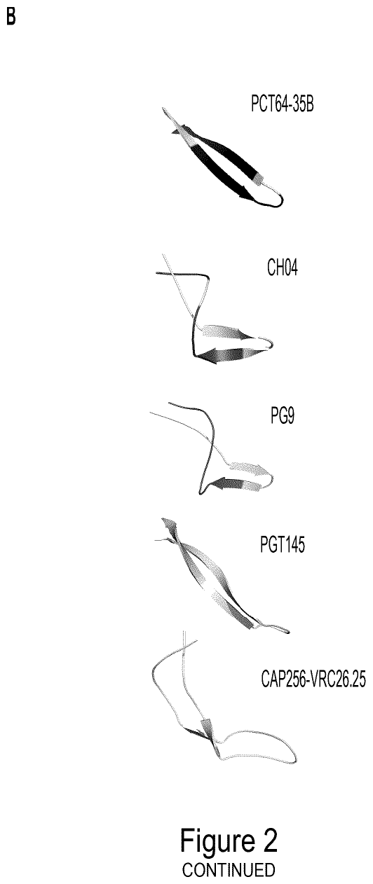

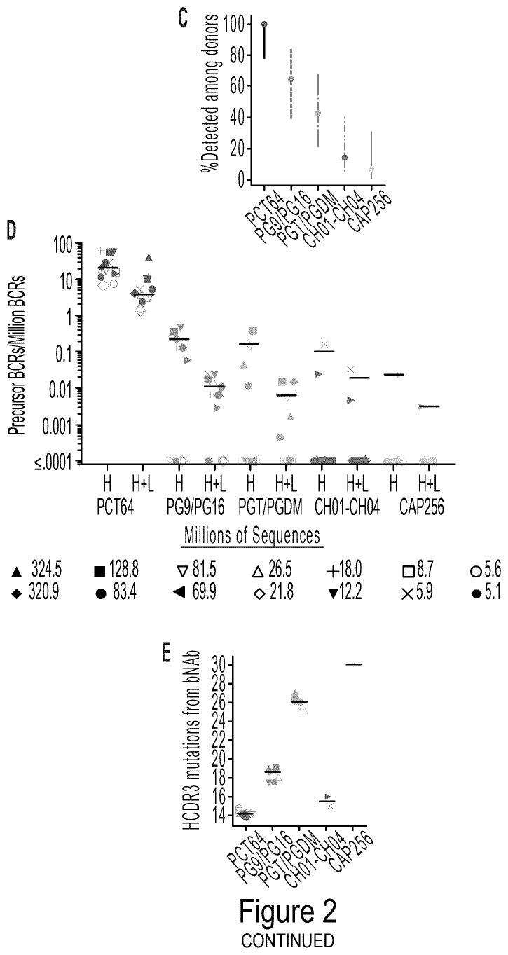

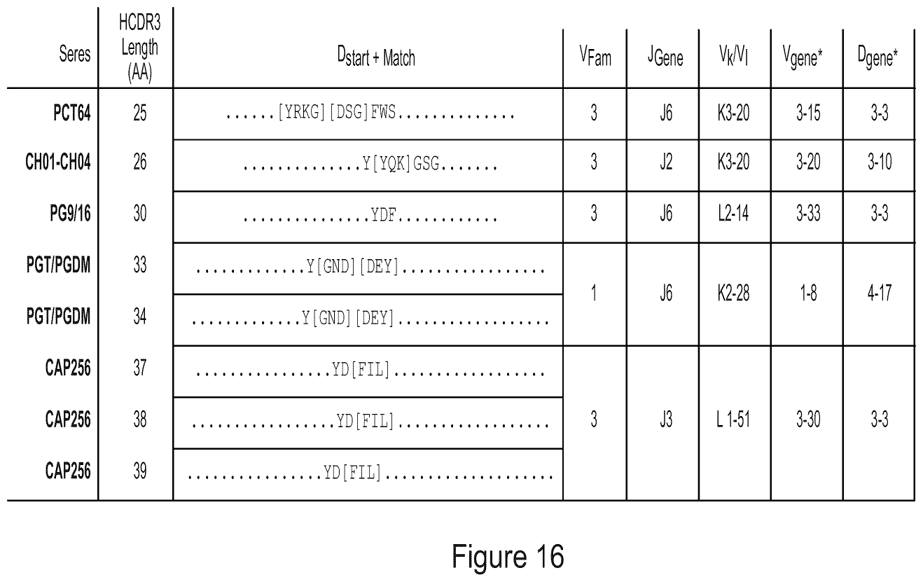

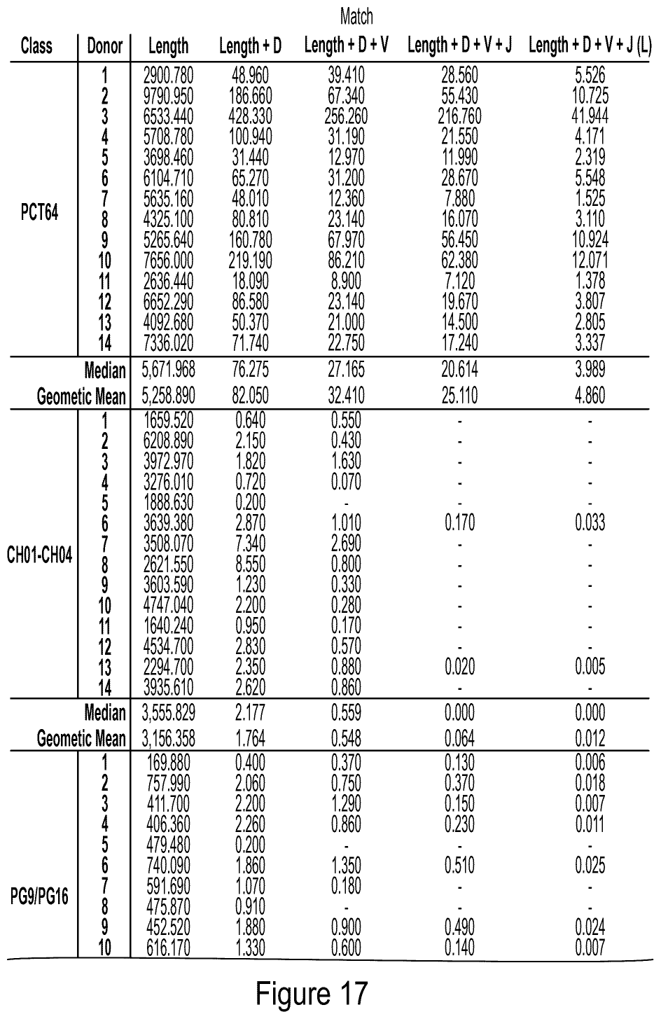

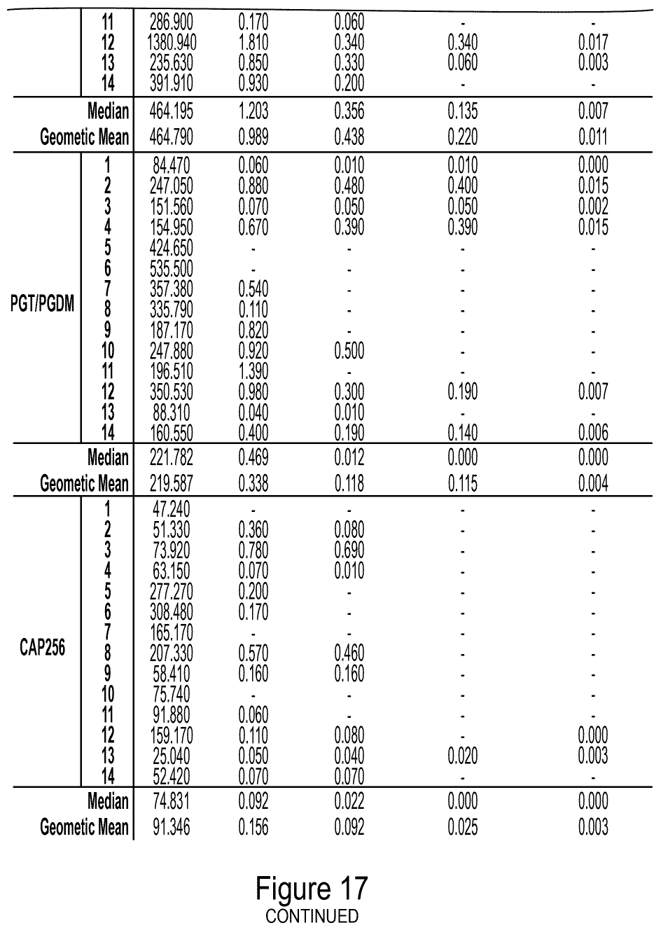

The precursor definitions represented the set of Ab features that Applicants hypothesized were necessary for an Ab to have strong potential to mature into a bnAb, with an Env-binding mode similar to a known bnAb. Applicants hypothesized that a potential bnAb-precursor heavy chain should share at least six characteristics with the bnAb: (i) HCDR3 length; (ii) D gene identity; (iii) D gene reading frame; (iv) D gene position within the HCDR3; (v) V_{H} gene family (e.g. V_{H}3 or V_{H}4); and (vi) JH gene (FIG. 2A-B, FIG. 16) (Steichen et al., 2019). Together these requirements ensured that potential precursors would share all or most germline-templated HCDR 3 amino acids with the original (unknown) precursor. Thus, the major differences between potential and original precursors would be in the non-templated junction regions at the V-D and D-J boundaries. Using these precursor definitions as search criteria, Applicants analyzed the frequency of five V2-apex bnAb classes in previously reported ultra-deep immunoglobulin heavy chain sequencing data from 14 HIV-naïve human donors (FIG. 2C-D, FIG. 17) (Steichen et al., 2019). Applicants found PCT64 HC precursors in all 14 donors (100%) (FIG. 2C) with a median frequency of 20.6 precursors per million BCRs (FIG. 2D). For PG9/PG16, Applicants found HC precursors in 9 of 14 (64%) of donors, with median frequencies of 0.23 precursors per million BCRs among donors with at least one precursor, and 0.135 precursors per million BCRs among all donors, lower than PCT64 by factors of 90 and 150, respectively (FIG. 2C-D, FIG. 17). For the remaining classes, PGT/PGDM, CH01-CH04, and CAP256, Applicants detected HC precursors in 6 (43%), 2 (14%), and 1 (7%) donor(s), respectively (FIG. 2C), resulting in median precursor frequencies of zero computed over all donors in all three cases (FIG. 2D, FIG. 17). For PGT/PGDM, the median frequency of HC precursors among donors with at least one precursor was 0.17 precursors per million BCRs, 121-fold lower than for PCT64. Applicants also calculated precursor frequencies for paired heavy and light chains (‘H+L’ frequencies in FIG. 2D) by scaling the heavy chain precursor frequency by the frequency of the heavy-light pairing obtained from paired NGS sequencing data (DeKosky et al., 2015).

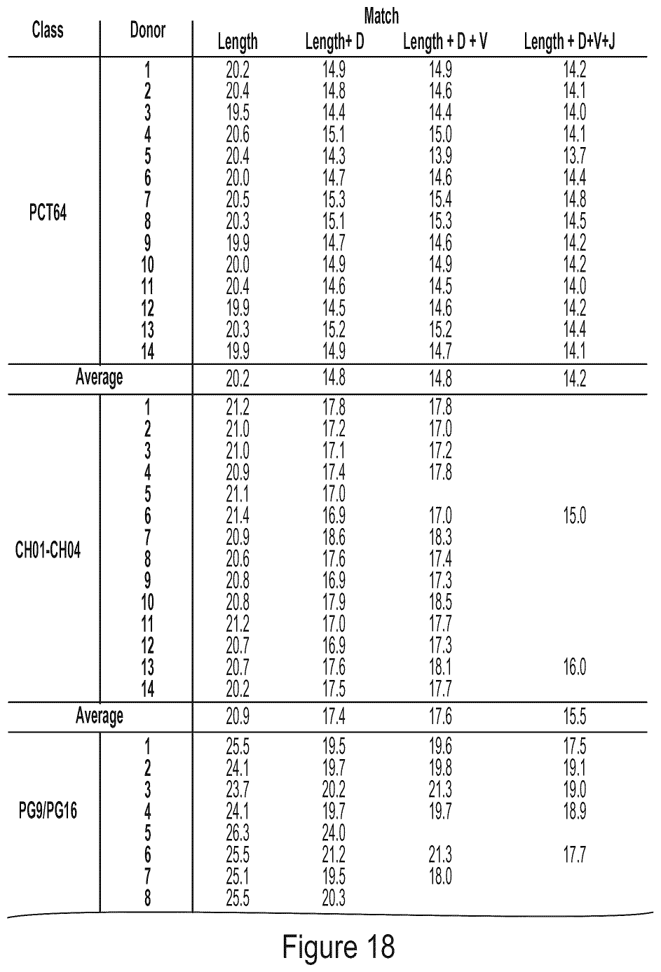

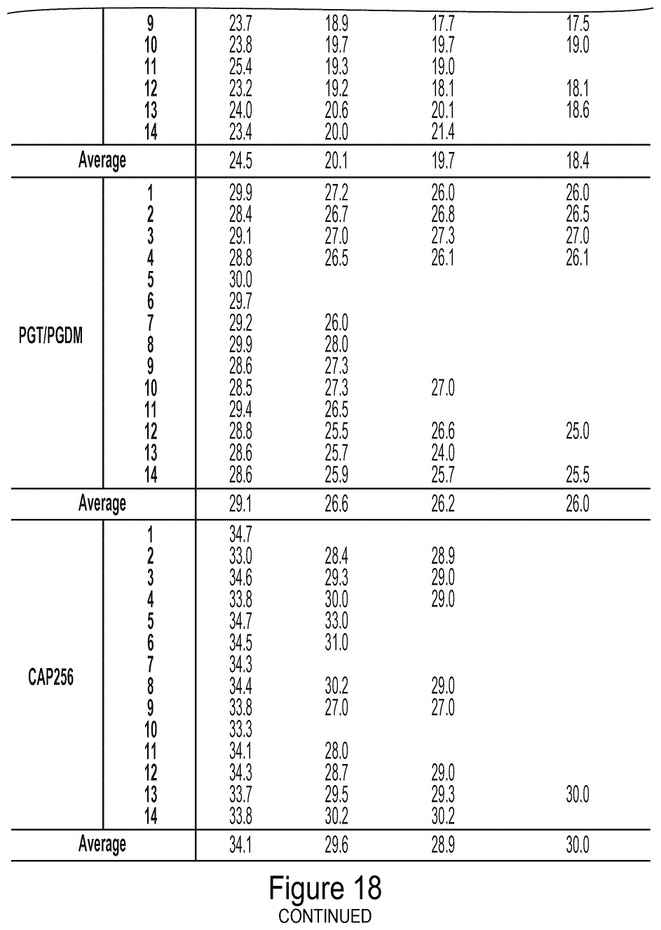

To assess the degree of similarity between potential precursors and known bnAbs, Applicants measured the number of mutations from each potential precursor HCDR3 to a known HCDR3 bnAb sequence (FIG. 2E, FIG. 18). PCT64 precursors had an average of 14.2 of a possible 25 mutations from known PCT64 lineage bnAb sequences, and PG9/PG16 had an average of 18.4 out of a possible 30 mutations from mature PG9 or PG16 (FIG. 2E, FIG. 18). For the PGT/PGDM class, which was detected in a smaller fraction of donors than PCT64 or PG9/PG16 but had a similar precursor frequency among positive donors as PG9/PG16 (FIG. 2C-D), the precursors were a median of 26 mutations from a bnAb in the class, substantially further from a bnAb than PCT64 or PG9/16. Taken together, Applicants' analyses indicated that PGT/PGDM, CH01-CH04, and CAP256 classes are relatively poor vaccine targets, due to their low precursor frequencies, and in some cases high HCDR3 distances to bnAb. In contrast, Applicants' analyses indicated that PCT64 and PG9/16 are more suitable vaccine targets, with PCT64 having the most favorable precursor frequency and HCDR3 distance to bnAb.

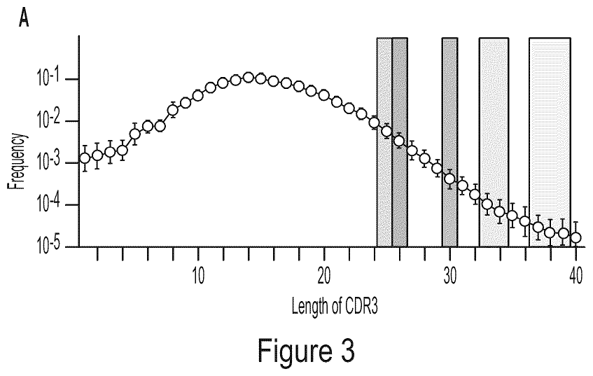

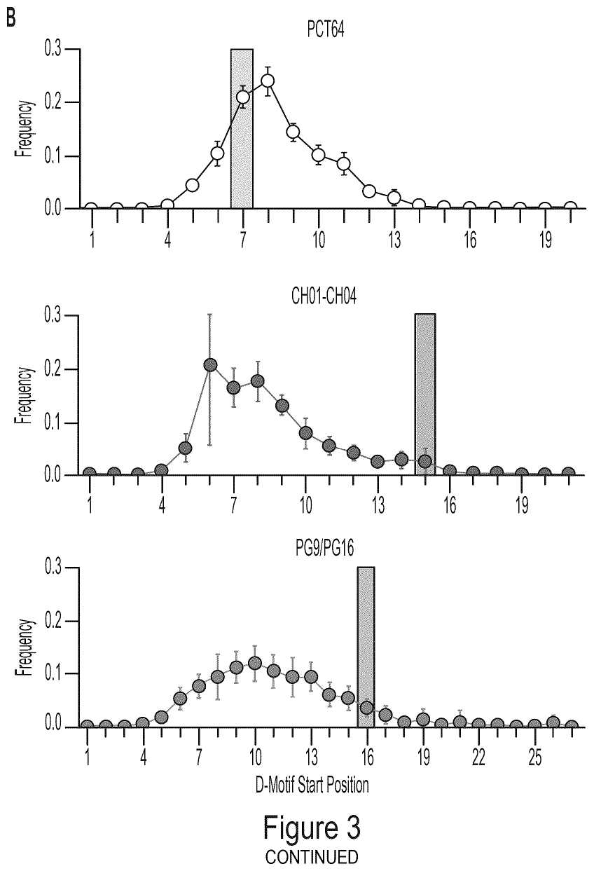

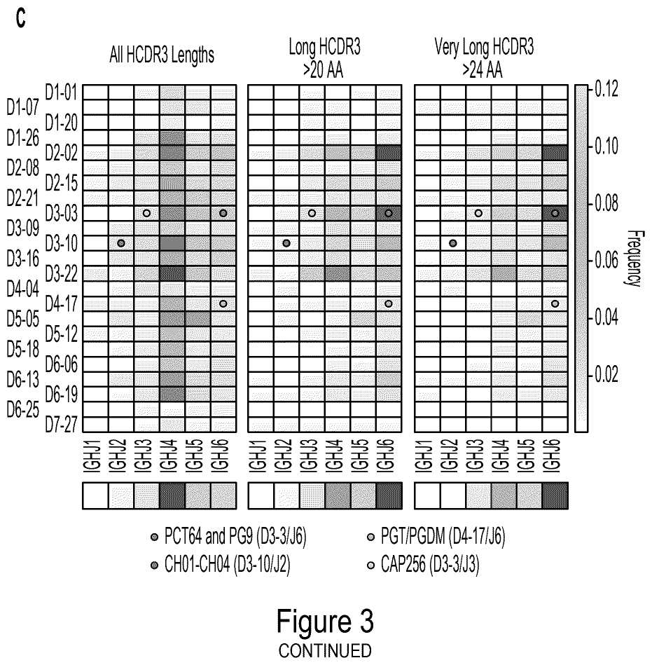

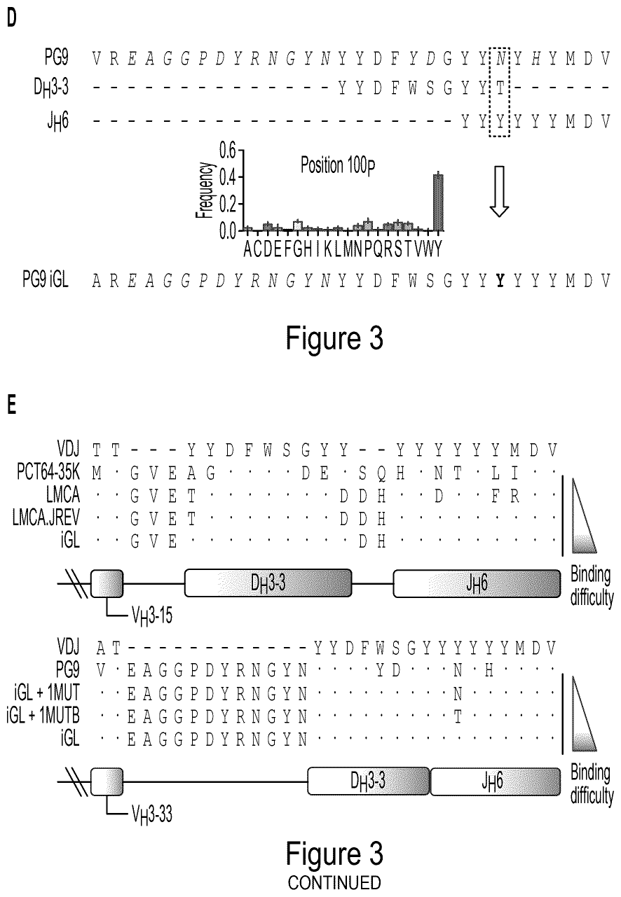

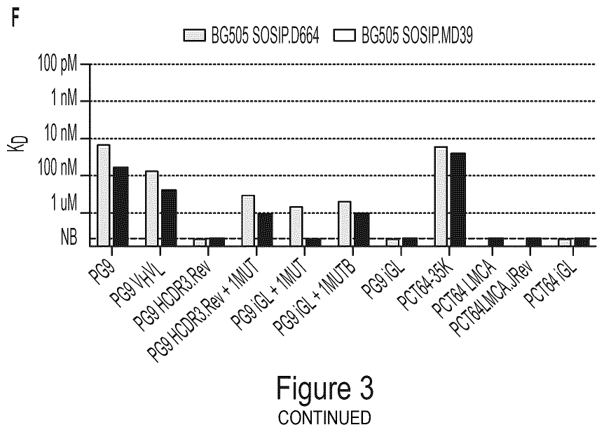

Determinants of Precursor Frequency for V2-apex bnAbs. To understand why precursor frequencies were significantly higher for PCT64 than for the other classes of V2-apex HCDR3-dependent bnAbs, Applicants investigated the impact of several factors (HCDR3 length, V_{H} family, D-gene motif start, and D-J pairing) on precursor frequency in Applicants' ultradeep heavy chain sequencing dataset from 14 HIV-unexposed donors. For PGT/PGDM and CAP256, the primary contributor to a low precursor frequency was the HCDR3 length (FIG. 3A). Applicants calculated a mean HCDR3 length among all donors of 15.5+4.8, in agreement with a previous analysis of 8 of the 14 HIV naïve donors (Briney et al., 2019). HCDR3 lengths of 33 or 34, required for PGT/PGDM precursors, were only found at a frequency of 1.87×10⁻⁴ (187 BCRs per million BCRs), and HCDR3 lengths of 37-39, required for CAP256 precursors, were found at a frequency of 8.66×10⁻⁵ (74 per million B cell sequences). The precursor frequency for PGT/PGDM was also reduced disproportionately compared to the other classes (39-fold versus 2-to 4-fold), owing to PGT/PGDM using the less common V_{H}1 and other classes using the more common V_{H}3 (FIG. 17).

Applicants next investigated the impact on precursor frequency due to the position of the D-gene sequence motif within the HCDR3. For all HCDR3s of length 25, required for PCT64, Applicants calculated the frequency of the start position for the D-gene sequence motif “YDFWS” (SEQ ID NO:235) (FIG. 3B). In PCT64 precursors, the “YDFWS” (SEQ ID NO:235) motif starts at position 7, which was the second highest positional frequency given the D gene motif. For all HCDR3s of length 30, required for PG9/16 precursors, Applicants calculated the frequency of the start position for the D-gene sequence “YDF” (FIG. 3B). In PG9 and PG16, the “YDF” motif starts at position 16, whereas the most common start position for that motif is 10. The sub-optimal motif start position reduced the PG9/16 precursor frequency by a factor of 3.3. For CH01-CH04, which use “YYGS” (SEQ ID NO:236) at position 15, the positional frequency for the D-gene motif was 8.3-fold lower than the ideal start position of 6. For PGT/PGDM and CAP256, Applicants had too few sequences of the required length to produce a frequency for this metric.

F Finally, Applicants determined the D-J pairing frequencies for (i) all HCDR3s; (ii) long HCDR3s (>20 amino acids); and (iii) very long HCDR3s (>24 amino acids) (FIG. 3C). PCT64 and PG9/PG16 both use DH3-3 and JH6, which are the most common D-J pairing for very long HCDR3s. D-J pairings for CH01-CH04 (D_{H}3-10/J_{H}2), PGT/PGDM (D_{H}4-17/J_{H}6), and CAP256 (D_{H}3-3/J_{H}3) were all found at low frequencies, especially among the long HCDR3 and very-long HCDR3 subsets.

Taken together, these analyses indicated that the biggest factor contributing to diminished precursor frequency was HCDR3 length, especially for PGT145/PGDM and CAP256 classes. While CH01-CH04 has a relatively shorter HCDR3 length, the D-gene placement within the HCDR3, along with an infrequently used JH2 gene, were the primary contributors to low precursor frequency. Use of the J H3 gene further reduced the CAP256 precursor frequency.

Need for Germline-targeting immunogens. To determine whether engineering of a germline-targeting immunogen with affinity for PCT64 and PG9/16 precursors might be needed, Applicants assessed the binding of mature and iGL variants of PCT64 and PG9 to native-like trimers BG505 SOSIP (Julien et al., 2013; Lyumkis et al., 2013; Sanders et al., 2013) and BG505 MD39 (Steichen et al., 2016). Previously reported PG9 iGL sequences used either asparagine (from the bnAb) or threonine (from the germline D gene) at position 100p (kabat numbering) (Medina-Ramirez et al., 2017; Sliepen et al., 2015), but Applicants' analysis of PG9-like junctions in the NGS data revealed that tyrosine was substantially more common than any other amino acid at 100p (FIG. 3D). Therefore Applicants employed an updated PG9 iGL with tyrosine at 100p that likely better represents PG9-like precursors (FIG. 3D-E). The iGL variants for PCT64 were the least mutated common ancestor (PCT64 LMCA), a more reverted version of the LMCA in which the J H gene somatic mutations were reverted to germline (PCT64 LMCA.JREV), and an inferred germline variant in which all unambiguous D and J gene templated mutations were reverted to germline (PCT64 iGL) (FIG. 3E). Applicants found that mature PCT64 bnAbs could bind BG505 native-like trimers, but the PCT64 LMCA, LMCA.JREV and iGL antibodies had no detectable binding up to 5 uM (FIG. 3F). Similarly, partially-reverted PG9 variants (FIG. 3E) could bind the native-like trimers, but PG9 iGL had no detectable binding (FIG. 3F). The lack of detectable binding between BG505 native-like trimers and PCT64 LMCA, PCT64 iGL and PG9 iGL indicated the need to engineer V2-apex germline-targeting (ApexGT) trimers with appreciable affinity for precursors.

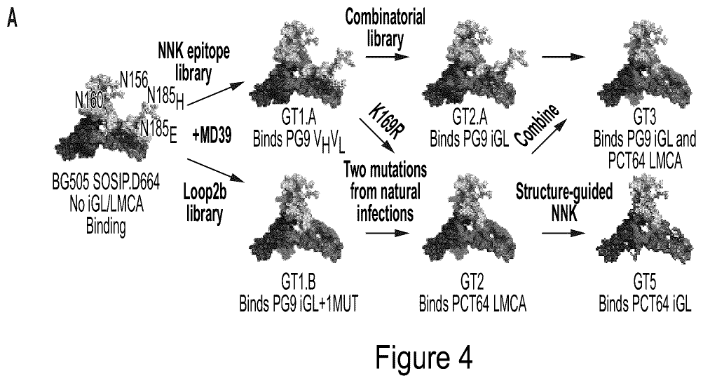

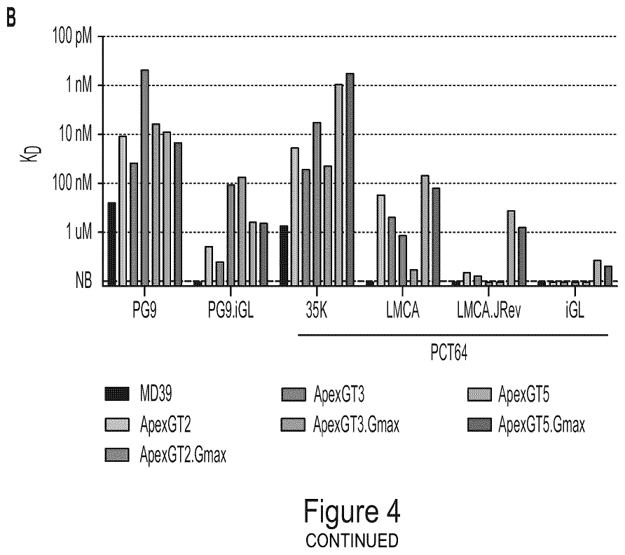

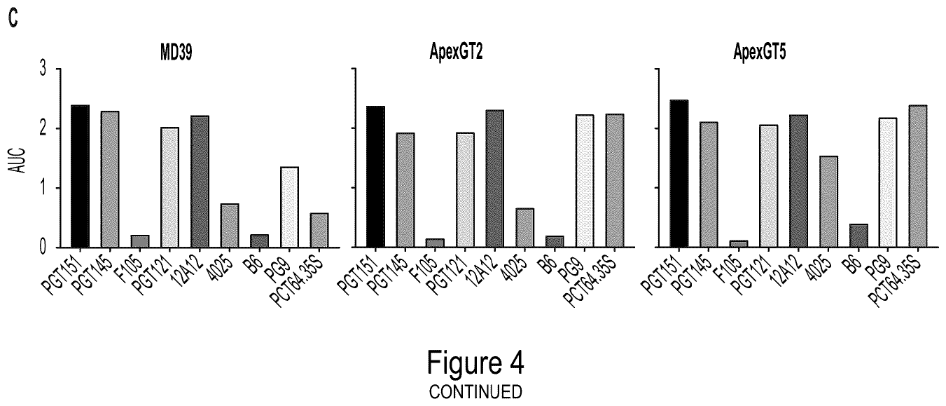

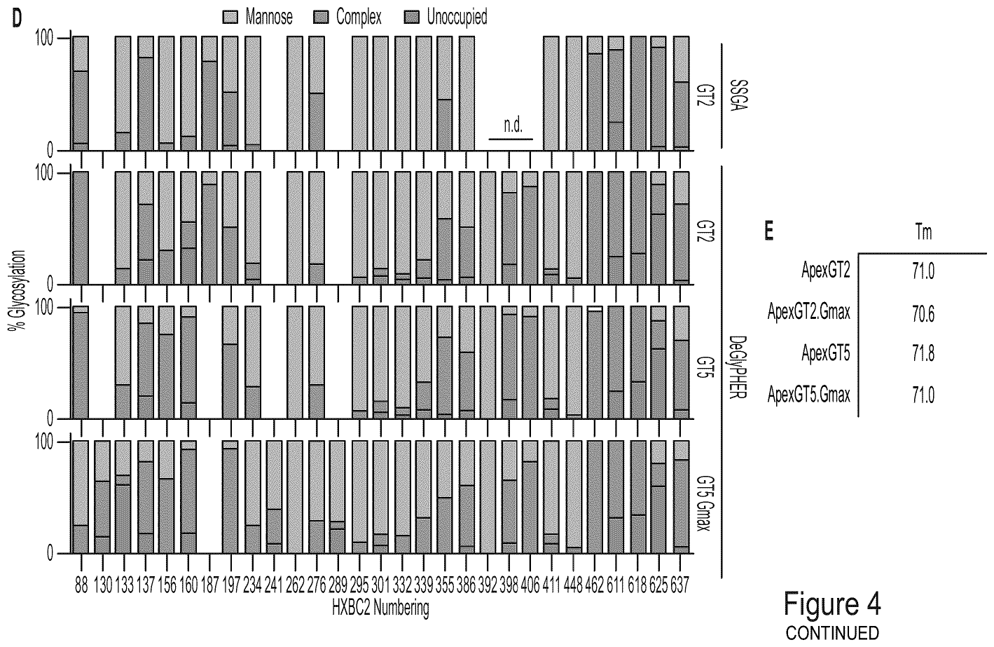

V2-apex Germline-Targeting Immunogen Design. A germline-targeting priming immunogen should have both (i) appreciable affinity for bnAb precursors, to enable precursor B cell activation and competitive fitness in germinal centers (GCs), and (ii) higher affinity for corresponding bnAbs, to provide an affinity gradient that may foster selection of mutations conducive for bnAb development (Jardine et al., 2013). To develop V2-apex germline-targeting immunogens with appreciable affinity for PCT64 and PG9 precursors and higher affinity for the corresponding bnAbs, Applicants used mammalian cell surface display-directed evolution with a BG505 SOSIP.D664 native-like trimer platform. Because PG9 iGL, PCT64 LMCA and PCT64 iGL had no detectable affinity for either BG505 SOSIP.D664 or BG505 MD39 (FIG. 3E-F), Applicants used a “bootstrapping” approach (Steichen et al., 2016) starting with partially reverted PG9 and PG16 variants for screening against multiple library types (FIG. 4A).

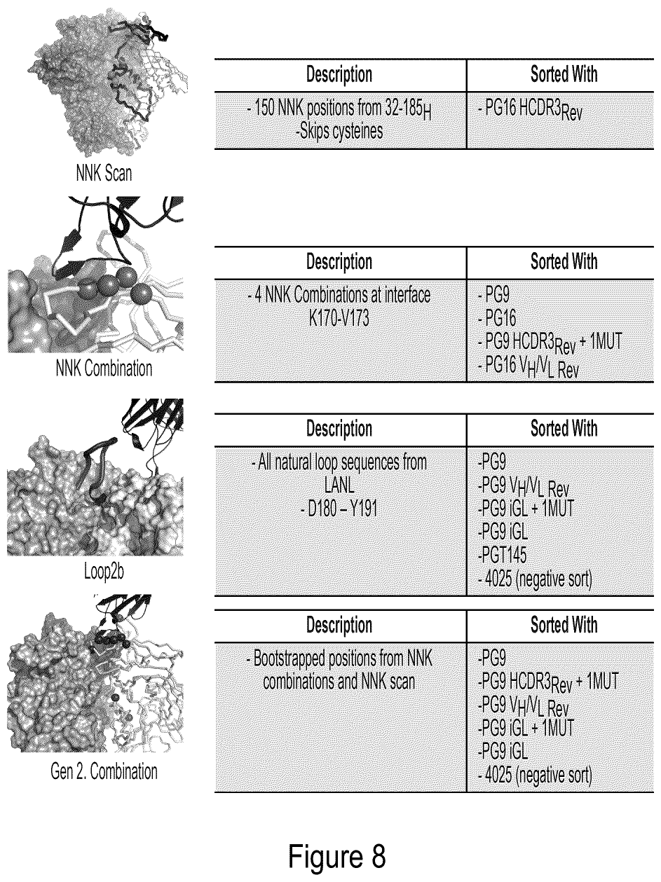

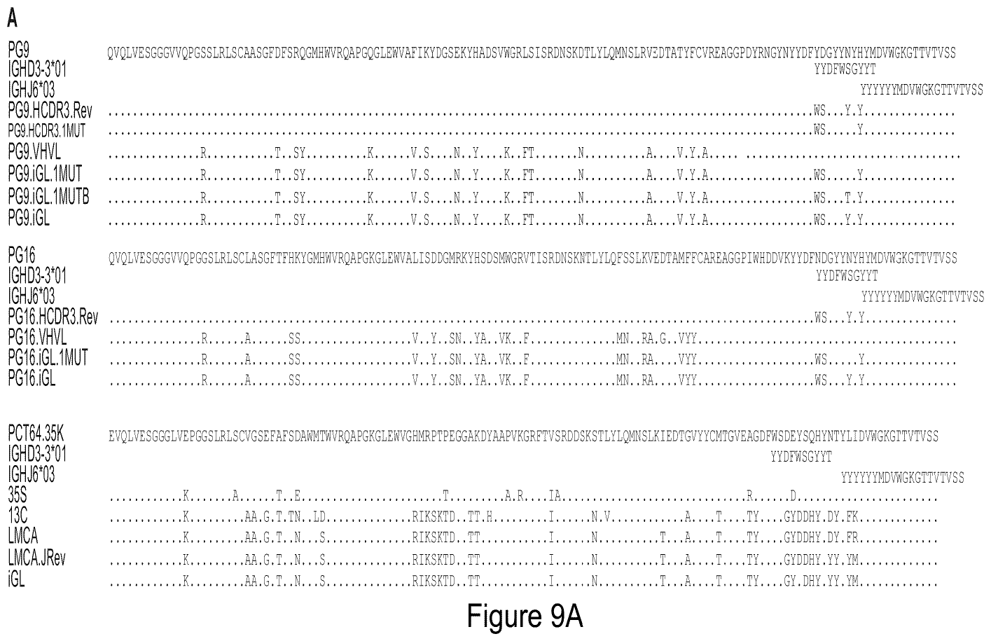

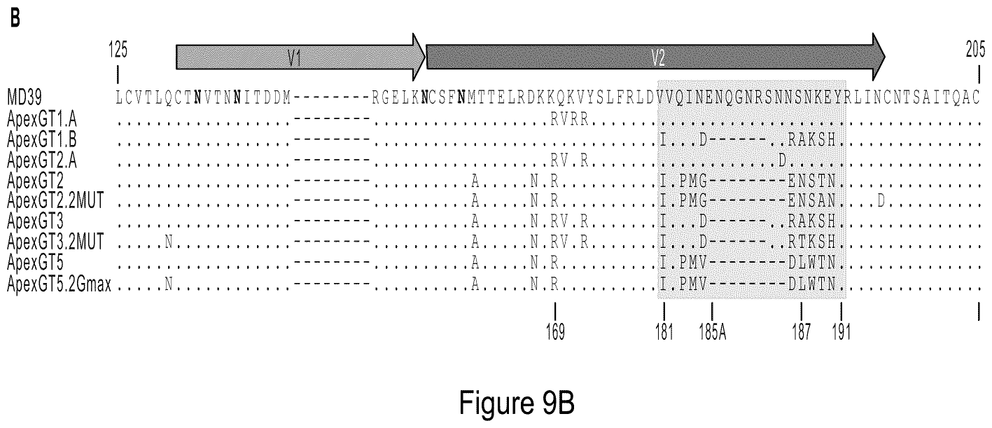

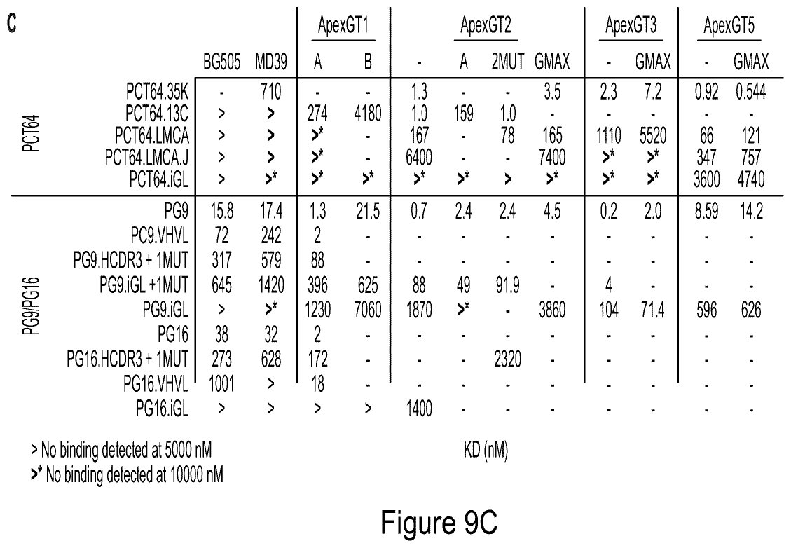

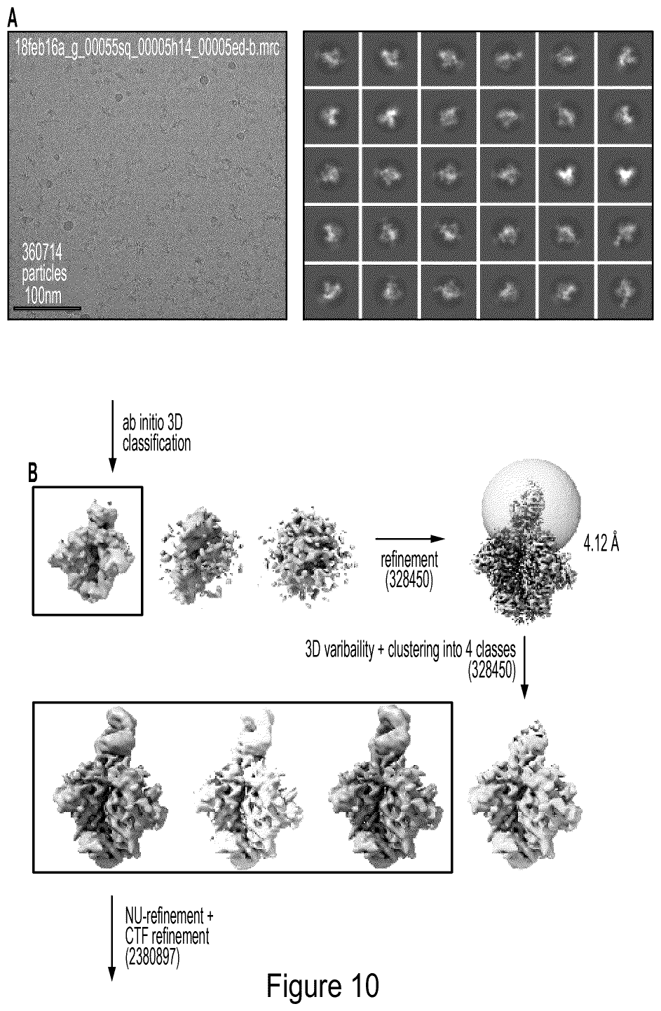

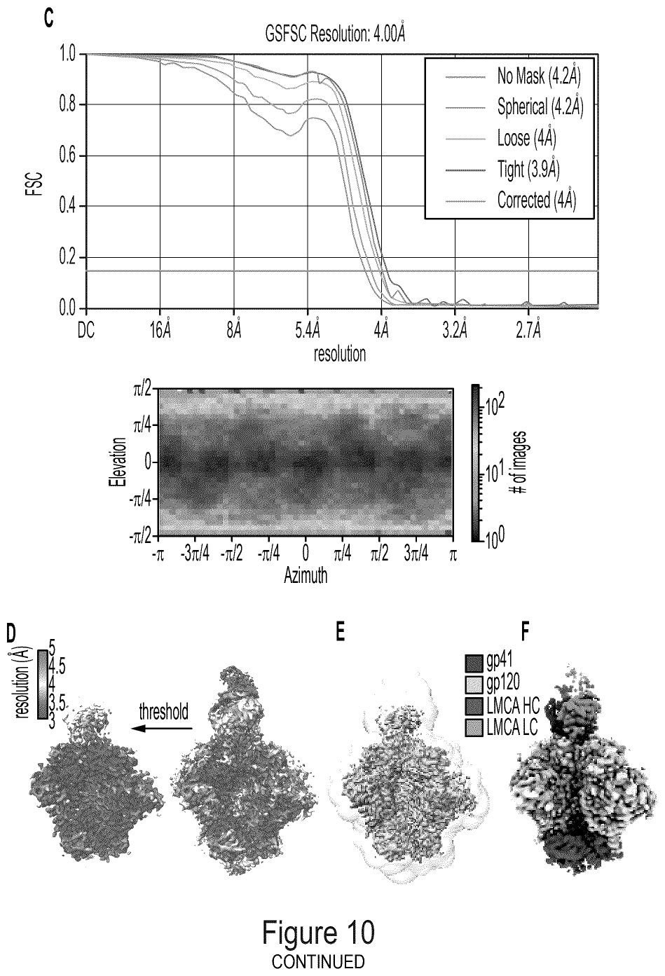

Applicants generated two NNK-type libraries (degenerate codons that encode all 20 amino acids with 32 codons), an NNK-scanning library and an NNK-combinatorial library. The NNK-scanning library contained sequential NNK codons at positions 32-185H (HXBC2 numbering) as described previously (Kulp et al., 2017; Steichen et al., 2016) (FIG. 8). The NNK-combinatorial library contained NNK combinations at contact residues between PG9 and BG505 SOSIP.D664, K 170-V173. These contact residues were identified by docking PG9 into the BG505 SOSIP crystal structure using the modeling suite Rosetta (Das and Baker, 2008). The NNK-scanning library showed enrichment after sorting with a fully reverted PG16 HCDR3 but a mature V_{H}/V_{L} (PG16-HCDR3_{Rev}). The NNK-combinatorial library showed enrichment after sorting with mature and partially reverted variants of PG9/PG16 (FIG. 9A). The enriched mutations for both libraries were combined and expressed on the background of MD39, a variant of BG505 SOSIP.D664 with improved stability, expression, and antigenicity (Steichen et al. 2016), yielding ApexGT1.A, a trimer with enhanced affinity to PG9 V_{H}V_{L} and PG9 iGL+1MUT (FIG. 4A and FIG. 9B-C). A second-generation combinatorial library combining enriched mutations from the first two libraries was sorted with fully and partially reverted PG9 and negatively sorted against the V3-directed non-neutralizing Ab 4025, to enrich for sequences of well-formed, “closed” trimers. This yielded ApexGT2A, a trimer with enhanced affinity to PG9 germline variants and with detectable affinity for fully reverted PG9 iGL (FIG. 8, FIG. 9C).

Applicants also pursued a library of Loop2b (D180-Y 191) because it was implicated as a major contact with PG9 in the Rosetta model (FIG. 8). To generate the Loop2b library, Applicants considered all natural sequences gathered from the Los Alamos National Laboratory (LANL) HIV Sequence Database (www.hiv.lanl.gov/). This search yielded 1.1×10⁴ unique Loop2b sequences which Applicants synthesized and cloned onto BG505 SOSIP.D664 to create a library for mammalian display. Applicants sorted this library using mature, partially reverted, and fully reverted PG9 iGL fragment antigen-binding domains (Fabs), and again used negative selection by 4025 to enrich for “closed” trimers. These efforts yielded ApexGT1B, a trimer that bound PG9 iGL+1M UT and enhanced affinity for mature PG9 (FIG. 9C).

To improve binding to PCT64 bnAbs and rescue binding to PCT64 LMCA, Applicants combined the enriched loop sequence from ApexGT1.B, the K 169R mutation from ApexGT1A, and two naturally occurring mutations from the PCT64 donor, M 161A and D167N (Landais et al., 2017). The resulting construct, ApexGT2, bound to PCT64 LMCA with a monovalent affinity of 167 nM, and had 25-fold and 39-fold enhanced affinity for PG9 and PCT64.35K, respectively, compared to MD39 (FIGS. 4B and S2C). By ELISA antigenic profiling, Applicants found that ApexGT2 had a similar profile to MD39 (FIG. 4C). By glycan composition analysis, ApexGT2 had an overall similar glycan profile to BG505 SOSIP.D664 (Behrens et al., 2016; Cao et al., 2017; Cao et al., 2018) across two separate glycan analysis techniques, single-site glycan analysis (SSGA) (Allen et al., 2021) and DeGlyPHER (Baboo et al., 2021). Position 187 within Loop2b was not glycosylated in MD39 but in ApexGT2 was well occupied and composed mainly of complex type glycans (FIG. 4D). ApexGT2 also showed good thermal stability by DSC, with a Tm of 71°, reduced by 6° from MD39 but still within the range for BG505 SOSIP.D664-based native-like trimers (Kulp, 2017; Sanders et al., 2013)

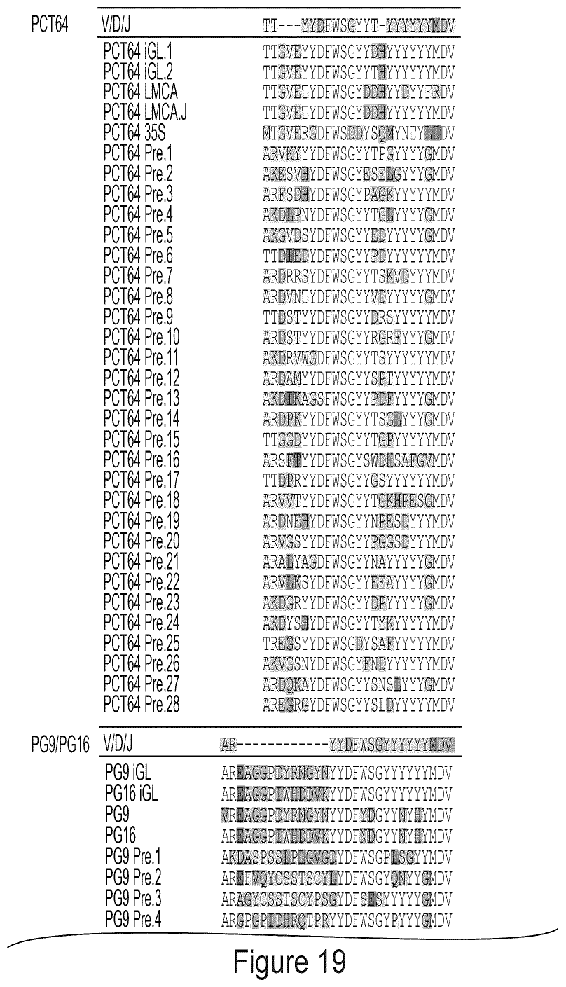

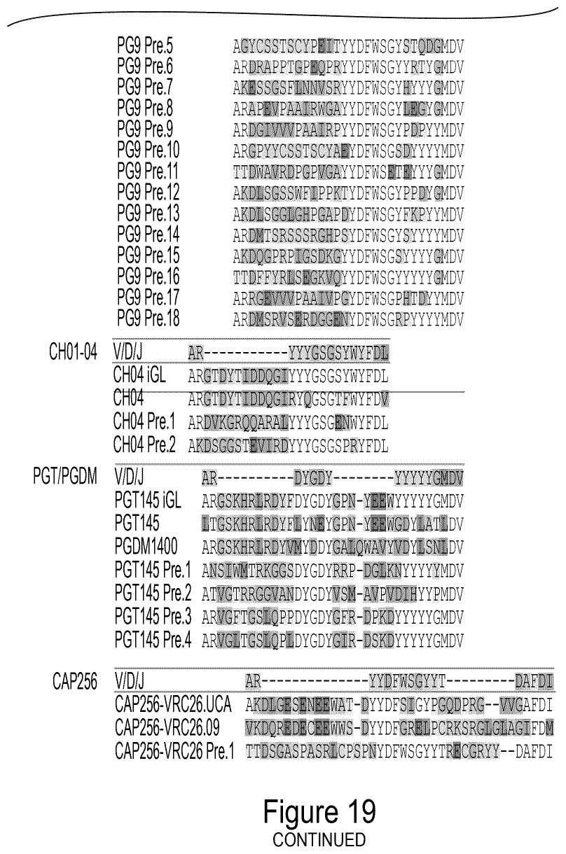

The third-generation trimer, ApexGT3, was a combination of ApexGT2A and ApexGT2. ApexGT3 showed improved PG9 iGL binding, with a K_{D} of 104 nM. However, the affinity for PCT64 LMCA was reduced by a factor of 7 compared to ApexGT2, necessitating the need for further engineering. To improve PCT64 LMCA affinity while maintaining PG9 iGL affinity, Applicants engineered ApexGT2 with three combinatorial-NNK libraries at contact positions identified at the antigen-antibody interface in the ApexGT2-PCT64 LMCA structure (described in the next section). PCT64 LMCA was used as a positive probe to select for mutations that improve binding affinity, while the non-neutralizing CD4-binding-site-directed B6 Fab was used as a negative probe to select for “closed” trimers. Enriched mutations were added to ApexGT2 to yield ApexGT5, Applicants' most advanced GT trimer with the best affinity profile to date. ApexGT5 had K_{D}s of 66 nM and 0.9 nM for PCT64 LMCA and PCT64.35K bnAb, respectively, and K_{D}s of 596 nM and 8.6 nM for PG9 iGL and mature PG9, respectively (FIG. 4B and FIG. 9C). Notably, ApexGT5 was the first of Applicants' GT trimers to acquire measurable affinity for PCT64 iGL, a substantially superior model precursor compared to the LMCA. The K_{D} for PCT64 iGL was 3.6 uM, within the range of affinities capable of stimulating responses from rare precursors using multivalent immunogens (Abbott et al., 2018; Huang et al. 2020; Kato et al., 2020; Wang et al., 2021). The binding affinity of ApexGT5 for PCT64 LMCA.JREV, also superior to LMCA as a model precursor, was 347 nM, 18-fold higher than for ApexGT2 (FIGS. 4B and 8). Thus, ApexGT5 had appreciable affinities for PCT64 and PG9 inferred precursors and higher affinities for the corresponding mature bnAbs. However, Applicants have not detected ApexGT5 affinity to a select set of PCT64 or PG9/PG16 NGS precursors (FIG. 19). The overall antigenic profile for ApexGT5, measured by ELISA, was in good agreement with MD39, except for elevated binding to the V3 non-nAb 4025 (FIG. 4C), and the melting temperature remained high, at 71.8° C. (FIG. 4D), both of which suggested an overall native-like structure albeit with a slightly more exposed V3 loop.

To minimize “holes” in the glycan shield, and to focus antibody responses to the V2-apex epitope, Applicants added the N130 glycosylation site, which was implicated in shaping the PCT64 lineage (Rantalainen et al., 2018), and N241 and N289 glycosylation sites, which are conserved across most HIV isolates but absent from the BG505 isolate (McCoy et al., 2016) (FIG. 9B). Adding these three glycosylation sites to ApexGT2 and ApexGT5, to give ApexGT2.Gmax and ApexGT5.Gmax, respectively, only modestly reduced affinity for PG9 and PCT64 variants tested, and modestly decreased thermostability (FIGS. 4B, 4E, and S2C). Glycosylation of ApexGT5 and ApexGT5.Gmax (FIG. 4D) was broadly similar to BG505 SOSIP except for substantially elevated levels of complex glycans at positions 156, 160, and 197 (Behrens et al., 2016; Cao et al., 2017; Cao et al., 2018).

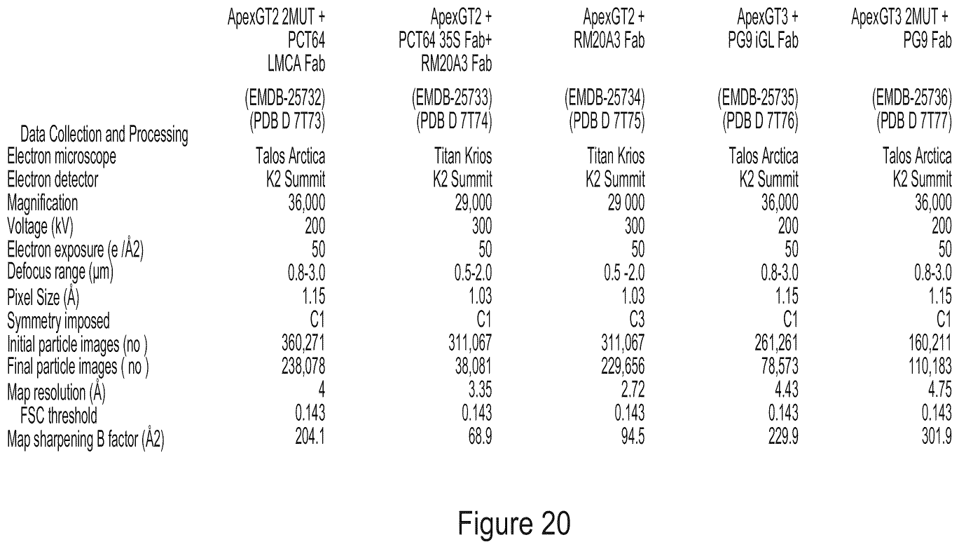

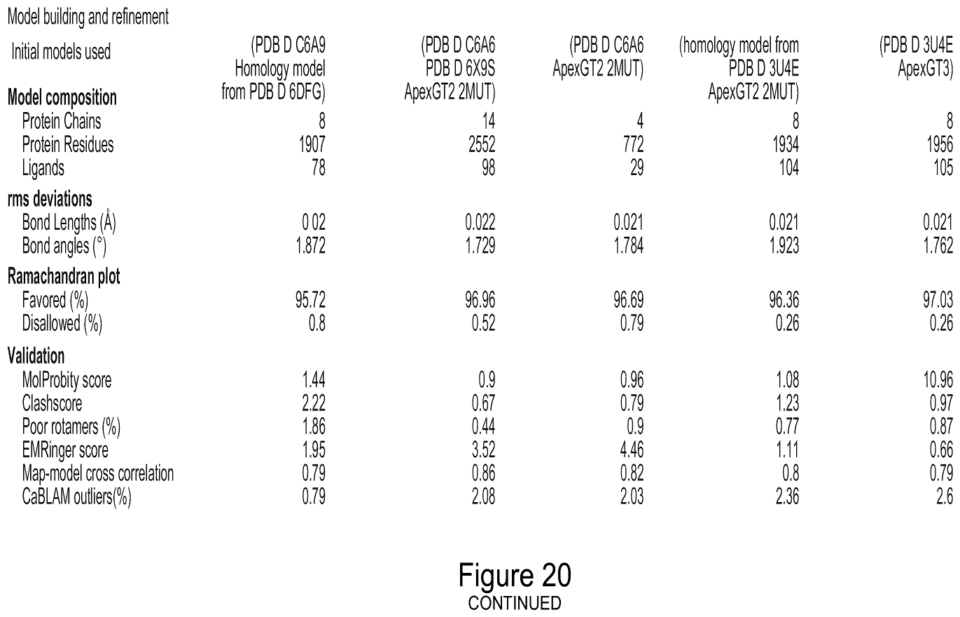

Structure of ApexGT Trimer-Fab Complexes. To gain structural insight into the capacity of ApexGT trimers to bind V2apex precursors with appreciable affinities and to bind mature bnAbs with higher affinities, Applicants determined single-particle cryo-EM structures of ApexGT trimers in complex with the Fabs of both PCT64 LMCA and PG9 iGL as well as their mature broadly neutralizing counterparts, PCT64 35S and PG9. Applicants determined the structure of PCT64 LMCA complexed with a version of ApexGT2 called ApexGT2.2MUT that had the Loop2b glycan at N187 knocked out (T189A) and a N195D mutation that modestly improved the affinity for PCT64 LMCA [K_{D} of 78 nM compared to a K_{D} of 167 nM for ApexGT2 (FIG. 9C)]. Applicants also determined structures for PG9 iGL in complex with ApexGT3, and for mature PG9 in complex with a Q130N variant, ApexGT3.2MUT, that contained a glycosylation site at position 130. A summary of all cryo-EM structures can be found in FIG. 20.

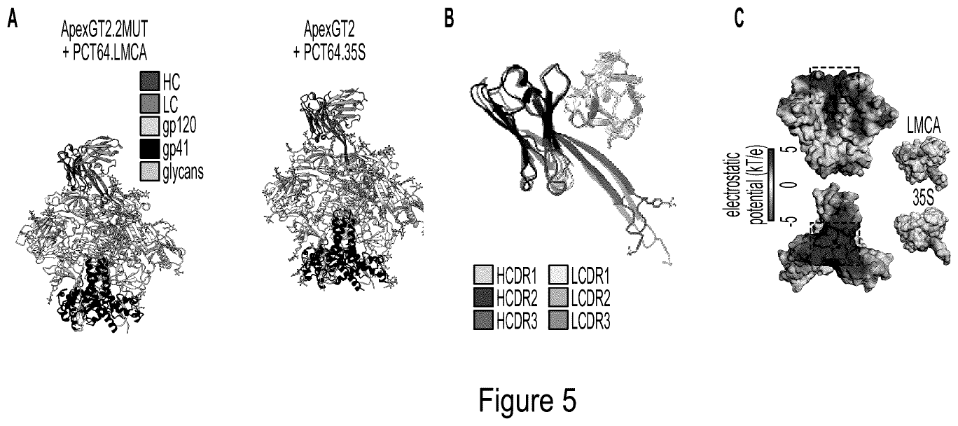

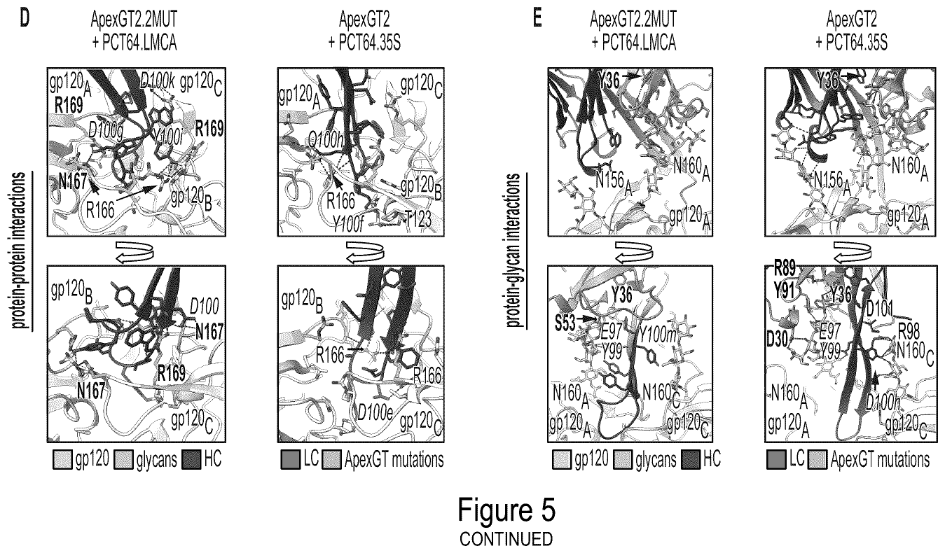

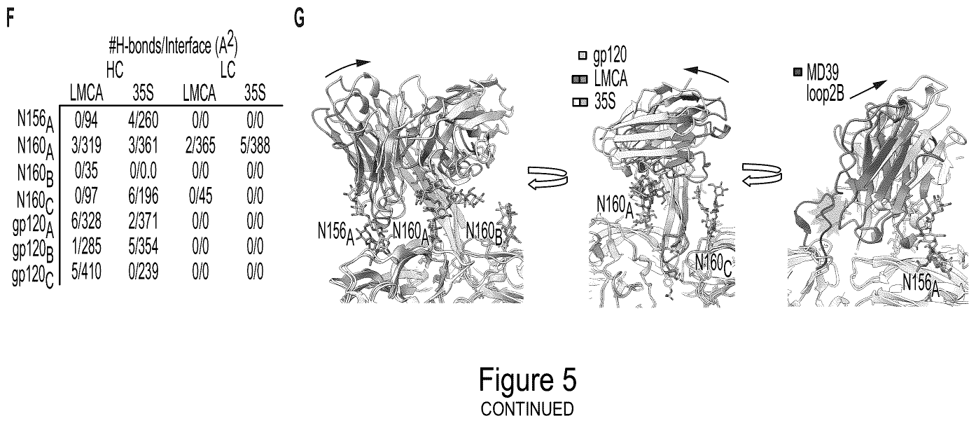

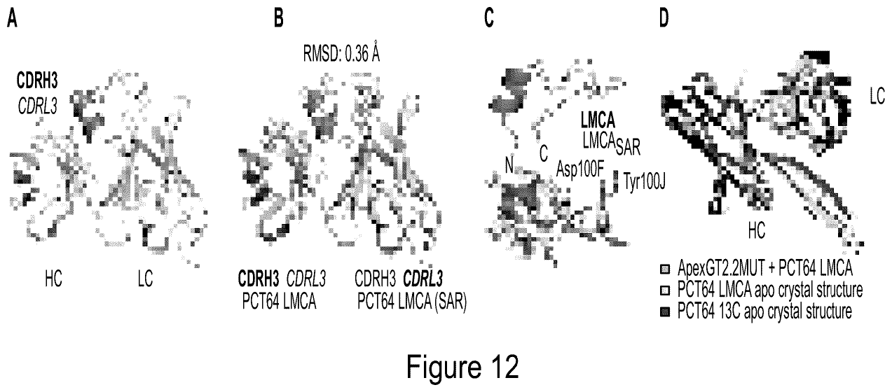

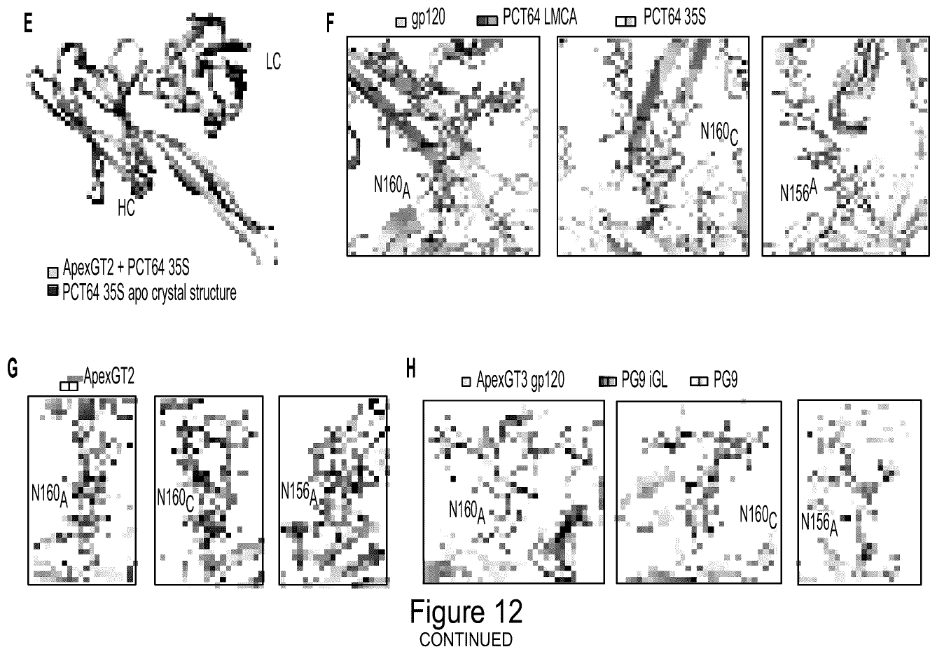

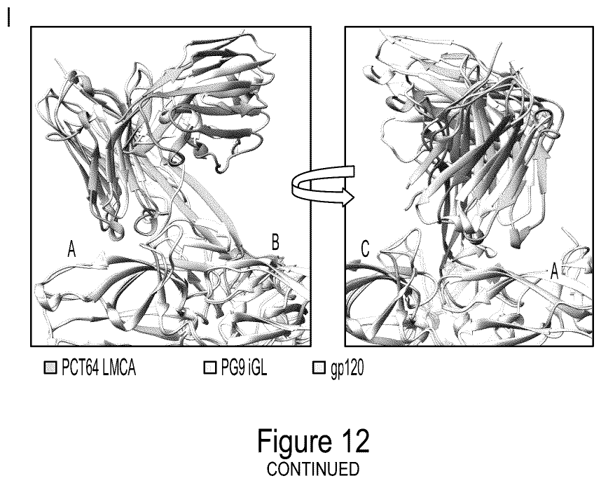

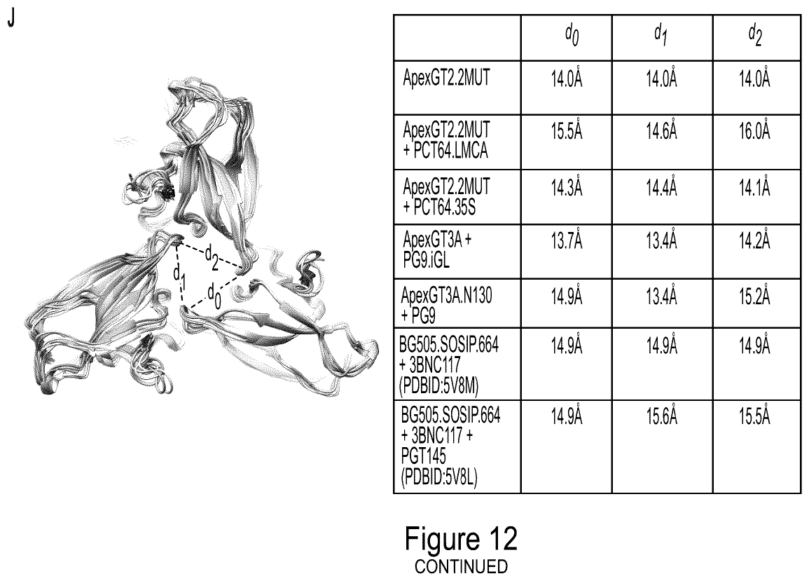

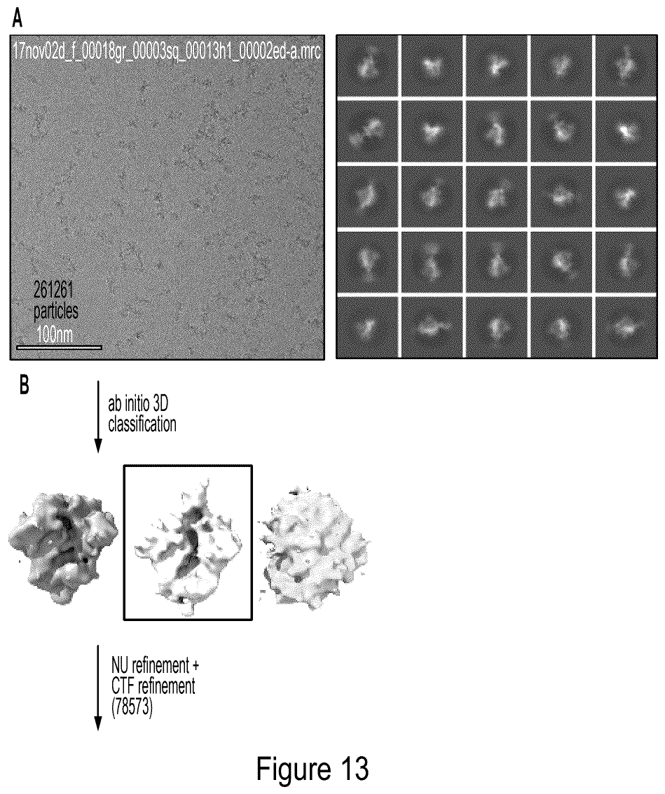

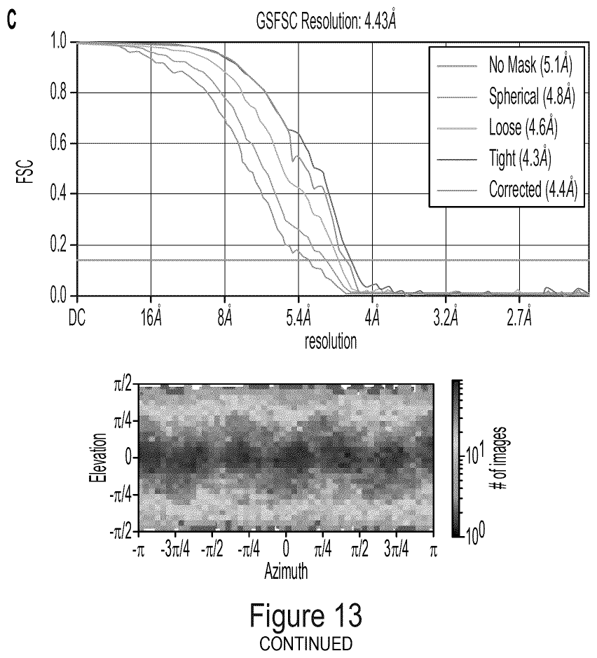

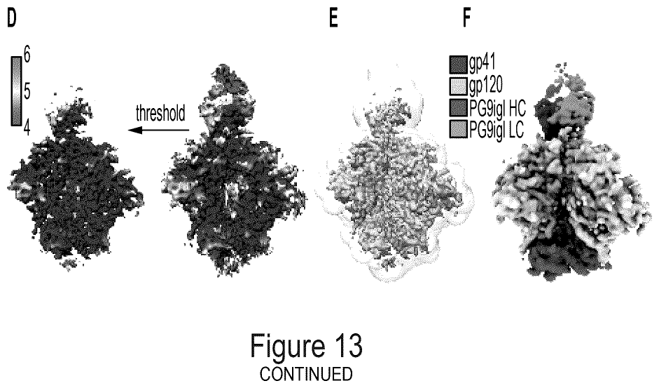

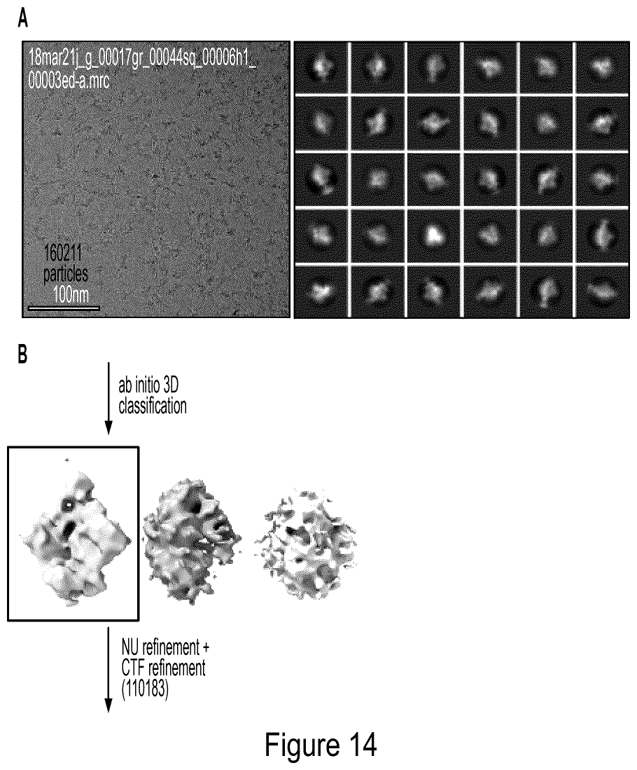

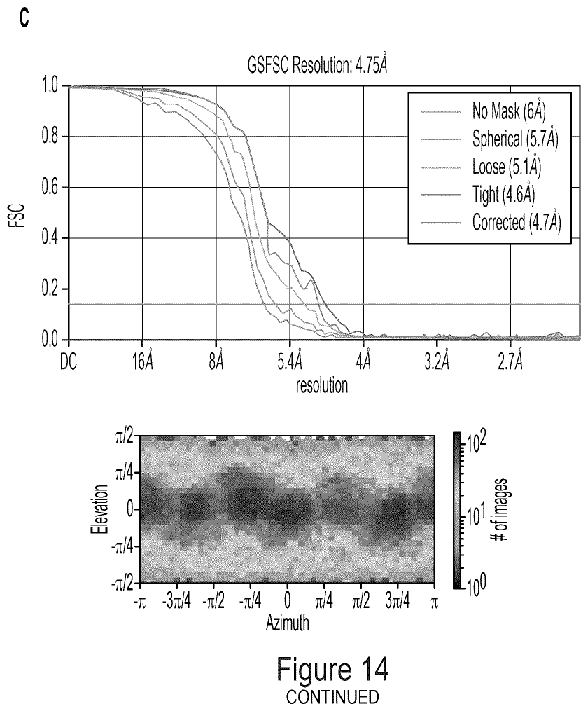

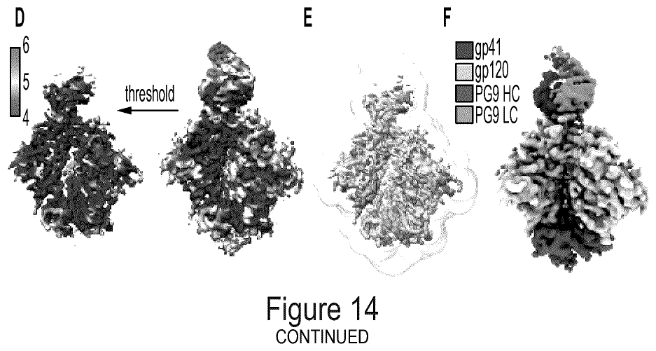

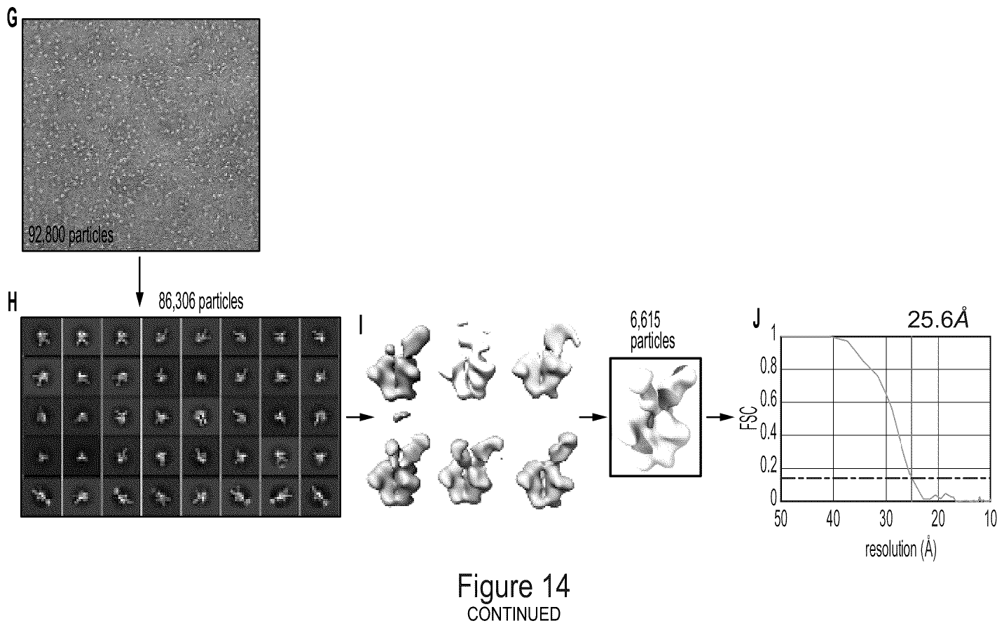

ApexGT2-PCT64 variant structures. The structures of ApexGT2.2M UT bound to PCT64 LMCA and ApexGT2 bound to PCT64 35S (FIGS. 5A, S3-S4), both showed the expected 1:1 stoichiometry and tilted angle of approach relative to the 3-fold axis as observed previously for autologous PCT64 complexes (Landais et al., 2017; Rantalainen et al., 2018), as well as the extended beta-hairpin conformation of the HCDR3 domain (FIG. 5B). In the previously reported apo crystal structure of a PCT64 LMCA variant (with three light chain mutations that confer neutralization to autologous viruses) the HCDR3 formed a collapsed coil (Rantalainen et al. 2018). Here, Applicants solved the apo crystal structure of the true LMCA Fab and found a similar collapsed coil/disordered HCDR3 conformation (FIG. 12A-C). In Applicants' PCT64 LMCA complex structure, the HCDR3 adopted a beta-hairpin conformation resembling more mature PCT64 antibodies (e.g. PCT64 13C, FIG. 12D), which suggested that either the apo coil conformation was a crystallization artifact or that PCT64 LMCA binds via an induced-fit mechanism. The HCDR3 of the mature bnAb PCT64 35S in complex with ApexGT2.2M UT showed only minor differences relative to the apo crystal structure of PCT64 35S Fab (PDB: 6CA6), adopting a slightly straighter beta-hairpin conformation (FIG. 12E).

Sulfated tyrosines, which play a critical role in PG9/PG16 (Pejchal et al., 2010) were resolved in the EM map density for both PCT64 LMCA (Y 100e and Y 100i) and 35S (Y 100f) (FIG. 5B) (Landais et al., 2016). Electrostatic potentials calculated with the Adaptive Poisson-Boltzman Sampling (APBS) algorithm (Jurrus et al., 2018) showed the positive and negative electrostatic potential of the ApexGT trimer binding surface and PCT64 HCDR3 domains, respectively (FIG. 5C). This electrostatic complementarity likely contributes to the binding affinity.

Applicants next examined protein-protein interactions between ApexGT mutations and both PCT64 LMCA and PCT64 35S (FIG. 5D). R169 was found to engage the LMCA through hydrogen bonding interactions on all three gp120 protomers, and N167 was found to engage two of the three protomers. R166, native to the BG505 isolate, was positioned in two of three protomers for potential cation-π interactions with the aromatic HCDR3 residues W100b and F100a. The PCT64 LMCA interactions with GT mutations R169 and N167 (FIG. 5D) were not found in the 35S complex, but the interactions with R166 were retained by 35S. The more extended HCDR3 conformation in the 35S complex positioned the sulfated Tyr100f residue deeper within the apex where it formed multiple new h-bonds with two protomers (FIG. 5D).

In addition to engaging gp120 through protein-protein interactions, both PCT64 LMCA and 35S engage apex-related glycans, in particular the N160gp120A glycan buried in the HC/LC interface (FIGS. 5E and 5F). Mature 35S forms a larger number of specific h-bonds with the N160 and N156 glycans on gp120A (FIG. 5F). These interactions are likely dynamic, as 3-D variability analysis (Punjani and Fleet, 2021) revealed flexibility of both Fabs relative to the trimer apex, involving shifting contacts with the N156gp120A glycan (Supplemental Movie 1).

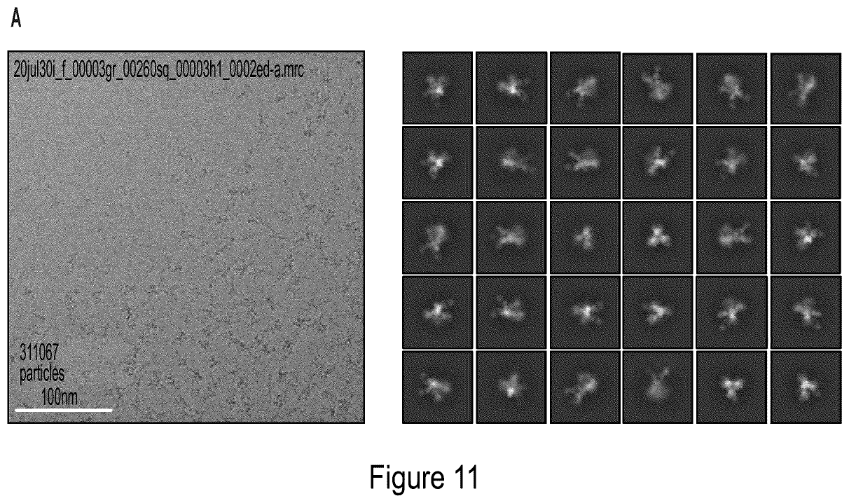

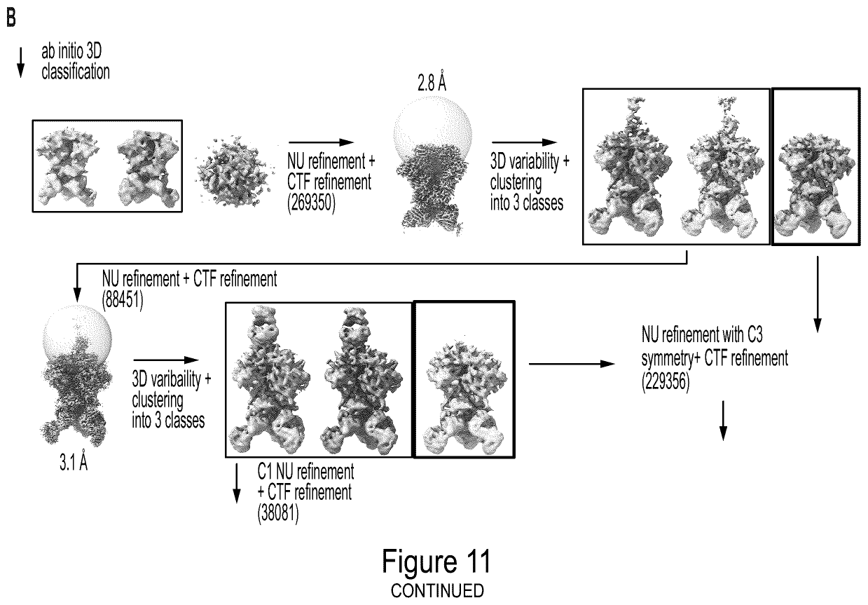

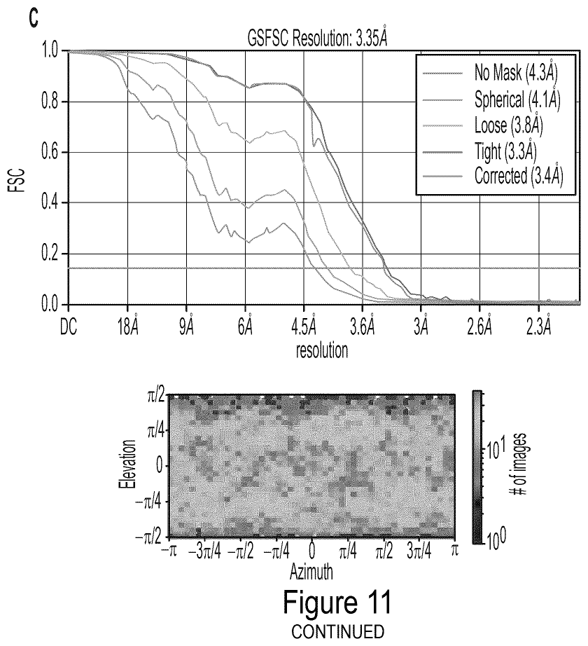

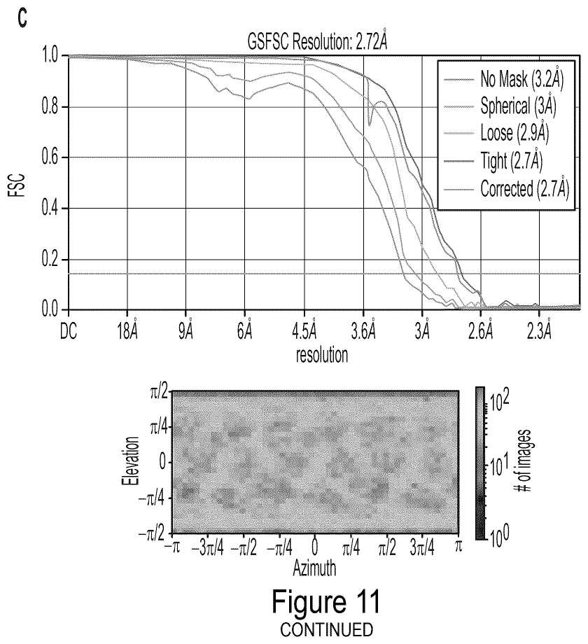

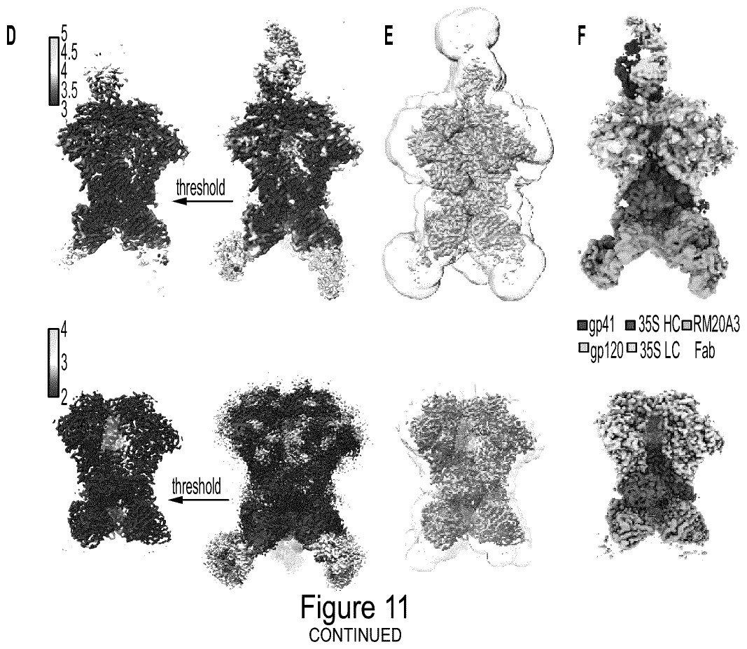

Finally, using a subset of apex-unliganded trimers identified from the PCT64 35S complex dataset, Applicants obtained a 2.7 Å-resolution reconstruction of ApexGT2 with unliganded trimer apex (FIG. 11C-F), from which it was determined how each glycan was being displaced in the complex relative to its average apex-unliganded position. Applicants found that three glycans, N156gp120_{A}, N160gp120_{A}, and N160gp120_{C}, were displaced less by the 35S Fab than by the LMCA (FIG. 12G), which is indicative of better glycan accommodation by 35S. This improved accommodation is a direct result of the steeper angle of approach of 35S relative to the trimer apex (FIG. 5G). The steeper angle of approach also relieves potential clashes with longer Loop2b regions, such as the one from wild-type BG505 (FIG. 5G). The conformation of Loop2B in both apo and PCT64-35-bound trimers, and the lack of clear map density for the N187 glycan, both suggest that the tip of the loop is predominantly oriented away from the 3-fold axis and the glycan itself is highly flexible, consistent with the N187 glycan having negligible effect on K_{D} (FIG. 8C).

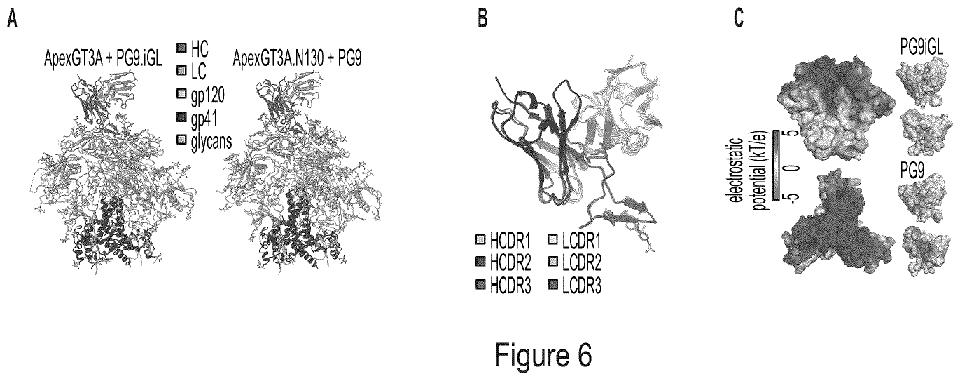

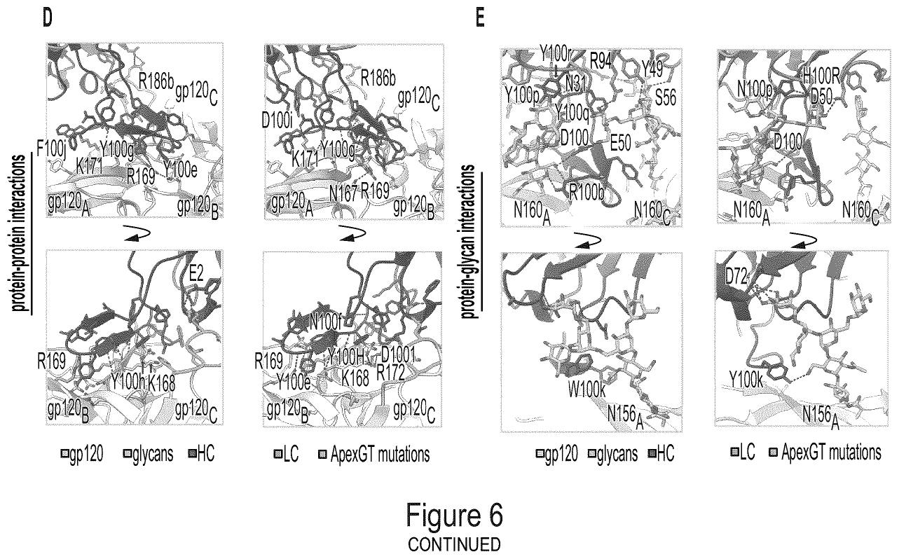

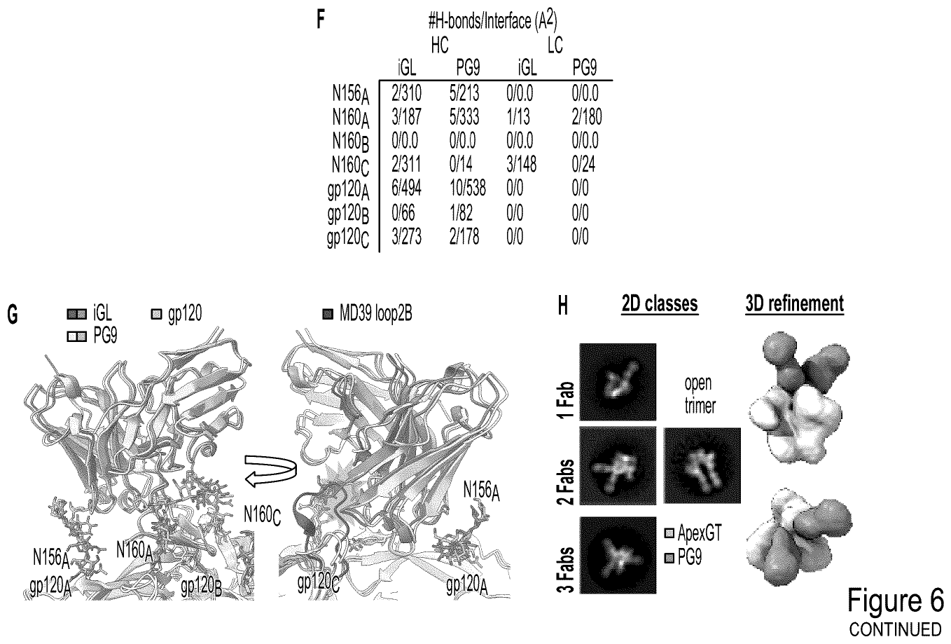

ApexGT3-PG9 variant structures. The structures of ApexGT3 bound to PG9 iGL, and ApexGT3.2M UT bound to PG9, shared characteristics with the PCT64 complexes described above. Both PG9 Abs engage the GT trimer with 1:1 stoichiometry, bind at a tilted angle of approach relative to the trimer apex (FIG. 6A), use an extended anionic HCDR3 containing sulfated tyrosines (FIG. 6B-D), and form extensive interactions with apex glycans (FIG. 6A-C). The additional glycans at positions N130 and N185H, present on ApexGT3.2M UT in the PG9 complex but not on ApexGT3 in the PG9 iGL complex, do not make direct contacts with the PG9 Fab. In contrast to PCT64 LMCA, PG9 iGL relies heavily on beta-sheet interactions with the C-strand of V2 on gp120_{A}, does not reach as far into the positively charged 3-fold symmetry axis (FIG. 6D), and utilizes a less negatively charged HCDR3 (FIG. 6C).

The beta-sheet main-chain interactions with the gp120 C-strand are in good agreement with the previously reported crystal structure of PG9 bound to a V1/V2 protein scaffold (McLellan et al., 2011) (FIG. 6D). The GT mutation R169 forms h-bonds with the HCDR3 on two different gp120 protomers and is also positioned well for co-planar cation-π interactions with Y 100e on both PG9 and PG9 iGL. The sulfated tyrosine at position 100ₑ on PG9 iGL interacts with R169 on gp120_{A}, while the mature PG9, which includes a sulfated Y 100_{g}, can also engage the N167gp120_{A} glycan.

Similar to the PCT64 complexes, the number of interactions with the glycan at N160gp120_{A} increases with maturation, with PG9 gaining four h-bonds with distal mannose residues (FIG. 6E,F). Specifically, the germline-encoded tyrosines at both 100p and 100r in PG9 iGL but not in PG9 displace N160gp120_{A} out of the HC/LC binding pocket (FIG. 6E) and likely contribute to the poor binding affinity of the iGL for most native Env trimers. In addition to the interactions with the N160gp120_{A} glycan, PG9 also acquires additional h-bonds with the N156g120_{A} glycan (FIG. 6E), one of which arises from the W100ₖY mutation in the HCDR3. PG9 iGL but not PG9 engages with the N160gp120c glycan, using both the HC and LC (FIG. 6E), indicating that a trimeric interface is necessary to engage iGL but not mature PG9. Indeed, evidence from negative stain EM (NSEM) suggests that two or three PG9 Fabs can bind a single ApexGT3 trimer (FIG. 6H), but due to steric hinderance the trimer would likely need to adopt an open conformation to accommodate the third Fab, as seen in some of the negative stain 2-D classes (FIG. 6H).

PCT64 LMCA and PG9 iGL show near identical binding angles with their respective ApexGT trimers (FIG. 121). This tilted binding angle could potentially cause clashes with the elongated Loop2b of BG505-derived trimers. Applicants' structures reveal that this clash is avoided by the shorter Loop2b of both ApexGT trimers (FIGS. 5G and 6G).

Inter-protomer distance measurements between gp120 subunits at the 3-fold symmetry axis indicate an asymmetric “swelling” of the V2-apex binding site upon antibody binding (FIG. 12J). This swelling only occurs upon binding for V2-apex bnAbs and is not observed for other classes of bnAbs. In addition, ApexGT trimers have a lower thermal stability (˜71° C., FIG. 4E) than MD39 (77° C., (Steichen et al., 2016) as well as higher V3 reactivity than MD39 (FIG. 4C), which may be indicative of reduced stability in the apex interface. This corroborates a previous hypothesis that an intrinsic lability of the trimer apex may be necessary for binding to V2-apex bnAbs (Lee et al., 2017).

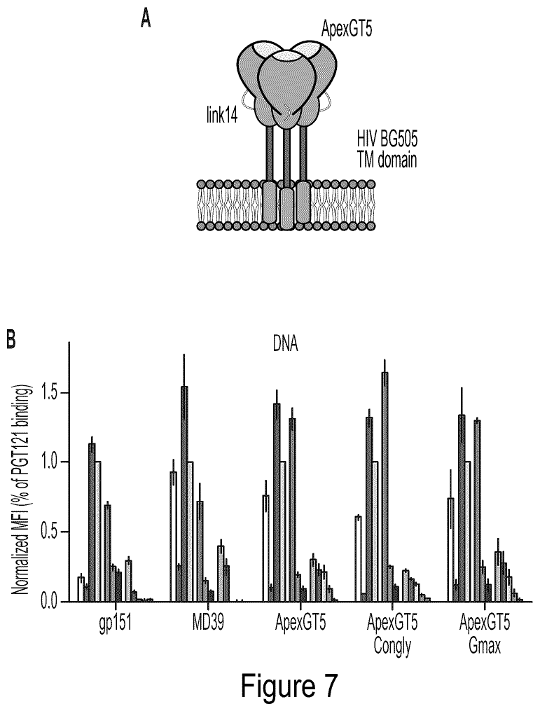

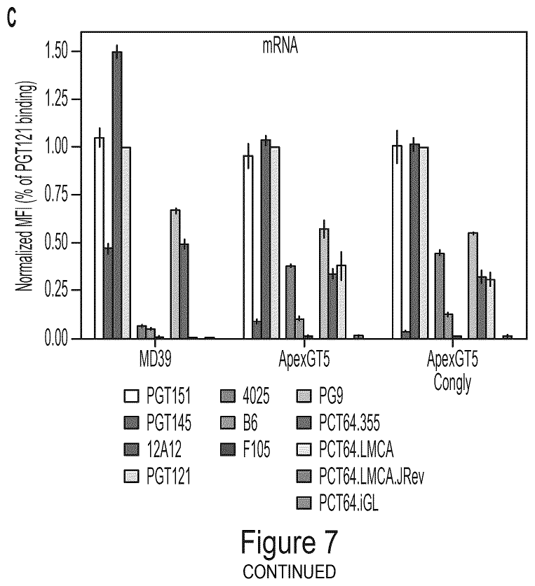

MRNA-delivered membrane-bound ApexGT trimers. The rapid development and high efficacy of the mRNA-based COVID-19 vaccines (Baden et al., 2021; Polack et al., 2020), which were based on delivery of mRNA coding for membrane-anchored full-length spike proteins, suggests that a similar approach to delivering HIV trimers (FIG. 7A) could potentially benefit HIV vaccine development (Aldon et al., 2018). Fully native HIV trimers are anchored to a lipid membrane on the virus or an infected cell, but most HIV vaccine discovery efforts have utilized trimers in a soluble format owing to the difficulties associated with production and purification of membrane proteins. MRNA-delivery of membrane-bound trimer immunogens would: (i) at least partially occlude the trimer base that when exposed on soluble trimers is immunodominant and elicits non-neutralizing and trimer-degrading antibodies (Bianchi et al., 2018; Cirelli et al., 2019; Hu et al., 2015; Nogal et al., 2020; Turner et al., 2021); (ii) offer potentially highly multivalent in vivo trimer presentation on microvesicles or exosomes (Bansal et al., 2021) or cell surfaces; and (iii) potentially be advantageous for trimer quaternary conformational sampling (Walker et al. 2009) and glycosylation (Cao et al., 2018).

Applicants sought to develop an mRNA vaccine platform encoding membrane-bound ApexGT trimer immunogens. Initial in vitro transfection experiments with DNA coding for membrane-bound BG505 MD39 native-like trimers used the link14 modification to eliminate the need for a furin cleavage (Steichen et al., 2016; Steichen et al., 2019) and tested two different truncations of the gp160 C-terminus.

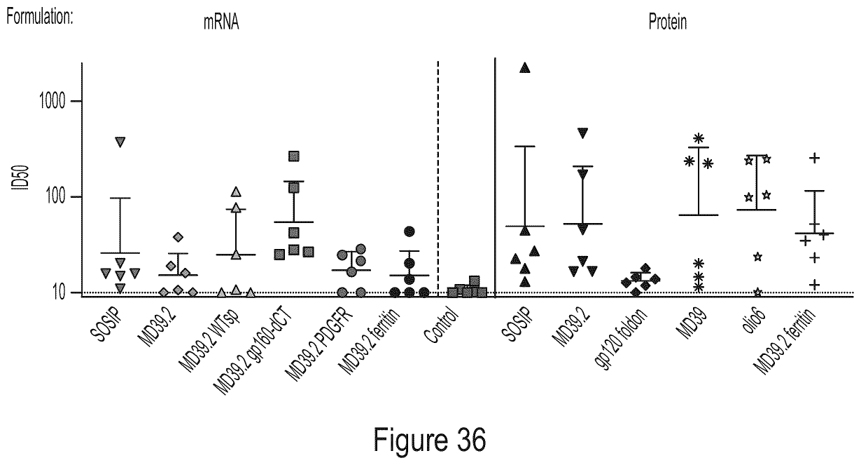

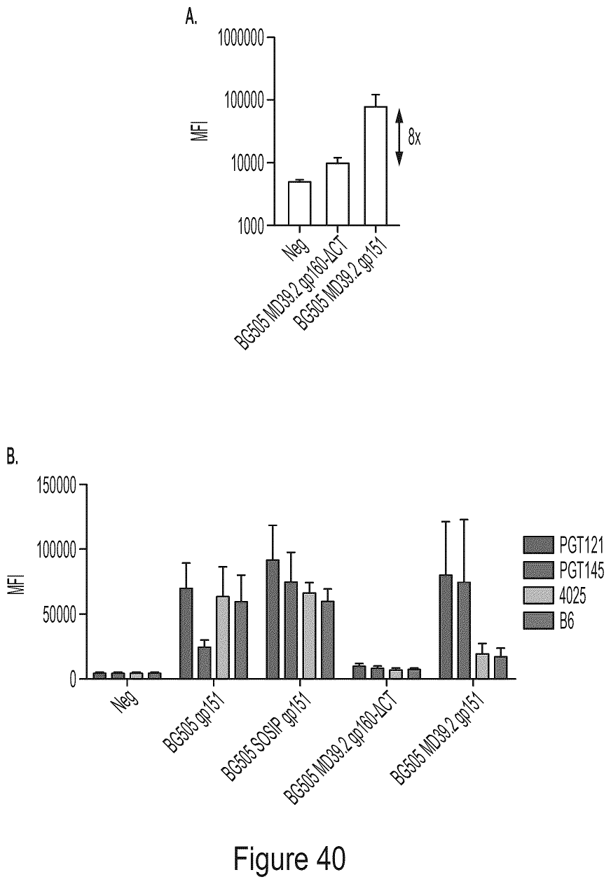

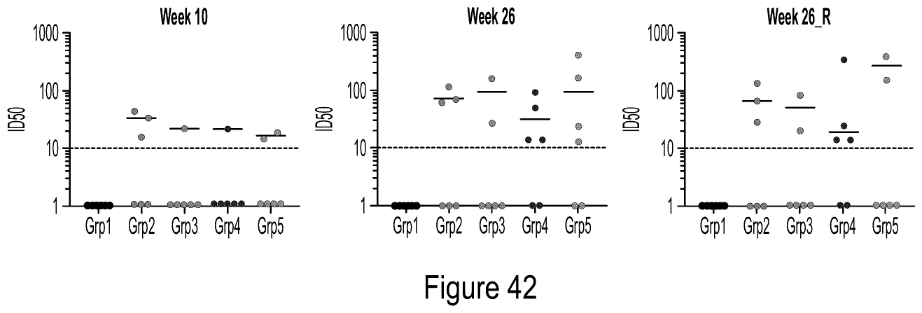

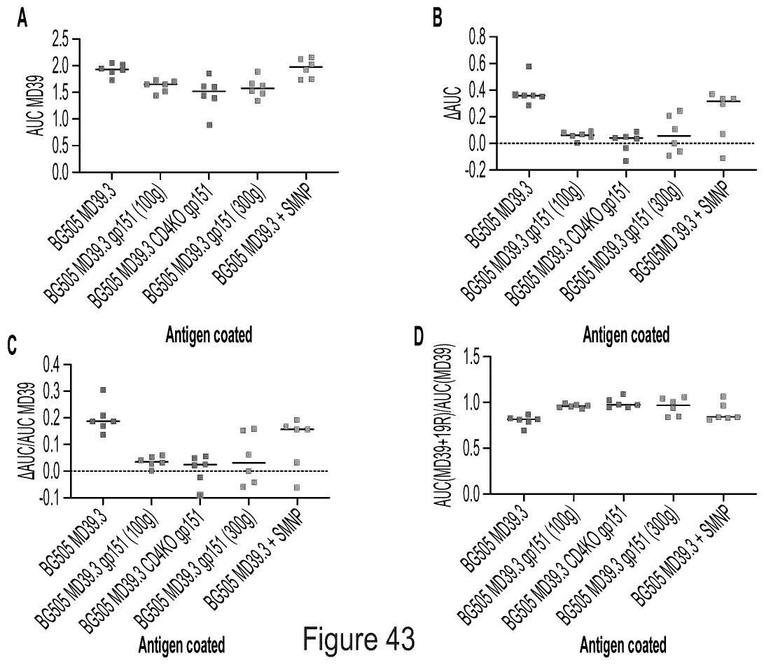

Applicants found that the shorter construct, termed MD39 link 14 gp151 and truncated at residue 709 (C-terminus, VIHRVR), (SEQ ID NO: 29) had 8-fold superior cell surface expression compared to the longer construct that contained a known endocytosis motif (GYXXØ) and was truncated at residue 716 (C-terminus, RVRQGYSPLS) (SEQ ID NO: 30) (FIG. 40A). The MD39 link14 gp151 construct also displayed a favorable cell-surface antigenic profile (FIG. 40B). Rabbit immunization experiments indicated that mRNA membrane-bound MD39 trimers elicited reduced base-directed responses and similar or better autologous neutralizing responses compared to mRNA- or protein-delivered soluble MD39 trimers (FIGS. 36-38). A non-human primate immunization experiment also showed that the mRNA-delivered, membrane-bound MD39 link14 gp151 trimer elicited: (a) reduced base-directed responses compared to soluble MD39 trimers delivered by RNA or protein (FIG. 43); (b) similar autologous neutralization responses as protein-delivered soluble MD39 (FIG. 42); and (c) improved autologous neutralization responses compared to mRNA-delivered soluble MD39 (FIG. 42). Applicants therefore developed ApexGT5 membrane-bound trimers utilizing the link14 linker and gp151 C-terminal truncation.

Cell-surface antigenic profiling of DNA-transfected cells showed that, relative to the base gp151 construct, the MD39 construct had stronger binding to trimer-specific bnAbs PGT151, PGT145, and PCT64 35S, and weaker binding to non-neutralizing mAbs 4025, B6 and F105, indicating that MD39 gp151 had a more native-like trimer structure than the base gp151 (FIG. 7B). ApexGT5 had a similar overall antigenic profile to MD39 except with modestly elevated binding to the non-neutralizing V3-directed Ab 4025, reduced binding to PGT145, PG9, and PCT64 V2-apex bnAbs, and increased binding to PCT64 LMCA and PCT64 LMCA.JREV (FIG. 7B). Antigenicity of the “congly” variant (with N241 and N389 glycosylation sites restored) and Gmax variant of ApexGT5 were similar to ApexGT5 (FIG. 7B). To enable antigenicity evaluation from mRNA-transfected cells, and eventual immunogenicity testing (Melzi et al., 2022), Applicants synthesized Moderna mRNAs for MD39 gp151, ApexGT5 gp151, and ApexGT5 congly gp151. The overall antigenic profiles of ApexGT5 and MD39 from mRNA-transfected cells were similar to those from DNA-transfected cells, with ApexGT5 having a similar overall profile to MD39 except for reduced PGT145, increased 4025, and increased PCT64 LMCA and PCT64 LMCA.JREV binding (FIG. 7C).

HIV bnAbs to the V2-apex epitope are important leads for vaccine design, but they present significant challenges for vaccine elicitation due to the dependence of their neutralizing activity on long HCDR3s. The first requirement for a vaccine to elicit a V2-apex bnAb will be to consistently prime potential bnAb-precursor B cells with genetic properties similar to the bnAb, including a long HCDR3. In general, HCDR3 sequences are diverse among precursors for any one class of bnAb, owing to junctional diversity of non-templated regions of HCDR3s. Thus, to define precursors for any particular class of bnAb, shared features must be defined that can be found in the shared human naive (or memory) B cell repertoire. Applicants previously developed a generalized germline-targeting vaccine design strategy for HCDR3-dominant antibodies, a strategy that leverages ultradeep sequencing data from the human heavy chain B cell repertoire and attempts to design immunogens that can bind diverse HCDR3s belonging to any single bnAb class (Steichen et al., 2019). Here Applicants began adapting that strategy to the exceptionally long HCDR3s for V2-apex class bnAbs, by performing a comprehensive frequency analysis for HCDR3-dominant V2-apex bnAb precursors in ultradeep sequencing data from 14 healthy donors. Applicants' analysis led us to prioritize PCT64 and PG9/16 for V2-apex vaccine targeting, with PCT64 of highest priority, owing to higher precursor frequencies and shorter HCDR3 mutational distances to a known bnAb. PCT64 has these favorable properties because a large portion (20 of 25 amino acids) of its HCDR3 is templated by D or J genes. Based on the analyses, Applicants hypothesize that germline-targeting vaccine design for long HCDR3 bnAbs will generally be more favorable for bnAbs with larger fractions of their HCDR3s templated. Conversely, bnAbs with large untemplated regions of HCDR3 will be difficult targets because precursors with shared features within those untemplated regions will be very rare. In general, the population frequencies of the templated D and J gene alleles will also be an important factor in precursor frequency.

Applicants engineered ApexGT trimer immunogens with affinity for precursors to both PCT64 and PG9/16, determined four high-resolution cryo-EM structures of ApexGT trimers with bnAb and precursor forms of PCT64 and PG9, and illustrated how one of the early-determined structures (ApexGT2+PCT64 LMCA) was employed to guide design of a higher affinity ApexGT trimer (ApexGT5).

From the PCT64 cryoEM structures, Applicants found that maturation of the PCT64 lineage was achieved through improved accommodation of apex-associated glycans, primarily N160 and N156 on gp120 protomer A, more extensive and less specific interaction between the HCDR3 and gp120 V2/V3 loops on gp120 protomer A and B, and reduced clashes with loop2B and loop2B glycans on gp120 protomer C. All these changes were made possible by the extension and rigidification of the beta-hairpin which allowed the sulfated tyrosine at the tip of the HCDR3 loop to reach deeper in 3-fold axis binding pocket and created a steeper the angle of approach relative to the trimer apex. Thus, in order to achieve similar maturation through sequential prime-boost vaccination, boosting immunogens will likely need to have fewer positively charged residues in the V2 loop and longer, more glycosylated loop2Bs. Taken together, Applicants' PG9 structures revealed that maturation of this lineage was achieved primarily through improved interactions with apex-associated glycans N160 and N156 on gp120 protomer A, similar to PCT64, and somewhat surprisingly, reduced interaction with the N160 glycan on gp120 protomer C. Unlike PCT64, the PG9 HCDR3 loop structure and its interactions with gp120 protein residues are more conserved between the GL and mature antibodies and there is no change in the angle of approach relative to the trimer apex. Thus, the maturation path from precursor to bnAb appears to be simpler for PG9 than for PCT64. That finding, together with the higher potency of PG9/16, encourages continued work to develop optimal priming immunogens for PG9/16, despite their lower precursor frequency compared to PCT64.

Applicants concluded this work by developing membrane-bound forms of the ApexGT trimers that are promising for mRNA vaccine delivery, characterizing them by cell-surface antigenic profiling from both DNA and mRNA transfection. In Example 2, Applicants report immunogenicity studies of ApexGT trimers delivered as soluble proteins and as mRNA-lipid nanoparticles (LNPs) encoding membrane-bound trimers (Melzi et al., 2022). These studies represent promising steps toward development of a germline-targeting immunogen capable of priming V2-apex precursor B cells in humans. The next step toward that goal is to develop even more advanced ApexGT trimers with affinity for diverse human precursors for PCT64 and/or PG9/16, allowing for diverse HCDR3 V-D and D-J junctions found in human naive and/or memory B cells.

### Key Resources

| | | |
|---|---|---|
| REAGENT or RESOURCE | SOURCE | IDENTIFIER |

Antibodies

| | | |
|---|---|---|
| 4025 | (Gorny et al., 2011) | NA |
| B6 | (Burton et al., 1991) | NA |
| F105 | (Posner et al., 1991) | NA |
| PGT151 | (Falkowska et al., 2014) | NA |
| PGT121 | (Walker et al., 2011) | NA |
| 12A12 | (Scheid et al., 2011) | NA |
| PGT145 | (Walker et al., 2011) | NA |
| PG9 | (Walker et al., 2009) | NA |
| PCT64.35S | (Landais et al., 2017) | NA |
| PCT64.35K | (Landais et al., 2017) | NA |
| PCT64.LMCA | (Landais et al., 2017) | NA |
| PCT64.iGL | (Landais et al., 2017) | NA |
| PCT64.LMCA.J Rev | This paper | NA |
| PG9.iGL | This paper | NA |

Bacterial and virus strains

| | | |
|---|---|---|
| Electromax Stb4 | ThermoFisher | Cat#11635018 |
| DH5a | ThermoFisher | Cat#18265017 |

Chemicals, peptides, and recombinant proteins

| | | |
|---|---|---|
| Microcon Ultracel PL-10 | Millipore Sigma | Cat#MRCPRT010 |
| centrifugal filters | | |
| Pierce TCEP HCl | Thermo Scientific | Cat#20490 |
| Ammonium acetate ≥98% | Sigma-Aldrich | Cat#A1542 |
| 2-Chloroacetamide ≥98% | Sigma-Aldrich | Cat#22790 |
| Endo H | NEB | Cat#P0702 |
| Calcium chloride | Sigma-Aldrich | Cat#C5670 |
| Proteinase K | Sigma-Aldrich | Cat#P2308 |
| PNGase F | NEB | Cat#P0705 |
| Ammonium bicarbonate ≥99.5% | Sigma Aldrich | Cat#09830 |
| H218O (97% 18O) | Sigma-Aldrich | Cat#329878 |
| Resin, Acquity UPLC ® | Waters | Cat#186004690 |
| BEH 1.7 μm C18 | | |
| Water (Optima LC-MS grade) | Fisher Scientific | Cat#W6-4 |
| Acetonitrile | Honeywell | Cat#AH015-4 |
| Formic acid 90% | J. T. Baker | Cat#0129-01 |
| Polymicro fused silica capillary | Molex | Cat#1068150023 |
| tubing 100 μm | | |
| 11 PP Vial Crimp/Snap 250 μL | Thermo Scientific | Cat#C4011-13 |
| Snap-it Seal | Thermo Scientific | Cat#C4011-53 |
| 0.2 mL Thin-walled Tubes | Thermo Scientific | Cat#AB-0620 |
| Protein LoBind Tube 1.5 mL | Eppendorf | Cat#022431081 |
| DeckWorks low binding pipette tips | Corning | Cat#4151 |
| rProteinA Fast Flow | GE | Cat# 17-1279-03 |
| IgG Elution Buffer | Thermo Scientific | Cat#210099 |
| Pierce HeLa protein digest | Thermo Scientific | Cat#88329 |
| standard | | |
| Opti-MEM | Thermo Scientific | Cat#31985-088 |
| PEI Max | FisherScientific | Cat#NC1038561 |
| Gibson Assembly Master Mix | NEB | Cat#E2611L |
| LR Clonase II | ThermoFisher | Cat#11791100 |
| Puromycin | ThermoFisher | Cat#A1113803 |
| Blasticidin | ThermoFisher | Cat#A1113903 |
| Advanced DMEM | Gibco | Cat#12491015 |
| Tet Approved FCS | Gibco | Cat#A4736301 |
| 2-mercaptoethanol | Gibco | Cat#21985023 |
| Antibiotic-Antimycotic | Gibco | Cat#15240096 |
| Fugene HD | Promega | Cat#32043 |
| FITC-labeled α-cMyc | Immunolgy Consultants Lab | Cat#CMYC-45F |
| APC Anti-Human IgG | Jackson | Cat#709-136-149 |
| PE anti-V5 Tag | ThermoFisher | Cat#12-6796-42 |
| 6x-His E pitope Tag Antibody | GenScript | Cat#10500-530 |
| HBS-EP + 20x, pH 7.6 | TekNova | Cat#H8022 |
| Human Antibody Capture Kit | GE | Cat#BR-1008-39 |
| Peroxidase AffiniPure Goat Anti- | Jackson | Cat#NC9752299 |
| Human IgG, Fcγ | | |
| Acetonitrile, 80%, 20% | Fisher Scientific | Cat#15431423 |
| Water with 0.1% Formic Acid | Fisher Scientific | Cat#LS118-212 |
| Acetonitrile | Fisher Scientific | Cat#10489553 |
| Trifluoroacetic acid | Fisher Scientific | Cat#10155347 |
| Procainamide hydrochloride | Abcam | Cat#ab120955 |
| Dithiothreitol | Sigma-Aldrich | Cat#43819 |
| Iodacetamide | Sigma-Aldrich | Cat#\|1149 |
| Sequencing grade chymotrypsin | Promega | Cat#V1061 |
| Alpha Lytic protease | Sigma Aldrich | Cat#A6362 |
| Mass spectrometry grade trypsin | Promega | Cat#V5280 |
| Lauryl maltose neopentyl glycol | Anatrace | Cat#NG310 1 GM |
| 293Fectin | ThermoFisher | Cat#12347500 |
| TransIT-mRNA transfection Kit | Mirus | Cat#MIR 2250 |
| Alexa Fluor 647-conjugated | Jackson | Cat#109-605-003 |
| antihuman IgG | | |
| 30KD Amicon Ultra | Millipore | Cat#Z717185 |
| 1.2/1.3 C-Flat holey carbon grids | Electron Microscopy Sciences | Cat#CDFT823-50 |

Deposited data

| | | |
|---|---|---|
| Mass spectrometry and proteomics | Pride | PXD033766 |
| analysis | | |
| NGS Dataset | (Steichen et al., 2019) | https://github.com/SchiefLab/SteichenScience2019 |
| ApexGT2.2MUT + PCT64 | RCSB | 7T73 |
| LMCA Fab | | |
| ApexGT2 + PCT64.35S Fab + | RCSB | 7T74 |
| RM20A3 Fab | | |
| ApexGT2 + RM20A3 Fab | RCSB | 7T75 |
| ApexGT3 + PG9 iGL Fab | RCSB | 7T76 |
| ApexGT3.2MUT + PG9 Fab | RCSB | 7T77 |
| ApexGT2.2MUT + PCT64 LMCA | EMDB | 25732 |
| Fab | | |
| ApexGT2 + PCT64.35S Fab + | EMDB | 25733 |
| RM20A3 Fab | | |
| ApexGT2 + RM20A3 Fab | EMDB | 25734 |
| ApexGT3 + PG9 iGL Fab | EMDB | 25735 |
| ApexGT3.2MUT + PG9 Fab | EMDB | 25736 |

Cell lines

| | | |
|---|---|---|
| HEK 293F | ThermoFisher | Cat#R79007 |

Experimental models: Cell Lines

| | |
|---|---|
| rtTA3G 293T | David Nemazee, Scripps |
| | Researsh, La J olla |
| | (Ota et al. 2012) |

Oligonucleotides

| | | |
|---|---|---|
| Loop2b OligoLibrary | CustomArray | NA |
| NNK Library 32-185H | SGI-DNA | Custom order |
| Long Ultramers NNK | IDT | 4 nmole Ultramer ® DNA Oligo |

Recombinant DNA

| | | |
|---|---|---|
| pHL-SEC | Ariescu et al 2006 | Addgene #99845 |
| pFUSE2ss-CHIg-hG1 | InvivoGen | Cat#pfusess-hchg1 |
| pFUSE2-CLIg-hL2 | InvivoGen | Cat#pfuse2-hcll2 |
| pFUSE2-CLIg-hK | InvivoGen | Cat#pfusess-hchg1 |
| pENTR/D-TOPO | David Nemazee, Scripps | |
| | Researsh, La J olla | |
| | 2(Ota et al. 012) | |
| pLenti-CMVTRE3G/Puro_Dest | David Nemazee, Scripps | |
| | Researsh, La J olla | |
| | (Ota et al., 2012) | |
| psPAX2 | Didier Trono, EPFL, | AddGene#12260 |
| | Lausanne Switzerland | |
| pMD2.G | Didier Trono, EPFL, | AddGene#12259 |
| | Lausanne Switzerland | |

Software and algorithms

| | | |
|---|---|---|
| ProLuCID | IP2, Bruker Scientific LLC | http://fields.scripps.edu/yates/wp/?page_id=821 |
| DTASelect2.0 | IP2, Bruker Scientific LLC | http://fields.scripps.edu/yates/wp/?page_id=816 |
| Census2.0 | IP2, Bruker Scientific LLC | http://fields.scripps.edu/yates/wp/?page_id=824 |
| GlycoMSQuant v.1.8.2 | John R. Yates III, Scripps | https://github.com/proteomicsyates/GlycoMSQuant |
| | Research, La J olla | |
| RAW Converter | John R. Yates III, Scripps | http://fields.scripps.edu/rawconv/ |
| | Research, La J olla | |
| Office 365 | Microsoft | https://www.office.com/ |
| XCalibur | Thermo Scientific | Cat#OPTON-30965-7 |
| Abstar | (Briney et al., 2019) | https://github.com/briney/abstar |
| Amazon EMR | Amazon Web Services | https://aws.amazon.com/emr/ |
| Rosetta | RosettaCommons | https://www.rosettacommons.org/software/license-and-download |
| ProteON Manager Software | Bio-RAD | NA |
| ByosTM (Version 3.9) | Protein Metrics Inc. | NA |
| GraphPad Prism v9 | GraphPad | NA |
| XCalibur Version v4.2 | Thermo Fisher | NA |
| Orbitrap Fusion Tune | Thermo Fisher | NA |
| application v3.2 | | |
| MotionCor2 | (Zheng et al., 2017) | NA |
| Relion-2/3 | (Kimanius et al., 2016; | NA |
| | Zivanov et al., 2018) | |
| SWISS-MODEL | (Waterhouse et al., 2018) | NA |
| SAbPred | (Dunbar et al., 2016) | NA |
| UCSF Chimera | (Pettersen et al., 2004) | NA |
| GCTF | (Zhang, 2016) | NA |
| COOT | (Emsley and Cowtan, 2004) | NA |
| MolProbity | (Chen et al., 2010) | NA |
| EMRinger | (Barad et al., 2015) | NA |
| Privateer | (Agirre et al., 2015) | NA |
| Segger | (Pintilie and Chiu, 2012) | NA |
| Illustrator | Adobe | NA |
| Leginon | (Suloway et al., 2005) | NA |
| BD FACS Diva 6 | BD | NA |
| FlowJ o v10.8 | BD | NA |

Other

| | | |
|---|---|---|
| Influx | BD | NA |
| ProteOn XPR36 | Bio-Rad | Cat#176-0100 |
| ProteOn GLC Sensor Chip | Bio-Rad | Cat#76-5011 |
| MicroCal VP-Capillary DSC | Malven | NA |
| Dawn HELEOS II | Wyatt | NA |
| Optilab T-REX | Wyatt | NA |
| GenWiz EZ Amplicon Service | Genewiz/Azenta | NA |
| HisTrap HP | Cytiva/GE | Cat#17371205 |
| Superdex 200 10/300 GL | Cytiva/GE | Cat#17517501 |
| EasySpray PepMap RSLC | Thermo Fisher Scientific | Cat#ES905 |
| C18 column | | |
| PVDF protein-binding membrane | Millipore | Cat#MAIPS4510 |
| C18 ZipTip | Merck Milipore | Cat#ZTC18S008 |
| Vivaspin 500, 3 kDa MWCO, | Sigma-Aldrich | Cat#GE28-9322-18 |
| Polyethersulfone | | |
| PepMap100 C18 3UM | Thermo Scientific | Cat#164946 |
| 75UM × 2CM Nanoviper | | |
| Vitrobot mark IV | Thermo Fisher Scientific | NA |
| Titan Krios | Thermo Fisher Scientific | NA |
| Talos Arctica | Thermo Fisher Scientific | NA |
| K2 Summit direct | Gatan | NA |
| electron detector | | |
| Unformulated mRNA | Moderna | NA |
| NovoCyte 3000 | Agilent | NA |

NGS dataset of human BCR HCs. This work utilized a large NGS dataset of 1.1×10⁹ amino acid sequences of BCR HCs from 14 healthy, HIV-uninfected donors as described previously (Steichen et al., 2019). Briefly, this dataset contains 255 million sequences from 10 donors obtained from VDJ heavy chain mRNA transcripts that were amplified with unique identifiers to minimize PCR bias and allow for error correction (Briney et al., 2019). In addition, 4 donors were sequenced from FW 3 to FW 4 using the NextSeq or HiSeq (2×150 bp) platform to obtain an additional 845 million sequences. Sequences were annotated with A bstar, converted to parquet format, and uploaded to AWS S3 storage platform. Multiple biological and technical replicates were recorded in the dataset as described previously (Briney et al., 2019).

Apex bnAb precursor frequency estimates. The NGS dataset was interrogated with the Spark analytics engine on AWS EM R platform using the precursor definitions defined in FIG. 16. PySpark scripts used in this analysis are available at github.com/SchiefLab/Willis2022. A precursor frequency was estimated by taking the total amount of precursors for each technical replicate that met the definitions in FIG. 16 divided by the total number of sequences in each replicate. The frequency per donor was averaged for each donor and the total frequency for each Apex bnAb precursor was reported as the median across all 14 donors for at least one precursor was found. To determine a precursor response, Applicants computed the frequency of donors which at least one precursor was found. 95% confidence intervals were estimated using the Wilson method for binomial proportions (Agresti and Coull, 1998).

Apex bnAb precursor frequency similarity. To calculate the similarity for each bnAb Apex class, precursors that matched Applicants' NGS queries were aligned to known bnAbs. The closest bnAbs was recorded for each precursor and the median number of mutations were recorded for each donor (mean edit distance).

Updating PG9 iGL. To determine the most appropriate amino acid at position 100p (kabat numbering) in PG9 iGL, Applicants searched long HCDR3s with the following regular expression “.* YYDF[WY][SD]GYY.YYYMDV$” (SEQ ID NO: 31). This regular expression queried Applicants' NGS dataset for the DJ recombination used by PG9 while leaving the ambiguous position as a “wild card”. Applicants then used the most frequent amino acid at position 100p.

DNA gene synthesis. Genes were synthesized at Genscript, Inc. ApexGT trimers in pHL see contained a C-terminal GTKHHHHHH (SEQ ID NO: 32) tag. Genes used in library design were cloned into pENTR which contained a C-terminal cMyc epitope followed by a PDGFR transmembrane domain. IgGs were cloned into pFUSEss-CHIg for heavy chains and pFUSE2-CLIg-hL 2/PFUSE2-CLIg-hK for light chains

ApexGT Protein Expression. BG505 based ApexGT variants were expressed in 293F cells grown in 293 Freestyle media by transient transfection with PEI Max. The protein was purified from the supernatant using a HIS-TRAP column, starting with a wash buffer (20 mM Imidazole, 500 mM NaCl, 20 mM Na2HPO4) and mixing with elution buffer (500 mM Imidazole, 500 mM NaCl, 20 mM Na2HPO4) using a linear gradient. The trimer fraction was collected and further purified on an S200Increase 10-300 column (GE) in HBS (10 mM HEPES, 150 mM NaCl). The oligomeric state of the SOSIP trimers were then confirmed by size exclusion chromatography-multi-angle light scattering using the DAWN HELEOS II multi-angle light scattering system with Optilab T-rEX refractometer (Wyatt Labs). The trimers were frozen in thin-walled PCR tubes at 1 mg/ml using liquid nitrogen and stored at −80° C.

Antibody Protein Expression. pFUSE heavy and light vectors were maxi-prepped using a BenchPro 2100. Heavy and light chains were transfected with PEI max in OptiM EM at a 3:1:1 PEI: heavy: light ratio into 293F freestyle cells. Cells were harvested on day 7, supernatant was clarified and purified by rProtein A Sepharose fast flow and eluted with IgG elution buffer.

ApexGT Immunogen Design. Lentiviral mammalian display and directed evolution were performed as described previously (Steichen et al., 2016) with modifications to sorting probes and libraries. Starting with BG505.SOSIP.D664 based construct, two library pathways were constructed. The first library used NNK degenerate codons at positions 32-185H (HXBC2 numbering, SGI). The second library had combinatorial 4 combinatorial NNK positions K 170-V173 using long ultramers (IDT). Both libraries were subsequently cloned into and the gateway entry vector pENTR/D-TOPO (Ota et al., 2012) using the Gibson Assembly Mix (NEB). The purified pENTR vector was then Gateway cloned to pLenti CMVTRE3G puro Dest (Ota et al., 2012) using the LR Clonase II enzyme mix. This plasmid DNA was purified and ready for use in transfection. 293T cells cultured in Advanced DMEM (Gibco) supplemented with 5% FCS, GlutaMAX (Gibco), 2-mercaptoethanol (Gibco) and Antibiotic-Antimycotic (Gibco) were co-transfected with NNK library in pLenti CMVTRE3G puro Dest (10.8 μg), psPAX2 (7.0 μg) and pMD2.G (3.8 μg) with fugeneHD in a T75 flask (Salmon and Trono, 2007). 293T cells stably expressing rtT A 3G from the pLenti CM V rtTA 3G Blast vector (Ota et al., 2012) were transduced at low moi (<0.1) in a T75 or T225 flask in the presence of 10 μg/mL blasticidin. The next day cells were selected with 2 μg/mL puromycin. 293T cells containing the stable library were induced with doxycycline (1 ug/mL) and the following day were harvested in FACS buffer (HBSS, 1 mM EDTA, 0.5% BSA). Cells were incubated with PG9 VHVL washed with FACS buffer, and then stained with fluorescein isothiocyanate (FITC)-labeled α-cMyc (Immunology Consultants Laboratory) and phycoerythrin APC-conjugated anti-human IgG (Jackson). Cells were sorted on a BD Influx (BD Biosciences) FACS sorter. Approximately 10K of the desired gates cells were collected and expanded for ˜one week in the presence of puromycin and blasticidin before the next round of enrichment was carried out.

For the Loop2b library (D180-Y 191) Applicants considered all natural sequences gathered from the Los Alamos National Laboratory (LANL) HIV Sequence Database (http://www.hiv.lanl.gov/) using semiconductor oligonucleotide arrays (CustomArray) and cloned into BG505 SOSIP.D664 to generate a library that could be sorted by mammalian display as described above but with PG9 iGL+1 MUT as the desired gate and 4025/V5 tag as negative gate to enrich for well formed “closed” trimers. The genomic DNA for the pre-sorted libraries, intermediate sorted rounds, and the final enriched libraries were extracted and PCR amplified Using partial adapters recommended by Gen Wiz “EZ amplicon”. Applicants determined enriched mutations as described previously (Kulp, 2017; Steichen et al., 2016).

Surface plasmon resonance (SPR). Kinetics and affinities of antibody-antigen interactions were measured on a ProteOn XPR36 using GLC Sensor Chip and 1×HBS-EP+pH 7.4 running buffer supplemented with BSA at 1 mg/ml. Human Antibody Capture Kit was used according to manufacturer's instructions to immobilize about 6000 RUs of capture mAb onto each flow cell. In a typical experiment, approximately 300-400 RUs of mA bs were captured onto each flow cell and analytes were passed over the flow cell at 30 L/min for 3 min followed by a 10 min dissociation time. Regeneration was accomplished using 3M Magnesium Chloride with 180 seconds contact time and injected four times per cycle. Raw sensograms were analyzed using ProteOn Manager software (Bio-Rad), including interspot and column double referencing, and either Equilibrium fits or Kinetic fits with Langmuir model with 1:1 binding stoichiometry, or both, were employed when applicable. Only data sets with Rmax-Ratio between 0.5 and 2 were accepted as correct. R max-Ratio was calculated by dividing theoretical R max-Expected by R max-Fit obtained from fitting the data. Rmax-Expected was calculated from ligand capture level assuming 2 binding sites per mAb-ligand molecule and one binding site per trimer-analyte molecule. For example if Applicants capture 100 RU of mAb and trimer molecular weight is 225 kDa the Rmax-Expected would be 225*2*100/150=300 RU. Analyte concentrations were measured on a NanoDrop 2000c Spectrophotometer using Absorption signal at 280 nm.

Differential scanning calorimetry (DSC). DSC experiments were performed on a MicroCal VP-Capillary differential scanning calorimeter (Malvern Instruments). The HEPES buffered saline (HBS) buffer was used for baseline scans and the protein samples were diluted into HBS buffer to adjust to 0.25 mg/ml. The system was allowed to equilibrate at 20° C. for 15 min and then heat up till 90° C. at a scan rate of 90° C./h. Buffer correction, normalization, and baseline subtraction were applied during data analysis using Origin 7.0 software. The non-two-state model was used for data fitting.

Antigenic profile with enzyme-linked immunosorbent assay (ELISA). 96-well plates were coated overnight at 4C with 6×-His Epitope Tag Antibody at 2 mg/ml in PBS. Plates were washed 3 times with PBS, 0.05% Tween (PBS-T), and blocked with 10% milk PBS for 1h. Subsequently, 2 mg/ml of the purified His-tagged ApexGT or MD39 trimers was added for 2 h in 1% milk PBS-T, after which the plates were washed three times with PBS-T. Serial dilutions of antigenic profiling mA bs PGT151, PG145, F105, PGT121, 12A 12, 4025, B6, PCT64.35S and PG9 in 1% milk PBS-T were added to the plates for 1 h, after which the plates were washed again three times with PBS-T before the addition of anti-human conjugated peroxidase at 1:1000 for 1 h. After four final washes, binding was detected by the addition of TMB substrate and measured by absorbance at 405 nm.

Cryo-EM Sample Preparation. Purified ApexGT2.2M UT, Apex3, or ApexGT3.N130 was incubated overnight at 4° with ˜6 molar excess purified PCT64.LMCA, PCT64.35S, PG9.iGL, PG9, and/or RM20A 3 Fab then purified via size exclusion chromatography on a Superdex 200 Increase column followed by concentration of pooled fractions with a 30 kD molecular weight cut-off using an Amicon Ultra centrifugal filter to a final concentration of ˜3-7 mg/ml. Concentrated sample was mixed with 0.5 ul of 0.04 mM lauryl maltose neopentyl glycol (LMNG; A natrace) to a final concentration of 0.005 mM and 4 μl of this solution was applied to plasma cleaned 1.2/1.3 C-Flat holey carbon grids (Electron Microscopy Sciences) using a Vitrobot mark IV (Thermo Fisher Scientific) with a 7 sec blot time, 0 blot force, and wait time of 0 sec. Prepared grids were then stored in liquid nitrogen until they were transfer to a microscope for imaging.

Cryo-EM data collection. A table of detailed imaging conditions and data statistics for all the EM datasets is presented in Supplemental Table 6. All datasets were collected with Legion automated microscopy software (Suloway et al., 2005) on either an FEI Titan K rios operating at 300 keV or an FEI Talos Arctica operating at 200 keV Thermo Fisher Scientific), both equipped with a K 2 Summit direct electron detector (Gatan) operated in counting mode.

Cryo-EM Data processing. All movie micrographs were aligned and dose-weighted using MotionCor2 (Zheng et al., 2017) and CTF parameters were estimated with GCTF (Zhang, 2016) Single-particle processing was carried out using a combination of either Relion-2/3 (Kimanius et al., 2016; Zivanov et al., 2018) and CryoSparc2 (Punjani and Fleet, 2021). The ApexGT2.2M UT+PCT64.LMCA, and both ApexGT3 datasets were collected several years earlier than the ApexGT2.2M UT+PCT64.35S dataset and were processed initially using exclusively Relion2. However, because improved software became available during the preparation of this paper, particles from each dataset were uploaded to CryoSparc2 and further processed as shown in Supplemental FIGS. 11,12,16 and 17. The ApexGT3A+PG9.iGL and ApexGT3A.N130+PG9 complexes were both imaged in two sessions on different microscopes and combined by down sampling the higher magnification data to match the final pixel size of the lower magnification data using Relion-2. The following general workflow was used for all datasets presented in this study. After frame alignment, dose-weighting, and CTF estimation, micrographs were sorted based on CTF fit parameters and particle picking was performed first using a gaussian blot template on a subset of micrographs. These particles were then extracted, aligned and classified in 2-D, and the class averages were then used for template picking of the full dataset. Picked particles were extracted and subjected to one or two rounds of 2D-classification followed by subset selection and re-extraction with re-centering. One round of ab initio classification was carried out followed by subset selection of all classes containing well refined trimers. After subset selection, 3D-auto refinement was performed with per-particle CTF estimation followed by another round of 3-D classification, this time using the newly developed 3-D variability algorithm implemented in CryoSparc2. A soft spherical mask that surrounds the trimer apex and large enough to accommodate the entire Fab was used to isolate variability in Fab occupancy followed by clustering into 3-6 classes. Clusters with clear density for Fab were then pooled and refined again together. 3-D variability was then employed again for both PCT64 datasets, this time to isolate variability in Fab binding angle followed by clustering and pooling of particles with similar angle of approach. Lastly, a single final round of 3-D non-uniform refinement with per-particle CTF estimation and correction were performed to generate the published reconstructions. For the apo ApexGT2.2M UT structure, C3 symmetry was imposed during refinement.

Model building and figure preparation. Model building was initiated by preparing a monomeric Env homology model with SWISS-MODEL (Waterhouse et al., 2018) using MD39-10MUTA (PDB: 5T3S) as a template. A homology model of PG9.iGL was generated using SAbPred (Dunbar et al., 2016), while crystal structures were used for PG9 (PDB: 3U4E), PCT64.LMCA (PDB: 6CA9), and PCT64.35S (PDB: 6CA6). Preliminary Env and Fab models were then fit into cryo-EM maps and combined into a single PDB file using UCSF Chimera (Pettersen et al., 2004). Initial refinement was done using Rosetta (Wang et al., 2016). Glycans were then added manually and refined using COOT (Wang et al., 2016). Fully glycosylated models were then refined again in Rosetta asking for ˜300 models. All models were validated using MolProbity (Chen et al., 2010) and EM Ringer (Barad et al., 2015) and the model with the best combined score was selected. All models were then checked and adjusted manually in COOT and re-refined with Rosetta, if necessary, then renumbered to match Kabat and HXB2 numbering schemes in the python scripting interface of COOT. Final models were then scored again with MolProbity and EM Ringer, while glycan structures were further validated with Privateer (Agirre et al., 2015). Figures were prepared with either UCSF Chimera or ChimeraX (Pettersen et al. 2021). Electrostatic potential surfaces were calculated according to Coulomb's law and visualized using Chimera. Hydrogen bonds were calculated and displayed with UCSF ChimeraX. Volume segmentation was performed with Segger (Pintilie et al., 2010) as implemented in UCSF ChimeraX. Interface surface area was calculated using PDB ePISA (Krissinel and Henrick, 2007). Figures were prepared in Adobe Illustrator (Adobe Inc.) and PowerPoint (Microsoft) and Supplemental movies were edited with Blender.

Glycan Analysis. Two methods were used in glycan analysis.

Method 1: DeGlyPHER (Baboo et al., 2021) was used to ascertain site-specific glycan occupancy and processivity on the examined glycoproteins.

Glycan Analysis Proteinase K Treatment and Deglycosylation: HIV Env glycoprotein was exchanged to water using Microcon Ultracel PL-10 centrifugal filter. Glycoprotein was reduced with 5 mM tris(2-carboxyethyl) phosphine hydrochloride (TCEP-HCl) and alkylated with 10 mM 2-Chloroacetamide in 100 mM ammonium acetate for 20 min at room temperature (RT, 24° C.). Initial protein-level deglycosylation was performed using 250 U of Endo H for 5 μg trimer, for 1 h at 37° C. Glycorotein was digested with 1:25 Proteinase K (PK) for 30 min at 37° C. PK was denatured by incubating at 90° C. for 15 min, then cooled to RT. Peptides were deglycosylated again with 250 U Endo H for 1 h at 37° C., then frozen at −80° C. and lyophilized. 100 U PNGase F was lyophilized, resuspended in 20 μl 100 mM ammonium bicarbonate prepared in H2180, and added to the lyophilized peptides. Reactions were then incubated for 1 h at 37° C., subsequently analyzed by LC-MS/MS.

Glycan Analysis LC-MS/MS. Samples were analyzed on an Q Exactive HF-X mass spectrometer. Samples were injected directly onto a 25 cm, 100 μm ID column packed with BEH 1.7 μm C18 resin. Samples were separated at a flow rate of 300 nL/min on an EASY-nLC 1200 UHPLC. Buffers A and B were 0.1% formic acid in 5% and 80% acetonitrile, respectively. The following gradient was used: 1-25% B over 160 min, an increase to 40% B over 40 min, an increase to 90% B over another 10 min and 30 min at 90% B for a total run time of 240 min. Column was re-equilibrated with solution A prior to the injection of sample. Peptides were eluted from the tip of the column and nanosprayed directly into the mass spectrometer by application of 2.8 kV at the back of the column. The mass spectrometer was operated in a data dependent mode. Full MS1 scans were collected in the Orbitrap at 120,000 resolution. The ten most abundant ions per scan were selected for HCD MS/MS at 25 NCE. Dynamic exclusion was enabled with exclusion duration of 10 s and singly charged ions were excluded.

Glycan analysis Data Processing: Protein and peptide identification were done with Integrated Proteomics Pipeline (IP2). Tandem mass spectra were extracted from raw files using RawConverter (He et al., 2015) and searched with ProLuCID (Xu et al., 2015) against a database comprising UniProt reviewed (Swiss-Prot) proteome for Homo sapiens (UP000005640), UniProt amino acid sequences for Endo H (P04067), PNGase F (Q9X BM 8), and Proteinase K (P06873), amino acid sequences for the examined proteins, and a list of general protein contaminants. The search space included no cleavage-specificity. Carbamidomethylation (+57.02146 C) was considered a static modification. Deamidation in presence of H2180 (+2.988261 N), GlcNAc (+203.079373 N), oxidation (+15.994915 M) and N-terminal pyroglutamate formation (−17.026549 Q) were considered differential modifications. Data was searched with 50 ppm precursor ion tolerance and 50 ppm fragment ion tolerance. Identified proteins were filtered using DTA Select2 (Tabb et al., 2002) and utilizing a target-decoy database search strategy to limit the false discovery rate to 1%, at the spectrum level (Peng et al., 2003). A minimum of 1 peptide per protein and no tryptic end per peptide were required and precursor delta mass cut-off was fixed at 15 ppm. Statistical models for peptide mass modification (modstat) were applied. Census2 (Park et al., 2008) label-free analysis was performed based on the precursor peak area, with a 15 ppm precursor mass tolerance and 0.1 min retention time tolerance. “Match between runs” was used to find missing peptides between runs. Data analysis using GlycoM SQuant (Baboo et al., 2021) was implemented to automate the analysis. GlycoM SQuant summed precursor peak areas across replicates, discarded peptides without NGS, discarded misidentified peptides when N-glycan remnant-mass modifications were localized to non-NGS asparagines and corrected/fixed N-glycan mislocalization where appropriate. The results were aligned to NGS in Env of HXB2 (Tian et al. 2016) HIV-1 variant.

Method 2: Single site glycan profiling (Allen et al., 2021) was used for ApexGT2 glycan analysis.

Three aliquots were denatured for 1h in 50 mM Tris/HCl, pH 8.0 containing 6 M of urea and 5 mM dithiothreitol (DTT). Next, Env proteins were reduced and alkylated by adding 20 mM iodoacetamide (IAA) and incubated for 1h in the dark, followed by a 1h incubation with 20 mM DTT to eliminate residual IAA. The alkylated Env proteins were buffer-exchanged into 50 mM Tris/HCl, pH 8.0 using Vivaspin columns (3 kDa) and two of the aliquots were digested separately overnight using trypsin, chymotrypsin (Mass Spectrometry Grade, Promega) or alpha lytic protease (Sigma Aldrich) at a ratio of 1:30 (w/w). The next day, the peptides were dried and extracted using C18 Zip-tip (MerckMilipore). The peptides were dried again, re-suspended in 0.1% formic acid and analyzed by nanoLC-ESI MS with an Ultimate 3000 HPLC (Thermo Fisher Scientific) system coupled to an Orbitrap Eclipse mass spectrometer (Thermo Fisher Scientific) using stepped higher energy collision-induced dissociation (HCD) fragmentation. Peptides were separated using an EasySpray PepMap RSLC C18 column (75 μm×75 cm). A trapping column (PepMap 100 C18 3 uM 75 uM×2 cm) was used in line with the LC prior to separation with the analytical column. The LC conditions were as follows: 280 minute linear gradient consisting of 4-32% acetonitrile in 0.1% formic acid over 260 minutes followed by 20 minutes of alternating 76% acetonitrile in 0.1% formic acid and 4% Acn in 0.1% formic acid, used to ensure all the sample had eluted from the column. The flow rate was set to 200 nL/min. The spray voltage was set to 2.7 kV and the temperature of the heated capillary was set to 40° C. The ion transfer tube temperature was set to 275° C. The scan range was 375-1500 m/z. Stepped HCD collision energy was set to 15, 25 and 45% and the MS2 for each energy was combined. Precursor and fragment detection were performed using an Orbitrap at a resolution MS1=120,000. MS2=30,000. The A GC target for MS1 was set to standard and injection time set to auto which involves the system setting the two parameters to maximize sensitivity while maintaining cycle time. Full LC and MS methodology can be extracted from the appropriate Raw file using X Calibur FreeStyle software or upon request.

Glycopeptide fragmentation data were extracted from the raw file using Byos (Version 3.5; Protein Metrics Inc.). The glycopeptide fragmentation data were evaluated manually for each glycopeptide; the peptide was scored as true-positive when the correct b and y fragment ions were observed along with oxonium ions corresponding to the glycan identified. The MS data was searched using the Protein Metrics 305 N-glycan library with sulfated glycans added manually. The relative amounts of each glycan at each site as well as the unoccupied proportion were determined by comparing the extracted chromatographic areas for different glycotypes with an identical peptide sequence. All charge states for a single glycopeptide were summed. The precursor mass tolerance was set at 4 ppm and 10 ppm for fragments. A 1% false discovery rate (FDR) was applied. The relative amounts of each glycan at each site as well as the unoccupied proportion were determined by comparing the extracted ion chromatographic areas for different glycopeptides with an identical peptide sequence. Glycans were categorized according to the composition detected.

HexNAc (2) Hex (10+) was defined as M9Glc, HexNAc (2) Hex (9-5) was classified as M9 to M3. Any of these structures containing a fucose were categorized as FM (fucosylated mannose). HexNAc (3) Hex (5-6) X was classified as Hybrid with HexNAc (3) Hex (5-6) Fuc (1) X classified as Fhybrid. Complex-type glycans were classified according to the number of HexNAc subunits and the presence or absence of fucosylation. As this fragmentation method does not provide linkage information compositional isomers are grouped, so for example a triantennary glycan contains HexNAc 5 but so does a biantennary glycans with a bisect. Core glycans refer to truncated structures smaller than M3. M9glc-M 4 were classified as oligomannose-type glycans. Glycans containing at least one sialic acid or one sulfate group were categorized as NeuAc and sulfated respectively.

Cell Surface Display Antigenic Profiling. Unformulated membrane bound mRNA immunogens (Moderna) were transfected into HEK 293F suspension cells grown in 293 Freestyle media (Life Technologies) by the Mirus TransIT-mRNA transfection Kit (MIR 2250) and incubated at 37° C., 125 rpm for 24 hrs. Each antibody solution for antigenic profile test (in FIG. 7C) was prepared at 10 μg/mL in fluorescence-activated cell sorting (FACS) buffer (HBSS, 1 mM EDTA, 1% BSA). To note, trimer-specific bnAbs (interface/FP: PGT151, V2 apex: PGT145, PG9 and PCT64.35S) and non-nAbs (V3:4025, CD4bs: B6 and F105) were selected to characterize the open vs. closed nature of the membrane bound trimer; not-trimer-specific bnAbs (N332: PGT121 and CD4bs: 12A 12) were selected to evaluate cell surface immunogen expression; germline reverted variants of PG9 and PCT64 (PG9.iGL, PCT64.LMCA, PCT64.LMCA.Jrev and PCT64.iGL) were selected to assess binding capacity of cell surface immunogen towards V2 bnAbs-like precursors.

Cell suspension was distributed onto a deep-well 96-well plate at 1 mL per well and harvested at 500 g for 5 min. Each well of cells was resuspended by 100 μL of 10 μg/ml mAb solution and incubated at 37° C., 125 rpm for 1 hr. Cells were washed twice with 150 ul FACS buffer and then stained with SYTOX™ Green Dead Cell Stain (Invitrogen) and Alexa Fluor 647-conjugated anti-human IgG (Jackson Immuno Research) at 37° C., 125 rpm for 20 min. Cells were analyzed on a NovoCyte 3000 with NovoSampler Pro FACS sorter (Agilent ACEA) by a BD FACS Diva 6 software (BD Biosciences). Approximately 50k live cells were collected per well. Data was analyzed using FlowJo™ v10.8 Software. The methods for cell surface antigenic profiling for DNA-transfected cells were similar to those for mRNA, except that DNA-encoded membrane bound trimers were transfected by the 293fectin Transfection Reagent (Gibco) and incubated for 2 days before FACS.

### REFERENCES

Abbott, R. K., Lee, J. H., Menis, S., Skog, P., Rossi, M., Ota, T., Kulp, D. W., Bhullar, D., Kalyuzhniy, O., Havenar-Daughton, C., et al. (2018). Precursor Frequency and Affinity Determine B Cell Competitive Fitness in Germinal Centers, Tested with Germline-Targeting HIV Vaccine Immunogens. Immunity 48, 133-146 e136. 10.1016/j.immuni.2017.11.023.
Agirre, J., Iglesias-Fernández, J., Rovira, C., Davies, G. J., Wilson, K. S., and Cowtan, K. D. (2015). Privateer: software for the conformational validation of carbohydrate structures. Nature Structural & Molecular Biology 22, 833-834. 10.1038/nsmb.3115.
Agresti, A., and Coull, B. A. (1998). Approximate Is Better than “Exact” for Interval Estimation of Binomial Proportions. The American Statistician 52, 119-126. 10.2307/2685469.
Alam, S. M., Dennison, S. M., Aussedat, B., Vohra, Y., Park, P. K., Fernandez-Tejada, A., Stewart, S., Jaeger, F. H., Anasti, K., Blinn, J. H., et al. (2013). Recognition of synthetic glycopeptides by HIV-1 broadly neutralizing antibodies and their unmutated ancestors. Proc Natl Acad Sci USA 110, 18214-18219. 10.1073/pnas.1317855110.
Aldon, Y., Mckay, P. F., Allen, J., Ozorowski, G., Felfodine Levai, R., Tolazzi, M., Rogers, P., He, L., de Val, N., Fabian, K., et al. (2018). Rational Design of DNA-Expressed Stabilized Native-Like HIV-1 Envelope Trimers. Cell Rep 24, 3324-3338 e3325. 10.1016/j.celrep.2018.08.051.
Allen, J. D., Chawla, H., Samsudin, F., Zuzic, L., Shivgan, A. T., Watanabe, Y., He, W. T., Callaghan, S., Song, G., Y ong, P., et al. (2021). Site-specific steric control of SARS-COV-2 spike glycosylation. bioRxiv. 10.1101/2021.03.08.433764.
Andrabi, R., Pallesen, J., Allen, J. D., Song, G., Zhang, J., de Val, N., Gegg, G., Porter, K., Su, C. Y., Pauthner, M., et al. (2019). The Chimpanzee SIV Envelope Trimer: Structure and Deployment as an HIV Vaccine Template. Cell Rep 27, 2426-2441 e2426. 10.1016/j.celrep.2019.04.082.
Andrabi, R., Su, C. Y., Liang, C. H., Shivatare, S. S., Briney, B., Voss, J. E., Nawazi, S. K., Wu, C. Y., Wong, C. H., and Burton, D. R. (2017). Glycans Function as Anchors for Antibodies and Help Drive HIV Broadly Neutralizing Antibody Development. Immunity 47, 524-537 e523. 10.1016/j.immuni.2017.08.006.
Andrabi, R., Voss, J. E., Liang, C. H., Briney, B., McCoy, L. E., Wu, C. Y., Wong, C. H., Poignard, P., and Burton, D. R. (2015). Identification of Common Features in Prototype Broadly Neutralizing Antibodies to HIV Envelope V2 Apex to Facilitate Vaccine Design. Immunity 43, 959-973. 10.1016/j.immuni.2015.10.014.
Baboo, S., Diedrich, J. K., Martinez-Bartolome, S., Wang, X., Schiffner, T., Groschel, B., Schief, W. R., Paulson, J. C., and Y ates, J. R., 3rd (2021). DeGlyPHER: An Ultrasensitive Method for the Analysis of Viral Spike N-Glycoforms. Anal Chem 93, 13651-13657. 10.1021/acs.analchem.1c03059.
Baden, L. R., El Sahly, H. M., Essink, B., Kotloff, K., Frey, S., Novak, R., Diemert, D., Spector, S. A., Rouphael, N., Creech, C. B., et al. (2021). Efficacy and Safety of the mRNA-1273 SARS-COV-2 Vaccine. N Engl J Med 384, 403-416. 10.1056/NEJMoa2035389.
Bansal, S., Perincheri, S., Fleming, T., Poulson, C., Tiffany, B., Bremner, R. M., and Mohanakumar, T. (2021). Cutting Edge: Circulating Exosomes with COVID Spike Protein Are Induced by BNT162b2 (Pfizer-BioNTech) Vaccination prior to Development of Antibodies: A Novel Mechanism for Immune Activation by mRNA Vaccines. J Immunol 207, 2405-2410. 10.4049/jimmunol.2100637.
Barad, B. A., Echols, N., Wang, R. Y., Cheng, Y., DiMaio, F., Adams, P. D., and Fraser, J. S. (2015). EM Ringer: side chain-directed model and map validation for 3D cryo-electron microscopy. Nat Methods 12, 943-946. 10.1038/nmeth.3541.
Behrens, A. J., Vasiljevic, S., Pritchard, L. K., Harvey, D. J., Andev, R. S., Krumm, S. A., Struwe, W. B., Cupo, A., Kumar, A., Zitzmann, N., et al. (2016). Composition and Antigenic Effects of Individual Glycan Sites of a Trimeric HIV-1 Envelope Glycoprotein. Cell Rep 14, 2695-2706. 10.1016/j.celrep.2016.02.058.
Bhiman, J. N., Anthony, C., Doria-Rose, N. A., Karimanzira, O., Schramm, C. A., Khoza, T., Kitchin, D., Botha, G., Gorman, J., Garrett, N. J., et al. (2015). Viral variants thatinitiate and drive maturation of V1V2-directed HIV-1 broadly neutralizing antibodies. Nat Med 21, 1332-1336. 10.1038/nm.3963.
Bianchi, M., Turner, H. L., Nogal, B., Cottrell, C. A., Oyen, D., Pauthner, M., Bastidas, R., Nedellec, R., McCoy, L. E., Wilson, I. A., et al. (2018). Electron-Microscopy-Based Epitope Mapping Defines Specificities of Polyclonal Antibodies Elicited during HIV-1 BG505 Envelope Trimer Immunization. Immunity 49, 288-300 e288. 10.1016/j.immuni.2018.07.009.
Bonsignori, M., Hwang, K.-K., Chen, X., Tsao, C.-Y., Morris, L., Gray, E., Marshall, D. J., Crump, J. A., Kapiga, S. H., Sam, N. E., et al. (2011). Analysis of a Clonal Lineage of HIV-1 Envelope V2/V3 Conformational Epitope-Specific Broadly Neutralizing Antibodies and Their Inferred Unmutated Common Ancestors. Journal of Virology 85, 9998-10009. 10.1128/jvi.05045-11.
Briney, B., Inderbitzin, A., Joyce, C., and Burton, D. R. (2019). Commonality despite exceptional diversity in the baseline human antibody repertoire. Nature 566, 393-397. 10.1038/s41586-019-0879-y.
Briney, B. S., Willis, J. R., and Crowe, J. E., Jr. (2012). Human peripheral blood antibodies with long HCDR3s are established primarily at original recombination using a limited subset of germline genes. PLOS One 7, e36750. 10.1371/journal.pone.0036750.
Burton, D. R., Barbas, C. F., 3rd, Persson, M. A., Koenig, S., Chanock, R. M., and Lerner, R. A. (1991). A large array of human monoclonal antibodies to type 1 human immunodeficiency virus from combinatorial libraries of asymptomatic seropositive individuals. Proc Natl Acad Sci USA 88, 10134-10137. 10.1073/pnas.88.22.10134.
Burton, D. R., and Hangartner, L. (2016). Broadly Neutralizing Antibodies to HIV and Their Role in Vaccine Design. Annu Rev Immunol 34, 635-659. 10.1146/annurev-immunol-041015-055515.
Burton, D. R., and Mascola, J. R. (2015). Antibody responses to envelope glycoproteins in HIV-1 infection. Nat Immunol 16, 571-576. 10.1038/ni.3158.
Cale, E. M., Gorman, J., Radakovich, N. A., Crooks, E. T., Osawa, K., Tong, T., Li, J., Nagarajan, R., Ozorowski, G., Ambrozak, D. R., et al. (2017). Virus-like Particles Identify an HIV V1V2 Apex-Binding Neutralizing Antibody that Lacks a Protruding Loop. Immunity 46, 777-791 e710. 10.1016/j.immuni.2017.04.011.
Cao, L., Diedrich, J. K., Kulp, D. W., Pauthner, M., He, L., Park, S. R., Sok, D., Su, C. Y., Delahunty, C. M., Menis, S., et al. (2017). Global site-specific N-glycosylation analysis of HIV envelope glycoprotein. Nat Commun 8, 14954. 10.1038/ncomms14954.
Cao, L., Pauthner, M., Andrabi, R., Rantalainen, K., Berndsen, Z., Diedrich, J. K., Menis, S., Sok, D., Bastidas, R., Park, S. R., et al. (2018). Differential processing of HIV envelope glycans on the virus and soluble recombinant trimer. Nat Commun 9, 3693. 10.1038/s41467-018-06121-4.
Chen, V. B., A rendall, W. B., 3rd, Headd, J. J., Keedy, D. A., Immormino, R. M., Kapral, G. J., Murray, L. W., Richardson, J. S., and Richardson, D. C. (2010). MolProbity: all-atom structure validation for macromolecular crystallography. Acta Crystallogr D Biol Crystallogr 66, 12-21. 10.1107/S0907444909042073.
Cirelli, K. M., Carnathan, D. G., Nogal, B., Martin, J. T., Rodriguez, O. L., Upadhyay, A. A., Enemuo, C. A., Gebru, E. H., Choe, Y., Viviano, F., et al. (2019). Slow Delivery Immunization Enhances HIV Neutralizing Antibody and Germinal Center Responses via Modulation of Immunodominance. Cell 177, 1153-1171 e1128. 10.1016/j.cell.2019.04.012.
Corey, L., Gilbert, P. B., Juraska, M., Montefiori, D. C., Morris, L., Karuna, S. T., E dupuganti, S., M godi, N. M., deCamp, A. C., Rudnicki, E., et al. (2021). Two Randomized Trials of Neutralizing Antibodies to Prevent HIV-1 Acquisition. N Engl J Med 384, 1003-1014. 10.1056/NEJMoa2031738. Das, R., and Baker, D. (2008). Macromolecular modeling with rosetta. Annu Rev Biochem 77, 363-382. 10.1146/annurev.biochem.77.062906.171838.
Dekosky, B. J., Kojima, T., Rodin, A., Charab, W., Ippolito, G. C., Ellington, A. D., and Georgiou, G. (2015). In-depth determination and analysis of the human paired heavy- and light-chain antibody repertoire. Nat Med 21, 86-91. 10.1038/nm.3743.
Doria-Rose, N. A., Bhiman, J. N., Roark, R. S., Schramm, C. A., Gorman, J., Chuang, G.-Y., Pancera, M., Cale, E. M., Ernandes, M. J., Louder, M. K., et al. (2016). New Member of the V1V2-Directed CAP256-VRC26 Lineage That Shows Increased Breadth and Exceptional Potency. Journal of Virology 90, 76-91. 10.1128/jvi.01791-15.
Doria-Rose, N. A., Schramm, C. A., Gorman, J., Moore, P. L., Bhiman, J. N., DeKosky, B. J., Ernandes, M. J., Georgiev, I. S., Kim, H. J., Pancera, M., et al. (2014). Developmental pathway for potent V1V2-directed HIV-neutralizing antibodies. Nature 509, 55-62. 10.1038/nature13036.
Dunbar, J., Krawczyk, K., Leem, J., Marks, C., Nowak, J., Regep, C., Georges, G., Kelm, S., Popovic, B., and Deane, C. M. (2016). SAbPred: a structure-based antibody prediction server. Nucleic Acids Res 44, W474-478. 10.1093/nar/gkw361.
Emsley, P., and Cowtan, K. (2004). Coot: model-building tools for molecular graphics. Acta Crystallogr D Biol Crystallogr 60, 2126-2132. 10.1107/S0907444904019158.
Falkowska, E., Le, K. M., Ramos, A., Doores, K. J., Lee, J. H., Blattner, C., Ramirez, A., Derking, R., van Gils, M. J., Liang, C. H., et al. (2014). Broadly neutralizing HIV antibodies define a glycan-dependent epitope on the prefusion conformation of gp41 on cleaved envelope trimers. Immunity 40, 657-668. 10.1016/j.immuni.2014.04.009.
Freund, N. T., Wang, H., Scharf, L., Nogueira, L., Horwitz, J. A., Bar-On, Y., Golijanin, J., Sievers, S. A., Sok, D., Cai, H., et al. (2017). Coexistence of potent HIV-1 broadly neutralizing antibodies and antibody-sensitive viruses in a viremic controller. Sci Transl Med 9. 10.1126/scitranslmed.aal2144.
Gorman, J., Soto, C., Yang, M. M., Davenport, T. M., Guttman, M., Bailer, R. T., Chambers, M., Chuang, G. Y., DeK osky, B. J., Doria-Rose, N. A., et al. (2016). Structures of HIV-1 Env V1V2 with broadly neutralizing antibodies reveal commonalities that enable vaccine design. Nat Struct Mol Biol 23, 81-90. 10.1038/nsmb.3144.
Gorny, M. K., Sampson, J., Li, H., Jiang, X., Totrov, M., Wang, X. H., Williams, C., O'Neal, T., Volsky, B., Li, L., et al. (2011). Human anti-V3 HIV-1 monoclonal antibodies encoded by the VH5-51/VL lambda genes define a conserved antigenic structure. PLOS One 6, e27780. 10.1371/journal.pone.0027780.
He, L., Diedrich, J., Chu, Y. Y., and Yates, J. R., 3rd (2015). Extracting Accurate Precursor Information for Tandem Mass Spectra by RawConverter. A nal Chem 87, 11361-11367. 10.1021/acs.analchem.5b02721.
www.unaids.org/en/resources/fact-sheet.
Hu, J. K., Crampton, J. C., Cupo, A., Ketas, T., van Gils, M. J., Sliepen, K., de Taeye, S. W., Sok, D., Ozorowski, G., Deresa, I., et al. (2015). M urine Antibody Responses to Cleaved Soluble HIV-1 Envelope Trimers Are Highly Restricted in Specificity. Journal of Virology 89, 10383-10398. 10.1128/jvi.01653-15.
Huang, D., Abbott, R. K., Havenar-Daughton, C., Skog, P. D., Al-Kolla, R., Groschel, B., Blane, T. R., Menis, S., Tran, J. T., Thinnes, T. C., et al. (2020). B cells expressing authentic naive human VRC01-class BCRs can be recruited to germinal centers and affinity mature in multiple independent mouse models. Proc Natl Acad Sci USA 117, 22920-22931. 10.1073/pnas.2004489117.
Jardine, J., Julien, J. P., Menis, S., Ota, T., Kalyuzhniy, O., McGuire, A., Sok, D., Huang, P. S., MacPherson, S., Jones, M., et al. (2013). Rational HIV immunogen design to target specific germline B cell receptors. Science 340, 711-716. 10.1126/science.1234150.
Jardine, J. G., Ota, T., Sok, D., Pauthner, M., Kulp, D. W., Kalyuzhniy, O., Skog, P. D., Thinnes, T. C., Bhullar, D., Briney, B., et al. (2015). Priming a broadly neutralizing antibody response to HIV-1 using a germline-targeting immunogen. Science 349, 156-161. 10.1126/science.aac5894.
Julien, J. P., Cupo, A., Sok, D., Stanfield, R. L., Lyumkis, D., Deller, M. C., Klasse, P. J., Burton, D. R., Sanders, R. W., Moore, J. P., et al. (2013). Crystal structure of a soluble cleaved HIV-1 envelope trimer. Science 342, 1477-1483. 10.1126/science.1245625.
Jurrus, E., Engel, D., Star, K., Monson, K., Brandi, J., Felberg, L. E., Brookes, D. H., Wilson, L., Chen, J., Liles, K., et al. (2018). Improvements to the A PBS biomolecular solvation software suite. Protein Sci 27, 112-128. 10.1002/pro.3280.
Kato, Y., Abbott, R. K., Freeman, B. L., Haupt, S., Groschel, B., Silva, M., Menis, S., Irvine, D. J., Schief, W. R., and Crotty, S. (2020). Multifaceted Effects of Antigen Valency on B Cell Response Composition and Differentiation In Vivo. Immunity 53, 548-563 e548. 10.1016/j.immuni.2020.08.001.
Kimanius, D., Forsberg, B. O., Scheres, S. H., and Lindahl, E. (2016). Accelerated cryo-EM structure determination with parallelisation using GPUs in RELION-2. Elife 5. 10.7554/eLife.18722.
Krissinel, E., and Henrick, K. (2007). Inference of Macromolecular Assemblies from Crystalline State. Journal of Molecular Biology 372, 774-797.//doi.org/10.1016/j.jmb.2007.05.022. Kulp, D. W., Steichen, J. M., Pauthner, M., Hu, X., Schiffner, T., Liguori, A., Cottrell,
C. A., Havenar-Daughton, C., Ozorowski, G., Georgeson, E., et al. (2017). Structure-based design of native-like HIV-1 envelope trimers to silence non-neutralizing epitopes and eliminate CD4 binding. Nat Commun 8, 1655. 10.1038/s41467-017-01549-6.
Kulp, D.W.a.S.M.a.P.M.a.H.X.a.S.T.a.L.A.a.C.C.A.a.H.-D.C.a.O.G. (2017). Structure-based design of native-like HIV-1 envelope trimers to silence non-neutralizing epitopes and eliminate CD4 binding. Nature communications 8, 1655. 10.1038/s41467-017-01549-6.
Kwong, P. D., and Mascola, J. R. (2018). HIV-1 Vaccines Based on Antibody Identification, B Cell Ontogeny, and Epitope Structure. Immunity 48, 855-871. 10.1016/j.immuni.2018.04.029.
Landais, E., Huang, X., Havenar-Daughton, C., Murrell, B., Price, M. A., Wickramasinghe, L., Ramos, A., Bian, C. B., Simek, M., Allen, S., et al. (2016). Broadly Neutralizing Antibody Responses in a Large Longitudinal Sub-Saharan HIV Primary Infection Cohort. Plos Pathogens 12, e1005369. 10.1371/journal.ppat.1005369.
Landais, E., Murrell, B., Briney, B., Murrell, S., Rantalainen, K., Berndsen, Z. T., Ramos, A., Wickramasinghe, L., Smith, M. L., Eren, K., et al. (2017). HIV Envelope Glycoform Heterogeneity and Localized Diversity Govern the Initiation and Maturation of a V2 Apex Broadly Neutralizing Antibody Lineage. Immunity 47, 990-1003 e1009. 10.1016/j.immuni.2017.11.002.
Lee, J. H., Andrabi, R., Su, C. Y., Yasmeen, A., Julien, J. P., Kong, L., Wu, N. C., McBride, R., Sok, D., Pauthner, M., et al. (2017). A Broadly Neutralizing Antibody Targets the Dynamic HIV Envelope Trimer Apex via a Long, Rigidified, and Anionic beta-Hairpin Structure. Immunity 46, 690-702. 10.1016/j.immuni.2017.03.017
Lyumkis, D., Doamekpor, S. K., Bengtson, M. H., Lee, J. W., Toro, T. B., Petroski, M. D., Lima, C. D., Potter, C. S., Carragher, B., and Joazeiro, C. A. (2013). Single-particle EM reveals extensive conformational variability of the Ltn1 E3 ligase. Proc Natl Acad Sci USA 110, 1702-1707. 10.1073/pnas.1210041110.
McCoy, L. E., van Gils, M. J., Ozorowski, G., Messmer, T., Briney, B., Voss, J. E., Kulp, D. W., Macauley, M. S., Sok, D., Pauthner, M., et al. (2016). Holes in the Glycan Shield of the Native HIV Envelope Are a Target of Trimer-Elicited Neutralizing Antibodies. Cell Rep 16, 2327-2338. 10.1016/j.celrep.2016.07.074.
Mclellan, J. S., Pancera, M., Carrico, C., Gorman, J., Julien, J. P., Khayat, R., Louder, R., Pejchal, R., Sastry, M., Dai, K., et al. (2011). Structure of HIV-1 gp120 V1/V2 domain with broadly neutralizing antibody PG9. Nature 480, 336-343. 10.1038/nature10696.
Medina-Ramirez, M., Garces, F., Escolano, A., Skog, P., de Taeye, S. W., Del Moral-Sanchez, I., McGuire, A. T., Yasmeen, A., Behrens, A. J., Ozorowski, G., et al. (2017). Design and crystal structure of a native-like HIV-1 envelope trimer that engages multiple broadly neutralizing antibody precursors in vivo. J Exp Med 214, 2573-2590. 10.1084/jem.20161160.
Melzi, E., Willis, J. R., Ma, K. M., Lin, Y.-C., Kratochvil, S., Berndsen, Z. T., Landais, E. A., Kalyuzhniy, O., Nair, U., Warner, J., et al. (2022). Priming HIV Envelope V2 Apex-directed broadly neutralizing antibody responses with protein or mRNA immunogens. Immunity Submitted.
Nogal, B., Bianchi, M., Cottrell, C. A., Kirchdoerfer, R. N., Sewall, L. M., Turner, H. L., Zhao, F., Sok, D., Burton, D. R., Hangartner, L., and Ward, A. B. (2020). Mapping Polyclonal Antibody Responses in Non-human Primates Vaccinated with HIV Env Trimer Subunit Vaccines. Cell Rep 30, 3755-3765 e3757. 10.1016/j.celrep.2020.02.061.
Ota, T., Doyle-Cooper, C., Cooper, A. B., Huber, M., Falkowska, E., Doores, K. J., Hangartner, L., Le, K., Sok, D., Jardine, J., et al. (2012). Anti-HIV B Cell lines as candidate vaccine biosensors. J Immunol 189, 4816-4824. 10.4049/jimmunol.1202165.
Pancera, M., Mclellan, J. S., Wu, X., Zhu, J., Changela, A., Schmidt, S. D., Yang, Y., Zhou, T., Phogat, S., Mascola, J. R., and Kwong, P. D. (2010). Crystal structure of PG16 and chimeric dissection with somatically related PG9: structure-function analysis of two quaternary-specific antibodies that effectively neutralize HIV-1. J Virol 84, 8098-8110. 10.1128/JVI.00966-10.
Pancera, M., Shahzad-ul-Hussan, S., Doria-Rose, N. A., Mclellan, J. S., Bailer, R. T., Dai, K., Loesgen, S., Louder, M. K., Staupe, R. P., Y ang, Y., et al. (2013). Structural basis for diverse N-glycan recognition by HIV-1-neutralizing V1-V2-directed antibody PG16. Nature Structural & Molecular Biology 20, 804-+. 10.1038/nsmb.2600.
Park, S. K., Venable, J. D., Xu, T., and Y ates, J. R., 3rd (2008). A quantitative analysis software tool for mass spectrometry-based proteomics. Nat Methods 5, 319-322. 10.1038/nmeth.1195.
Parks, K. R., MacCamy, A. J., Trichka, J., Gray, M., Weidle, C., Borst, A. J., K hechaduri, A., Takushi, B., Agrawal, P., Guenaga, J., et al. (2019). Overcoming Steric Restrictions of VRC01 HIV-1 Neutralizing Antibodies through Immunization. Cell Rep 29, 3060-3072 e3067. 10.1016/j.celrep.2019.10.071.
Pegu, A., Borate, B., Huang, Y., Pauthner, M. G., Hessell, A. J., Julg, B., Doria-Rose, N. A., Schmidt, S. D., Carpp, L. N., Cully, M. D., et al. (2019). A Meta-analysis of Passive Immunization Studies Shows that Serum-Neutralizing Antibody Titer Associates with Protection against SHIV Challenge. Cell Host Microbe 26, 336-346 e333. 10.1016/j.chom.2019.08.014.
Pegu, A., Hessell, A. J., Mascola, J. R., and Haigwood, N. L. (2017). Use of broadly neutralizing antibodies for HIV-1 prevention. Immunol Rev 275, 296-312. 10.1111/imr.12511.
Pejchal, R., Walker, L. M., Stanfield, R. L., Phogat, S. K., Koff, W. C., Poignard, P., Burton, D. R., and Wilson, I. A. (2010). Structure and function of broadly reactive antibody PG16 reveal an H3 subdomain that mediates potent neutralization of HIV-1. Proc Natl Acad Sci USA 107, 11483-11488. 10.1073/pnas.1004600107.
Peng, J., Elias, J. E., Thoreen, C. C., Licklider, L. J., and Gygi, S. P. (2003). Evaluation of multidimensional chromatography coupled with tandem mass spectrometry (LC/LC-MS/MS) for large-scale protein analysis: the yeast proteome. J Proteome Res 2, 43-50. 10.1021/pr025556v.
Pettersen, E. F., Goddard, T. D., Huang, C. C., Couch, G. S., Greenblatt, D. M., Meng, E. C., and Ferrin, T. E. (2004). UCSF Chimera-A visualization system for exploratory research and analysis. Journal of Computational Chemistry 25, 1605-1612.//doi.org/10.1002/jcc.20084.
Pettersen, E. F., Goddard, T. D., Huang, C. C., Meng, E. C., Couch, G. S., Croll, T. I., Morris, J. H., and Ferrin, T. E. (2021). UCSF ChimeraX: Structure visualization for researchers, educators, and developers. Protein Science 30, 70-82.//doi.org/10.1002/pro.3943.
Pintilie, G., and Chiu, W. (2012). Comparison of Segger and other methods for segmentation and rigid-body docking of molecular components in cryo-EM density maps. Biopolymers 97, 742-760. 10.1002/bip.22074.
Pintilie, G. D., Zhang, J., Goddard, T. D., Chiu, W., and Gossard, D. C. (2010). Quantitative analysis of cryo-EM density map segmentation by watershed and scale-space filtering, and fitting of structures by alignment to regions. J Struct Biol 170, 427-438. 10.1016/j.jsb.2010.03.007.
Polack, F. P., Thomas, S., Kitchin, N., Absalon, J., Gurtman, A., Lockhart, S., Perez, J. L., Perez Marc, G., Moreira, E. D., Zerbini, C., et al. (2020). Safety and Efficacy of the BNT162b2 mRNA Covid-19 Vaccine. N Engl J Med 383, 2603-2615. 10.1056/NEJMoa2034577.
Posner, M. R., Hideshima, T., Cannon, T., Mukherjee, M., M ayer, K. H., and Byrn, R. A. (1991). An IgG human monoclonal antibody that reacts with HIV-1/GP120, inhibits virus binding to cells, and neutralizes infection. J Immunol 146, 4325-4332.
Punjani, A., and Fleet, D. J. (2021). 3D variability analysis: Resolving continuous flexibility and discrete heterogeneity from single particle cryo-EM. J Struct Biol 213, 107702. 10.1016/j.jsb.2021.107702.
Rantalainen, K., Berndsen, Z. T., Murrell, S., Cao, L., Omorodion, O., Torres, J. L., Wu, M., Umotoy, J., Copps, J., Poignard, P., et al. (2018). Co-evolution of HIV Envelope and Apex-Targeting Neutralizing Antibody Lineage Provides Benchmarks for Vaccine Design. Cell Rep 23, 3249-3261. 10.1016/j.celrep.2018.05.046.
Salmon, P., and Trono, D. (2007). Production and titration of lentiviral vectors. Curr Protoc Hum Genet Chapter 12, Unit 12 10. 10.1002/0471142905.hg1210s54.
Sanders, R. W., Derking, R., Cupo, A., Julien, J. P., Yasmeen, A., de Val, N., Kim, H. J., Blattner, C., de la Pena, A. T., Korzun, J., et al. (2013). A next-generation cleaved, soluble HIV-1 Env trimer, BG505 SOSIP.664 gp140, expresses multiple epitopes for broadly neutralizing but not non-neutralizing antibodies. PLOS Pathog 9, e1003618. 10.1371/journal.ppat.1003618.
Scheid, J. F., Mouquet, H., Ueberheide, B., Diskin, R., Klein, F., Oliveira, T. Y., Pietzsch, J., Fenyo, D., Abadir, A., Velinzon, K., et al. (2011). Sequence and structural convergence of broad and potent HIV antibodies that mimic CD4 binding. Science 333, 1633-1637. 10.1126/science.1207227.
Sliepen, K., Medina-Ramirez, M., Yasmeen, A., Moore, J. P., Klasse, P. J., and Sanders, R. W. (2015). Binding of inferred germline precursors of broadly neutralizing HIV-1 antibodies to native-like envelope trimers. Virology 486, 116-120. 10.1016/j.virol.2015.08.002.
Sok, D., Briney, B., Jardine, J. G., Kulp, D. W., Menis, S., Pauthner, M., Wood, A., Lee, E. C., Le, K. M., Jones, M., et al. (2016). Priming HIV-1 broadly neutralizing antibody precursors in human Ig loci transgenic mice. Science 353, 1557-1560. 10.1126/science.aah3945.
Sok, D., van Gils, M. J., Pauthner, M., Julien, J.-P., Saye-Francisco, K. L., Hsueh, J., Briney, B., Lee, J. H., Le, K. M., Lee, P. S., et al. (2014). Recombinant HIV envelope trimer selects for quaternary-dependent antibodies targeting the trimer apex. Proceedings of the National Academy of Sciences of the United States of America 111, 17624-17629. 10.1073/pnas.1415789111.
Steichen, J. M., Kulp, D. W., Tokatlian, T., Escolano, A., Dosenovic, P., Stanfield, R. L., McCoy, L. E., Ozorowski, G., Hu, X., K alyuzhniy, O., et al. (2016). HIV Vaccine Design to Target Germline Precursors of Glycan-Dependent Broadly Neutralizing Antibodies. Immunity 45, 483-496. 10.1016/j.immuni.2016.08.016.
Steichen, J. M., Lin, Y. C., Havenar-Daughton, C., Pecetta, S., Ozorowski, G., Willis, J. R., Toy, L., Sok, D., Liguori, A., Kratochvil, S., et al. (2019). A generalized HIV vaccine design strategy for priming of broadly neutralizing antibody responses. Science 366. 10.1126/science.aax4380.
Suloway, C., Pulokas, J., Fellmann, D., Cheng, A., Guerra, F., Quispe, J., Stagg, S., Potter, C. S., and Carragher, B. (2005). Automated molecular microscopy: The new Leginon system. Journal of Structural Biology 151, 41-60.//doi.org/10.1016/j.jsb.2005.03.010.
Tabb, D. L., McDonald, W. H., and Y ates, J. R., 3rd (2002). DTA Select and Contrast: tools for assembling and comparing protein identifications from shotgun proteomics. J Proteome Res 1, 21-26. 10.1021/pr015504q.
Tian, M., Cheng, C., Chen, X., Duan, H., Cheng, H. L., Dao, M., Sheng, Z., Kimble, M., Wang, L., Lin, S., et al. (2016). Induction of HIV Neutralizing Antibody Lineages in Mice with Diverse Precursor Repertoires. Cell 166, 1471-1484 e1418. 10.1016/j.cell.2016.07.029.
Turner, H. L., Andrabi, R., Cottrell, C. A., Richey, S. T., Song, G., Callaghan, S., Anzanello, F., Moyer, T. J., A braham, W., Melo, M., et al. (2021). Disassembly of HIV envelope glycoprotein trimer immunogens is driven by antibodies elicited via immunization. Sci Adv 7. 10.1126/sciadv.abh2791.
Walker, L. M., Huber, M., Doores, K. J., Falkowska, E., Pejchal, R., Julien, J.-P., Wang, S.-K., Ramos, A., Chan-Hui, P.-Y., Moyle, M., et al. (2011). Broad neutralization coverage of HIV by multiple highly potent antibodies. Nature 477, 466-U 117. 10.1038/nature10373.
Walker, L. M., Phogat, S. K., Chan-Hui, P. Y., Wagner, D., Phung, P., Goss, J. L., Wrin, T., Simek, M. D., Fling, S., Mitcham, J. L., et al. (2009). B road and potent neutralizing antibodies from an African donor reveal a new HIV-1 vaccine target. Science 326, 285-289. 10.1126/science.1178746.
Walker, L. M., Simek, M. D., Priddy, F., Gach, J. S., Wagner, D., Zwick, M. B., Phogat, S. K., Poignard, P., and Burton, D. R. (2010). A limited number of antibody specificities mediate broad and potent serum neutralization in selected HIV-1 infected individuals. PLOS Pathog 6, e1001028. 10.1371/journal.ppat.1001028.
Wang, H., Gristick, H. B., Scharf, L., West, A. P., Galimidi, R. P., Seaman, M. S., Freund, N. T., Nussenzweig, M. C., and Bjorkman, P. J. (2017). A symmetric recognition of HIV-1 Envelope trimer by V1V2 loop-targeting antibodies. Elife 6. 10.7554/eLife.27389.
Wang, R. Y., Song, Y., Barad, B. A., Cheng, Y., Fraser, J. S., and DiMaio, F. (2016). Automated structure refinement of macromolecular assemblies from cryo-EM maps using Rosetta. Elife 5. 10.7554/eLife.17219.
Wang, X., Ray, R., Kratochvil, S., Melzi, E., Lin, Y. C., Giguere, S., Xu, L., Warner, J., Cheon, D., Liguori, A., et al. (2021). Multiplexed CRISPR/CAS9-mediated engineering of pre-clinical mouse models bearing native human B cell receptors. EMBO J 40, e105926. 10.15252/embj.2020105926.
Waterhouse, A., Bertoni, M., Bienert, S., Studer, G., Tauriello, G., Gumienny, R., Heer, F. T., de Beer, T. A P., Rempfer, C., Bordoli, L., et al. (2018). SWISS-MODEL: homology modelling of protein structures and complexes. Nucleic Acids Research 46, W296-W303. 10.1093/nar/gky427.
Xu, T., Park, S. K., Venable, J. D., Wohlschlegel, J. A., Diedrich, J. K., Cociorva, D., Lu, B., Liao, L., Hewel, J., Han, X., et al. (2015). ProLuCID: An improved SEQUEST-like algorithm with enhanced sensitivity and specificity. J Proteomics 129, 16-24. 10.1016/j.jprot.2015.07.001.
Zhang, K. (2016). Gctf: Real-time CTF determination and correction. Journal of Structural Biology 193, 1-12.//doi.org/10.1016/j.jsb.2015.11.003.
Zheng, S. Q., Palovcak, E., Armache, J.-P., Verba, K. A., Cheng, Y., and A gard, D. A. (2017). MotionCor2: anisotropic correction of beam-induced motion for improved cryo-electron microscopy. Nature Methods 14, 331-332. 10.1038/nmeth.4193.
Zivanov, J., Nakane, T., Forsberg, B. O., Kimanius, D., Hagen, W. J., Lindahl, E., and Scheres, S. H. (2018). New tools for automated high-resolution cryo-EM structure determination in RELION-3. Elife 7. 10.7554/eLife.42166.

### Example 2: Priming HIV Envelope V2 Apex-Directed Broadly Neutralizing Antibody Responses with Protein or mRNA Immunogens

Eliciting broadly neutralizing antibodies (bnAbs) is the core of HIV vaccine design. BnAbs specific for the V2-apex region of the HIV envelope acquire breadth and potency with modest somatic hypermutation, making them attractive vaccination targets. To evaluate ApexGT vaccine candidates, Applicants engineered knock-in (KI) mouse models expressing the germline B cell receptor (BCR) of the PCT64 bnAb. Applicants found that high affinity of ApexGT for the PCT64-germline BCRs was necessary to specifically activate KI B cells at human physiological frequencies, recruit them to germinal centers, and select for mature bnAb mutations. Furthermore, Applicants demonstrated that, relative to protein, mRNA-encoded membrane-anchored ApexGT immunization significantly increased activation and recruitment of PCT64 precursors to germinal centers and lowered their affinity threshold. Applicants have thus developed new models for HIV vaccine research, validated ApexGT immunogens for priming V2-apex bnAb precursors and identified mRNA-LNP as a suitable approach to substantially improve the B cell response.

Despite nearly four decades of research, the elicitation of protective and durable immune responses against HIV through vaccination remains elusive (Ng'uni, Chasara and Ndhlovu, 2020). In the past ten years, the discovery that some HIV-infected individuals can develop broadly neutralizing antibodies (bnAbs) capable of potently neutralizing a high proportion of HIV-1 isolates revolutionized the field (Klein et al., 2013; Flemming, 2018; Sok and Burton, 2018). These bnAbs target highly conserved regions of HIV Env: the CD4 binding site (CD4bs), the high mannose patch (N332 glycan supersite), the GP120/GP41 interface, the membrane proximal external region (M PER) and the V2-apex region (Burton and Hangartner, 2016; Sok and Burton, 2018).

Elicitation of HIV bnAbs by vaccination is challenging, as these antibodies generally exhibit one or more unusual traits: long heavy-chain third complementarity-determining regions (HCDR3s), high levels of somatic hypermutation (SHM), insertions and deletions (indels) and poly- or autoreactivity (Klein et al., 2013; Burton and Hangartner, 2016; Kelsoe and Haynes, 2017). In HIV-infected individuals, these features result from B cells co-evolving with the virus, undergoing multiple rounds of hypermutation and selection inside the germinal centers (GCs) in response to viral escape mutations (Doria-Rose and Landais, 2019). Although the inferred precursors of some bnAbs have affinity for Env from particular HIV isolates (Pancera et al., 2010; Liao et al., 2013; Doria-Rose et al., 2014; Andrabi et al., 2015; Gorman et al., 2016), many germline-reverted bnAb precursors have no detectable affinity for Env (Xiao et al., 2009; Zhou et al., 2010; Mouquet et al., 2012; Hoot et al., 2013; Jardine et al., 2013; Sok et al., 2013; Steichen et al., 2019), providing a possible explanation as to why traditional immunization strategies using native Env trimers have failed (Sanders and Moore, 2017; Ng'uni, Chasara and Ndhlovu, 2020). A germline-targeting (GT) approach using rationally designed immunogens to prime B cells encoding germline antibodies with the potential to develop into bnAbs might overcome this bottleneck (Jardine et al., 2013; Rappuoli et al., 2016; Steichen et al., 2019). Post-activation, these precursor B cells could be shepherded toward breadth and potency by consecutive booster immunogens increasingly resembling native Env (Jardine et al., 2013; McGuire et al., 2013; Briney et al., 2016; Escolano et al., 2016; Steichen et al., 2016; Tian et al., 2016; Stamatatos, Pancera and McGuire, 2017; Chen et al., 2021).

Pre-clinical in vivo models that allow for rapid iterations to characterize the activation and maturation of specific human B cell clones are required to reproducibly study antibody evolution trajectories induced by GT immunogens at the systemic, cellular and molecular levels (Dosenovic et al., 2015; Jardine et al., 2015; Escolano et al., 2016; Tian et al., 2016). Mouse models expressing human Ig have proven effective in evaluating HIV immunization regimens (Ota et al., 2013; Dosenovic et al., 2015; Tian et al., 2016; Lin et al., 2018). They have been used to validate GT immunogens designed to activate precursors for CD4bs− (VRC01-class bnAbs) (Dosenovic et al., 2015; Jardine et al., 2015, 2016; Tian et al., 2016) and N332-supersite-binding bnAbs (PGT121- and BG18-class bnAbs) (Escolano et al., 2016; Steichen et al., 2016, 2019).

Previously, Applicants reported an approach to design GT immunogens to bind precursors of HCDR3-dominant bnAbs, which triggered robust responses from BG18 precursors (Steichen et al., 2019). V2-apex directed antibodies, such as PG9, PG16, and CAP256, are also heavily reliant on HCDR3 for neutralization, providing ideal targets to determine whether the immunogen design strategy is generalizable to another Env epitope. Most V2-apex bnAbs have long (>25 residues), protruding, anionic, and often tyrosine-sulphated HCDR3 loops to penetrate the glycan shield and reach a positively charged glycopeptide epitope on the Apex of Env (Walker et al., 2009; Pancera et al., 2013; Doria-Rose et al., 2014; Andrabi et al., 2015). Notably, V2-apex bnAbs are among the most commonly identified bnAb families in patient serum samples, arise early post-infection (Walker et al., 2010; Georgiev et al., 2013; Landais et al., 2016), and require only moderate levels of SHM, making them highly desirable targets for immunization (Moore et al., 2017).

A V2-apex-directed bnAb of particular interest is PCT64, isolated from an HIV-1 subtype A-infected donor (Landais et al., 2016, 2017). PCT64 neutralizes 29% of all HIV isolates with moderate potency, and up to 56% and 48% of subtype A and C viruses, respectively (Landais et al., 2017). It has an HCDR3-length of 25 amino acids and SHM-levels of 10-12%; compared to other V2-Apex bnAbs, its HC precursors are relatively common in the human repertoire (upper limit of ˜20 in 1×10⁶ B cells) (Willis et al., 2021). Furthermore, the maturation trajectory of the PCT64 antibody line has been described in a three year-long coevolutionary study that offers a blueprint for recapitulation (Haynes et al., 2016; Landais et al., 2017; Rantalainen et al., 2018).

Herein, Applicants used PCT64 as a prototype model to study GT immunization strategies aimed at triggering V2-apex bnAb precursors. Applicants developed two preclinical mouse models with B cells expressing two distinct early ancestors of PCT64 and used them to assess the capacity of new protein- and mRNA-based ApexGT immunogens (Willis et al. 2021) to activate PCT64 precursors, induce GC responses and trigger SHM. Applicants' results demonstrated that high affinity ApexGT immunogens activate PCT64 precursors at human physiological frequencies and induce on-track mature PCT64-like mutations, with PCT64 evolution driven primarily by the HC. Furthermore, Applicants found that mRNA-LNP mediated in vivo expression of a membrane-anchored Apex-GT trimer immunogen is a particularly promising avenue for priming PCT64-like responses.

Generation of a PCT64 precursor knock-in mouse. To study immune responses against the Apex of HIV-1 Env in vivo, Applicants generated a knock-in (KI) mouse with B cells bearing the least mutated common ancestor (LMCA) heavy chain (IGH) and light chain (IGK) of the PCT64 bnAb, isolated from the human donor (Landais et al., 2017). PCT64 LMCA IGH exhibits high germline sequence identity (99.4%), with a fully reverted germline V gene (VH3-15*01), and a J gene (JH6*03) containing three amino acid mutations, while the PCT64LMCA IGK is encoded by a fully germline human V gene (Vκ3-20*01) and J gene (Jκ3*01) (Landais et al., 2017).

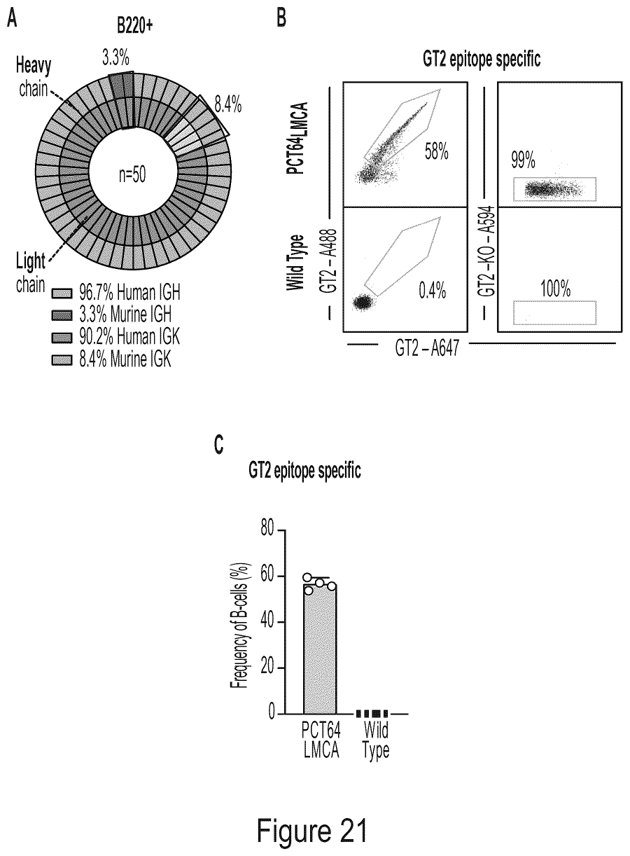

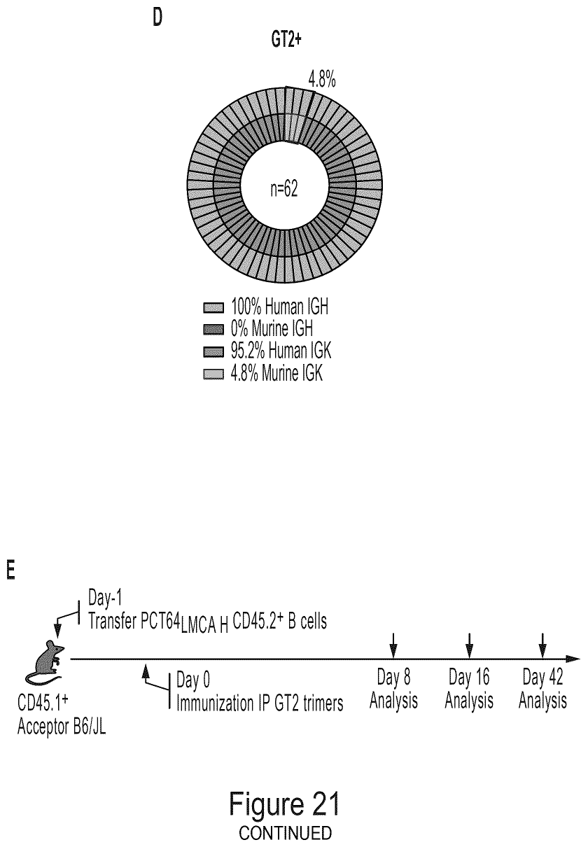

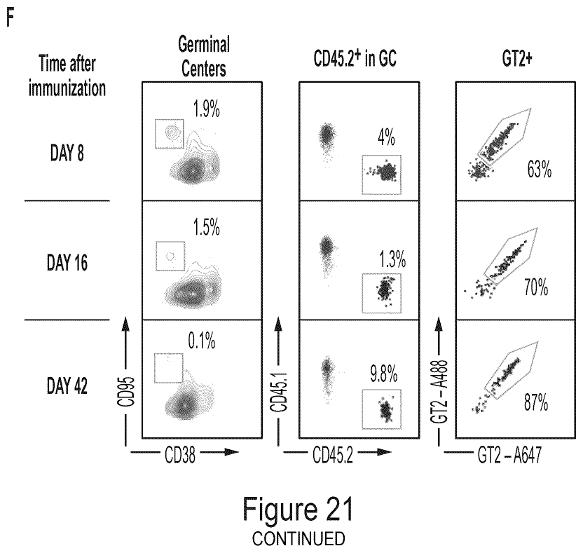

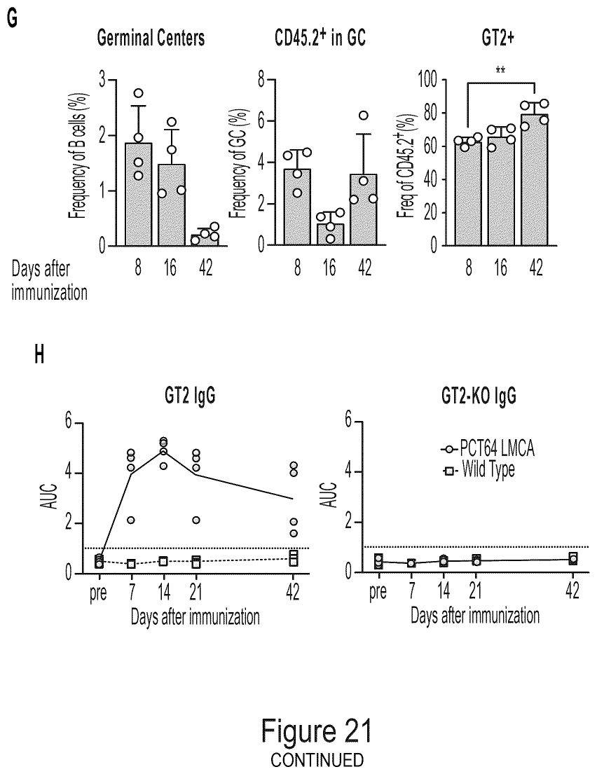

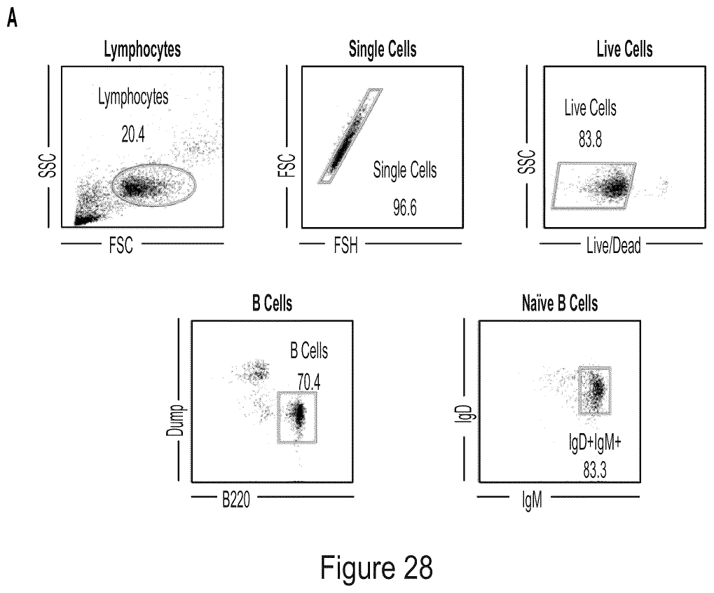

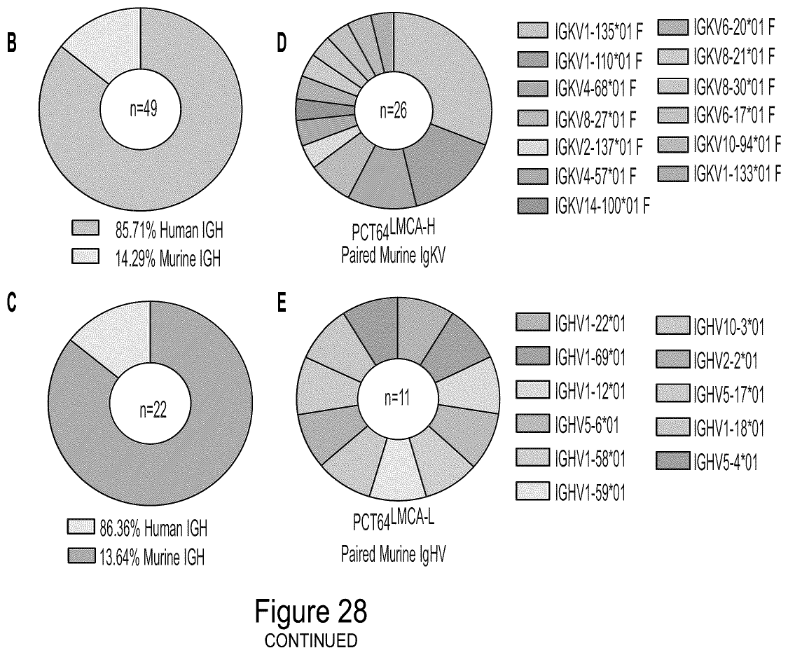

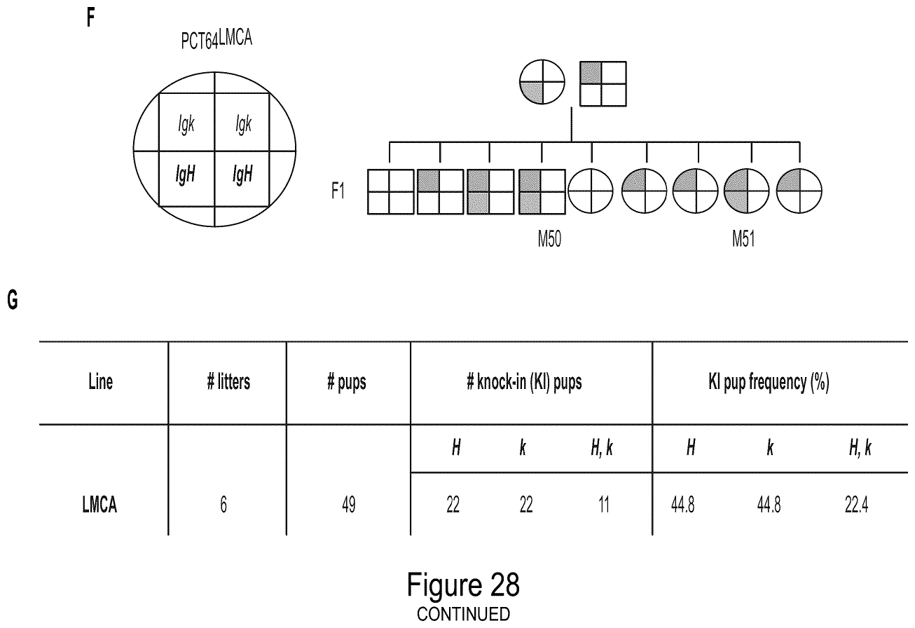

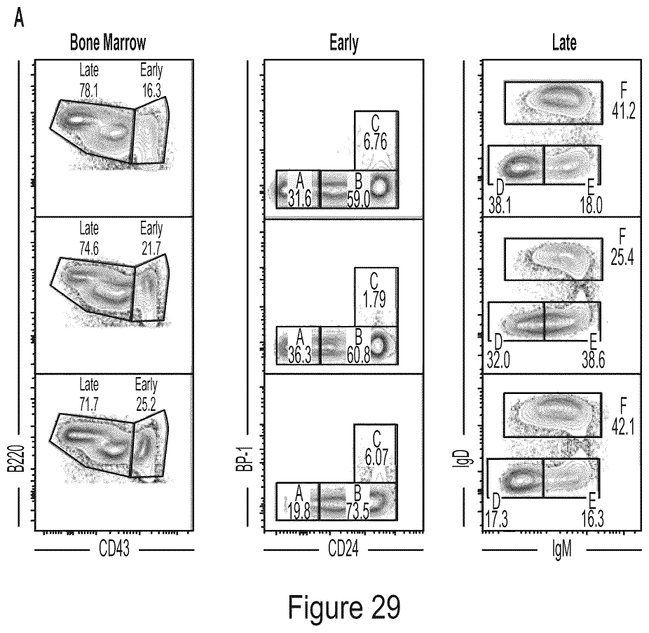

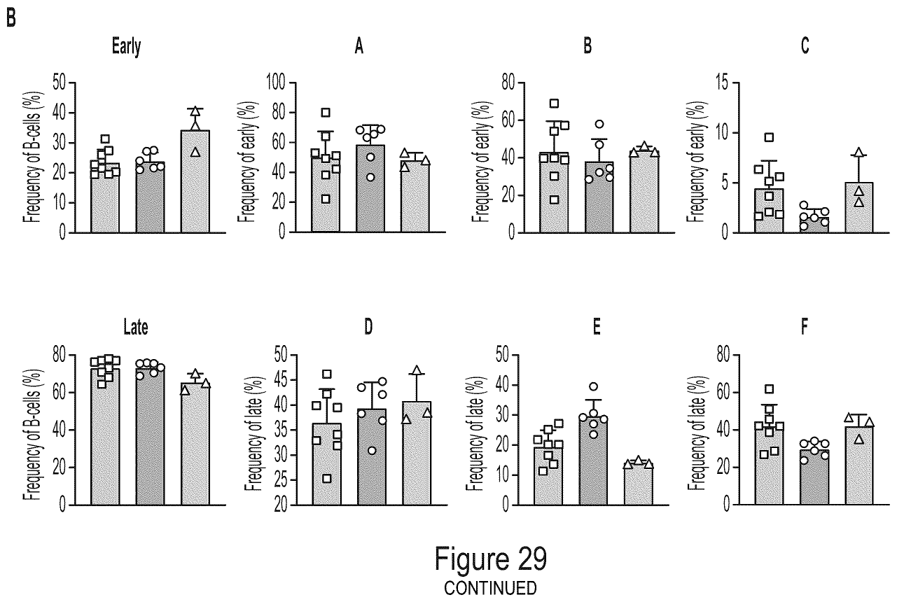

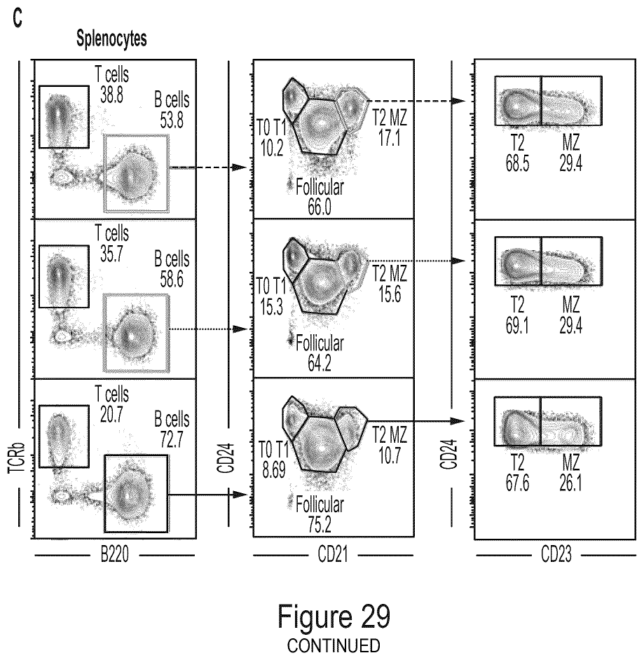

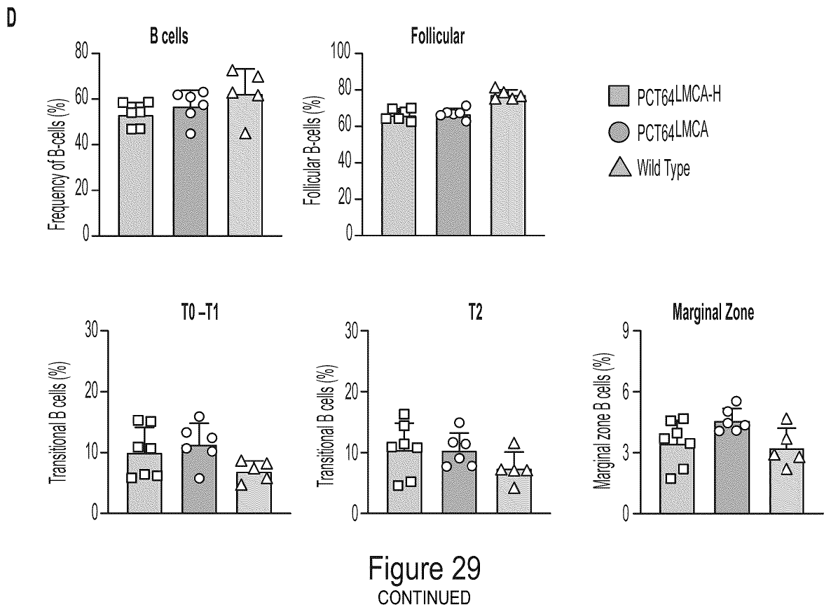

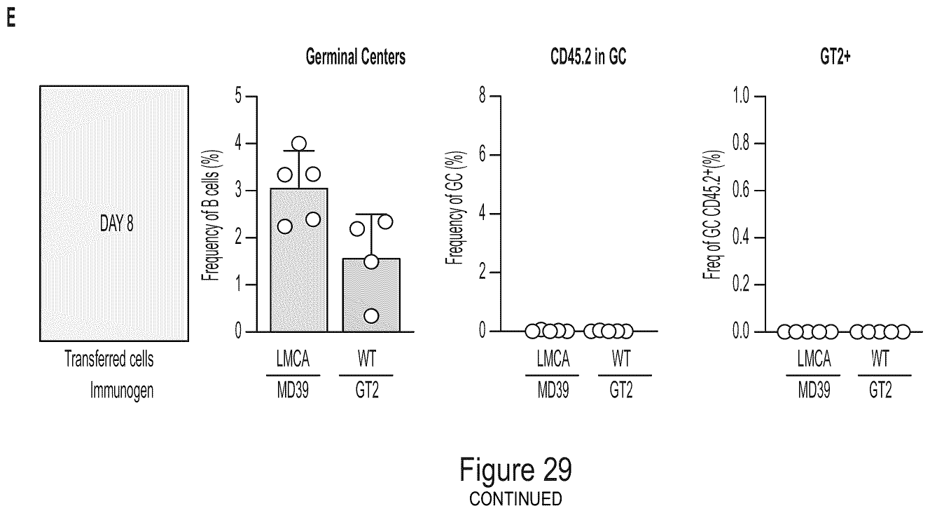

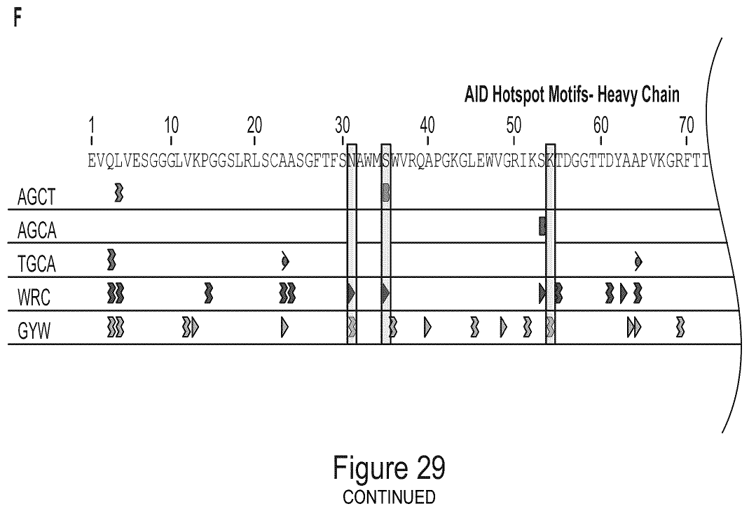

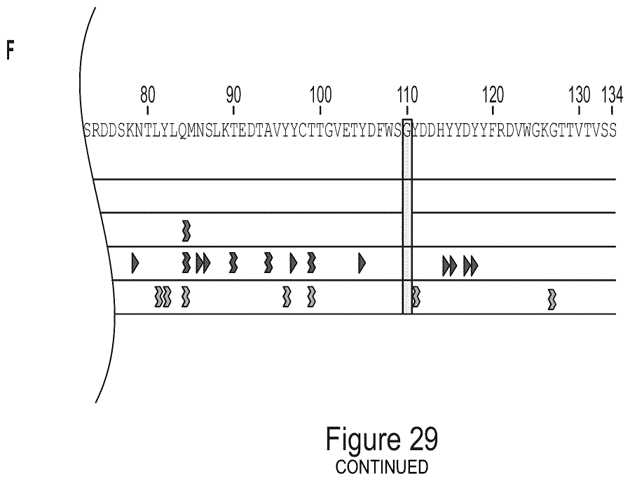

Using Applicants' CRISPR/Cas9 protocols (Lin et al., 2018; Wang et al., 2021), Applicants inserted the PCT64LMCA IGH and IGK variable regions into their respective native murine loci and confirmed successful insertion by genotyping. Next, Applicants established the frequency of cells expressing the PCT64 KI sequences by sorting and sequencing B220+naïve peripheral B cells (FIG. 28A). PCT64LMCA IGH was expressed by 85.7% of naive B cells (FIG. 28B) and PCT64LMCA IGK by 86.3% (FIG. 28C). Paired sequences showed that both the human PCT64LMCA IGH and IGK could form hybrid humanized BCRs by pairing with a variety of murine heavy and light chains (HC/LCs) (FIG. 28D-E). To generate mice carrying the full PCT64 LMCA antibody (PCT64 LMCA), Applicants crossed PCT64 IGH (PCT64LMCA-H) and PCT64 IGK (PCT64LMCA-K) mice and obtained offspring where both human IGH (96-100% of expression) and IGK (86-90%) paired with each other (˜81%) (FIGS. 21A and 28F-G). By screening differentiation stages in the bone marrow (FIG. 29A-B) and in the spleen (FIG. 29C-D), Applicants confirmed that B cells underwent normal development in both the PCT64 LMCA-H and PCT64LMCA mice (Hardy et al., 1991). To assess whether PCT64LMCA B cells express functional BCRs, Applicants measured antigen specific binding to an Apex-GT2 trimer probe (GT2 below) engineered to bind PCT64 LMCA with moderate affinity (monovalent KD=167 nM) (Willis et al. 2021). Approximately 58% of peripheral blood B cells bound to GT2 in a double fluorophore staining assay (˜0.04% of B cells in C57BL/6) mice were GT2-reactive); using an epitope KO probe (GT2-KO), Applicants confirmed that 99% of the binders were epitope-specific (FIG. 21B-C). Subsequent BCR sequencing confirmed that GT2-reactive B cells were positive for both the human PCT64LMCA IGH (100%) and corresponding human PCT64LMCA IGK (95%) (FIG. 21D). In summary, PCT64LMCA B cells exhibit high levels of allelic exclusion, undergo normal development and express functional paired human KI BCRs which bind the Apex-GT2 probe.

GT2 immunization activates PCT64 LMCA B cells and generates durable GCs. Next, Applicants tested the capacity of GT2 to activate PCT64LMCA B cells in vivo. Given the high frequency of PCT64LMCA B cells in the KI mouse line, Applicants used an adoptive transfer system where 500,000 CD45.2+PCT64LMCA or CD45.2+WT control B cells were transferred into congenic CD45.1+C57BL/6J recipient mice (FIG. 21E) to decrease precursor frequency and facilitate the tracking of PCT64LMCA responses. Twenty-four hours after adoptive transfer, Applicants immunized mice with either GT2 or BG505-MD39 SOSIP trimers intraperitoneally (IP)—an established route for SOSIP immunogens (Escolano et al., 2016; Steichen et al., 2016)-formulated in Sigma adjuvant system (Sigma) and measured immune responses at 8 days post immunization (dpi) in the spleen (FIG. 21E-G and FIG. 29E). GC responses (GC; CD95hiCD38lo) were detected at 8 dpi in all groups; at this timepoint, CD45.2+PCT64LMCA B cells represented on average 3.7% of the activated GC B cells in GT2 immunized mice, and their specificity was confirmed by GT2-probe binding (63%; FIG. 21F). CD45.2+ cells were not detected in GCs in mice immunized with BG505-MD39 SOSIP trimers or in mice that received WT cells (FIG. 29E). At 16 and 42 dpi, CD45.2+GC responses were still ongoing. An increase in GT2 specificity was detected in GC cells from 8 to 42 dpi (from 62.8% to 79.4%, P=0.0038), suggesting an ongoing selection process leading to the expansion of clones with higher affinity for GT2 (FIG. 21G). Finally, Applicants monitored GT2-specific IgG serum responses in PCT64LMCA recipient mice. Epitope-specific titers were detectable at 7 dpi and gradually declined after a peak at 14 dpi (FIG. 21H). WT mice immunized with GT2 trimers failed to develop epitope-specific IgG antibodies. No off-target antibody responses against epitope-deficient GT2-trimers (GT2-KO) were detected in either group (FIG. 21H), indicating that serum IgG antibody responses were attributable to adoptively transferred PCT64LMCA B cells.

Overall, these results show that PCT64 precursors were successfully activated by GT2 immunization, formed sustained GC reactions, and generated epitope-specific IgG responses.

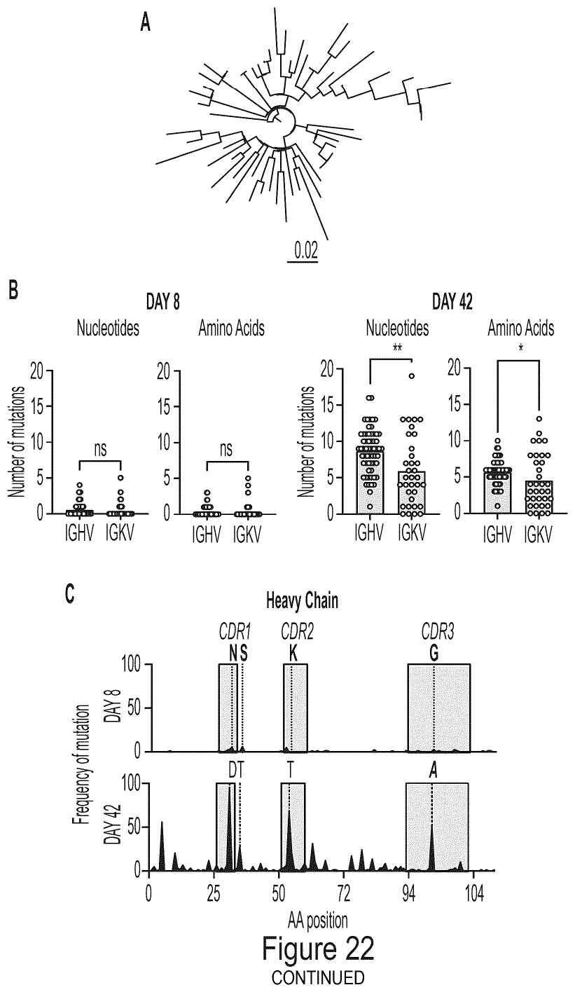

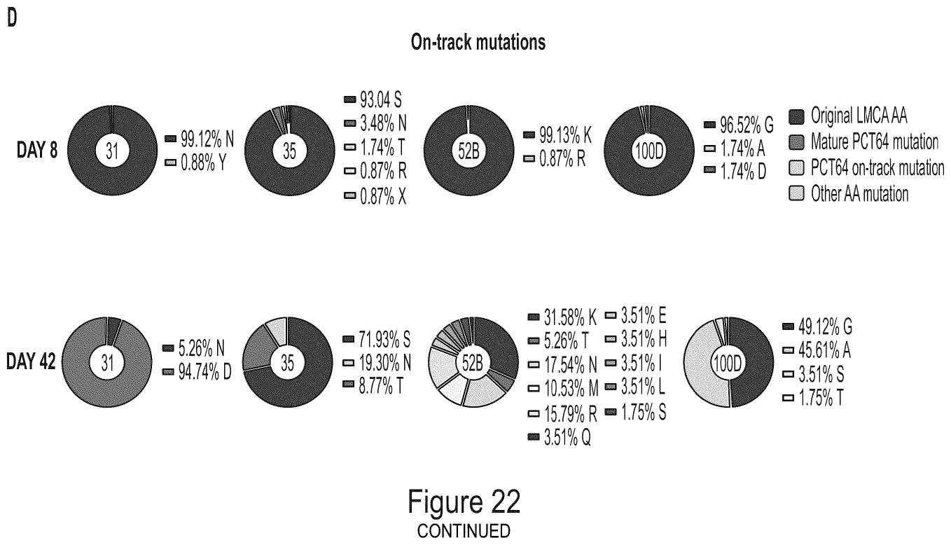

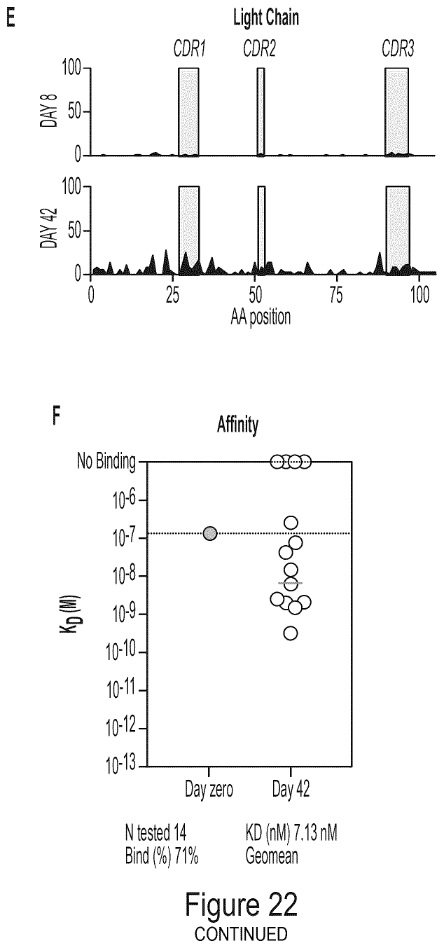

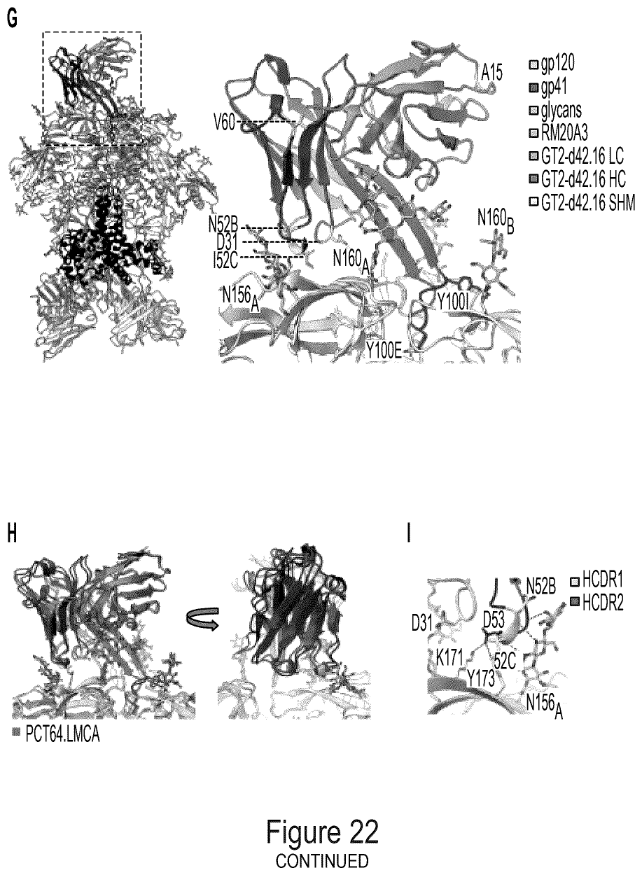

GT2-primed PCT64LMCA BCR heavy chains acquire bnAb-like mutations. The primary goal of GT is to expand precursors and drive SHM toward the progressive acquisition of breadth and potency. Applicants sought to determine whether PCT64 LMCA B cells underwent SHM and accumulated PCT64-like mutations in response to GT2-trimer immunization by sorting class-switched GT2+PCT64LMCA B cells at 8 and 42 dpi for single-cell BCR sequencing. Tracing lineage evolution of the PCT64LMCA IGH with a phylogenetic model highlighted broad diversification at 42 dpi (FIG. 22A). SHM was minimal in the PCT64LMCA IGH and IGK V-region at 8 dpi but increased over time (FIG. 22B). At 42 dpi, there were significantly higher levels of SHM in the IGH V region (8.6 nt, 5.6 aa on average) than in the corresponding IGK V region (5.9 nt, 4.5aa on average), both at the nucleotide (nt) (P=0.0013) and amino acid (aa) levels (P=0.037) (FIG. 22B). Mutations accumulated over time in recurring positions in the HCDR1, HCDR2 and HCDR3 (FIG. 22C). Substitutions at these sites were enriched both for aa present in the mature PCT64 bnAb (V-region position 31, 35, 52B) and for aa present in early PCT64-line isolates (in HCDR3 position 100D) (FIG. 22D) (Landais et al., 2017). N31D, which is also present in the mature PCT64 bnAb (Landais et al., 2017), was acquired in 98% of the isolated HC sequences, indicating positive selection. Interestingly, while enriched mutations in the HC V-region were likely facilitated by the presence of AID binding sites, no underlying A ID hotspot was identified for the HCDR3 mutation (100D) (FIG. 29F).

In contrast, no mutations were enriched in the PCT64LMCA IGK (FIG. 22E), suggesting an HC-driven immunogen interaction. To confirm that these mutations drove antibody maturation, Applicants expressed 14 representative Fabs isolated at 42 dpi and quantified their affinity for GT2. Of the 10 Fabs with detectable affinity for GT2, nine showed increased affinity over the PCT64 LMCA Fab (KD, 130 nM). The geomean KD for all 10 Fabs (4.3 nM) indicated a 30-fold improvement; one Fab exhibited a 394-fold affinity gain over the native LMCA Fab. (FIG. 22F).

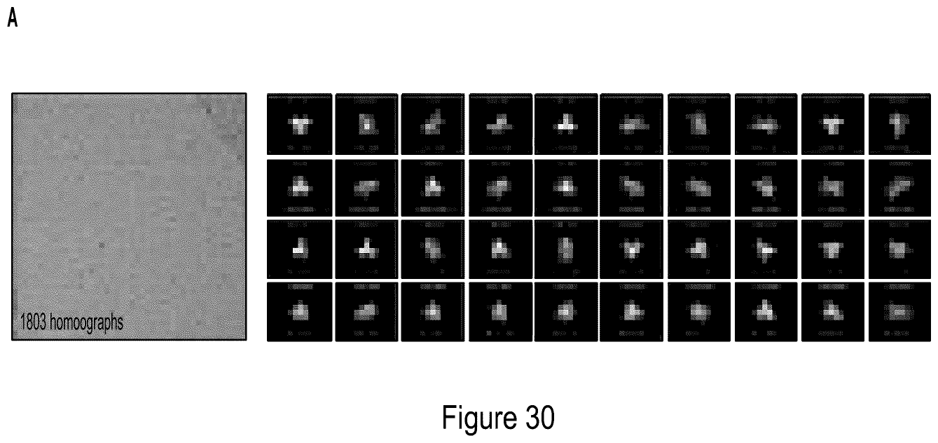

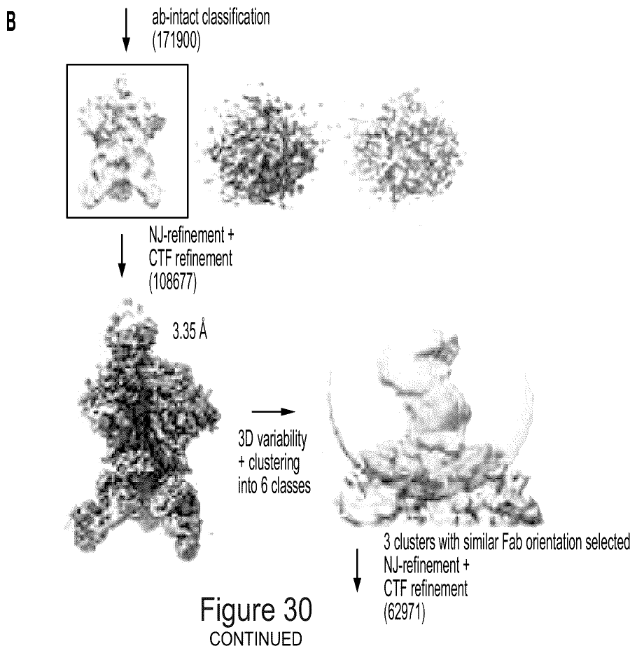

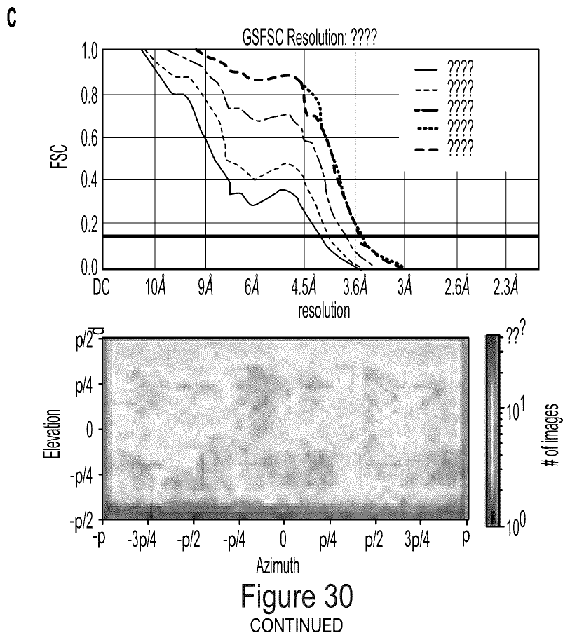

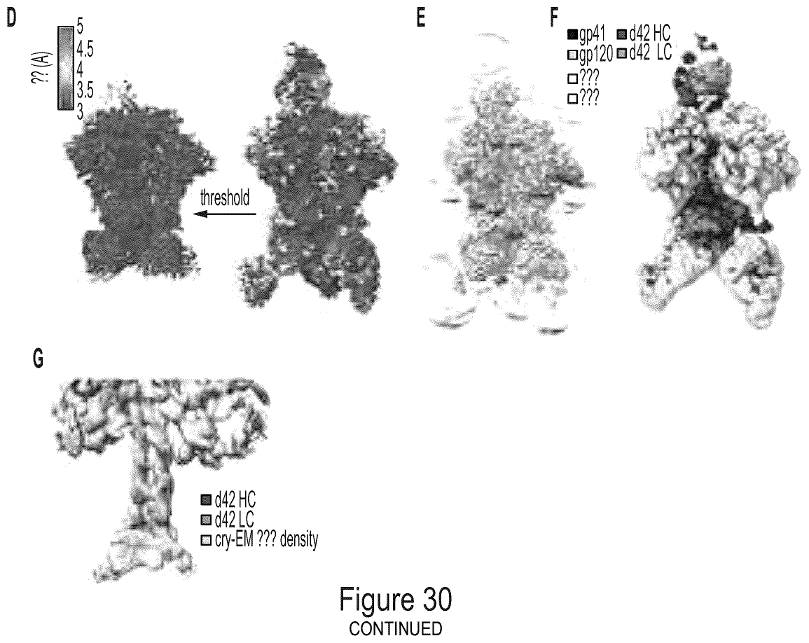

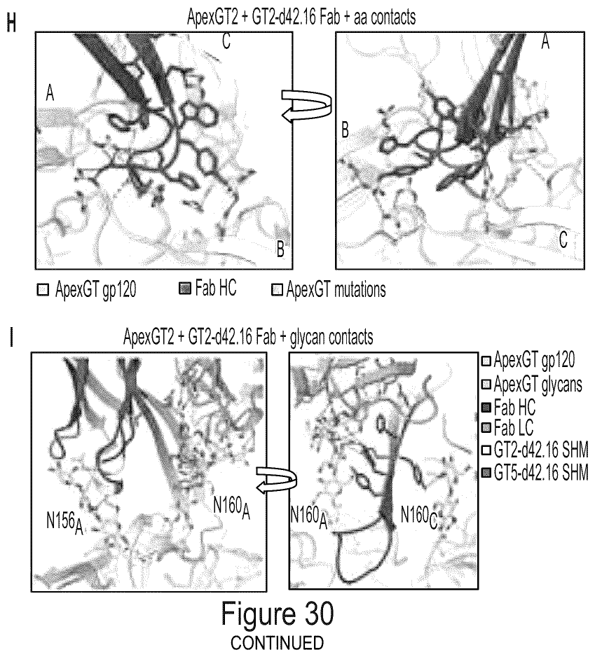

To interrogate the interaction between the acquired mutations and GT2, Applicants determined the cryo-EM structure of GT2 in complex with a high affinity day 42 Fab (GT2-d42.16) (FIG. 30A-C). The Env base-binding Fab RM20A 3 was included to improve particle angular distribution. Applicants' ˜3.5 Å-resolution reconstruction (FIG. 30D-G) allowed us to build atomic models of the complex (FIG. 22G), which confirmed that GT2-d42.16 adopts a nearly identical structure and angle of approach to PCT64.LMCA (FIG. 22H) with the characteristic elongated and anionic HCDR3 beta hairpin loop that extends inward toward the 3-fold axis of the trimer apex and extensive engagement with positively charged residues in the V1/V2 loop and the glycan at N160gp120A (FIG. 22G; FIG. 30H-1). Although the map resolution is lower due to inherent flexibility in the distal regions of the Fab outside of the paratope (FIG. 30C), docking of the high-resolution PCT64.LMCA crystal structure allowed us to reliably identify the sites of SHM acquired during affinity maturation (FIG. 22H). The only positions of SHM within the paratope are in the HCDR1 and 2 domains, which are responsible for engaging/accommodating the glycan at N156gp120A as well as the C strand of V2 (FIG. 221) (Willis et al., 2021). Although the N156gp120A glycan does not engage in any specific sidechain contacts, it forms several backbone hydrogen-bonds (H-bonds) (FIG. 221) that could be enhanced by the HCDR1 and/or HCDR2 mutations by stabilizing the small helical turn in the HCDR2. Both the K 52bN and T52cl mutations are present in early PCT64 lineage members such as PCT64.13B, although both sites were further mutated in the mature PCT64 bnAbs (Landais et al., 2017). The N31D mutation, located near the interface with the C strand, is present in the majority of mature PCT64 lineage members and could be contributing to a more favorable electrostatic interaction with the positively charged apex. Taken together, Applicants' results indicate that GT2 immunization can successfully initiate PCT64LMCA maturation towards a higher affinity and mature PCT64-like antibody.

PCT64 precursor responses to GT2 are driven by the heavy chain. In Apex-directed bnAbs, the HCDR3 is a major binding determinant (Pancera et al., 2010; Pejchal et al., 2010; Mclellan et al., 2011; Julien et al., 2013; Andrabi et al., 2015). Mammalian display directed evolution was used to engineer the GT2 trimer to target the PCT64^{LMCA} HC, similar to the strategy used for targeting N332-dependent bnAbs (Steichen 2016). To validate the specificity of the GT2 immunogen for PCT64 IGH, Applicants used a PCT64^{LMCA} HC-only mouse model (PCT64LMCA-H) in which the human PCT64^{LMCA} IGH was paired with native murine LCs.

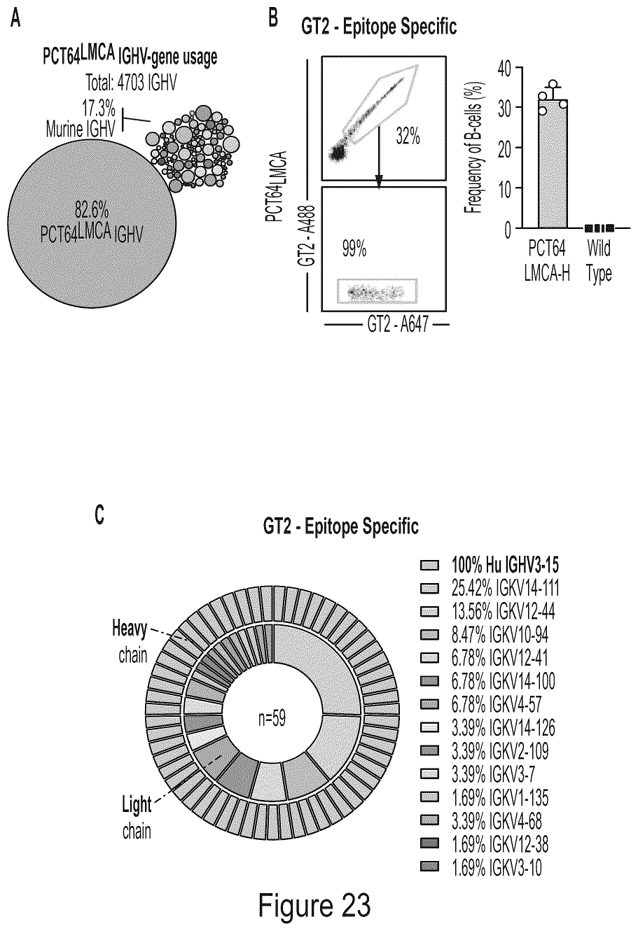

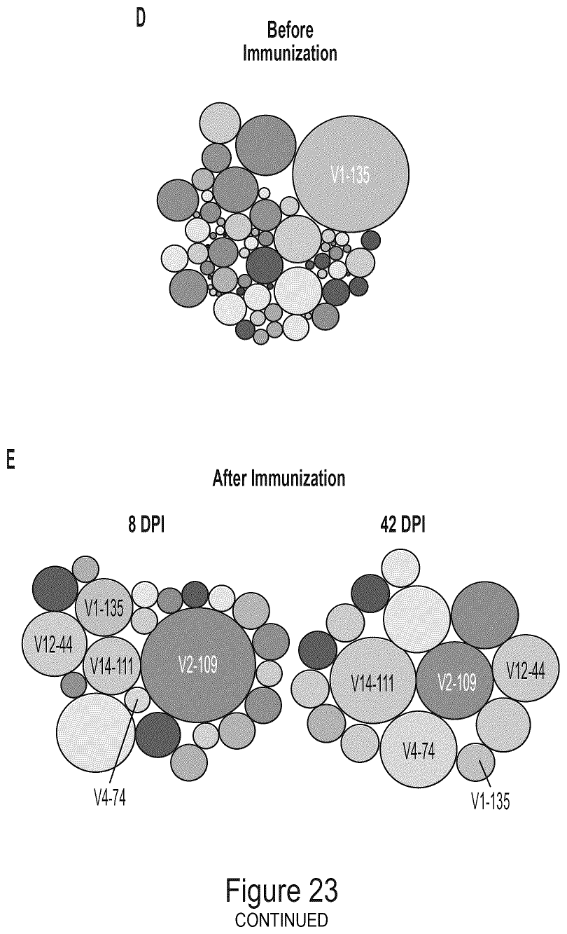

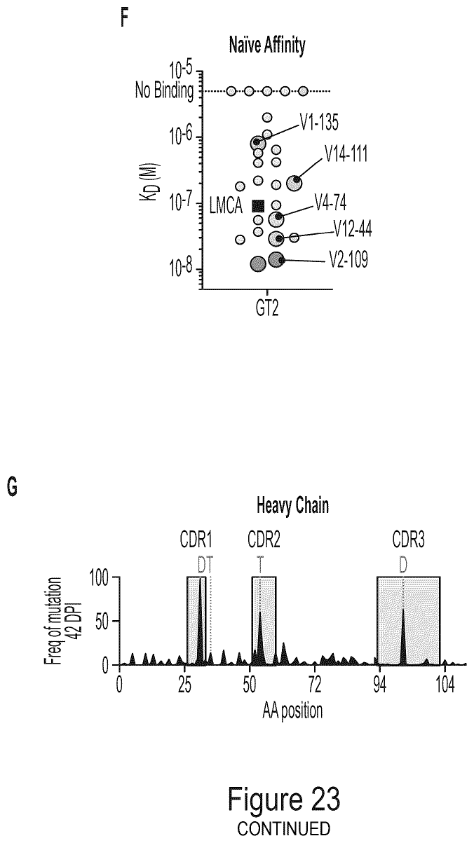

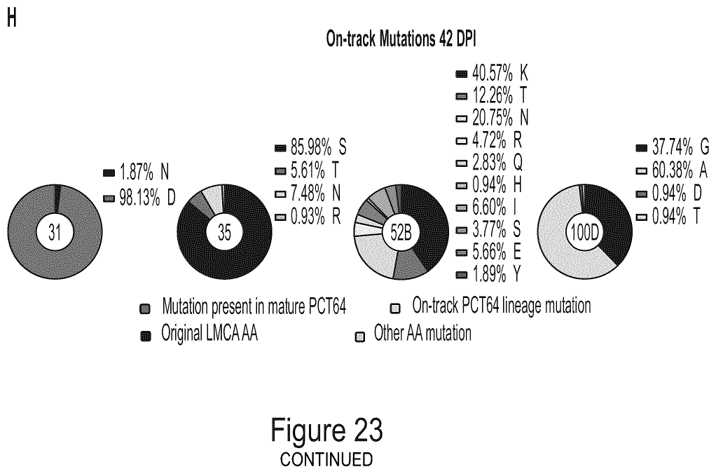

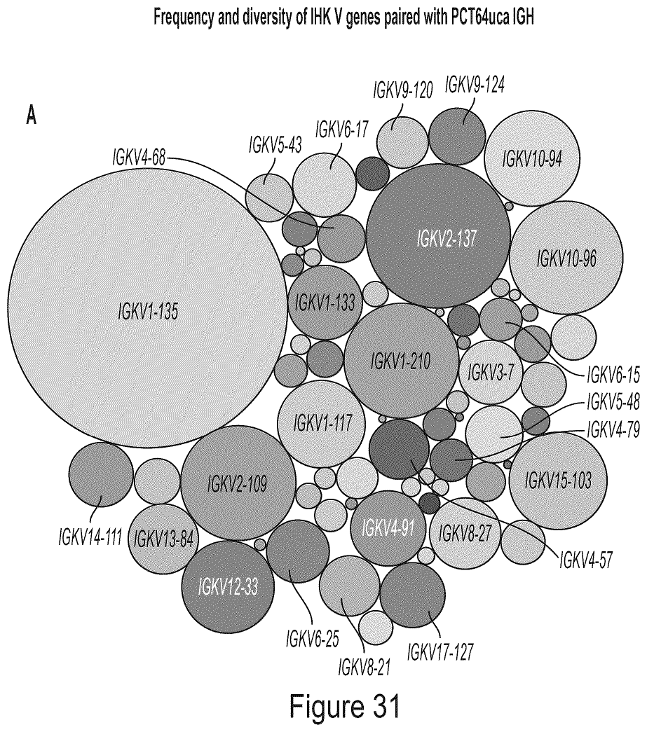

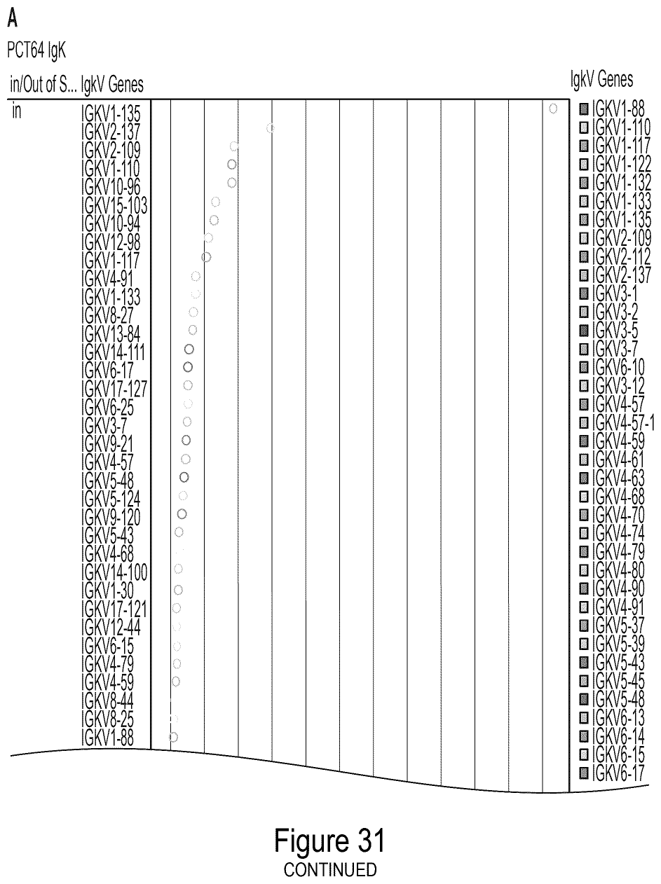

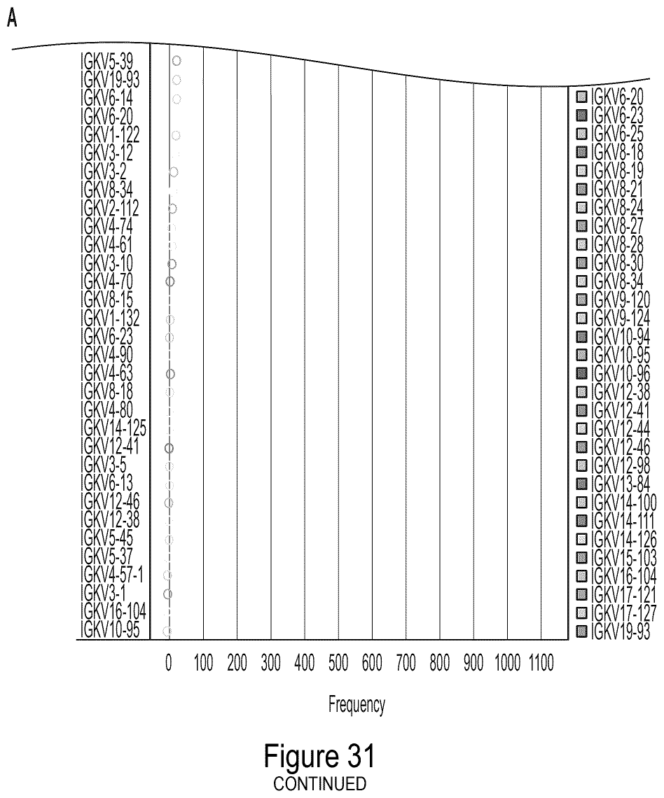

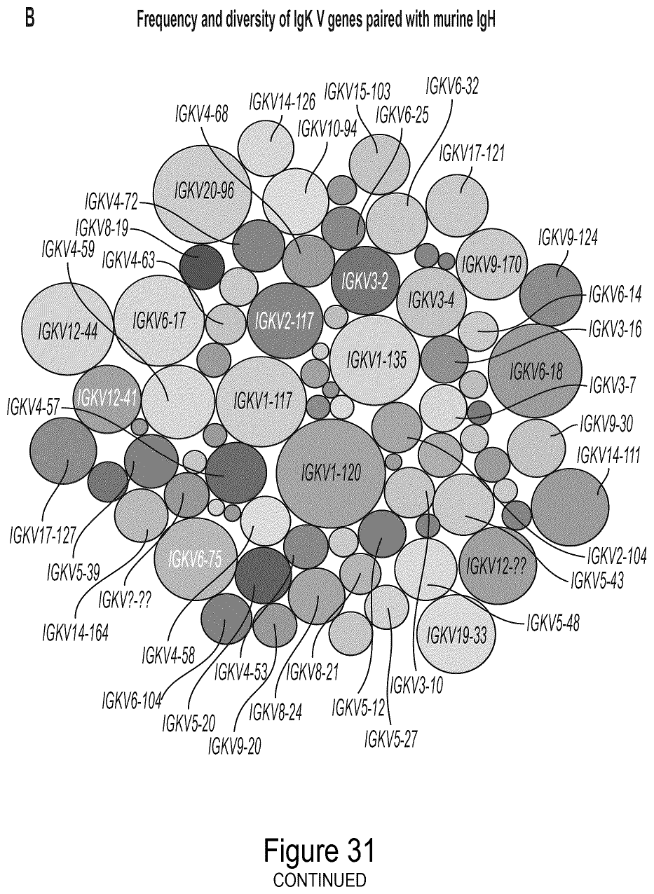

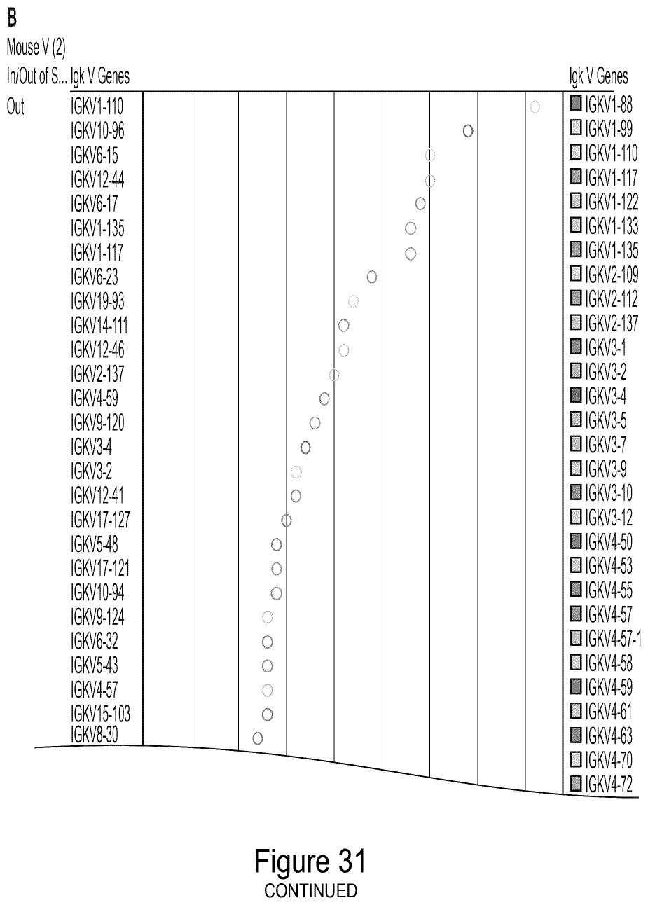

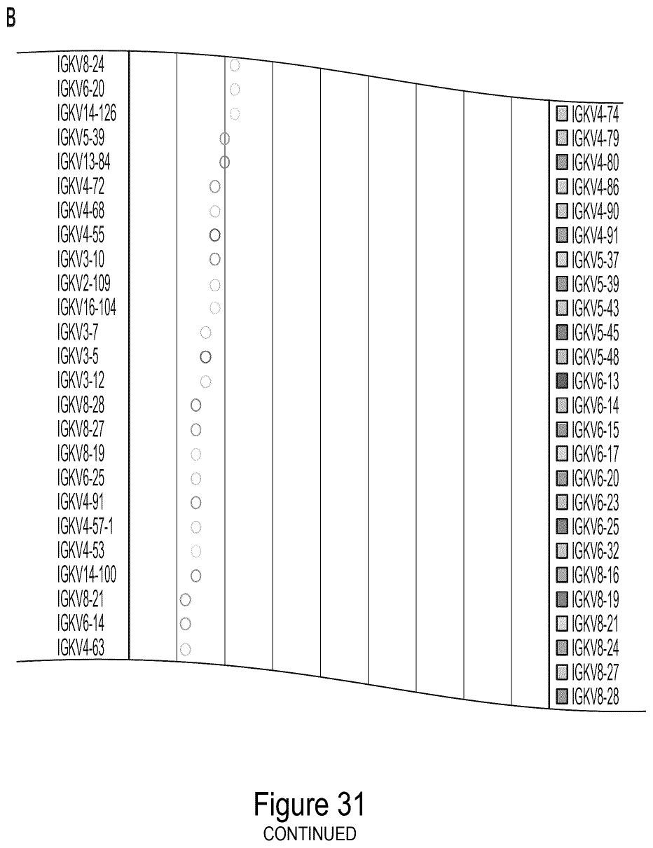

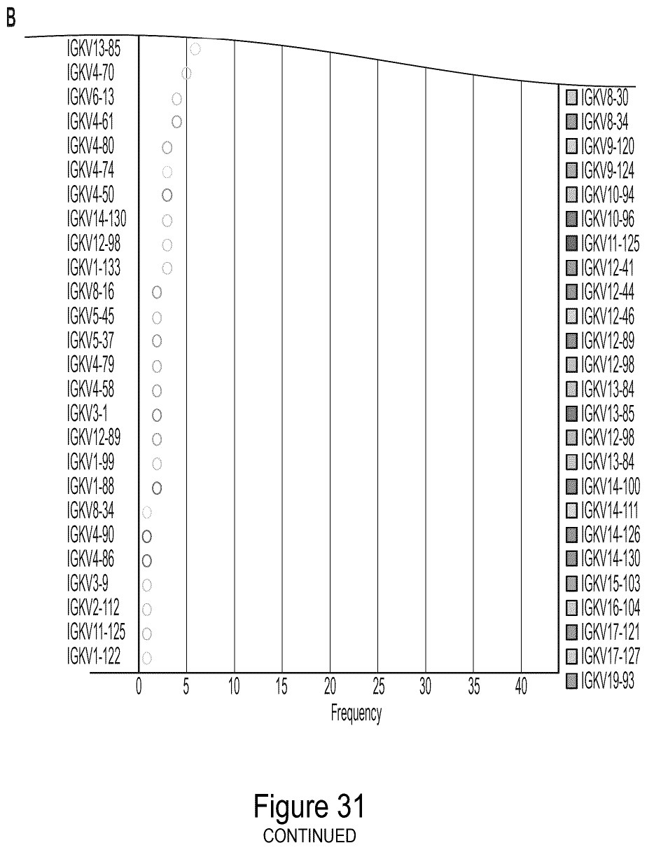

Applicants first characterized the BCR repertoire in PCT64^{LMCA-H} heterozygous mice using 10× Genomics single-cell BCR sequencing of naive splenic B cells and found that 82.6% of murine B cells expressed the PCT64LMCA IGH sequence (FIG. 23A). PCT64^{LMCA} IGH could successfully pair with a large variety of murine IGK V genes, but there was evidence of selectivity: the most frequent pairings were with IGK V1-135 (28.8% of paired IGK V genes), V2-137 (7.74%), V2-109 (4.94%) and V1-110 (4.81%) (FIG. 31A). In contrast, in the remaining fully murine BCRs, IGK V genes were more evenly distributed, with the most frequent IGK V gene (V1-110) found at only 5.03% (FIG. 31B).

Next, Applicants quantified antigen-specific binding to GT2 trimer probes in naïve PCT64LMCA-H mice. Approximately 32% of peripheral blood B cells bound the GT2 probe in a double fluorophore staining assay (FIG. 23B); binders consisted of 100% human PCT64 LMCA IGH paired with a variety of murine LCs (FIG. 23C); PCT64 IGH paired with murine LCs is thus capable of GT2 binding.

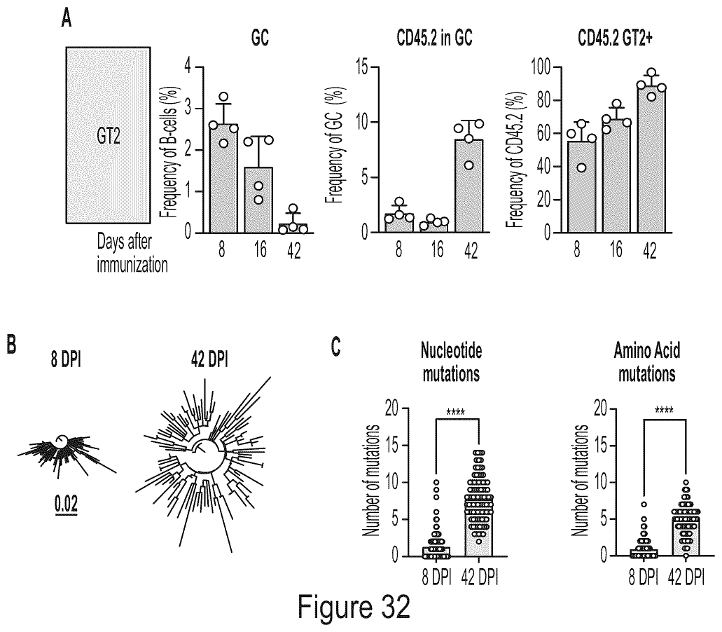

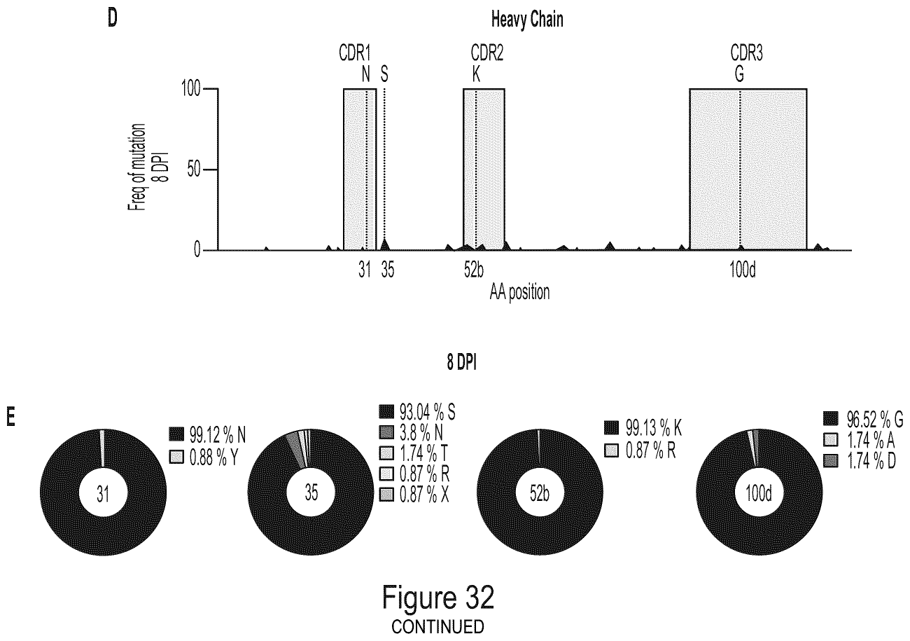

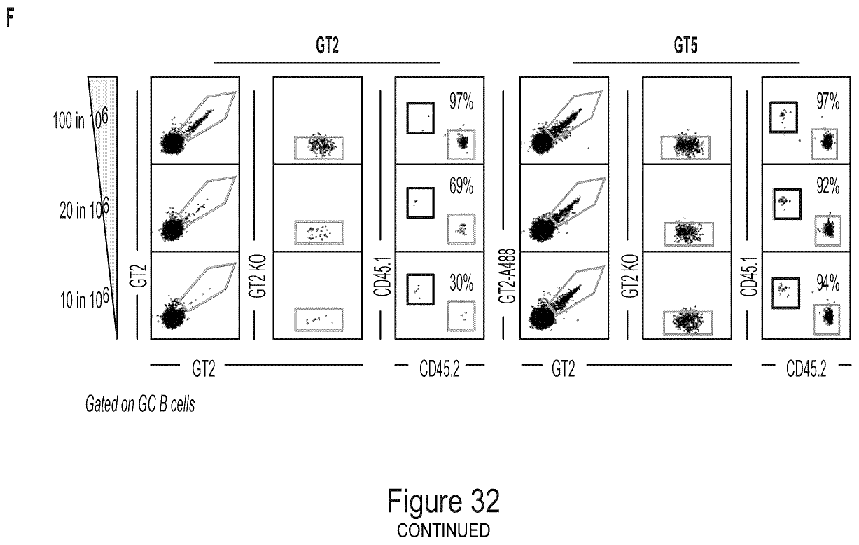

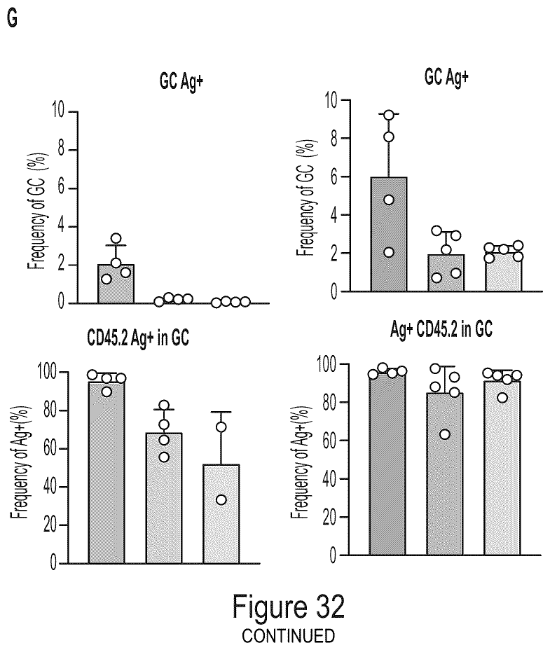

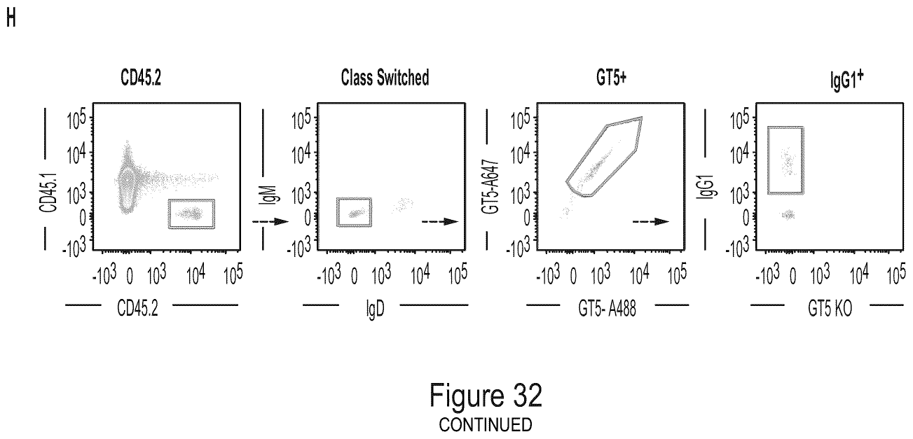

Immunization with GT2 trimers as above activated CD45.2+PCT64 LMCA-HB cells and generated long-lasting GCs (FIG. 32A). To determine the contributions of IGK to affinity maturation, Applicants single-cell sorted class-switched, GT2-specific, CD45.2+ B cells at 8 and 42 dpi for BCR sequencing. All isolated HC were derived exclusively from the KI PCT64LMCA but paired with a variety of murine LCs at both 8 and 42 dpi, demonstrating ongoing multiclonal selection (FIG. 23D-E). Some IGK V-genes were enriched during GC selection relative to their frequency in the naive repertoire: ˜28% of the isolated LCs in the GC at 8 dpi expressed IGK V2-109 (4.9% of the total IGK V-genes paired with PCT64LMCA-H in the naïve repertoire) (FIG. 23D-E). Similarly, other initially low frequency V genes such as V4-74 (0.23% in the naive repertoire), V14-111 (1.59%) and V12-44 (0.75%), were enriched over time (FIG. 23D-E).

Due to this enrichment, Applicants reasoned that certain murine IGK V genes may favor GT2-binding. Therefore, Applicants measured the affinities of PCT64LMCA IGH paired with different naïve murine IGK (FIG. 23F). Hybrid antibodies isolated from naïve PCT64LMCA-H mice, which constituted a polyclonal population generated through the variability of the LC, had affinities (KDs) from 1.6×10−6 to 2.5×10−8 M. Some IGK V genes, such as IgK V2-109 and IgK V12-44 (both enriched in the GC), had higher affinity for GT2 than PCT64^{LMCA} paired with its natural human LC by up to 2.6-fold. In contrast, lower affinity was found with IGK V1-135 and IGK V2-137, which were at far lower frequencies in the GC than in the naïve repertoire (FIG. 23F).

Applicants then investigated whether IGH could acquire PCT64-like mutations in the absence of the human LC. SHM and diversification increased over time (FIG. 32B). By 8 dpi, human VH3-15 acquired an average of n=1.2□1.6 nt mutations (translating to n=0.89□1.1 aa mutations) (FIG. 32C) and reached an average of n=7.7□2.4 nt (n=4.9□1.9 aa) by day 42 (FIG. 32C), similar to the levels observed in the full PCT64LMCA KI model. Enriched residues in PCT64LMCA-H IGH matched those identified for PCT64LMCA (FIG. 23G-H and FIG. 32D-E), and the aa mutations at these sites included previously identified PCT64-like mutations in positions 31, 35, 52B and 100D (FIG. 23G-H), suggesting that IGH evolution in response to GT2 trimers is both consistent and independent of the associated LC. These results demonstrate that B cells bearing the PCT64LMCA HC in conjunction with diverse LCs can respond with great specificity to GT2 immunization.

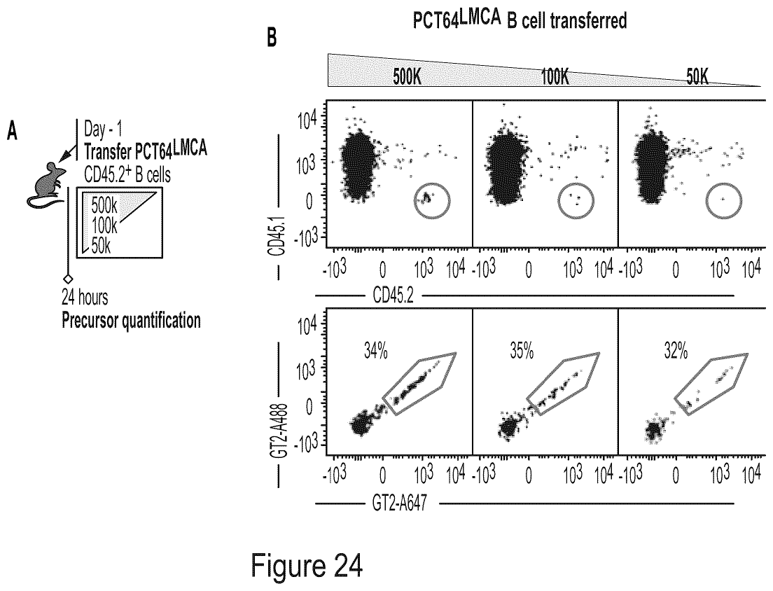

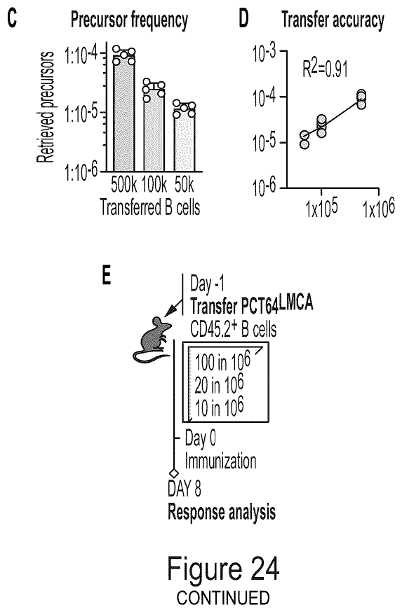

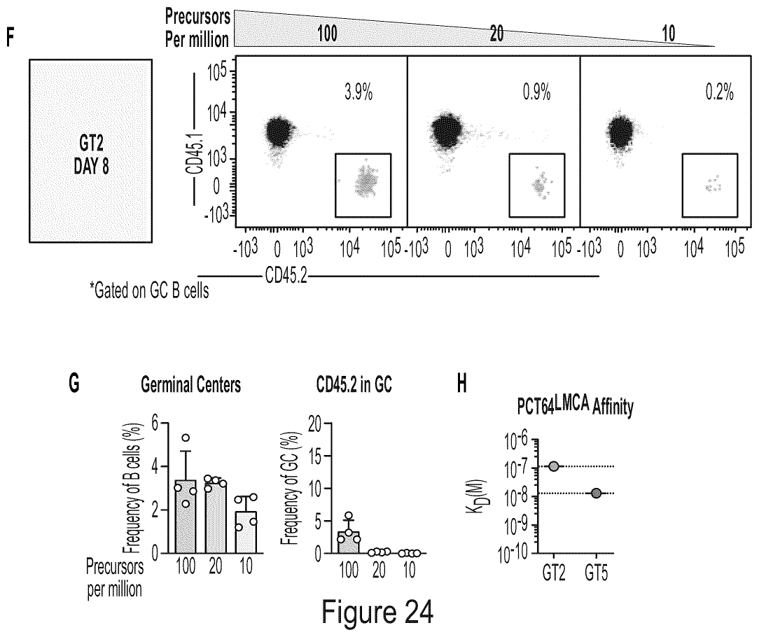

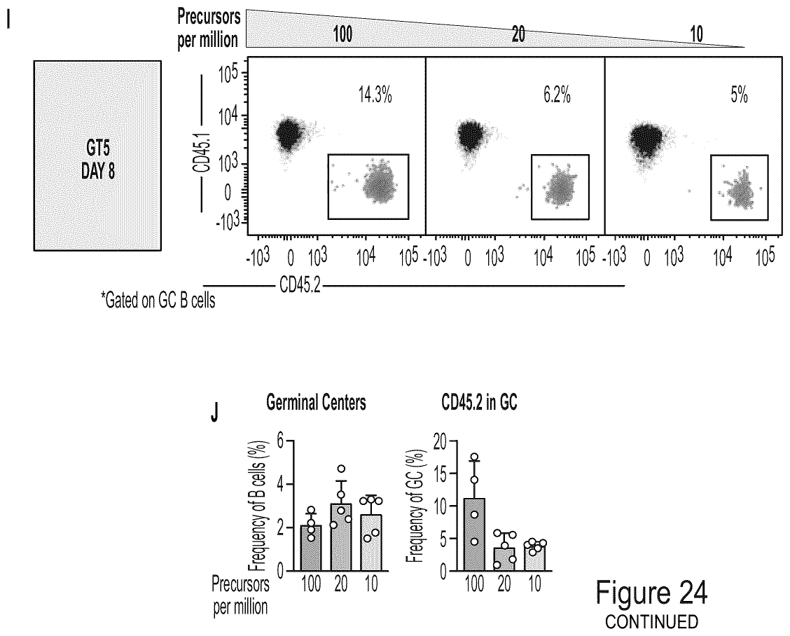

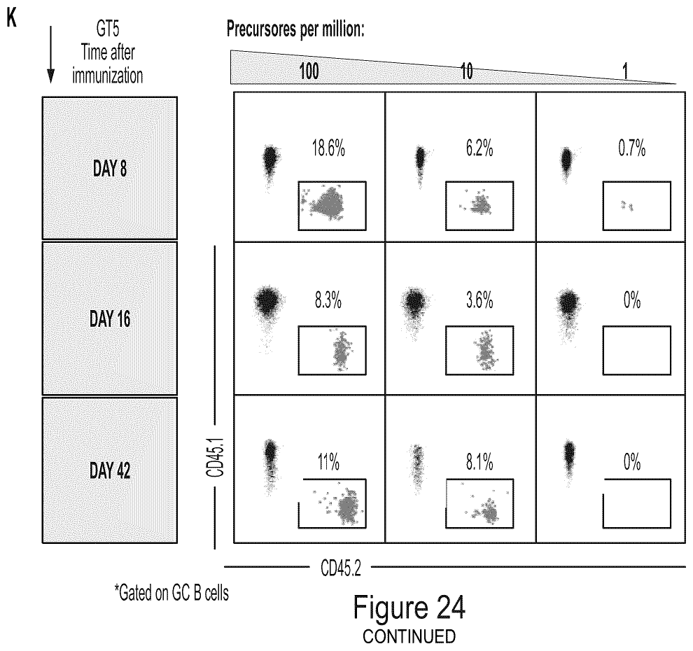

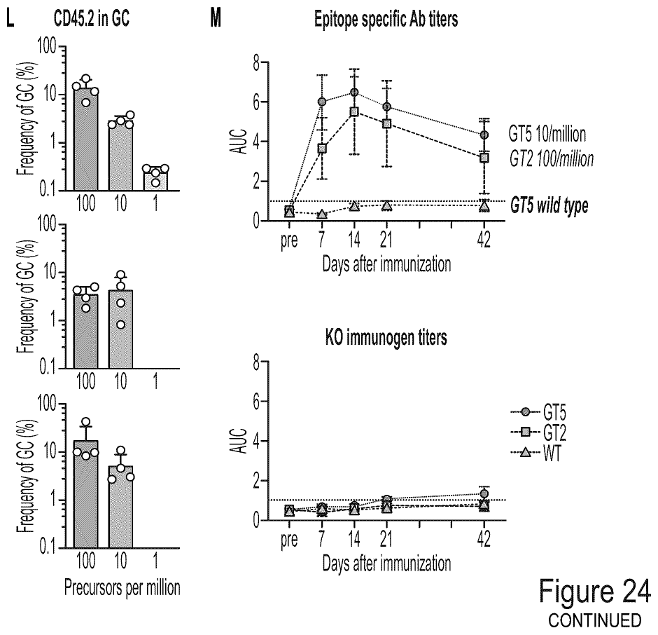

A high affinity immunogen is required to activate rare PCT64 precursors. Human repertoire data suggests that a suitable PCT64-immunogen needs to reproducibly trigger B cells at precursor frequencies lower than ˜20 per 106 (Willis et al., 2021). To evaluate the capacity of GT2 to activate PCT64 precursors at human physiological frequencies, Applicants calculated the frequencies of GT2+ PCT64LMCA-H B cells in the spleens of recipient mice at the time of immunization, 24 h after the adoptive transfer of 500,000, 100,000 or 50,000 CD45.2+ B cells (FIG. 24A-B). The resulting GT2-specific CD45.2+ B cell frequencies were 100, 20 or 10 per million splenic B cells, respectively (FIG. 24C-D). Responses in immunized recipient mice with defined numbers of PCT64LMCA B cells were analyzed 8 dpi by FACS (FIG. 24E). While PCT64LMCA frequency did not affect total GC size, it did significantly affect the proportion of CD45.2+ cells in GCs, from 1.2% CD45.2+ at 100 precursors per 106 to barely 0.2% at 10:106 (FIG. 24F-G).

Immunogen affinity is key to the activation of rare B cells (Dosenovic et al., 2015; Sok et al., 2016; Tian et al., 2016; Abbott et al., 2018). To assess the effect of affinity on activation, Applicants immunized mice with the range of PCT64 precursors defined above with ApexGT5 (GT5 below), an ApexGT trimer with higher affinity for PCT64LMCA (KD, 66 nM compared to 167 nM for GT2) (FIG. 24H) (Willis et al., 2021), and compared responses at 8 dpi to GT2 (FIG. 24E). Applicants observed a relative increase in CD45.2+ B cell recruitment to GCs in all GT5-immunized groups. The gap was most pronounced at the lowest precursor frequency (10 per 106), in which GT5 immunization activated 20 times more PCT64 LMCA B cells than GT2 (4% CD45.2 vs. 0.2%) (FIG. 24I-J). GT5-specific responses were dominated by CD45.2+ B cells (>90%) at all tested precursor frequencies; in contrast GT2-specific responses were directly proportional to the initial precursor number and were outnumbered in the GC by competitor CD45.1+murine B cells (FIG. 32F-G).

To assess whether GT5 immunization could generate sustained immune responses at even lower precursor frequencies, Applicants established a new comparative range of 100, 10 or 1 precursor(s) per 106. While GC responses decreased over time, CD45.2+ cells persisted in the GC from 8 until 242 dpi at both 100 and 10 per 106 (FIG. 24K-L). However, at 1 per 106, only a weak CD45.2+ response was generated at 8 dpi, and none was detected by 42 dpi. Epitope-specific IgG responses were detected by ELISA from 7 to 42 dpi in mice that received 10 PCT64LMCA B cells per 106; GT2-immunization produced comparable titers in mice that received 100 PCT64LMCA B cells per 106. WT control mice immunized with GT5 did not develop detectable epitope-specific IgG responses, and no off-target response was detected in any group (FIG. 24M).

By enhancing the stringency of the precursor frequencies in the preclinical model, Applicants demonstrated that the higher affinity GT5 protein trimer was capable of specifically activating PCT64 precursors at frequencies approaching the estimated human physiological range.

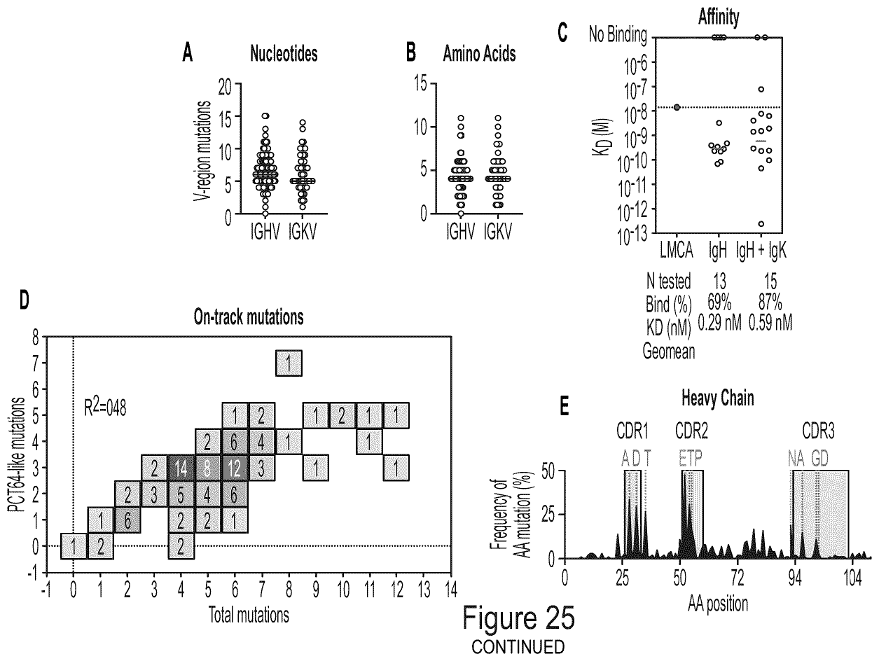

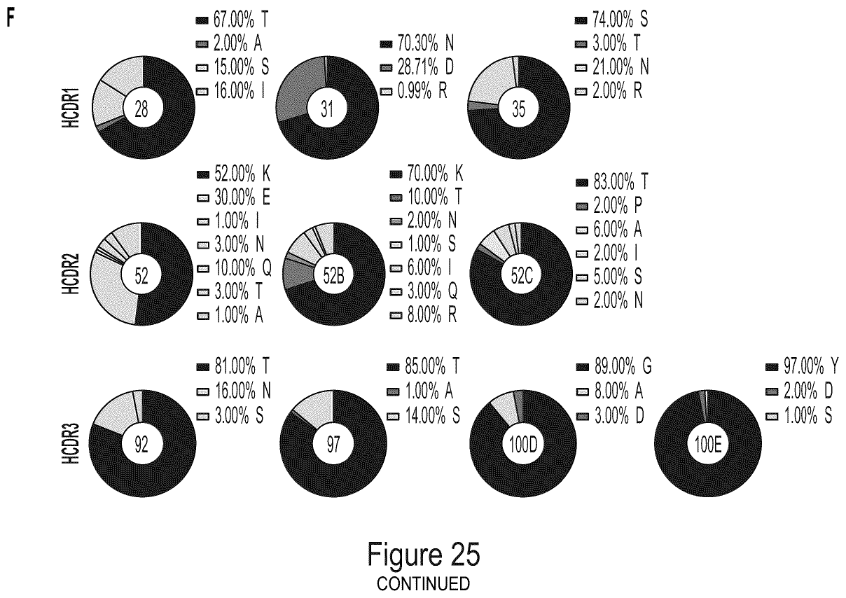

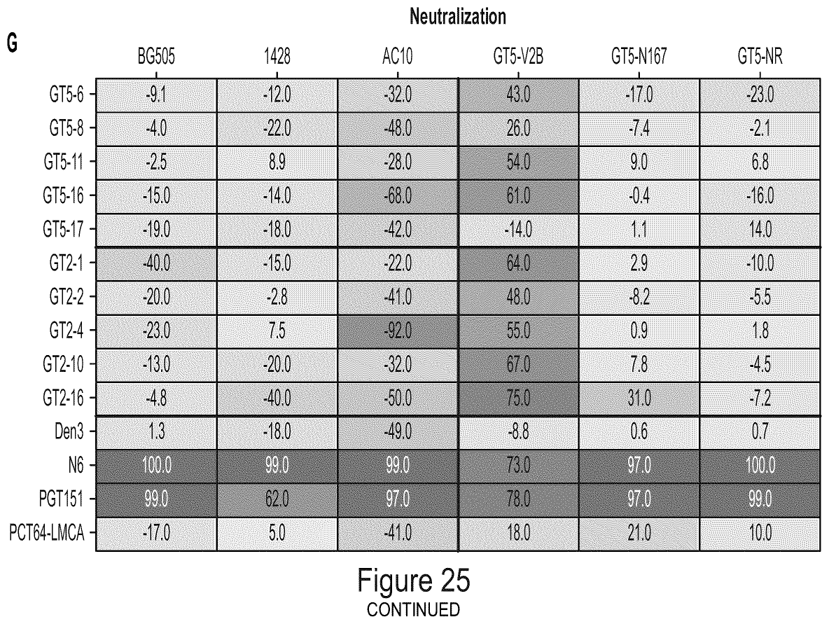

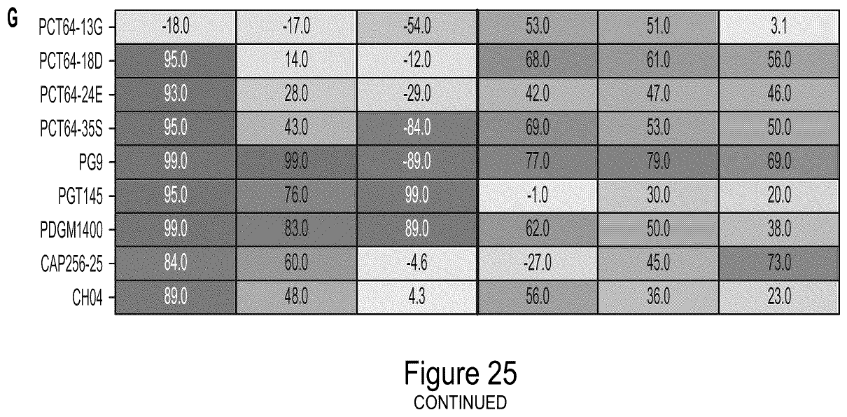

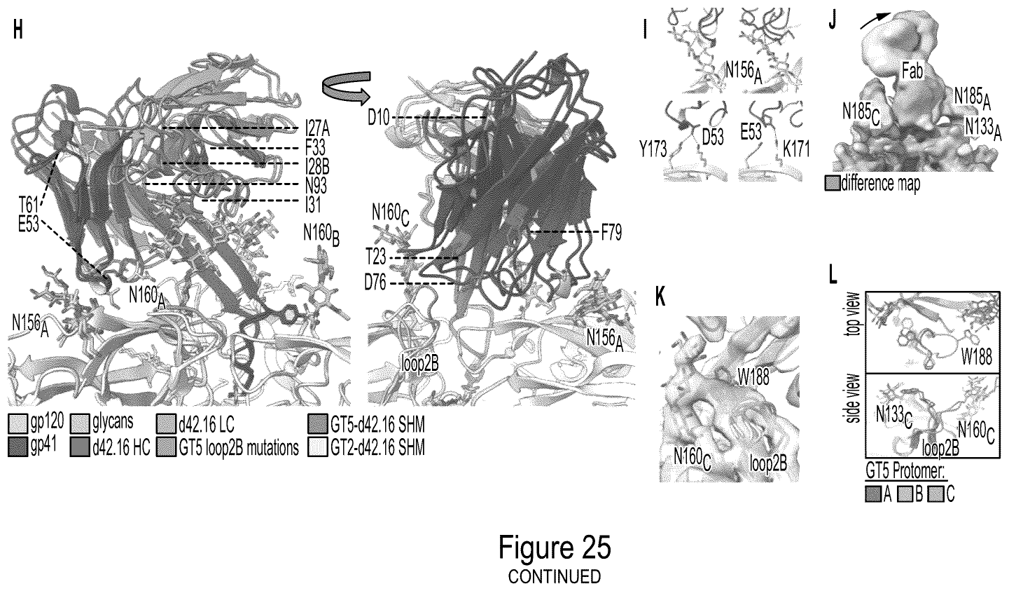

GT5 immunization induces mature PCT64-like mutations in IGH. To determine whether GT5 could induce on-track SHM in PCT64 precursors, Applicants sorted CD45.2+GT5+IgG1+ B cells at 42 dpi (FIG. 32H) and performed single-cell BCR sequencing. SHM-induced diversification was evident in both PCT64^{LMCA} IGH and IGK sequences (FIG. 25A); the average number of acquired mutations in the V-region was 6.6 nt/4.4 aa for IGH and 6.2 nt/4.6 aa for IGK (FIG. 25B). From this antibody library, Applicants expressed 15 representative Fabs for affinity measurements; 13 of 15 (87%) showed detectable binding to GT5 (KD<10 uM), with a geomean affinity of 0.59 nM among binders, representing an approximately 110-fold increase over the affinity of the LMCA (66 nM). Applicants also produced 13 Fabs with PCT64LMCA IGH paired with murine IGK chains; 9 of 13 (69%) had detectable affinity, and the geomean affinity among binders was 0.29 nM, representing an even larger affinity gain over the LMCA (FIG. 25C).

To determine whether antibody evolutionary trajectory was similar to that observed in the PCT64 donor, Applicants compared mutations acquired by IGH in the model with those from human PCT64 precursors isolated between 8 and 35 months post-infection (Landais et al., 2017). IGH sequences isolated at 42 dpi carried an average of 3-4 PCT64-like mutations and a peak of 7 PCT64-like mutations (FIG. 25D). The total number of mutations acquired and PCT64-donor-like mutations (R2=0.4869) were positively correlated. GT5 immunization led to the generation of a broader and more diverse repertoire than GT2: aa mutations in IGH were distributed across more numerous sites, though relatively less enriched (FIG. 25E-F). This plasticity promoted PCT64-like mutations at 3 sites in HCDR1 (position 28, 31 and 35), 3 sites in HCDR2 (position 52, 52B and 52C) and 4 sites in HCDR3 (position 92, 97, 100C and 100D) (FIG. 25F). As with GT2, no enrichment site was identified in the LC.

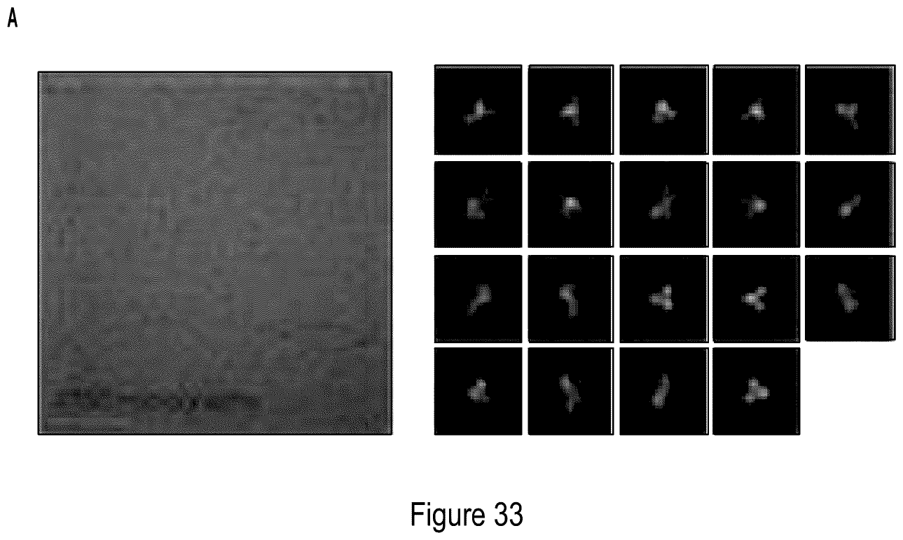

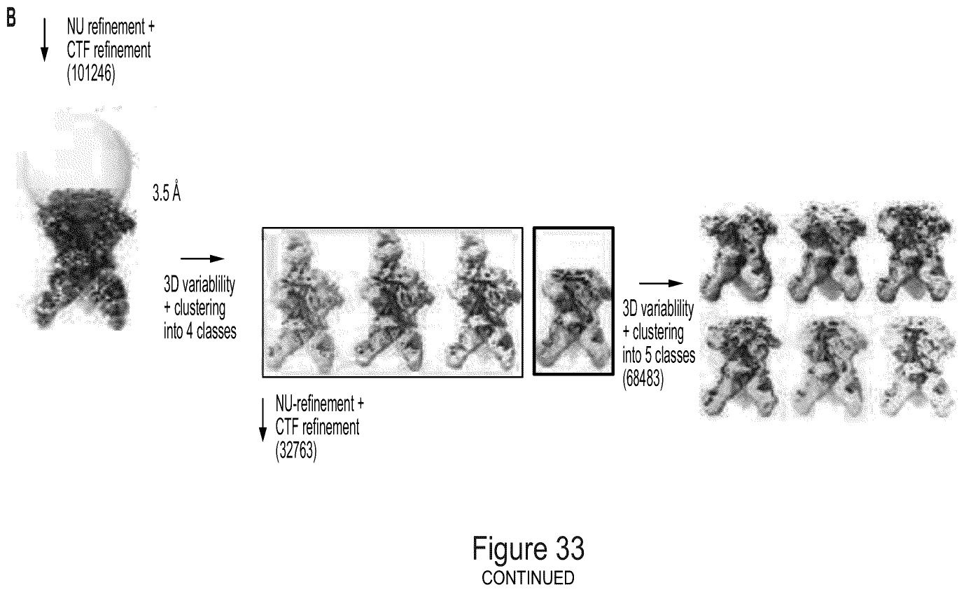

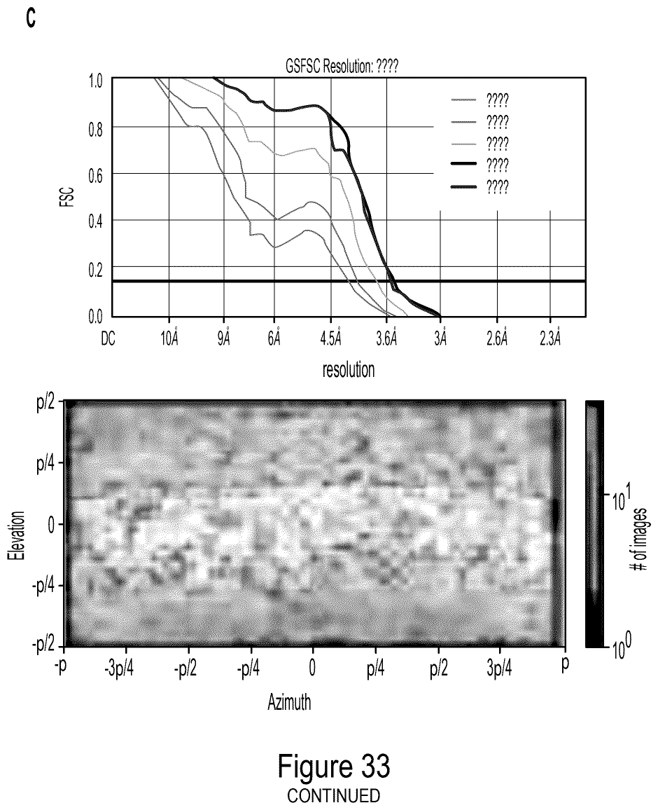

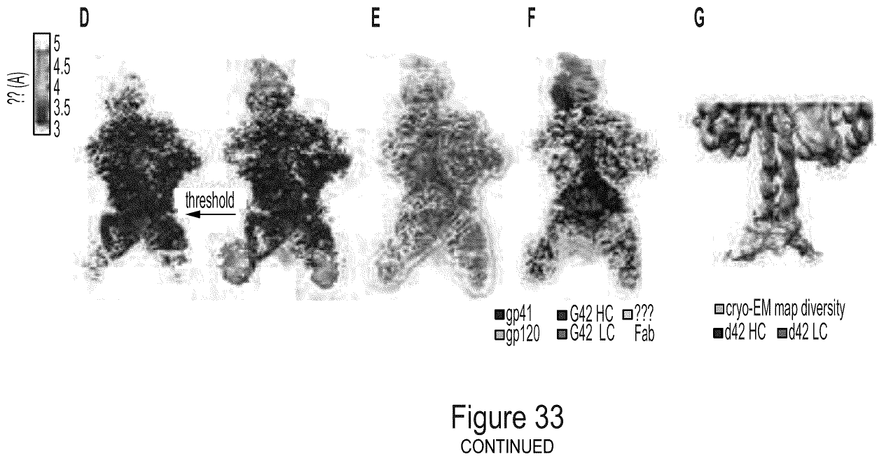

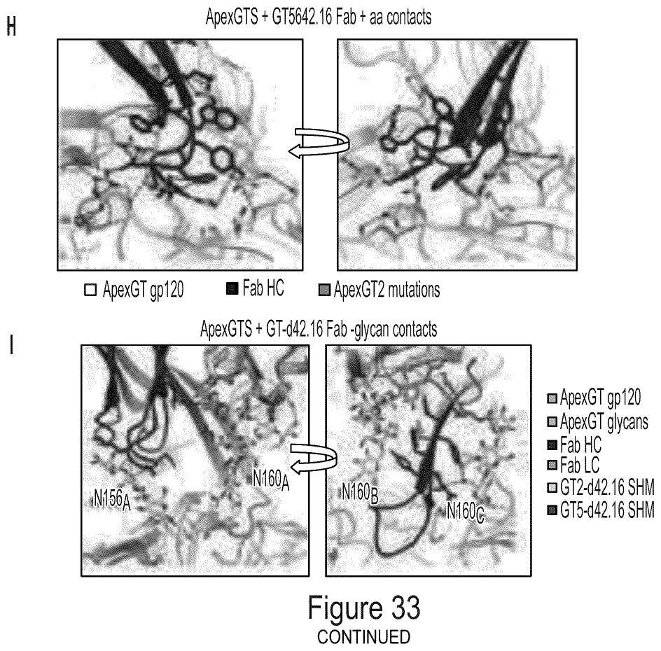

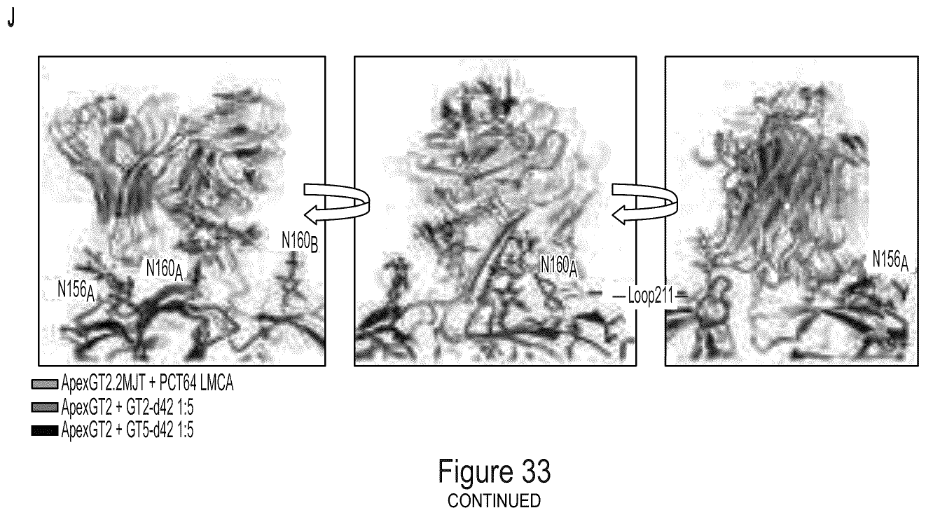

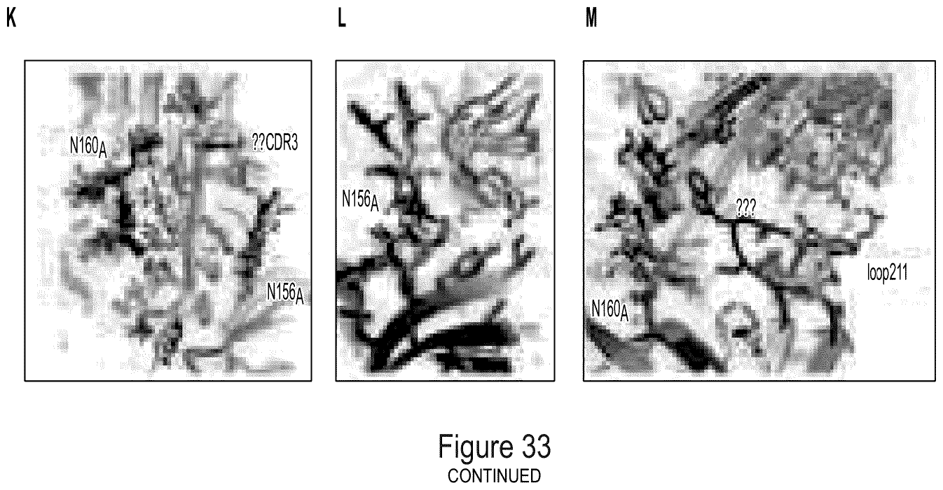

To assess whether SHM acquired from priming conferred a degree of neutralization, five GT2- and five GT5-induced day 42 mAbs were tested against a series of WT and modified (with ApexGT mutations) HIV pseudo viruses (PSVs) based on isolates neutralized by PCT64 (FIG. 25G) (Landais et al., 2017). None of the day 42 mA bs could neutralize the WT PSV s, but some showed partial neutralization of PSVs with ApexGT mutations, especially PSVs with the K 169R mutation (PSVs GT5-V2B and GT5-N167) (FIG. 25G). This is consistent with the binding mode of LMCA (Willis et al., 2021) and day 42 Fabs (FIG. 22G-K and FIG. 25H-L), which includes strong electrostatic interactions between their acidic residues and R169 of ApexGTs. As R169 is rare among WT HIV isolates (<10%), it will be important to test in future experiments whether heterologous boost immunization can reduce the neutralization dependence on R169. To clarify the molecular details of GT5-induced SHM, Applicants determined the cryo-EM structure of GT5 in complex with a high-affinity day 42 Fab (GT5-d42.16) (FIG. 25H and FIG. 33A-G). Despite positional differences in the location of SHM sites (magenta), no structural differences were detected between GT5-d42.16 and either PCT64 LMCA or GT2-d42.16 (FIG. 25H; FIG. 33H-M). GT5-d42.16 has five more sites of SHM than GT2-d42.16 while both Fabs present SHM in the HCDR2 domain, which is responsible for engaging/accommodating the N156gp120A glycan and the C strand of V2 (FIG. 25H-1). The glycan at N156 engages in multiple backbone H-bonds with GT5-d42.16, while E53 forms an H-bond with GT5 residue K 171, as in GT2-d42.16. Notably, the D53E mutation is present in the vast majority of PCT64 lineage members. Unlike GT2-d42.16, GT5-d42.16 has one site of SHM in the HCDR3 domain, T93N, which is located at the very beginning of the loop near the N160gp120A glycan binding pocket and is also found in the PCT64.35M bnAb lineage. All remaining mutations in the HC are located outside the paratope and thus not directly involved in affinity maturation. GT5-d42.16 also has several LC mutations, with one, S311, located near the N160gp120A glycan binding interface in the LCDR1 domain (FIG. 25H). However, this residue converges on either an Asn or Asp in all the mature PCT64 antibodies.

At the immunogen level, the only difference between GT2 and GT5 is the identity of loop2B (FIG. 25H). The GT5 loop2B has the N187 glycan knocked out and includes a mutation to a bulky tryptophan residue at position 188 at the tip of the loop, among other mutations. Using difference mapping, Applicants observed that the removal of the N187 glycan creates a hole in the density surrounding the PCT64 binding site that results in a slight change in the average binding angle of the Fab, presumably by relieving steric restrictions from the glycan (FIG. 25J), which is in line with the SPR results showing a slightly faster on and off rate of PCT64.LMCA for GT5 (Willis et al., 2021). The conformation of the loop is also affected, especially on protomer C, where it is folded inward toward the Fab and N160gp120C glycan (FIG. 25K, L). Although W188 does not appear to interact with the GT5-d42.16 HCDR3, there is clear EM map density extending from the tip of the loop to the N160 glycan resulting from the W188 residue, suggesting it could be stabilizing the glycan, and in turn, its interactions with the Fab (FIG. 25K).

These results indicate that immunization with GT5 can induce affinity maturation in rare PCT64 precursors and support the acquisition of PCT64-like mutations that neutralize autologous virus.

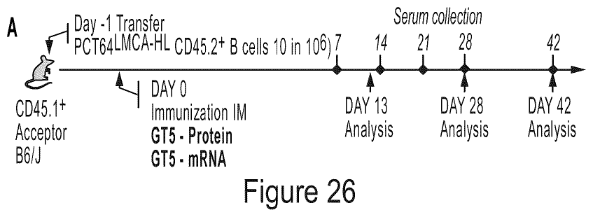

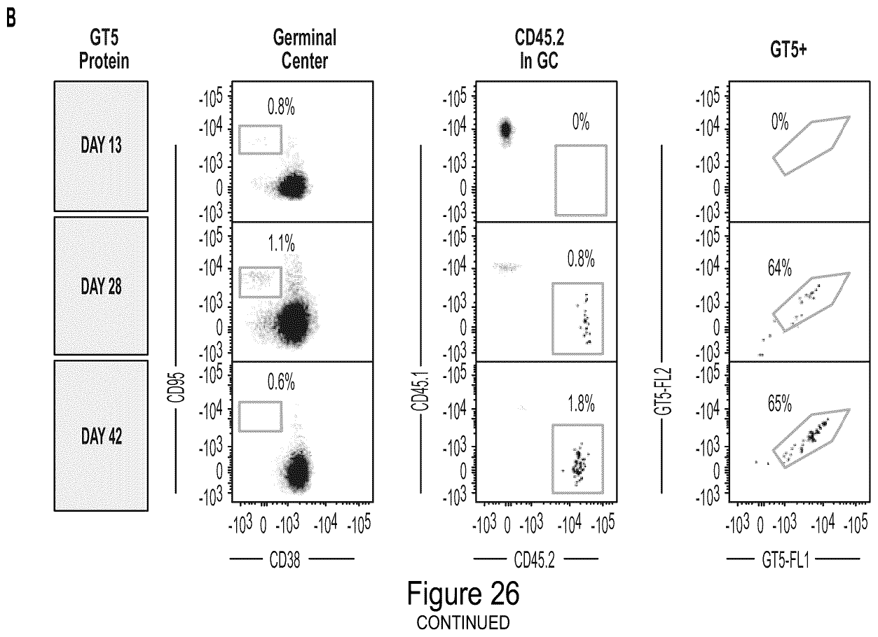

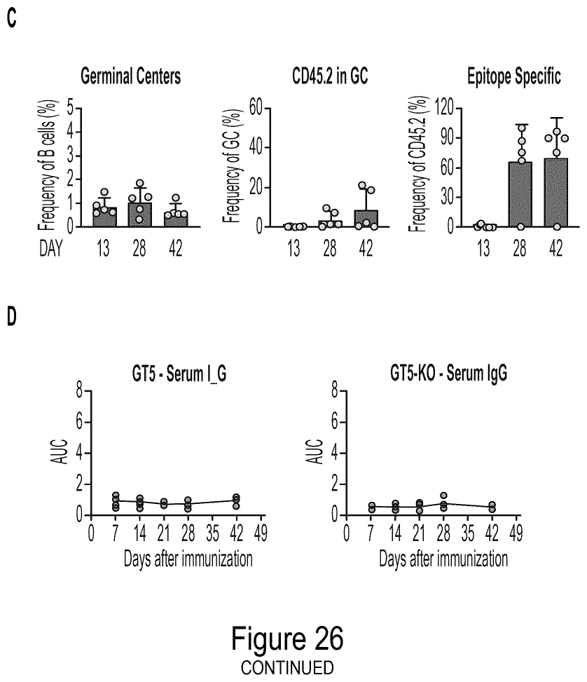

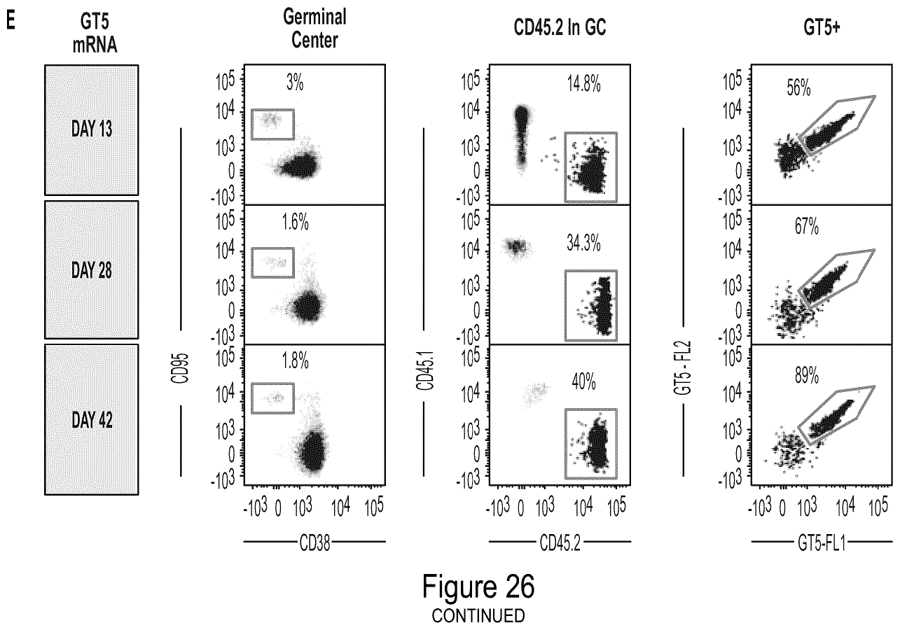

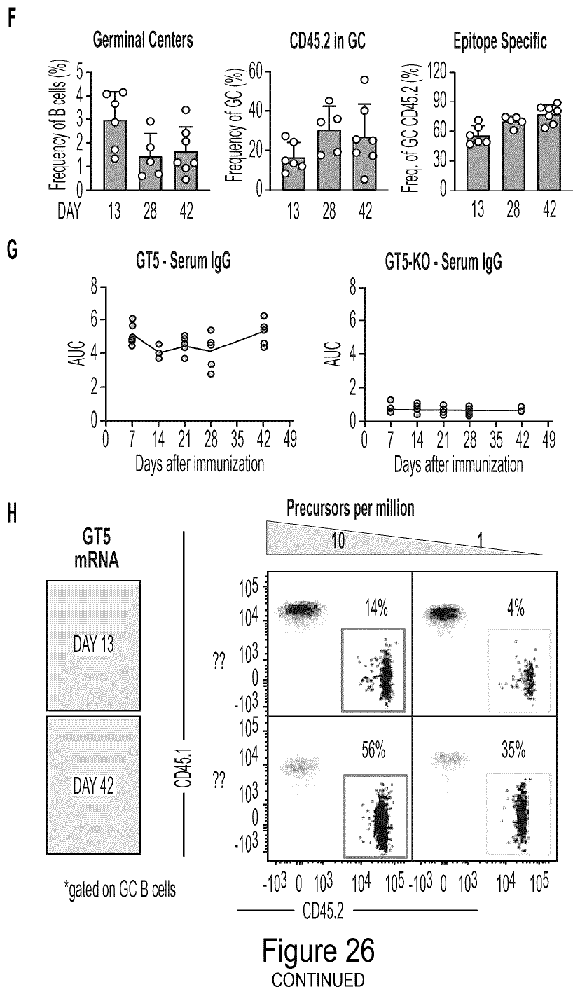

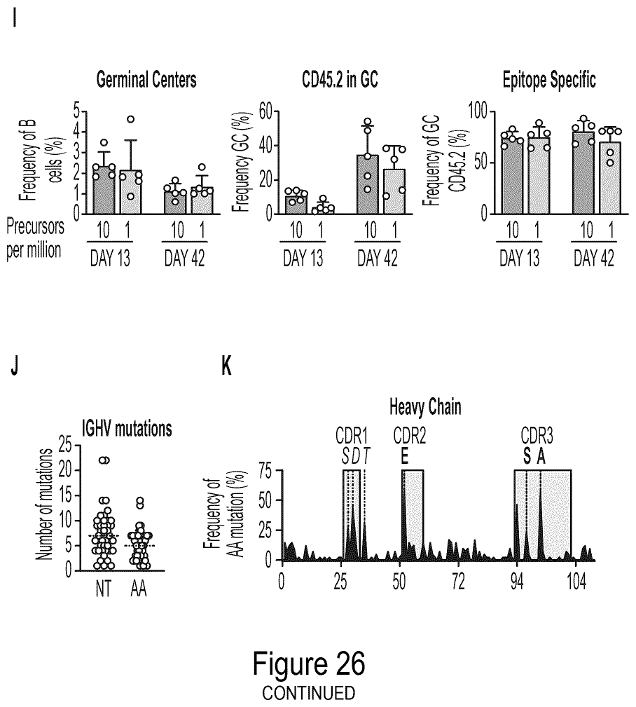

mRNA-LNP membrane-bound GT5-trimers potently activate PCT64 precursors. Nucleoside-modified mRNA vaccines are a promising alternative to conventional approaches. Indeed, mRNA vaccines for SARS-COV-2 have proven safe and highly efficacious in humans (Baden et al., 2021; Thomas et al., 2021). Thus, GT5 was further developed as a membrane-bound trimer with appropriate antigenic profile expressed from DNA or mRNA (Willis et al. 2021). As human vaccines are frequently administered intramuscularly (IM) (Zhang, Wang and Wang, 2015), Applicants first assessed IM GT5 protein trimer delivery. Applicants established PCT64 LMCA at 10 per 106 B cells in recipient mice, immunized IM with GT5 in Sigma adjuvant and measured the response in the inguinal lymph nodes at 13, 28 and 42 dpi (FIG. 26A). GT5-specific CD45.2+ B cells were not present in the GCs at 13 dpi and only present in small quantities at later timepoints (FIG. 26B-C). To assess whether B cell responses could have occurred at different sites, Applicants quantified GT5-specific IgG serum by ELISA, retrieving no detectable titers (FIG. 26D). The difference with Applicants' GT5 IP-immunization data suggests that this GT5 formulation might not be suited for IM delivery in mice.

Applicants then evaluated immune responses to mRNA-mediated expression of membrane-bound GT5 trimers. Recipient mice (10 in 106 PCT64LMCA B cell frequency) which were immunized IM with a single dose of mRNA-LNP encoding GT5 had significantly larger GCs and high recruitment of CD45.2 PCT64LMCA B cells at 13 dpi (FIG. 26E-F). PCT64LMCA B cells were maintained in GCs up to 28 and 42 dpi, and their GT5-specificity increased from 60% to 90% of CD45.2 GC B cells over time (FIG. 26E-F). ELISA of serum IgG titers confirmed that IM mRNA-LNP immunization induces long-lasting, GT5-specific antibodies (FIG. 26G).

Given the magnitude of the response generated after mRNA immunization, Applicants tested whether GT5-mRNA could generate consistent responses after a 10-fold precursor reduction to 1 per million B cells (FIG. 26H-1). Even at this extremely rare starting frequency, PCT64LMCA averaged 4% of GC B cells at 13 dpi (9.9% at 10 precursors per million) and expanded to 26.6% at 42 dpi (FIG. 261).

To confirm that mRNA-LNP GT5 maintained the capacity to induce PCT64-like mutations, Applicants performed BCR sequencing of class switched CD45.2+GT5+ B cells at 42 dpi. Isolated IGHV genes acquired an average of 7 nt/5 aa mutations (FIG. 26I). Enriched sites occurred in similar positions previously identified with protein trimer immunization (FIG. 26K).

Thus, Applicants demonstrated that mRNA-LNP-encoded GT5-trimers activate ultra-rare PCT64 precursors more potently than soluble protein trimers and induce the acquisition of similar bnAb-like mutations.

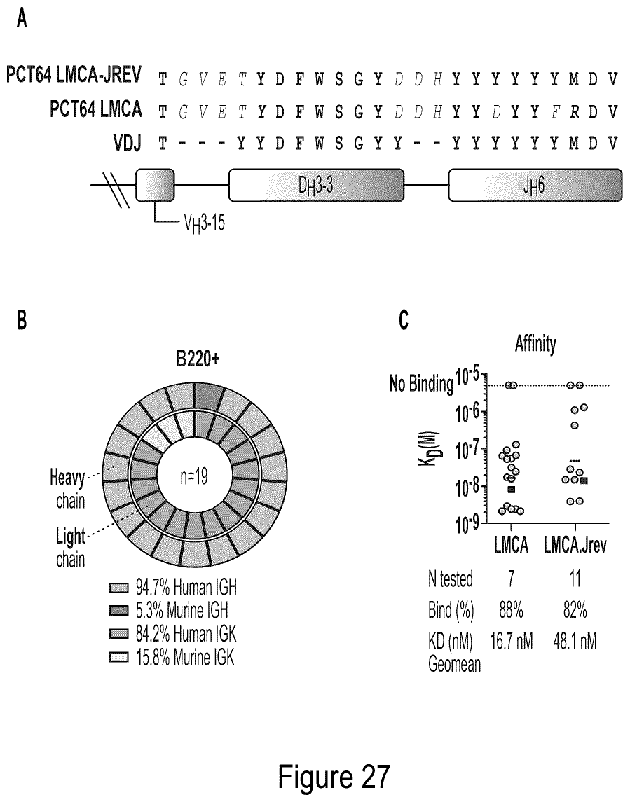

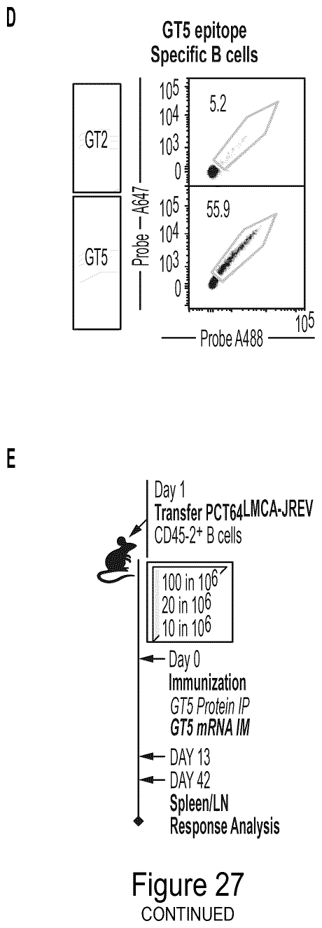

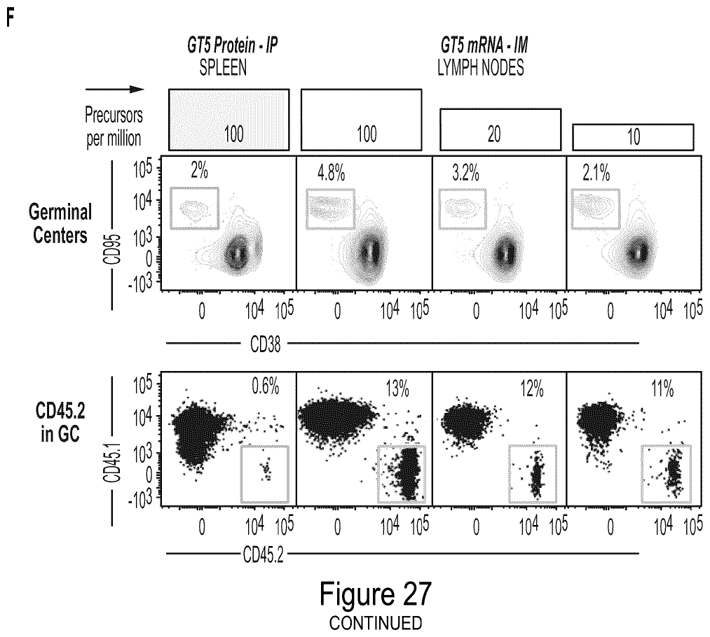

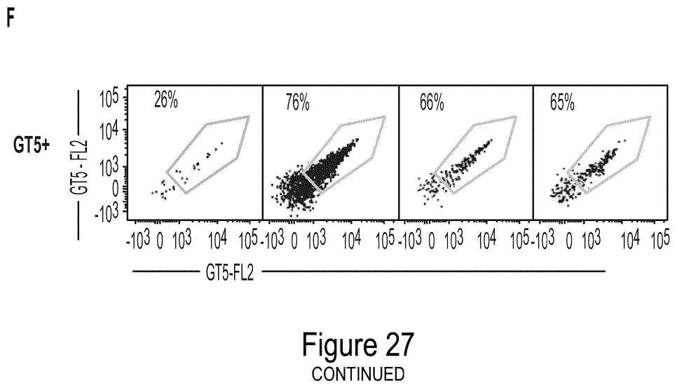

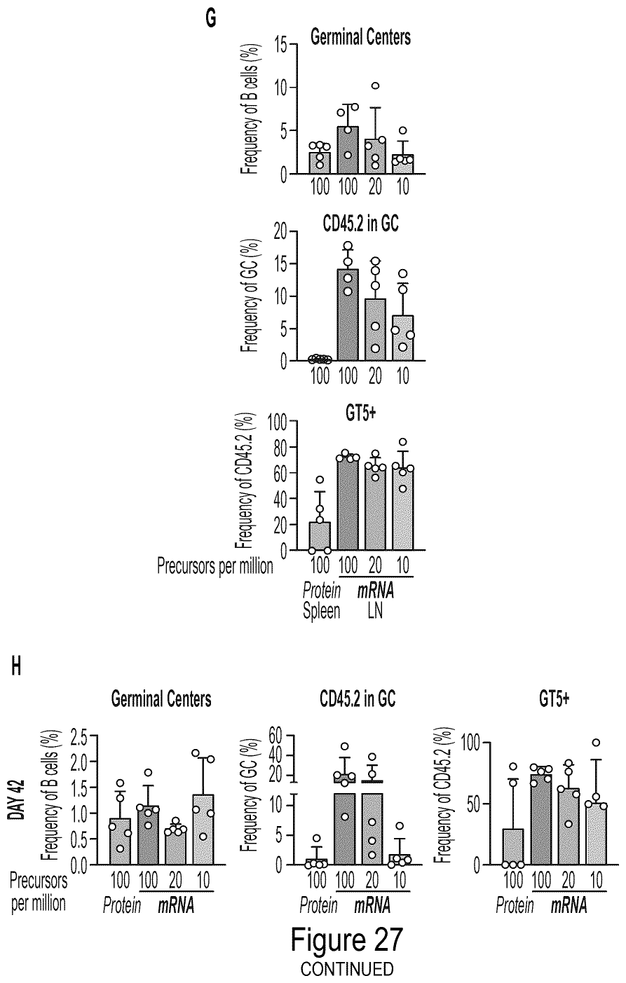

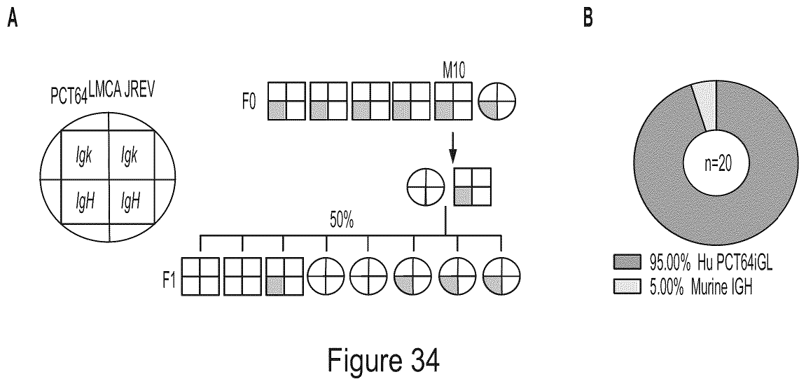

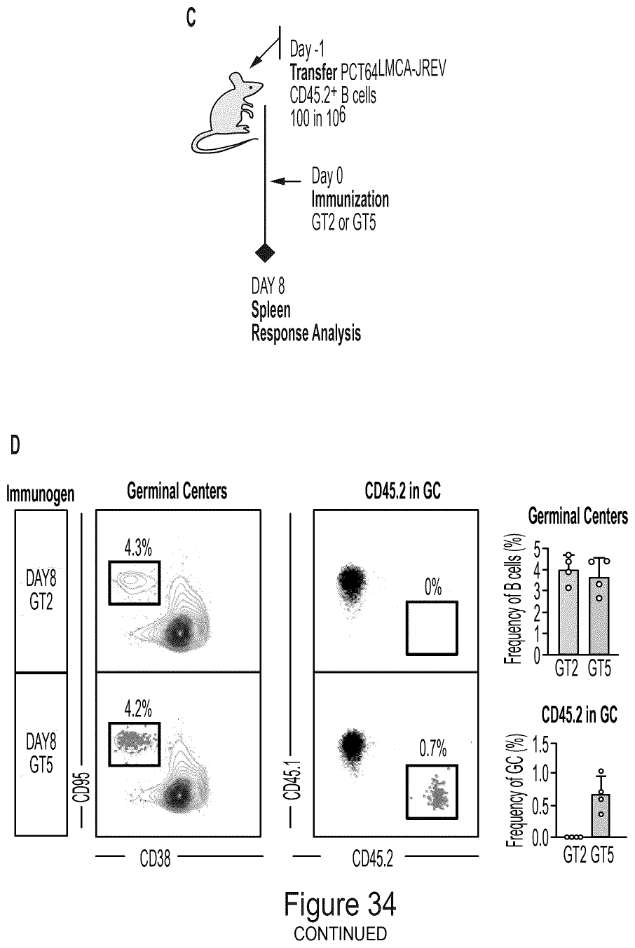

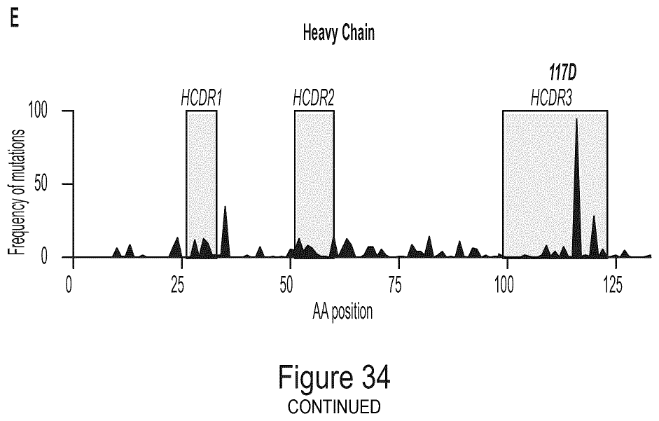

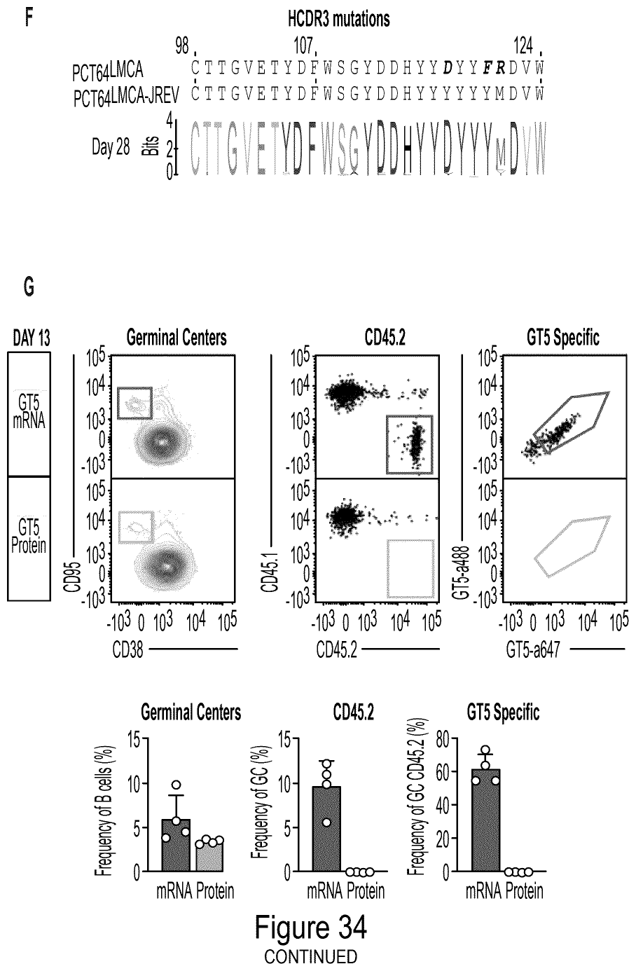

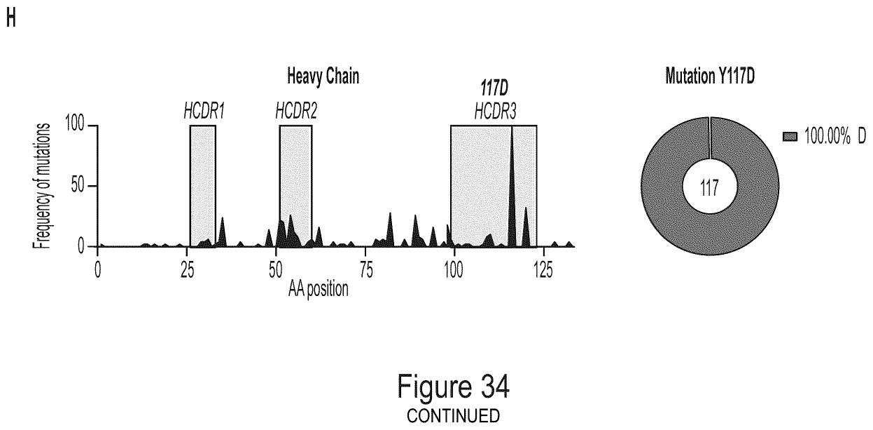

mRNA-LNP-encoded GT5-trimers lower the precursor activation affinity threshold. GT5 was engineered using Applicants' mammalian display bootstrapping approach, wherein GT mutations are identified first against more mature-like antibodies and then modified to increase the immunogen affinity for progressively more germline-like antibodies (Steichen et al., 2016). The PCT64 LMCA sequence, which was isolated from the human donor early after infection, still possesses a minimal degree of SHM in IGH-three aa mutations in the J gene and two aa from the full-length D gene (FIG. 27A). Therefore, to test the capacity of the GT immunogens to activate more reverted germline IGHs, Applicants generated a second mouse model with B cells bearing the HC of PCT64 LMCA.JREV, where the J gene is fully reverted (PCT64LMCA.JREV-H) (FIG. 34A-B) (Willis et al. 2021). Through genotyping and single cell BCR sequencing, Applicants confirmed that the PCT64LMCA.JREV-H sequence was expressed by ˜95% of the B cell repertoire (FIG. 34B). Through crossing with PCT64LMCA-L Applicants obtained mice where PCT64LMCA.JREV IGH and IGK were paired in ˜84% of the repertoire (FIG. 27B).

Reverting the LMCA J-gene mutations significantly decreased the affinity of PCT64LMCA.JREV for GT2 (KD=6.4 μM) and GT5 (KD=347 nM) (Willis et al. 2021). Antibodies with PCT64LMCA.JREV IGH paired with murine IGK had a geomean affinity of 48.1 nM among binders, lower than the 16.7 nM geomean affinity of binders for PCT64LMCA IGH paired with the same murine IGK (FIG. 27C). The lower affinity for PCT64 LMCA.JREV was reflected in the low frequencies of naïve B cells binding GT2 (5%) and GT5 (55%) probes (FIG. 27D). To determine whether precursors in these affinity ranges could be triggered by immunization, PCT64LMCA.JREV-H B cells were transferred into CD45.1 WT mice at a frequency of 100 precursors per 106 prior to IP-immunization with either GT2 or GT5 protein trimers (FIG. 34C). GCs developed in response to both immunogens, but no PCT64LMCA.JREV-H B cell activation was detected after GT2 immunization at 8 dpi. However, PCT64LMCA.JREV B cells were present in GCs after GT5 immunization (FIG. 34D), demonstrating that the improved GT5 immunogen can activate PCT64 precursors with LMCA.JREV BCRs. At 28 dpi, mutation frequency analysis identified enriched sites that were different from the ones induced in LMCA B cell by GT5 (FIG. 34E). In particular, an aspartic acid (D) was acquired in position 117 in the HCDR3 in 98% of the GT5+class switched B cells (FIG. 34E); this heavily selected mutation was present in the original LMCA sequence (FIG. 34F) thus indicating a converging HCDR3-region maturation pathway. The precursor frequency necessary to induce activation in the LMCA.JREV experiments was far from the estimated human range. Therefore, Applicants tested whether GT5-mRNA trimers could elicit stronger responses. Applicants transferred PCT64 LMCA.JREV B cells (frequencies of 100, 20 and 10 per 106) and immunized recipient mice IM with 10 μg of mRNA-LNP encoding GT5 or 10 μg of GT5 soluble protein. At 13 dpi, GC responses with strong PCT64LMCA.JREV-H B cell recruitment were present in mRNA-immunized mice but no PCT64 LMCA.JREV-H B cell activation was detected in protein-immunized mice (FIG. 34G). Applicants then directly compared mRNA-GT5 IM and GT5 protein IP immunization (FIG. 27E). Responses were analyzed 13 and 42 dpi in inguinal LNs or spleen. At 13 dpi CD45.2 LMCA.JREV B cell responses to mRNA were 10-15 times higher than to protein (FIG. 27F-G). Furthermore, strong CD45.2 responses were present in the low frequency groups (20 and 10 per 106 B cells) (FIG. 27F-G). LMCA.JREV GC responses to mRNA were maintained and expanded (reaching ˜20% of GC B cells) at 42 dpi in mice with a starting precursor frequency of 100 and 20 per 106. Responses faded in mice with lower precursor numbers and in mice immunized with protein GT5 (FIG. 27H). Analysis of GT5+ class switched B cells at 28 dpi revealed a mutation pattern overlapping with that induced by protein in JREV, characterized by an enrichment of aspartic acid (D) in position 117 in the HCDR3 (FIG. 34H). These results suggest that mRNA-encoded GT5 may act as a stronger activator of V2-apex B cell responses, possibly by lowering the affinity threshold of B cell activation (Batista and Neuberger, 1998, 2000; Fleire et al., 2006). Thus, mRNA-LNP encoded membrane-bound GT5 trimers may be promising vaccine candidates for the development of priming immunogens for PCT64-like precursors with diverse junctions and affinities.

Inducing bnAbs by immunization is a key objective of the quest for an HIV vaccine (Haynes and Burton, 2017). Central to these efforts is the structure-based design and validation of GT immunogens, which must activate rare precursor B cells, trigger durable GC responses, and induce desired mutations (Jardine et al., 2013). Here, Applicants engineered two BCR KI mice that express two different precursors of the V2-apex bnAb PCT64 to evaluate GT immunogens (Landais et al., 2017). In Applicants' preclinical models, high affinity ApexGT5 was superior to ApexGT2 at activating rare PCT64 precursors. Applicants then directly compared protein-based and nucleoside-modified mRNA immunogens and determined that, at the relative doses administered, the latter improved activation. BCR immunogen affinity is one of the major determinants of rare B cell activation (Abbott et al., 2018; Dosenovic et al., 2018), and V2-apex precursors are notably rare. Most HIV-1 bnAbs specific to the V2-apex rely on long HCDR3 loops ranging from 24 to 39 amino acids to interact with their epitopes (Andrabi et al., 2015), and B cells bearing long HCDR3s (>24 aa) represent only 3.5% of the human repertoire (Briney, Willis, and Crowe 2012). PCT64 precursors, with 25-residue HCDR3s, are estimated to have an upper frequency limit of 20 per 106 of B cells. In comparison, VRC01 precursors specific for the eOD-GT8 immunogen circulate at a frequency of 3.3 per 106 of B cells (Jardine et al., 2016; Havenar-Daughton et al., 2018; Lee et al., 2021). The novel GT5 immunogen initiated strong, sustained precursor activation, with a striking 10-fold increase over GT2 in GC recruitment at the lowest precursor frequency investigated. Remarkably, in addition to the expansion of specific B cell clones, ApexGT immunogens (GT2 and GT5) elicited on-track SHM. However, while the PCT64-like mutations acquired in this study conferred autologous virus neutralization, subsequent booster immunizations with more native-like immunogens would likely be needed to achieve cross-clade neutralization.

In Applicants' models, where various murine LCs pair with the germline PCT64 IGHs, immunizations with GT2 and GT5 both produced reproducible HC mutation fingerprints and did not display stringent LC restriction. This suggests a predominantly IGH-dependent selection process, and validates the applicability of trimer design targeting HCDR3-dominant bnAb precursors, previously tested only for BG18 (Steichen et al., 2019). There does, however, appear to be a marginal role for LC binding: specific murine IGK pairings can be so detrimental as to completely abrogate immunogen binding. At the same time, we observed advantageous IGH-IGK pairings that increased the affinity of the original germline antibody. The capacity of a single immunogen to engage multiple B cell clones that share similar characteristics of PCT64 IGH independently from the paired LC greatly expands the range of precursors that can be elicited through immunization, potentially raising the odds of successfully developing bnAbs.

Applicants also found that immunization with mRNA-LNP coding for membrane-bound GT5 was superior to soluble GT5 protein at triggering low-frequency PCT64-precursor B cells. The greater capacity of mRNA-LNP to promote GC responses may be due to a combination of factors. First, V2-apex bnAbs tightly bind to quaternary epitopes, recognizing the N-linked glycan at residue 160 (N160) and interacting with a protein surface of the V2 domain of gp120 encompassing multiple protomers; they do not show significant binding to monomeric GP120 (Andrabi et al., 2015, 2017; Gift et al., 2017; Moore et al., 2017). The presentation of a fully formed trimeric structure to B cells is thus a fundamental condition for their activation. IM immunization may negatively impact the successful presentation of an intact trimer structure; a similar effect has been previously reported in mice (Hu et al., 2015). Nucleoside-modified mRNA-LNP vaccines allow the antigen to be translated into protein directly into the host's cells, minimizing trimer processing by protease in antigen presenting cells and increasing the chances that B cells encounter a fully formed, well-folded trimer. Finally, the amount of trimer produced after mRNA immunization may be larger than the protein dose, and antigen availability may be increased as a consequence of protein expression kinetics (Pardi et al., 2015, 2018; Lederer et al., 2020). Indeed, similar responses were observed when immunogen administration was extended through an osmotic pump (Tam et al., 2016; Cirelli et al., 2019).

To confer broad protection against a wide variety of strains, an all-around HIV vaccine will likely require the elicitation of several lines of antibodies to target multiple vulnerability sites on Env. In this study, Applicants validated HCDR3-focused GT immunogen design for a novel epitope and demonstrated the utility of mRNA-LNP for bnAb precursor activation. As the use of mRNA could simplify the delivery of multiple structurally sound immunogens, both arms of this study, alongside the array of GT vaccine candidates in various stages of clinical testing, may contribute to the development of a multi-component HIV vaccine.

Cryo-EM maps and refined atomic models of GT2+GT2-d42.16 and GT5+GT5-d42.16 have been deposited in the EM DB and PDB under the accession IDs 25754/7T9A and 25755/7T9B, respectively.

Mice and immunizations. For experiments male B6.SJL-Ptprc^{a}pepc^{b}/BoyJ mice (CD45.1^{+/+}) between 7-12 weeks of age were purchased from The Jackson Laboratory (Bar Harbor, ME). FO-mice from the PCT64^{LMCA} KI mouse (CD45.2+/+) colony were bred at the animal facility of the Gene Modification Facility (Harvard University) and breeding for colony expansion and experimental procedures was subsequently performed at the Ragon Institute of MGH, MIT and Harvard. Ear or tail snips from PCT64^{LMCA} KI mice were used for genotyping by TaqM an assay for a fee for service agreement (TransnetYX). TaqM an probes for the genotyping assay were developed by TransnetY X. CD45.2⁺ B cells from PCT64^{LMCA} donor KI mice were enriched using the Pan B Cell Isolation Kit II (Miltenyi Biotec), counted, diluted to desired cell numbers in PBS and adoptively transferred into CD45.1⁺ recipient mice as reported previously (Abbott et al., 2018).

Preparations of immunogens (GT2 and GT5 at 10 μg/mouse) were diluted in PBS at a volume of 100 μl/mouse for intraperitoneal (IP) injection or 50 μl/mouse for intramuscular (IM) injection and then mixed at a 1:1 ratio with Sigma adjuvant system (Sigma) for at least 25 min. The final formulation was injected IP (total volume of 200 μl/mouse) or IM in the thigh muscles of the hind limb (total volume of 100 μl/mouse). For mRNA-LNP GT5 immunization (10 μg/mouse), preparation was defrosted and immediately diluted in PBS at a volume of 100 μl/mouse, and then injected IM in the thigh muscles of the hind limb.

All experiments were performed under the approval by the Institutional Animal Care and Use Committee (IA CUC) of Harvard University and the Massachusetts General Hospital (MGH) and conducted in accordance with the regulations of the American Association for the Accreditation of Laboratory Animal Care (AAALAC). All animals were cared for in accordance with AAALAC standards in accredited facilities. All animal procedures were performed according to protocols approved by IACUC, specifically: Animal Study Protocols 2016N000022 and 2016N000286 (MGH).

Generation of PCT64^{LMCA} knock-in (KI) mice. PCT64^{LMCA} KI mice were generated following published protocols (Lin et al., 2018; Wang et al., 2021). In brief, the targeting vector 4E 10 (Ota et al., 2013) was modified by the incorporation of human rearranged PCT64^{LMCA} VDJ (heavy chain construct) or VJ (light chain construct) sequences downstream of the promoter region and by elongation of the 5′ and 3′ homology regions using the Gibson assembly method (NEB). The targeting vector DNA was confirmed by Sanger sequencing (Eton Bioscience Inc.).

Next, fertilized mouse oocytes were microinjected with a donor plasmid containing either the pre-rearranged PCT64^{LMCA} IGH with the mouse VHJ558 promoter, or the pre-rearranged PCT64^{LMCA} IGK with the mouse VK4-53 promoter (200 ng/ul); two pair of single-guided RNAs (sgRNAs, 25 ng/μl) targeting either the H or the K locus; and AltR-Cas9 protein (50 ng/ul) and injection buffer (Wang et al., 2021). Following culture, resulting zygotes were implanted into the uteri of pseudopregnant surrogate C57B L/6J mothers.

Immunogen and FACS probe production. For flow cytometric probe binding Apex-GT2 or -GT5 was biotinylated by BirA enzymatic reaction (Avidity, Inc) according to the manufacturer's protocol. Biotinylated GT2 or GT5 probes and respective-KOs were pre-reacted in independent tubes for at least 30 min in a 4:1 molar ratio with fluorescently labeled streptavidin (SA-A 488 and/or SA-647). Reagents were then combined with fluorescently labeled antibodies for FACS-staining.

ELISA. Antigen specific antibody titers were detected by ELISA, using anti-His Ab (2 μg/ml) to capture GT2, GT5 or -KO antigen (2 μg/ml) on 96-well. Plates were washed 5 times with 0.05% Tween 20 in PBS, blocked with 100 μl of 3% BSA in PBS for 1 h at room temperature (RT), and washed again prior to incubation with 1:2 or 1:5 serially diluted mouse serum samples for 1 hour at RT. Wells were washed and incubated with Alkaline Phosphatase AffiniPure Goat Anti-Mouse IgG (Jackson Immuno Research, #115-055-071) at 1:1,000 in PBS with 0.5% BSA for 1 h at RT. p-Nitrophenyl phosphate (Sigma, #N2770) dissolved in ddH₂O (50 μl/well, RT, 25 min) was used for detection. Absorbance at 405 nm was determined with a plate reader (Synergy Neo2, BioTek). ELISA curves were calculated and analyzed using GraphPad Prism 8.4.3 (GraphPad).

Flow cytometry. At selected time points following immunization, whole spleens were mechanically dissociated to generate single-cell suspensions. A CK lysis buffer was used to remove red blood cells and splenocytes were then resuspended in FACS buffer (2% FBS/PBS), Fc-blocked (clone 2.4G2, BD Biosciences) and stained for viability with Live/Dead Blue (Thermo Fisher Scientific) for 20 min at 4° C. For surface staining GT2 or GT5 probes (described above), as well as antibodies against CD4-A PC-eF780, CD8-A PC-eF780, Gr-1-A PC-eF780, F4/80-A PC-eF780, B220-B510, CD95-PE-Cy7, CD38-A 700, CD45.1-PerCP-Cy5.5, CD45.2-PE, IgD-BV786, IgM-BUV395 and IgG1-BV421, were used. Cells were acquired by a BD LSR Fortessa (BD Biosciences) for flow cytometric analysis and sorted using a BD FACS Aria II instrument (BD Biosciences). Data was analyzed using FlowJo software (Tree Star). B cells were single-cell dry-sorted into 96-well PCR plates, rapidly frozen on dry ice and stored at −80° C. until processing.

BCR sequencing. Following single-cell sorting of antigen-specific B cells, the genes encoding the variable region of the heavy and light chains of IgG were amplified through RT-PCR. In brief, first strand cDNA synthesis was carried out using SuperScript III Reverse Transcriptase (Invitrogen) according to manufacturer's instructions. Nested PCR reactions consisting of PCR-1 and PCR-2 were performed as 25 μl reactions using HotStarTaq enzyme (QIAGEN), 10 mM dNTPS (Thermo Fisher Scientific) and cocktails of IgG- and IgK-specific primers and thermocycling conditions described previously (von Boehmer et al., 2016). PCR products were run on precast E-Gels 96 2% with SYBR Safe (Thermo Fisher Scientific) and wells with bands of the correct size were submitted to GENEWIZ company for Sanger sequencing. HC products were sequenced using the HC reverse primer from PCR-2 (5′ GCTCAGGGAARTAGCCCTTGAC 3′) (SEQ ID NO: 33) and the LC was sequenced using the LC reverse primer (5′ TGGGAAGATGGATACAGTT 3′) (SEQ ID NO: 34) from PCR-2.

Reads were quality-checked, trimmed, aligned, and analyzed using the Geneious software (Biomatters Ltd, New Zealand). IMGT/V-QUEST (www.imgt.org) was used for mouse/Human Ig gene assignments. PCT64^{LMCA}-like mutation calculation were done as described previously (Briney et al., 2019; Soto et al., 2019).

10× Genomics B cell repertoire analysis. Naïve splenic B cells were isolated from the PCT64 H/L knock-in mouse line using the mouse, immunomagnetic negative selection, Pan-B cell Isolation Kit (Miltenyi Biotec, San Diego, CA). The B cell suspension was diluted 1:10 and cell density was manually determined using a hemocytometer. Cell viability was determined to be >90% by automated cell counter (Nucleocounter, Chemometec). Approximately 10,000 were loaded into the 10× Genomics Chromium Controller and encapsulated in gel beads in emulsion. Single-cell gene expression libraries were prepared using the Chromium Single-cell 5′ Library and Gel Bead Kit following the manufacturer's user guide (10× Genomics, Pleasanton, CA). The integrity of the library was determined using the D1000 high sensitivity ScreenTape assay (Agilent, Santa Clara, CA) and quantified using the Qubit fluorometry assay (AAT Bioquest, Sunnyvale, CA). BCR libraries were sequenced on the MiSeq System (Illumina, San Diego, CA) with 2×150 paired end reads (Genewiz, South Plainfield, NJ). Sequencing data produced from the Chromium Single Cell 5′ V (D) J library was analyzed using a customized the 10× Genomics Cellranger pipeline that includes the least mutated common ancestor PCT64 heavy chain sequence (Genewiz, South Plainfield, NJ).

Phylogenetic analysis. Single paired amino acid sequences were joined and aligned using MUSCLE. Clonal lineage trees were generated using FastTree (Price, Dehal and Arkin, 2010) and a Jones Taylor Thornton (Jones, Taylor and Thornton, 1992) model for A A evolution.

Surface plasmon resonance (SPR). Kinetics and affinities of antibody-antigen interactions were measured on a ProteOn XPR36 (Bio-Rad) using GLC Sensor Chip (Bio-Rad) and 1×HBS-EP+pH 7.4 running buffer (20× stock from Teknova, Cat. No H8022) supplemented with BSA at 1 mg/ml. Human Antibody Capture Kit was used according to manufacturer's instructions (Cat. No BR-1008-39 from GE) to immobilize about 6000 RUs of capture mA b onto each flow cell. In a typical experiment, approximately 300-400 RUs of mA bs were captured onto each flow cell and analytes were passed over the flow cell at 50 μL/min for 3 min followed by a 5 min dissociation time. Regeneration was accomplished using 3M Magnesium Chloride with 180 seconds contact time and injected four times per cycle. Raw sensograms were analyzed using ProteOn Manager software (Bio-Rad), including interspot and column double referencing, and either Equilibrium fits or Kinetic fits with Langmuir model, or both, were employed when applicable. Analyte concentrations were measured on a NanoDrop 2000c Spectrophotometer using Absorption signal at 280 nm (Jardine et al. 2015).

Neutralization assay. Plasma and monoclonal antibodies neutralizing activity was assessed using single round of replication in TZM-bl target cells, as described previously (Landais et al., 2016) in presence of DEAE-dextran. Briefly, wild-type (WT) and mutant pseudoviruses were generated by co-transfection of 293T cells with an Env-expressing plasmid and an Env-deficient genomic backbone plasmid (pSG3ΔEnv). Pseudoviruses were harvested 72 h post transfection for use in neutralization assays. Mutant pseudoviruses incorporating sets of amino acid mutations were generated by de novo gene synthesis and cloning (GeneScript).

Quantification and statistical analysis. For all mAb pseudovirus neutralization assays the IC50 (the concentration of mAb needed to obtain 50% neutralization against a given pseudovirus) was calculated from the non-linear regression of the neutralization curve. All neutralization assays were repeated at least twice, and data shown are from representative experiments.

Cryo-EM sample preparation. Purified ApexGT2 or ApexGT5 was incubated overnight at 4° with ˜6 molar excess purified GT2-d42.16 or GT5-d42.16 Fab along with RM20A 3 Fab then purified via size exclusion chromatography on a Superdex 200 Increase column followed by concentration of pooled fractions with a 30 kD molecular weight cut-off Amicon Ultra centrifugal filter to a final concentration of ˜3-7 mg/ml. Concentrated sample was mixed with 0.5 μl of 0.04 mM lauryl maltose neopentyl glycol (LM NG; A natrace) to a final concentration of 0.005 mM and 4 μl of this solution was applied to plasma cleaned 1.2/1.3 C-Flat holey carbon grids (Electron Microscopy Sciences) using a Vitrobot mark IV (Thermo Fisher Scientific) with a 7 sec blot time, 0 blot force, and wait time of 0 sec. Prepared grids were then stored in liquid nitrogen until they were transfer to a microscope for imaging.

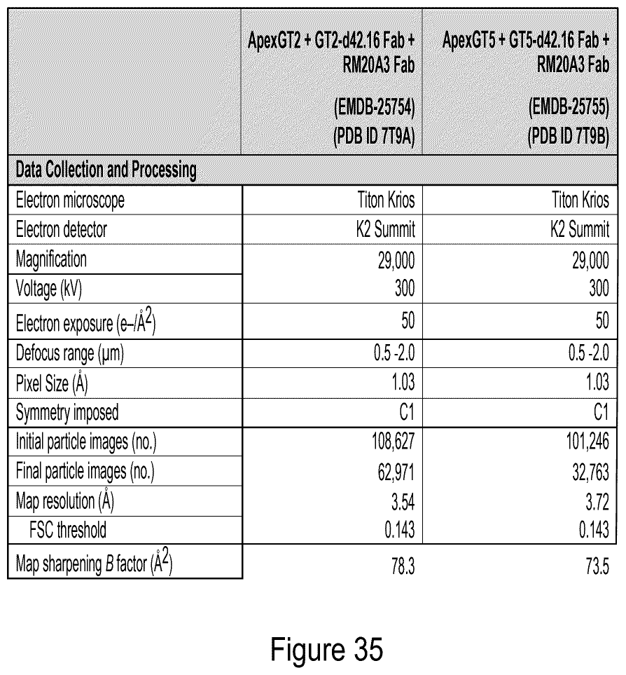

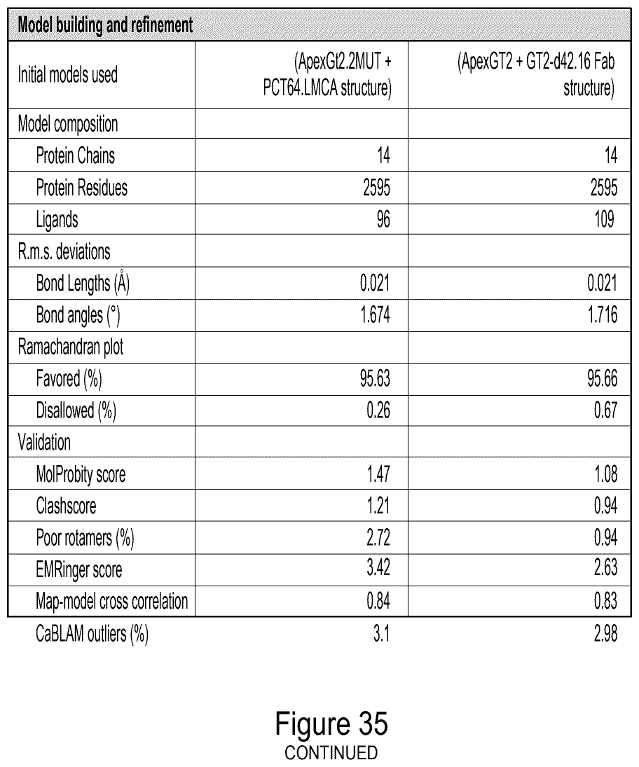

Cryo-EM data collection. A table of detailed imaging conditions and data statistics for all the EM datasets is presented in FIG. 35. All datasets were collected with Leginon automated microscopy software (Suloway et al., 2005) on either an FEI Titan Krios operating at 300 keV or an FEI Talos Arctica operating at 200 keV Thermo Fisher Scientific), both equipped with a K2 Summit direct electron detector (Gatan) operated in counting mode.

Cryo-EM data processing. All movie micrographs were aligned and dose-weighted using MotionCor2 (Zheng et al., 2017) and CTF parameters were estimated with GCTF (Zhang, 2016). Single-particle processing was carried out using a combination of Relion-3 (Kimanius et al., 2016; Zivanov et al., 2018) and CryoSparc2 (Punjani et al., 2017). The following general workflow was used for both datasets presented in this study. After frame alignment, dose-weighting, and CTF estimation, a subset of micrographs were selected based on CTF fit parameters and particle picking was performed, first using a gaussian blob, then templates from 2-D class averages. These particles were then subjected to one-two rounds of 2-D classification followed by subset selection, then one round of ab initio classification followed by subset. After subset selection, 3-D autorefinement was performed with per-particle CTF correction followed by another round of 3-D classification using 3-D variability analysis. A soft spherical mask that surrounds the trimer apex and is large enough to accommodate the entire Fab was used to isolate variability in Fab occupancy followed by clustering into 3-6 classes. Clusters with clear density for Fab were then pooled and refined again together. 3-D variability was then employed again, this time to isolate variability in Fab binding angle followed by clustering and pooling of particles with similar angle of approach. Lastly, a final round of 3-D non-uniform refinement was performed to generate the final reconstruction.

Model building and figure preparation. For the GT2+GT2-d42.16 Fab complex, the previously refined ApexGT2 (Willis et al., 2021) model was docked into the EM density map using UCSF Chimera (Pettersen et al., 2004) along with the previously refined atomic model of PCT64 LMCA and combined into a single PDB file. Mutations associated with GT5 relative to GT2 and d42.16 relative to the LMCA were then manually generated along with manual adjustment of glycans using COOT (Emsley and Crispin, 2018). This initial model was then relaxed into the EM density map using Rosetta (Wang et al., 2016) asking for ˜300 models. All models were validated using MolProbity (Chen et al., 2010) and EM Ringer (Barad et al., 2015) and the model with the best combined score was selected. All models were then checked and adjusted manually in COOT and re-refined with Rosetta, if necessary. Final models were then scored again with MolProbity and EM Ringer, while glycan structures were further validated with Privateer (A girre et al., 2015). Figures were prepared with either UCSF Chimera or ChimeraX (Pettersen et al., 2021). Hydrogen bonds were calculated and displayed with UCSF ChimeraX. Volume segmentation was performed with Segger (Pintilie et al., 2010) as implemented in UCSF ChimeraX. Figures were prepared in Adobe Illustrator (A dobe Inc.) and PowerPoint (Microsoft).

### REFERENCES

Abbott, R. K. et al. (2018) ‘Precursor Frequency and Affinity Determine B Cell Competitive Fitness in Germinal Centers, Tested with Germline-Targeting HIV Vaccine Immunogens’, Immunity. Elsevier Inc., 48 (1), pp 133-146.e6. doi: 10.1016/j.immuni.2017.11.023.
Agirre, J. et al. (2015) ‘Privateer: Software for the conformational validation of carbohydrate structures’, Nature Structural and Molecular Biology. Nature Publishing Group, 22 (11), pp. 833-834. doi: 10.1038/nsmb.3115.
Andrabi, R. et al. (2015) ‘Identification of Common Features in Prototype Broadly Neutralizing Antibodies to HIV Envelope V2 Apex to Facilitate Vaccine Design’, Immunity. Elsevier Inc., 43 (5), pp. 959-973. doi: 10.1016/j.immuni.2015.10.014.
Andrabi, R. et al. (2017) ‘Glycans Function as Anchors for Antibodies and Help Drive HIV Broadly Neutralizing Antibody Development’, Immunity. Elsevier Inc., 47 (3), pp. 524-537.e3. doi: 10.1016/j.immuni.2017.08.006.
Baden, L. R. et al. (2021) ‘Efficacy and Safety of the mRNA-1273 SARS-COV-2 Vaccine’, New England Journal of Medicine, 384 (5), pp. 403-416. doi: 10.1056/NEJMoa2035389.
Barad, B. A. et al. (2015) ‘EMRinger: Side chain-directed model and map validation for 3D cryo-electron microscopy’, Nature Methods, 12 (10), pp. 943-946. doi: 10.1038/nmeth.3541.
Batista, F. D. and Neuberger, M. S. (1998) ‘Affinity Dependence of the B Cell Response to Antigen: A Threshold, a Ceiling, and the Importance of Off-Rate’, Immunity, 8 (6), pp. 751-759. doi: 10.1016/S1074-7613 (00) 80580-4.
Batista, F. D. and Neuberger, M. S. (2000) ‘B cells extract and present immobilized antigen: implications for affinity discrimination.’, The EMBO journal, 19 (4), pp. 513-20. doi: 10.1093/emboj/19.4.513.
von Boehmer, L. et al. (2016) ‘Sequencing and cloning of antigen-specific antibodies from mouse memory B cells’, Nature Protocols, 11 (10), pp. 1908-1923. doi: 10.1038/nprot.2016.102.
Briney, B. et al. (2016) ‘Tailored Immunogens Direct Affinity Maturation toward HIV Neutralizing Antibodies’, Cell. The Authors, 166 (6), pp. 1459-1470.e11. doi: 10.1016/j.cell.2016.08.005.
Briney, B. et al. (2019) ‘Commonality despite exceptional diversity in the baseline human antibody repertoire’, Nature. Springer US, p. 1. doi: 10.1038/s41586-019-0879-y.
Briney, B. S., Willis, J. R. and Crowe, J. E. (2012) ‘Human peripheral blood antibodies with long HCDR3s are established primarily at original recombination using a limited subset of germline genes.’, PloS one, 7 (5), p. e36750. doi: 10.1371/journal.pone.0036750.
Burton, D. R. and Hangartner, L. (2016) ‘Broadly Neutralizing Antibodies to HIV and Their Role in Vaccine Design.’, Annual review of immunology, 34 (1), pp. 635-59. doi: 10.1146/annurev-immunol-041015-055515.
Chen, V. B. et al. (2010) ‘MolProbity: All-atom structure validation for macromolecular crystallography’, Acta Crystallographica Section D: Biological Crystallography. International Union of Crystallography, 66 (1), pp. 12-21. doi: 10.1107/S0907444909042073.
Chen, X. et al. (2021) ‘Vaccination induces maturation in a mouse model of diverse unmutated VRC01-class precursors to HIV-neutralizing antibodies with &gt;50% breadth’, Immunity. Elsevier Inc., 54 (2), pp. 324-339.e8. doi: 10.1016/j.immuni.2020.12.014.
Cirelli, K. M. et al. (2019) ‘Slow Delivery Immunization Enhances HIV Neutralizing Antibody and Germinal Center Responses via Modulation of Immunodominance’, Cell. Elsevier Inc., 177 (5), pp. 1153-1171.e28. doi: 10.1016/j.cell.2019.04.012.
Doria-Rose, N. A. et al. (2014) ‘Developmental pathway for potent VIV2-directed HIV-neutralizing antibodies’, Nature. Nature Publishing Group, 508 (7498), pp. 55-62. doi: 10.1038/nature13036.
Doria-Rose, N. A. and Landais, E. (2019) ‘Coevolution of HIV-1 and broadly neutralizing antibodies’, Current Opinion in HIV and AIDS, 14 (4), pp. 286-293. doi: 10.1097/COH.0000000000000550.
Dosenovic, P. et al. (2015) ‘Immunization for HIV-1 Broadly Neutralizing Antibodies in Human Ig Knockin Mice’, Cell, 161 (7), pp. 1505-1515. doi: 10.1016/j.cell.2015.06.003.
Dosenovic, P. et al. (2018) ‘Anti-HIV-1 B cell responses are dependent on B cell precursor frequency and antigen-binding affinity’, Proceedings of the National Academy of Sciences, 115 (18), p. 201803457. doi: 10.1073/pnas.1803457115.
Emsley, P. and Crispin, M. (2018) ‘Structural analysis of glycoproteins: Building N-linked glycans with coot’, Acta Crystallographica Section D: Structural Biology. International Union of Crystallography, 74 (4), pp. 256-263. doi: 10.1107/S2059798318005119.
Escolano, A. et al. (2016) ‘Sequential Immunization Elicits Broadly Neutralizing Anti-HIV-1 Antibodies in Ig Knockin Mice’, Cell. Elsevier, 166 (6), pp. 1445-1458.e12. doi: 10.1016/j.cell.2016.07.030.
Fleire, S. J. et al. (2006) ‘B cell ligand discrimination through a spreading and contraction response’, Science, 312 (5774), pp. 738-741. doi: 10.1126/science.1123940.
Flemming, A. (2018) ‘Needles in a haystack: the quest for bnAbs’, Nature Research 2020, 861 (December), p. 2018.
Georgiev, I. S. et al. (2013) ‘Delineating antibody recognition in polyclonal sera from patterns of HIV-1 isolate neutralization’, Science, 340 (6133), pp. 751-756. doi: 10.1126/science.1233989.
Gift, S. K. et al. (2017) ‘Functional Stability of HIV-1 Envelope Trimer Affects Accessibility to Broadly Neutralizing Antibodies at Its Apex’, Journal of Virology, 91 (24), pp. 7-10. doi: 10.1128/jvi.01216-17.
Gorman, J. et al. (2016) ‘Structures of HIV-1 Env V1V2 with broadly neutralizing antibodies reveal commonalities that enable vaccine design’, Nature Structural and Molecular Biology. Nature Publishing Group, 23 (1), pp. 81-90. doi: 10.1038/nsmb.3144.
Hardy, R. R. et al. (1991) ‘Resolution and characterization of pro-B and pre-pro-B cell stages in normal mouse bone marrow.’, Journal of Experimental Medicine, 173 (5), pp. 1213-1225. doi: 10.1084/jem.173.5.1213.
Havenar-Daughton, C. et al. (2018) ‘The human naive B cell repertoire contains distinct subclasses for a germline-targeting HIV-1 vaccine immunogen’, Science Translational Medicine, 10 (448), pp. 1-16. doi: 10.1126/scitranslmed.aat0381.
Haynes, B. F. et al. (2016) ‘HIV-Host Interactions: Implications for Vaccine Design’, Cell Host and Microbe. Elsevier Inc., 19 (3), pp. 292-303. doi: 10.1016/j.chom.2016.02.002.
Haynes, B. F. and Burton, D. R. (2017) ‘Developing an HIV vaccine’, Science, 355 (6330), pp. 1129-1130. doi: 10.1126/science.aan0662.
Hoot, S. et al. (2013) ‘Recombinant HIV Envelope Proteins Fail to Engage Germline Versions of Anti-CD4bs bNAbs’, PLOS Pathogens, 9 (1). doi: 10.1371/journal.ppat.1003106.
Hu, J. K. et al. (2015) ‘Murine Antibody Responses to Cleaved Soluble HIV-1 Envelope Trimers Are Highly Restricted in Specificity’, Journal of Virology, 89 (20), pp. 10383-10398. doi: 10.1128/jvi.01653-15.
Jardine, J. et al. (2013) ‘Rational HIV Immunogen Design to Target Specific Germline B Cell Receptors’, Science, 340 (6133), pp. 711-716. doi: 10.1126/science.1234150.
Jardine, J. G. et al. (2015) ‘Priming a broadly neutralizing antibody response to HIV-1 using a germline-targeting immunogen’, Science, 349 (6244), pp. 156-161. doi: 10.1126/science.aac5894.
Jardine, J. G. et al. (2016) ‘Minimally Mutated HIV-1 Broadly Neutralizing Antibodies to Guide Reductionist Vaccine Design’, pp. 1-33. doi: 10.1371/journal.ppat.1005815.
Jones, D. T., Taylor, W. R. and Thornton, J. M. (1992) ‘The rapid generation of mutation data matrices from protein sequences’, Bioinformatics, 8 (3), pp. 275-282. doi: 10.1093/bioinformatics/8.3.275.
Julien, J. P. et al. (2013) ‘Asymmetric recognition of the HIV-1 trimer by broadly neutralizing antibody PG9’, Proceedings of the National Academy of Sciences of the United States of America, 110 (11), pp. 4351-4356. doi: 10.1073/pnas.1217537110.
Kelsoe, G. and Haynes, B. F. (2017) ‘Host controls of HIV broadly neutralizing antibody development’, Immunological Reviews, 275 (1), pp. 79-88. doi: 10.1111/imr.12508.
Kimanius, D. et al. (2016) ‘Accelerated cryo-EM structure determination with parallelisation using GPUS in RELION-2’, eLife, 5, pp. 1-21. doi: 10.7554/eLife.18722.
Klein, F. et al. (2013) ‘Antibodies in HIV-1 vaccine development and therapy’, Science, 341 (6151), pp. 1199-1204. doi: 10.1126/science.1241144.
Landais, E. et al. (2016) ‘Broadly Neutralizing Antibody Responses in a Large Longitudinal Sub-Saharan HIV Primary Infection Cohort’, PLOS Pathogens, 12 (1), pp. 1-22. doi: 10.1371/journal.ppat.1005369.
Landais, E. et al. (2017) ‘HIV Envelope Glycoform Heterogeneity and Localized Diversity Govern the Initiation and Maturation of a V2 Apex Broadly Neutralizing Antibody Lineage’, Immunity. Elsevier Inc., 47 (5), pp. 990-1003.e9. doi: 10.1016/j.immuni.2017.11.002.
Lederer, K. et al. (2020) ‘SARS-COV-2 mRNA Vaccines Foster Potent Antigen-Specific Germinal Center Responses Associated with Neutralizing Antibody Generation’, Immunity, 53 (6), pp. 1281-1295.e5. doi: 10.1016/j.immuni.2020.11.009.
Lee, J. H. et al. (2021) ‘Vaccine genetics of IGHV1-2 VRC01-class broadly neutralizing antibody precursor naïve human B cells’, npj Vaccines. Springer US, 6 (1), pp. 1-12. doi: 10.1038/s41541-021-00376-7.
Liao, H. X. et al. (2013) ‘Vaccine Induction of Antibodies against a Structurally Heterogeneous Site of Immune Pressure within HIV-1 Envelope Protein Variable Regions 1 and 2’, Immunity. Elsevier, 38 (1), pp. 176-186. doi: 10.1016/j.immuni.2012.11.011.
Lin, Y. et al. (2018) ‘One. step CRISPR/Cas9 method for the rapid generation of human antibody heavy chain knock. in mice’, The EMBO Journal, p. e99243. doi: 10.15252/embj.201899243.
McGuire, A. T. et al. (2013) ‘Engineering HIV envelope protein to activate germline B cell receptors of broadly neutralizing anti-CD4 binding site antibodies’, Journal of Experimental Medicine, 210 (4), pp. 655-663. doi: 10.1084/jem.20122824.
Mclellan, J. S. et al. (2011) ‘Structure of HIV-1 gp120 V1/V2 domain with broadly neutralizing antibody PG9’, Nature. Nature Publishing Group, 480 (7377), pp. 336-343. doi: 10.1038/nature10696.
Moore, P. L. et al. (2017) ‘Ontogeny-based immunogens for the induction of V2-directed HIV broadly neutralizing antibodies’, Immunological Reviews, 275 (1), pp. 217-229. doi: 10.1111/imr.12501.
Mouquet, H. et al. (2012) ‘Complex-type N-glycan recognition by potent broadly neutralizing HIV antibodies’, Proceedings of the National Academy of Sciences of the United States of America, 109 (47). doi: 10.1073/pnas.1217207109.
Ng'uni, T., Chasara, C. and Ndhlovu, Z. M. (2020) ‘Major Scientific Hurdles in HIV Vaccine Development: Historical Perspective and Future Directions’, Frontiers in Immunology, 11 (October), pp. 1-17. doi: 10.3389/fimmu.2020.590780.
Ota, T. et al. (2013) ‘B Cells from Knock-in Mice Expressing Broadly Neutralizing HIV Antibody b12 Carry an Innocuous B Cell Receptor Responsive to HIV Vaccine Candidates’, The Journal of Immunology, 191 (6), pp. 3179-3185. doi: 10.4049/jimmunol.1301283.
Pancera, M. et al. (2010) ‘Crystal Structure of PG16 and Chimeric Dissection with Somatically Related PG9: Structure-Function Analysis of Two Quaternary-Specific Antibodies That Effectively Neutralize HIV-1’, Journal of Virology, 84 (16), pp. 8098-8110. doi: 10.1128/jvi.00966-10.
Pancera, M. et al. (2013) ‘Structural basis for diverse N-glycan recognition by HIV-1-neutralizing V1-V2-directed antibody PG16’, Nature Structural and Molecular Biology. Nature Publishing Group, 20 (7), pp. 804-813. doi: 10.1038/nsmb.2600.
Pardi, N. et al. (2015) ‘Expression kinetics of nucleoside-modified mRNA delivered in lipid nanoparticles to mice by various routes’, Journal of Controlled Release. Elsevier B.V., 217, pp. 345-351. doi: 10.1016/j.jconrel.2015.08.007.
Pardi, N. et al. (2018) ‘mRNA vaccines-a new era in vaccinology’, Nature Reviews Drug Discovery. Nature Publishing Group, 17 (4), pp. 261-279. doi: 10.1038/nrd.2017.243.
Pejchal, R. et al. (2010) ‘Structure and function of broadly reactive antibody PG16 reveal an H3 subdomain that mediates potent neutralization of HIV-1’, Proceedings of the National Academy of Sciences of the United States of America, 107 (25), pp. 11483-11488. doi: 10.1073/pnas.1004600107.
Pettersen, E. F. et al. (2004) ‘UCSF Chimera-A visualization system for exploratory research and analysis’, Journal of Computational Chemistry, 25 (13), pp. 1605-1612. doi: 10.1002/jcc.20084.
Pettersen, E. F. et al. (2021) ‘UCSF ChimeraX: Structure visualization for researchers, educators, and developers’, Protein Science, 30 (1), pp. 70-82. doi: 10.1002/pro.3943.
Pintilie, G. D. et al. (2010) ‘Quantitative analysis of cryo-EM density map segmentation by watershed and scale-space filtering, and fitting of structures by alignment to regions’, Journal of Structural Biology. Elsevier Inc., 170 (3), pp. 427-438. doi: 10.1016/j.jsb.2010.03.007.
Price, M. N., Dehal, P. S. and Arkin, A. P. (2010) ‘FastTree 2-Approximately Maximum-Likelihood Trees for Large Alignments’, PLOS ONE. Edited by A. F. Y. Poon, 5 (3), p. e9490. doi: 10.1371/journal.pone.0009490.
Punjani, A. et al. (2017) ‘CryoSPARC: Algorithms for rapid unsupervised cryo-EM structure determination’, Nature Methods, 14 (3), pp. 290-296. doi: 10.1038/nmeth.4169.
Rantalainen, K. et al. (2018) ‘Co-evolution of HIV Envelope and Apex-Targeting Neutralizing Antibody Lineage Provides Benchmarks for Vaccine Design’, Cell Reports. ElsevierCompany., 23 (11), pp. 3249-3261. doi: 10.1016/j.celrep.2018.05.046.
Rappuoli, R. et al. (2016) ‘Reverse vaccinology 2.0: Human immunology instructs vaccine antigen design’, Journal of Experimental Medicine, 213 (4), pp. 469-481. doi: 10.1084/JEM.20151960.
Sanders, R. W. and Moore, J. P. (2017) ‘Native-like Env trimers as a platform for HIV-1 vaccine design’, Immunological Reviews, 275 (1), pp. 161-182. doi: 10.1111/imr.12481.
Sok, D. et al. (2013) ‘The Effects of Somatic Hypermutation on Neutralization and Binding in the PGT121 Family of Broadly Neutralizing HIV Antibodies’, PLOS Pathogens, 9 (11). doi: 10.1371/journal.ppat.1003754.
Sok, D. et al. (2016) ‘Priming HIV-1 broadly neutralizing antibody precursors in human Ig loci transgenic mice’, Science, 353 (6307), pp. 1557-1560. doi: 10.1126/science.aah3945.
Sok, D. and Burton, D. R. (2018) ‘Recent progress in broadly neutralizing antibodies to HIV’, Nature Immunology. Springer US, 19 (11), pp. 1179-1188. doi: 10.1038/s41590-018-0235-7.
Soto, C. et al. (2019) ‘High frequency of shared clonotypes in human B cell receptor repertoires’, Nature. Nature Publishing Group, 566 (7744), pp. 398-402. doi: 10.1038/s41586-019-0934-8.
Stamatatos, L., Pancera, M. and McGuire, A. T. (2017) ‘Germline-targeting immunogens’, Immunological Reviews, 275 (1), pp. 203-216. doi: 10.1111/imr.12483.
Steichen, J. M. et al. (2016) ‘HIV Vaccine Design to Target Germline Precursors of Glycan-Dependent Broadly Neutralizing Antibodies’, Immunity. The Authors, 45 (3), pp. 483-496. doi: 10.1016/j.immuni.2016.08.016.
Steichen, J. M. et al. (2019) ‘A generalized HIV vaccine design strategy for priming of broadly neutralizing antibody responses’, Science, 366 (6470). doi: 10.1126/science.aax4380.
Suloway, C. et al. (2005) ‘Automated molecular microscopy: The new Leginon system’, Journal of Structural Biology, 151 (1), pp. 41-60. doi: 10.1016/j.jsb.2005.03.010.
Tam, H. H. et al. (2016) ‘Sustained antigen availability during germinal center initiation enhances antibody responses to vaccination’, Proceedings of the National Academy of Sciences, 113 (43), pp. E 6639-E 6648. doi: 10.1073/pnas.1606050113.
Thomas, S. J. et al. (2021) ‘Safety and Efficacy of the BNT162b2 mRNA Covid-19 Vaccine through 6 Months’, New England Journal of Medicine, 385 (19), pp. 1761-1773. doi: 10.1056/nejmoa2110345.
Tian, M. et al. (2016) ‘Induction of HIV Neutralizing Antibody Lineages in Mice with Diverse Precursor Repertoires’, Cell. Elsevier, 166 (6), pp. 1471-1484.e18. doi: 10.1016/j.cell.2016.07.029.
Walker, L. M. et al. (2009) ‘Broad and potent neutralizing antibodies from an African donor reveal a new HIV-1 vaccine target.’, Science (New York, N.Y.), 326 (5950), pp. 285-9. doi: 10.1126/science.1178746.
Walker, L. M. et al. (2010) ‘A limited number of antibody specificities mediate broad and potent serum neutralization in selected HIV-1 infected individuals’, PLOS Pathogens, 6 (8), pp. 11-12. doi: 10.1371/journal.ppat.1001028.
Wang, R. Y. R. et al. (2016) ‘Automated structure refinement of macromolecular assemblies from cryo-EM maps using Rosetta’, eLife, 5 (September2016), pp. 1-22. doi: 10.7554/eLife.17219.
Wang, X. et al. (2021) ‘Multiplexed CRISPR/CAS9. mediated engineering of pre-clinical mouse models bearing native human B cell receptors’, The EMBO Journal, 40 (2), pp. 1-20. doi: 10.15252/embj.2020105926.
Willis, J. R. et al. (2021) ‘Human Immunoglobulin Repertoire-Guided Design of Immunogens Targeting HIV V2-Apex Broadly Neutralizing Antibody Precursors’, Submitted.
Xiao, X. et al. (2009) ‘Germline-like predecessors of broadly neutralizing antibodies lack measurable binding to HIV-1 envelope glycoproteins: Implications for evasion of immune responses and design of vaccine immunogens’, Biochemical and Biophysical Research Communications. Elsevier Inc., 390 (3), pp. 404-409. doi: 10.1016/j.bbrc.2009.09.029.
Zhang, K. (2016) ‘Gctf: Real-time CTF determination and correction’, Journal of Structural Biology. Elsevier Inc., 193 (1), pp. 1-12. doi: 10.1016/j.jsb.2015.11.003.
Zhang, L., Wang, W. and Wang, S. (2015) ‘Effect of vaccine administration modality on immunogenicity and efficacy’, Expert Review of Vaccines. Informa UK, Ltd., 14 (11), pp. 1509-1523. doi: 10.1586/14760584.2015.1081067.
Zheng, S. Q. et al. (2017) ‘MotionCor2: Anisotropic correction of beam-induced motion for improved cryo-electron microscopy’, Nature Methods. Nature Publishing Group, 14 (4), pp. 331-332. doi: 10.1038/nmeth.4193.
Zhou, T. et al. (2010) ‘Structural basis for broad and potent neutralization of HIV-1 by antibody VRC01’, Science, 329 (5993), pp. 811-817. doi: 10.1126/science.1192819.
Zivanov, J. et al. (2018) ‘New tools for automated high-resolution cryo-EM structure determination in RELION-3’, eLife, 7, pp. 1-22. doi: 10.7554/eLife.42166.

### Example 3: Native-Like Trimers Delivered by mRNA

Nomenclature. The three most advanced native-like trimers Applicants have developed for mRNA delivery are as follows:

(1) A prefusion-stabilized, cleavage-independent, glycan-hole-masked soluble trimer (BG505 MD39.3). MD39 is an improved version of BG505 SOSIP with improved antigenicity, trimer yield, and thermal stability, that Applicants engineered via structure-guided mammalian display directed evolution (Steichen et al. Immunity 2016). MD39.3 includes a linker (“link14”) across the gp120-gp41 cleavage site that Applicants also engineered by directed evolution (Steichen et al. Science 2019), and mutations to introduce glycosylation sites in the well-known 241/289 glycan holes of BG505 as described in Kulp et al. Nat. Comms 2017.

(2) The matched trimer in a membrane-bound format including a transmembrane domain but lacking a C-terminal domain (BG505 MD39.3 gp151). This gp151 construct was designed not to include an endocytosis motif that was inadvertently included in Applicants' original gp160-dCT constructs tested with Moderna and as a result this gp151 construct displays 8-fold improved expression.

(3) The same membrane-bound trimer with a point mutation in the CD4bs that reduces CD4 binding to undetectable levels by SPR and reduces binding to CD4+ T cells by 1000-fold (BG505 MD39.3 CD4KO gp151). The point mutation, G473T, was described in Kulp et al. Nat. Comms. 2017.

Rabbit studies. In a large rabbit immunization study with IAVI/BMGF/Moderna, Applicants tested their BG505 MD39 link14 cleavage-independent native-like trimer (MD39.2) delivered as Moderna mRNA or purified soluble protein in multiple formats (Table 2). The different formats included soluble trimer and ferritin nanoparticle (tested as protein and mRNA), and two types of membrane-bound trimers tested only as mRNA, membrane-tethered trimer and natively membrane-bound trimer. The membrane-tethered construct mimicked the format that Applicants use in mammalian display, whereas the natively membrane-bound format was a gp160-dCT. As controls, Applicants included purified proteins for BG505 SOSIP, olio6 (a variant of MD39), and gp120 foldon. Applicants are currently conducting a smaller follow-up rabbit study to repeat the test of MD39.2 membrane-bound and soluble trimers delivered as mRNA. In this second study Applicants have also included mRNA delivery of membrane-bound and soluble forms of MD39.3 corresponding to MD39.2 with the 241 and 289 glycosylation sites intact, as well as a mRNA and protein forms of their circularly-permuted soluble trimer CPG9 (Kulp et al Nat. Comms. 2017) that provides an alternative strategy to eliminate the cleavage site and also affords a degree of glycan masking on the bottom (Table 3). In both experiments, rabbits were immunized by the IM route at weeks 0, 8, and 24, with either 30 μg mRNA/LNP or 100 μg protein +75U ISCOM IT, with the adjuvant provided by Darrell Irvine.

Applicants hypothesize that autologous neutralization responses generally provide a readout on the degree to which the trimer constructs maintain a native-like state in vivo. As shown in FIG. 36, the Moderna mRNA-launched MD39.2 gp160-dCT membrane-bound trimer elicited autologous neutralizing antibodies as well or better than any other mRNA group (left side of FIG. 36), including two different groups representing the matched soluble trimer (MD39.2 WTsp and MD39.2), a ferritin nanoparticle presenting the matched trimer (MD39.2 ferritin), and the matched trimer tethered to the membrane via a long flexible linker (MD39.2 PDGFR).

FIG. 36 also shows that the gp160-dCT construct performed approximately as well as any of the purified protein groups (right side of FIG. 36), including the matched trimer (MD39.2) and its cleavage-site-intact variant (MD39), the matched trimer presented on ferritin (MD39.2 ferritin), and SOSIP. Using a Kruskal-Wallis statistical test that corrects for multiple comparisons, the MD39.2 gp160-dCT group was not significantly different from any other mRNA or protein group except the MD39.2 ferritin (mRNA) and gp120 foldon (protein). The caveat with these data is that the neutralization titers for both protein and mRNA groups are low compared to historical data for three administrations of BG505 SOSIP protein plus adjuvant in rabbits (Sanders et al. Science 2015; de Taeye et al. Cell 2016). But if Applicants assume that an unknown factor has reduced titers in all groups relatively equally, then it seems fair to conclude that the membrane-bound trimer performed surprisingly well against all other mRNA or protein groups.

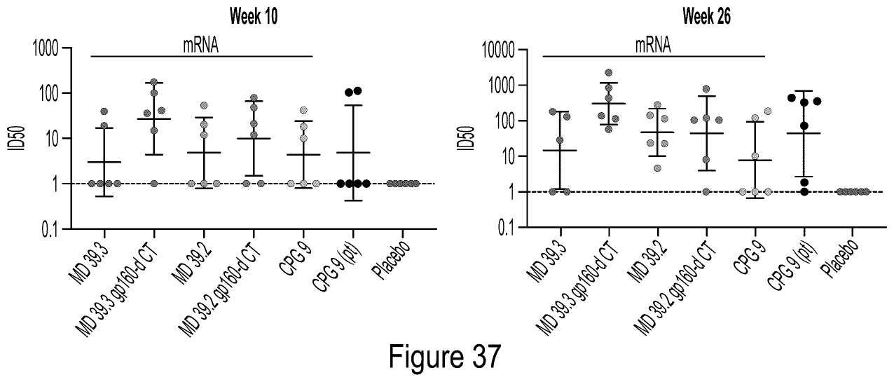

The conclusion that gp160-dCT performs as well or better than soluble trimer when both are mRNA-launched was reproduced for both MD39.2 and MD39.3 in Applicants' second rabbit experiment in the autologous neutralization data for serum IgG taken from week 10 (post two vaccinations) and week 26 (post three vaccinations) (FIG. 37).

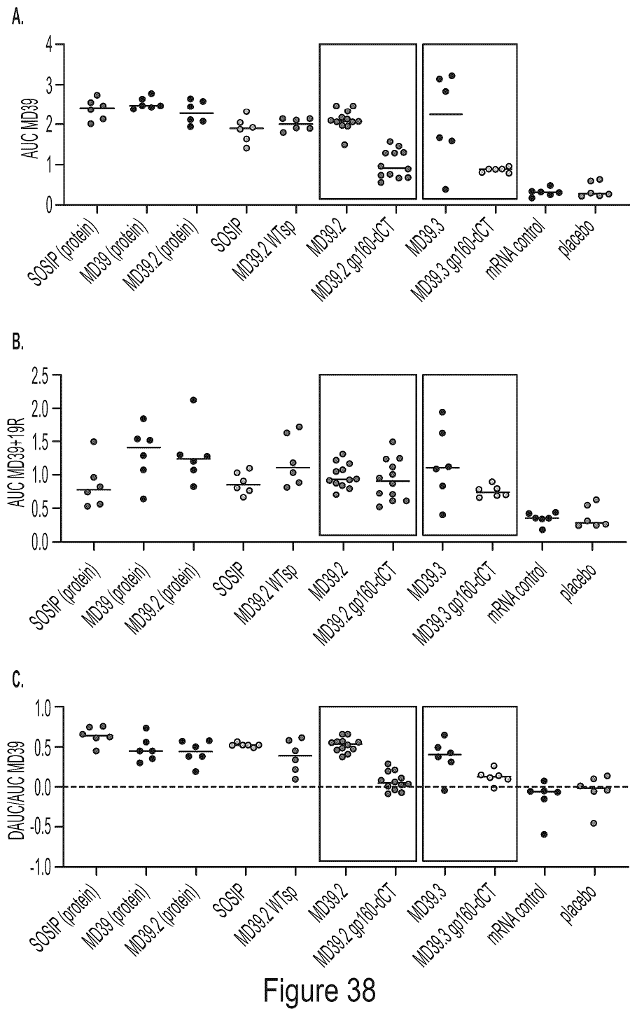

One hypothetical benefit of employing membrane-bound trimer immunogens would be a reduction of non-neutralizing bottom-directed responses and a potential increase of responses to other epitopes on the trimer. To investigate this, Applicants carried out ELISA analyses on the week 10 sera from rabbits in both experiments. Applicants characterized responses by the area under the curve (AUC) of the serum titration. Responses were measured against MD39 captured by PGT128 Fab, a protocol in which MD39 has a native antigenic profile with good binding to bnAbs including N332 bnAbs and reduced binding to non-nAbs directed to V3 or the CD4bs (not shown). By capturing MD39 with PGT128, the bottom of the trimer was well-exposed in the ELISA assay. All of the soluble trimer immunogens, whether protein or mRNA, elicited stronger responses to MD39 than the either of the membrane-bound gp160-dCT immunogens (FIG. 38A). To determine the degree of binding to epitopes other than the bottom, Applicants also measured responses to MD39 in the presence of a high concentration (20 μg/mL) of a bottom-directed antibody, 19R (also known as 1E6). In that competition assay, there was no significant difference in the responses to membrane-bound trimers compared to soluble trimers (FIG. 38B), indicating that membrane-bound trimers elicit comparable responses to the “top” of the trimer as soluble trimers. From these two assays, Applicants computed the fraction of the response to MD39 that was directed to the bottom (FIG. 38C). By this measure, approximately 50% of the response to any of the soluble trimers was directed to the bottom, whereas the membrane-bound trimers elicited a near zero anti-bottom response. Applicants conclude that mRNA-delivered, membrane-bound trimers can elicit significantly reduced anti-bottom responses while eliciting similar levels of responses to other trimer epitopes.

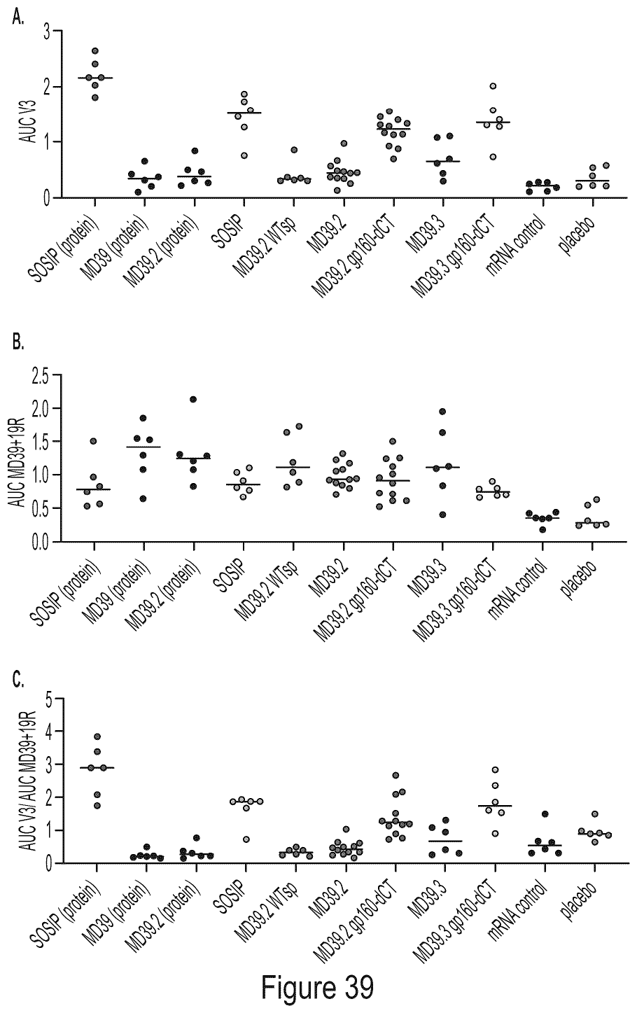

A potential disadvantage of mRNA-delivery is the inability to purify immunogens prior to in vivo exposure. If a trimer immunogen cannot fold and assemble with high fidelity in vivo without purification, one might expect to elicit both a lower level of autologous neutralizing responses compared to purified protein and a higher level of responses to epitopes like the V3 present on open trimers or incompletely assembled trimers. Applicants have already provided evidence in FIGS. 36 and 37 that mRNA-delivered gp160-dCT trimers can elicit autologous responses at comparable levels to purified proteins. To investigate the elicitation of V3 responses, Applicants carried out a V3 ELISA assay using a BG505 V3 peptide (FIG. 39A). In this assay, the MD39.2 soluble trimer showed low levels of V3 responses whether the immunogen was protein or mRNA. These V3 responses were also lower than the V3 response to BG505 SOSIP (whether protein or mRNA), consistent with the fact that MD39 exhibits substantially lower binding to V3 monoclonals in vitro (Steichen et al. Immunity 2016). These findings indicated that the lack of immunogen purification in mRNA delivery did not cause a significant degradation of immune responses for soluble trimer immunogens. The membrane-bound trimers did elicit a higher V3 response compared to their matched mRNA-delivered soluble trimer counterparts MD39.2 and MD39.3, and this may reflect the increased complexity of folding and assembly of a membrane-bound trimer. However, the V3 responses elicited by the membrane-bound trimers remained lower than the V3 responses elicited by BG505 SOSIP protein, indicating that the degree of improperly assembled membrane-bound trimer remained relatively low. As responses to the V3 peptide will vary due to both V3 exposure and overall immunogenicity, Applicants computed the ratio of the V3 response to the MD39 response apart from the bottom (FIG. 39B-C). The findings by this measure track with the findings from the V3 AUC: (i) mRNA-launched soluble trimers elicited similarly low levels of V3 responses as purified protein trimers; and (ii) membrane-bound trimers elicited elevated levels of V3 responses compared to matched soluble trimers; but (iii) the V3 responses induced by membrane-bound MD39.2 and MD39.3 trimers remained lower than the V3 response elicited by BG505 SOSIP purified protein.

Improvement of cell surface expression. Applicants' rabbit experiments were carried out with a gp160-dCT design that inadvertently included a known endocytosis motif (GY XX Ø) at the C-terminus. Applicants have since modified the design to eliminate that motif, and Applicants refer to their new construct as a gp151 design. The new gp151 design exhibited approximately 8-fold higher cell surface expression compared to the original gp160-dCT design, as measured by in vitro transfection and cytometry (FIG. 40A). The new gp151 also showed a favorable antigenic profile (FIG. 40B). Applicants propose to manufacture membrane-bound trimers based on the new gp151 format, as Applicants expect the higher cell surface expression combined with favorable antigenicity to lead to stronger favorable immune responses.

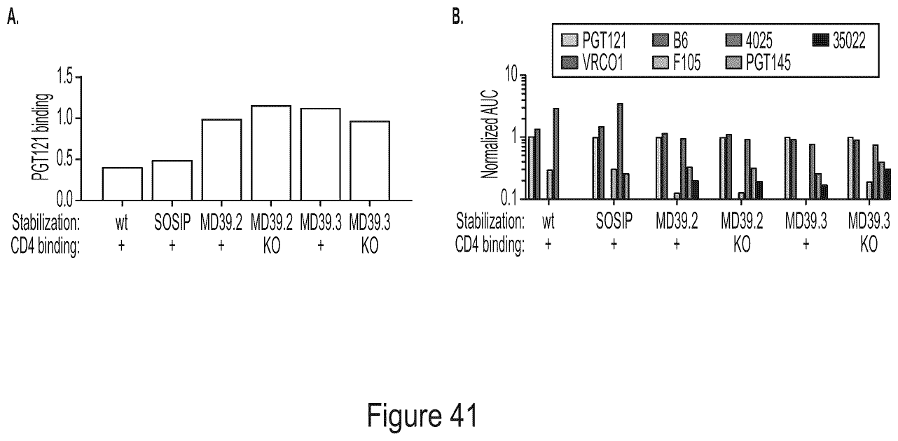

Applicants have assessed cell surface expression and antigenic profiles for two MD39.3 gp151 constructs (FIG. 41). In this case the test was done by using a Gag-VLP ELISA, which is significantly less time-consuming than cytometry. In the Gag-VLP ELISA, the indicated constructs were co-transfected with a modified variant of HIV Gag into freestyle 293F cells. Day 6 supernatants were clarified by centrifugation, filtered (0.45 uM), concentrated 100-fold using LentiX (Takara Bio), and finally coated directly onto high-binding ELISA plates. The results show that the BG505 MD39 constructs exhibit (i) ˜2-fold or better expression than BG505 wt or SOSIP (FIG. 41A); and (ii)>3-fold reduction of V3 responses as indicated by 4025 binding (FIG. 41B). The MD39.3 constructs show similar expression and antigenic profiles as the MD39.2 constructs, with slightly increased binding of PGT145, 35022, and F105 in MD39.3 KO compared to MD39.3.

NHP immunization testing. Applicants carried out an immunization experiment in rhesus macaques to evaluate responses to mRNA immunization with MD39.3 gp140, MD39.3 gp151, and MD39.3 CD4KO gp151, and to compare responses to protein vaccination with MD39.3 gp140 (Table 4). The essential findings in this NHP experiment were the same as in the rabbit experiments: the mRNA-delivered membrane-bound trimers (MD39.3 gp151 and MD39.3 CD4KO gp151) induced superior autologous neutralization and reduced base-directed responses compared to mRNA-delivered soluble trimer (MD39.3 gp140), and the mRNA-delivered membrane-bound trimers also induced similar autologous neutralization compared to purified protein soluble trimer (protein MD39.3 gp140) (FIGS. 42 and 43).

TABLE 2
Design of rabbit experiment #1.

| Group | n | antigen | Route | RNA/Protein | Dose level | | no. of doses | Dosing regimen | Adjuvant |
|---|---|---|---|---|---|---|---|---|---|
| 1 | 6 | BG505 MD39.2 PDGFR | IM | mRNA | 100 | μg | 3 | 0, 8, 24 wks | NA |
| 2 | 6 | BG505 MD39.2 gp160-dCT | IM | mRNA | 100 | μg | 3 | 0, 8, 24 wks | NA |
| 3 | 6 | BG505 MD39.2 WTsp | IM | mRNA | 100 | μg | 3 | 0, 8, 24 wks | NA |
| 4 | 6 | BG505 SOSIP D664 | IM | mRNA | 100 | μg | 3 | 0, 8, 24 wks | NA |
| 5 | 6 | BG505 MD39.2 | IM | mRNA | 100 | μg | 3 | 0, 8, 24 wks | NA |
| 6 | 6 | BG505 MD39.2 ferritin | IM | mRNA | 100 | μg | 3 | 0, 8, 24 wks | NA |
| 7 | 6 | mRNA control | IM | mRNA | 100 | μg | 3 | 0, 8, 24 wks | NA |
| 8 | 6 | BG505 SOSIP D664 | IM | Protein | 30 | μg | 3 | 0, 8, 24 wks | ISCOMIT |
| 9 | 6 | BG505 MD39.2 | IM | Protein | 30 | μg | 3 | 0, 8, 24 wks | ISCOMIT |
| 10 | 6 | BG505 gp120 foldon | IM | Protein | 30 | μg | 3 | 0, 8, 24 wks | ISCOMIT |
| 11 | 6 | BG505 MD39 | IM | Protein | 30 | μg | 3 | 0, 8, 24 wks | ISCOMIT |
| 12 | 6 | BG505 olio6 | IM | Protein | 30 | μg | 3 | 0, 8, 24 wks | ISCOMIT |
| 13 | 6 | BG505 MD39.2 ferritin | IM | Protein | 30 | μg | 3 | 0, 8, 24 wks | ISCOMIT |

TABLE 3
Design of rabbit experiment #2.

| Group | n | antigen | Route | RNA/Protein | Dose level | no. of doses | Dosing regimen | Adjuvant |
|---|---|---|---|---|---|---|---|---|
| 1 | 6 | BG505 MD39.3 | IM | mRNA | 100 μg | 3 | 0, 8, 24 wks | NA |
| 2 | 6 | BG505 MD39.3 gp160-dCT | IM | mRNA | 100 μg | 3 | 0, 8, 24 wks | NA |
| 3 | 6 | BG505 MD64 CPG9 | IM | mRNA | 100 μg | 3 | 0, 8, 24 wks | NA |
| 4 | 6 | BG505 MD39.2 gp160-dCT | IM | mRNA | 100 μg | 3 | 0, 8, 24 wks | NA |
| 5 | 6 | BG505 MD39.2 | IM | mRNA | 100 μg | 3 | 0, 8, 24 wks | NA |
| 6 | 6 | BG505 MD64 CPG9 | IM | protein | 30 μg | 3 | 0, 8, 24 wks | ISCOMIT |
| 7 | 6 | placebo | IM | NA | NA | 3 | 0, 8, 24 wks | NA |

FIG. 36. BG505-T332N autologous neutralization measured in a TZM-bl assay, for purified serum IgG from week 26 from rabbits immunized at weeks 0, 8, 24 with the indicated constructs in rabbit experiment #1.

FIG. 37. BG505-T332N autologous neutralization measured in a TZM-bl assay, for purified serum IgG from week 10 (left) or week 26 (right) from rabbits immunized at weeks 0, 8, 24 with the indicated constructs in rabbit experiment #2.

FIG. 38. ELISA analysis of serum antibody binding responses from week 10 (post two vaccinations) for rabbit experiments 1 and 2 combined. A: Response to MD39; B: Response to MD39 in presence of base-directed antibody 19R, to reveal responses to epitopes other than the base; C: Fraction of response to MD39 that is directed to the base. Immunogens were mRNA unless indicated otherwise. Boxes indicate data for matched pairs of mRNA-delivered soluble and membrane-bound trimers.

FIG. 39. ELISA analysis of anti-V3 serum antibody binding responses from week 10 (post two vaccinations) for rabbit experiments 1 and 2 combined. A: Response to V3 peptide; B: Response to MD39 in presence of base-directed IgG 19R, to reveal responses to epitopes other than the base; C: Ratio of V3 response to MD39 response other than base. Immunogens were mRNA unless indicated otherwise.

FIG. 40. Cell surface expression and antigenic profile for gp160-dCT and gp151

FIG. 41. Relative expression levels and antigenicity for gp151 constructs assessed by Gag-VLP ELISA. A: Relative Env expression levels as measured by PGT121 reactivity. B: Antigenic profiles normalized by PGT121 reactivity.

TABLE 4
Design of NHP experiment.

| | | | | RNA/ | Total Dose level | no. of | | |
|---|---|---|---|---|---|---|---|---|
| Group | n | antigen | Route | Protein | (including both sides) | doses | Dosing regimen | Adjuvant |
| 1 | 6 | BG505 MD39.3 | IM/Deltoid/bilateral | mRNA | 100 μg | 3 | 0, 8, 24 wks | NA |
| 2 | 6 | BG505 MD39.3 gp151 | IM/Deltoid/bilateral | mRNA | 100 μg | 3 | 0, 8, 24 wks | NA |
| 3 | 6 | BG505 MD39.3 CD4KO gp151 | IM/Deltoid/bilateral | mRNA | 100 μg | 3 | 0, 8, 24 wks | NA |
| 4 | 6 | BG505 MD39.3 gp151 | IM/Deltoid/bilateral | mRNA | 300 μg | 3 | 0, 8, 24 wks | NA |
| 5 | 6 | BG505 MD39.3 | IM/Deltoid/bilateral | protein | 100 μg | 3 | 0, 8, 24 wks | SMNP (750 μg |
| | | | | | | | | total dose) |

FIG. 42. BG505-T332N autologous neutralization measured in a TZM-bl assay, for purified serum IgG from week 10 (left) or week 26 (middle and right, with week 26 repeat at right) from rhesus macaques immunized at weeks 0, 8, 24 with the constructs shown in Table 4.

FIG. 43. ELISA analysis of NHP serum antibody binding responses from week 26 (post three vaccinations). A: MD39 AUC, the response to MD39. B: Delta AUC, the response to MD39 minus the response to MD39 in presence of base-directed antibody 19R. This reveals responses to the base. C: Fraction of response to MD39 that is directed to the base. D: Fraction of the MD39 response that is directed to epitopes other than the base.

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

1. A non-naturally occurring protein comprising the sequence of:
a) ApexGT5.2_link14:
(SEQ ID NO: 2)
AENLWVTVYYGVPVWKDAETTLFCASDAKAYETEKHNVWATHACVPTDPN
PQEIHLENVTEEFNMWKNNMVEQMHEDIISLWDQSLKPCVKLTPLCVTLQ
CTNVTNNITDDMRGELKNCSFNATTELRNKRQKVYSLFYRLDIVPMVDLW
TNYRLINCNTSAITQACPKVSFEPIPIHYCAPAGFAILKCKDKKFNGTGP
CPSVSTVQCTHGIKPVVSTQLLINGSLAEEEVIIRSENITNNAKNILVQL
NTPVQINCTRPNNNTVKSIRIGPGQAFYYTGDIIGDIRQAHCNVSKATWN
ETLGKVVKQLRKHFGNNTIIRFAQSSGGDLEVTTHSFNCGGEFFYCNTSG
LFNSTWISNTSVQGSNSTGSNDSITLPCRIKQIINMWQRIGQAMYAPPIQ
GVIRCVSNITGLILTRDGGSTNSTTETFRPGGGDMRDNWRSELYKYKVVK
IEPLGVAPTRCKRRVVGSHSGSGGSGSGGHAAVGIGAVSLGFLGAAGSTM
GAASMTLTVQARNLLSGIVQQQSNLLRAPEPQQHLLKDTHWGIKQLQARV
LAVEHYLRDQQLLGIWGCSGKLICCTNVPWNSSWSNRNLSEIWDNMTWLQ
WDKEISNYTQIIYGLLEESQNQQEKNEQDLLALD;
b) ApexGT6.2_link14:
(SEQ ID NO: 3)
AENLWVTVYYGVPVWKDAETTLFCASDAKAYETEKHNVWATHACVSTDPN
PQEIHLENVTEEFNMWKNNMVEQMHEDIISLWDQSLKPCVKLTPLCVGLQ
CTNVINNITDDMRGELKNCSFNATTELRNKRQKVYSLFYRLDIVPMVDLW
TNYRLISCNTSAITQACPKVSFEPIPIHYCAPAGFAILKCKDKKFNGTGP
CPSVSTVQCTHGIKPVVSTQLLINGSLAEEEVIIRSENITNNAKNILVQL
NTPVQINCTRPNNNTVKSIRIGPGQAFYYTGDIIGDIRQAHCNVSKATWN
ETLGKVVKQLRKHFGNNTIIRFAQSSGGDLEVTTHSFNCGGEFFYCNTSG
LENSTWISNTSVQGSNSTGSNDSITLPCRIKQIINMWQRIGQAMYAPPIQ
GVIRCVSNITGLILTRDGGSTNSTTETFRPGGGDMRDNWRSELYKYKVVK
IEPLGVAPTRCKRRVVGSHSGSGGSGSGGHAAVGIGAVSLGFLGAAGSTM
GAASMTLTVQARNLLSGIVQQQSNLLRAPEPQQHLLKDTHWGIKQLQARV
LAVEHYLRDQQLLGIWGCSGKLICCTNVPWNSSWSNRNLSEIWDNMTWLQ
WDKEISNYTQIIYGLLEESQNQQEKNEQDLLALD;
c) BG505.ApexGT2:
(SEQ ID NO: 4)
AENLWVTVYYGVPVWKDAETTLFCASDAKAYETEKHNVWATHACVPTDPN
PQEIHLENVTEEFNMWKNNMVEQMHEDIISLWDQSLKPCVKLTPLCVTLQ
CTNVTNNITDDMRGELKNCSFNATTELRNKRQKVYSLFYRLDIVPMGENS
TNYRLINCNTSAITQACPKVSFEPIPIHYCAPAGFAILKCKDKKFNGTGP
CPSVSTVQCTHGIKPVVSTQLLLNGSLAEEEVIIRSENITNNAKNILVQL
NTPVQINCTRPNNNTVKSIRIGPGQAFYYTGDIIGDIRQAHCNVSKATWN
ETLGKVVKQLRKHFGNNTIIRFAQSSGGDLEVTTHSFNCGGEFFYCNTSG
LENSTWISNTSVQGSNSTGSNDSITLPCRIKQIINMWQRIGQAMYAPPIQ
GVIRCVSNITGLILTRDGGSTNSTTETFRPGGGDMRDNWRSELYKYKVVK
IEPLGVAPTRCKRRVVGRRRRRRAVGIGAVSLGFLGAAGSTMGAASMTLT
VQARNLLSGIVQQQSNLLRAPEPQQHLLKDTHWGIKQLQARVLAVEHYLR
DQQLLGIWGCSGKLICCTNVPWNSSWSNRNLSEIWDNMTWLQWDKEISNY
TQIIYGLLEESQNQQEKNEQDLLALD;
d) BG505_ApexGT5.2_congly_CD4KO_gp151_link14:
(SEQ ID NO: 5)
AENLWVTVYYGVPVWKDAETTLFCASDAKAYETEKHNVWATHACVPTDPN
PQEIHLENVTEEFNMWKNNMVEQMHEDIISLWDQSLKPCVKLTPLCVTLQ
CTNVTNNITDDMRGELKNCSFNATTELRNKRQKVYSLFYRLDIVPMVDLW
TNYRLINCNTSAITQACPKVSFEPIPIHYCAPAGFAILKCKDKKFNGTGP
CQNVSTVQCTHGIKPVVSTQLLINGSLAEEEVIIRSENITNNAKNILVQL
NTSVQINCTRPNNNTVKSIRIGPGQAFYYTGDIIGDIRQAHCNVSKATWN
ETLGKVVKQLRKHFGNNTIIRFAQSSGGDLEVTTHSFNCGGEFFYCNTSG
LFNSTWISNTSVQGSNSTGSNDSITLPCRIKQIINMWQRIGQAMYAPPIQ
GVIRCVSNITGLILTRDGGSTNSTTETFRPGGTDMRDNWRSELYKYKVVK
IEPLGVAPTRCKRRVVGSHSGSGGSGSGGHAAVGIGAVSLGFLGAAGSTM
GAASMTLTVQARNLLSGIVQQQSNLLRAPEPQQHLLKDTHWGIKQLQARV
LAVEHYLRDQQLLGIWGCSGKLICCTNVPWNSSWSNRNLSEIWDNMTWLQ
WDKEISNYTQIIYGLLEESQNQQEKNEQDLLALDKWASLWNWFDISNWLW
YIKIFIMIVGGLIGLRIVFAVLSVIHRVR;
e) BG505_ApexGT5.2_congly_CD4KO_link14:
(SEQ ID NO: 6)
AENLWVTVYYGVPVWKDAETTLFCASDAKAYETEKHNVWATHACVPTDPN
PQEIHLENVTEEFNMWKNNMVEQMHEDIISLWDQSLKPCVKLTPLCVTLQ
CTNVTNNITDDMRGELKNCSFNATTELRNKRQKVYSLFYRLDIVPMVDLW
TNYRLINCNTSAITQACPKVSFEPIPIHYCAPAGFAILKCKDKKFNGTGP
CQNVSTVQCTHGIKPVVSTQLLINGSLAEEEVIIRSENITNNAKNILVQL
NTSVQINCTRPNNNTVKSIRIGPGQAFYYTGDIIGDIRQAHCNVSKATWN
ETLGKVVKQLRKHFGNNTIIRFAQSSGGDLEVTTHSFNCGGEFFYCNTSG
LFNSTWISNTSVQGSNSTGSNDSITLPCRIKQIINMWQRIGQAMYAPPIQ
GVIRCVSNITGLILTRDGGSTNSTTETFRPGGTDMRDNWRSELYKYKVVK
IEPLGVAPTRCKRRVVGSHSGSGGSGSGGHAAVGIGAVSLGFLGAAGSTM
GAASMTLTVQARNLLSGIVQQQSNLLRAPEPQQHLLKDTHWGIKQLQARV
LAVEHYLRDQQLLGIWGCSGKLICCTNVPWNSSWSNRNLSEIWDNMTWLQ
WDKEISNYTQIIYGLLEESQNQQEKNEQDLLALD;
f) BG505_ApexGT5.2_congly_gp151_link_14:
(SEQ ID NO: 7)
AENLWVTVYYGVPVWKDAETTLFCASDAKAYETEKHNVWATHACVPTDPN
PQEIHLENVTEEFNMWKNNMVEQMHEDIISLWDQSLKPCVKLTPLCVTLQ
CTNVTNNITDDMRGELKNCSFNATTELRNKRQKVYSLFYRLDIVPMVDLW
TNYRLINCNTSAITQACPKVSFEPIPIHYCAPAGFAILKCKDKKFNGTGP
CQNVSTVQCTHGIKPVVSTQLLINGSLAEEEVIIRSENITNNAKNILVQL
NTSVQINCTRPNNNTVKSIRIGPGQAFYYTGDIIGDIRQAHCNVSKATWN
ETLGKVVKQLRKHFGNNTIIRFAQSSGGDLEVTTHSFNCGGEFFYCNTSG
LFNSTWISNTSVQGSNSTGSNDSITLPCRIKQIINMWQRIGQAMYAPPIQ
GVIRCVSNITGLILTRDGGSTNSTTETFRPGGGDMRDNWRSELYKYKVVK
IEPLGVAPTRCKRRVVGSHSGSGGSGSGGHAAVGIGAVSLGFLGAAGSTM
GAASMTLTVQARNLLSGIVQQQSNLLRAPEPQQHLLKDTHWGIKQLQARV
LAVEHYLRDQQLLGIWGCSGKLICCTNVPWNSSWSNRNLSEIWDNMTWLQ
WDKEISNYTQIIYGLLEESQNQQEKNEQDLLALDKWASLWNWFDISNWLW
YIKIFIMIVGGLIGLRIVFAVLSVIHRVR;
g) BG505_ApexGT5.2_congly_link_14:
(SEQ ID NO: 8)
AENLWVTVYYGVPVWKDAETTLFCASDAKAYETEKHNVWATHACVPTDPN
PQEIHLENVTEEFNMWKNNMVEQMHEDIISLWDQSLKPCVKLTPLCVTLQ
CTNVTNNITDDMRGELKNCSFNATTELRNKRQKVYSLFYRLDIVPMVDLW
TNYRLINCNTSAITQACPKVSFEPIPIHYCAPAGFAILKCKDKKFNGTGP
CQNVSTVQCTHGIKPVVSTQLLINGSLAEEEVIIRSENITNNAKNILVQL
NTSVQINCTRPNNNTVKSIRIGPGQAFYYTGDIIGDIRQAHCNVSKATWN
ETLGKVVKQLRKHFGNNTIIRFAQSSGGDLEVTTHSFNCGGEFFYCNTSG
LENSTWISNTSVQGSNSTGSNDSITLPCRIKQIINMWQRIGQAMYAPPIQ
GVIRCVSNITGLILTRDGGSTNSTTETFRPGGGDMRDNWRSELYKYKVVK
IEPLGVAPTRCKRRVVGSHSGSGGSGSGGHAAVGIGAVSLGFLGAAGSTM
GAASMTLTVQARNLLSGIVQQQSNLLRAPEPQQHLLKDTHWGIKQLQARV
LAVEHYLRDQQLLGIWGCSGKLICCTNVPWNSSWSNRNLSEIWDNMTWLQ
WDKEISNYTQIIYGLLEESQNQQEKNEQDLLALD;
h) BG505_ApexGT5.2_gp151_link_14:
(SEQ ID NO: 9)
AENLWVTVYYGVPVWKDAETTLFCASDAKAYETEKHNVWATHACVPTDPN
PQEIHLENVTEEFNMWKNNMVEQMHEDIISLWDQSLKPCVKLTPLCVTLQ
CTNVTNNITDDMRGELKNCSFNATTELRNKRQKVYSLFYRLDIVPMVDLW
TNYRLINCNTSAITQACPKVSFEPIPIHYCAPAGFAILKCKDKKFNGTGP
CPSVSTVQCTHGIKPVVSTQLLINGSLAEEEVIIRSENITNNAKNILVQL
NTPVQINCTRPNNNTVKSIRIGPGQAFYYTGDIIGDIRQAHCNVSKATWN
ETLGKVVKQLRKHFGNNTIIRFAQSSGGDLEVTTHSFNCGGEFFYCNTSG
LFNSTWISNTSVQGSNSTGSNDSITLPCRIKQIINMWQRIGQAMYAPPIQ
GVIRCVSNITGLILTRDGGSTNSTTETFRPGGGDMRDNWRSELYKYKVVK
IEPLGVAPTRCKRRVVGSHSGSGGSGSGGHAAVGIGAVSLGFLGAAGSTM
GAASMTLTVQARNLLSGIVQQQSNLLRAPEPQQHLLKDTHWGIKQLQARV
LAVEHYLRDQQLLGIWGCSGKLICCTNVPWNSSWSNRNLSEIWDNMTWLQ
WDKEISNYTQIIYGLLEESQNQQEKNEQDLLALDKWASLWNWFDISNWLW
YIKIFIMIVGGLIGLRIVFAVLSVIHRVR;
i) BG505_ApexGT6.2_gp151_link14:
(SEQ ID NO: 10)
AENLWVTVYYGVPVWKDAETTLFCASDAKAYETEKHNVWATHACVSTDPN
PQEIHLENVTEEFNMWKNNMVEQMHEDIISLWDQSLKPCVKLTPLCVGLQ
CTNVINNITDDMRGELKNCSFNATTELRNKRQKVYSLFYRLDIVPMVDLW
TNYRLISCNTSAITQACPKVSFEPIPIHYCAPAGFAILKCKDKKFNGTGP
CPSVSTVQCTHGIKPVVSTQLLLNGSLAEEEVIIRSENITNNAKNILVQL
NTPVQINCTRPNNNTVKSIRIGPGQAFYYTGDIIGDIRQAHCNVSKATWN
ETLGKVVKQLRKHFGNNTIIRFAQSSGGDLEVTTHSFNCGGEFFYCNTSG
LENSTWISNTSVQGSNSTGSNDSITLPCRIKQIINMWQRIGQAMYAPPIQ
GVIRCVSNITGLILTRDGGSTNSTTETFRPGGGDMRDNWRSELYKYKVVK
IEPLGVAPTRCKRRVVGSHSGSGGSGSGGHAAVGIGAVSLGFLGAAGSTM
GAASMTLTVQARNLLSGIVQQQSNLLRAPEPQQHLLKDTHWGIKQLQARV
LAVEHYLRDQQLLGIWGCSGKLICCTNVPWNSSWSNRNLSEIWDNMTWLQ
WDKEISNYTQIIYGLLEESQNQQEKNEQDLLALDKWASLWNWFDISNWLW
YIKIFIMIVGGLIGLRIVFAVLSVIHRVR;
j) BG505_SOSIP_D664_m:
(SEQ ID NO: 11)
AENLWVTVYYGVPVWKDAETTLFCASDAKAYETEKHNVWATHACVPTDPN
PQEIHLENVTEEFNMWKNNMVEQMHTDIISLWDQSLKPCVKLTPLCVTLQ
CTNVTNNITDDMRGELKNCSFNMTTELRDKKQKVYSLFYRLDVVQINENQ
GNRSNNSNKEYRLINCNTSAITQACPKVSFEPIPIHYCAPAGFAILKCKD
KKFNGTGPCPSVSTVQCTHGIKPVVSTQLLINGSLAEEEVMIRSENITNN
AKNILVQFNTPVQINCTRPNNNTRKSIRIGPGQAFYATGDIIGDIRQAHC
NVSKATWNETLGKVVKQLRKHFGNNTIIRFANSSGGDLEVTTHSFNCGGE
FFYCNTSGLFNSTWISNTSVQGSNSTGSNDSITLPCRIKQIINMWQRIGQ
AMYAPPIQGVIRCVSNITGLILTRDGGSTNSTTETFRPGGGDMRDNWRSE
LYKYKVVKIEPLGVAPTRCKRRVVGRRRRRRAVGIGAVFLGFLGAAGSTM
GAASMTLTVQARNLLSGIVQQQSNLLRAPEAQQHLLKLTVWGIKQLQARV
LAVERYLRDQQLLGIWGCSGKLICCTNVPWNSSWSNRNLSEIWDNMTWLQ
WDKEISNYTQIIYGLLEESQNQQEKNEQDLLALD;
k) BG505_MD39_p140-PDGFR_link14_m:
(SEQ ID NO: 12)
AENLWVTVYYGVPVWKDAETTLFCASDAKAYETEKHNVWATHACVPTDPN
PQEIHLENVTEEFNMWKNNMVEQMHEDIISLWDQSLKPCVKLTPLCVTLQ
CTNVTNNITDDMRGELKNCSFNMTTELRDKKQKVYSLFYRLDVVQINENQ
GNRSNNSNKEYRLINCNTSAITQACPKVSFEPIPIHYCAPAGFAILKCKD
KKFNGTGPCPSVSTVQCTHGIKPVVSTOLLLNGSLAEEEVIIRSENITNN
AKNILVQLNTPVQINCTRPNNNTVKSIRIGPGQAFYYTGDIIGDIRQAHC
NVSKATWNETLGKVVKQLRKHFGNNTIIRFAQSSGGDLEVTTHSFNCGGE
FFYCNTSGLFNSTWISNTSVQGSNSTGSNDSITLPCRIKQIINMWQRIGQ
AMYAPPIQGVIRCVSNITGLILTRDGGSTNSTTETFRPGGGDMRDNWRSE
LYKYKVVKIEPLGVAPTRCKRRVVGSHSGSGGSGSGGHAAVGIGAVSLGF
LGAAGSTMGAASMTLTVQARNLLSGIVQQQSNLLRAPEPQQHLLKDTHWG
IKQLQARVLAVEHYLRDQQLLGIWGCSGKLICCTNVPWNSSWSNRNLSEI
WDNMTWLQWDKEISNYTQIIYGLLEESQNQQEKNEQDLLALDGGGSGGSG
GSEQKLISEEDLGGSGGSGGSNAVGODTQEVIVVPHSLPFKVVVISAILA
LVVLTIISLIILIMLWQKKPR;
l) BG505_MD39_gp160-dCT_link14_congly (MD39.3 gp160-dCT);
(SEQ ID NO: 13)
AENLWVTVYYGVPVWKDAETTLFCASDAKAYETEKHNVWATHACVPTDPN
PQEIHLENVTEEFNMWKNNMVEQMHEDIISLWDQSLKPCVKLTPLCVTLQ
CTNVTNNITDDMRGELKNCSFNMTTELRDKKQKVYSLFYRLDVVQINENQ
GNRSNNSNKEYRLINCNTSAITQACPKVSFEPIPIHYCAPAGFAILKCKD
KKFNGTGPCQNVSTVQCTHGIKPVVSTQLLINGSLAEEEVIIRSENITNN
AKNILVQLNTSVQINCTRPNNNTVKSIRIGPGQAFYYTGDIIGDIRQAHC
NVSKATWNETLGKVVKQLRKHFGNNTIIRFAQSSGGDLEVTTHSFNCGGE
FFYCNTSGLFNSTWISNTSVQGSNSTGSNDSITLPCRIKQIINMWQRIGQ
AMYAPPIQGVIRCVSNITGLILTRDGGSTNSTTETFRPGGGDMRDNWRSE
LYKYKVVKIEPLGVAPTRCKRRVVGSHSGSGGSGSGGHAAVGIGAVSLGF
LGAAGSTMGAASMTLTVQARNLLSGIVQQQSNLLRAPEPQQHLLKDTHWG
IKQLQARVLAVEHYLRDQQLLGIWGCSGKLICCTNVPWNSSWSNRNLSEI
WDNMTWLQWDKEISNYTQIIYGLLEESQNQQEKNEQDLLALDKWASLWNW
FDISNWLWYIKIFIMIVGGLIGLRIVFAVLSVIHRVRQGYSPLS;
m) BG505_MD39_gp160-dCT_link14_m (MD39.2 gp160-dCT):
(SEQ ID NO: 14)
AENLWVTVYYGVPVWKDAETTLFCASDAKAYETEKHNVWATHACVPTDPN
PQEIHLENVTEEFNMWKNNMVEQMHEDIISLWDQSLKPCVKLTPLCVTLQ
CTNVTNNITDDMRGELKNCSFNMTTELRDKKQKVYSLFYRLDVVQINENQ
GNRSNNSNKEYRLINCNTSAITQACPKVSFEPIPIHYCAPAGFAILKCKD
KKFNGTGPCPSVSTVQCTHGIKPVVSTQLLLNGSLAEEEVIIRSENITNN
AKNILVQLNTPVQINCTRPNNNTVKSIRIGPGQAFYYTGDIIGDIRQAHC
NVSKATWNETLGKVVKQLRKHFGNNTIIRFAQSSGGDLEVTTHSFNCGGE
FFYCNTSGLFNSTWISNTSVQGSNSTGSNDSITLPCRIKQIINMWQRIGQ
AMYAPPIQGVIRCVSNITGLILTRDGGSTNSTTETFRPGGGDMRDNWRSE
LYKYKVVKIEPLGVAPTRCKRRVVGSHSGSGGSGSGGHAAVGIGAVSLGF
LGAAGSTMGAASMTLTVQARNLLSGIVQQQSNLLRAPEPQQHLLKDTHWG
IKQLQARVLAVEHYLRDQQLLGIWGCSGKLICCTNVPWNSSWSNRNLSEI
WDNMTWLQWDKEISNYTQIIYGLLEESQNQQEKNEQDLLALDKWASLWNW
FDISNWLWYIKIFIMIVGGLIGLRIVFAVLSVIHRVRQGYSPLS;
n) BG505_MD39_link_14_2JD6_m (MD39.2 ferritin):
(SEQ ID NO: 15)
AENLWVTVYYGVPVWKDAETTLFCASDAKAYETEKHNVWATHACVPTDPN
PQEIHLENVTEEFNMWKNNMVEQMHEDIISLWDQSLKPCVKLTPLCVTLQ
CTNVTNNITDDMRGELKNCSFNMTTELRDKKQKVYSLFYRLDVVQINENQ
GNRSNNSNKEYRLINCNTSAITQACPKVSFEPIPIHYCAPAGFAILKCKD
KKFNGTGPCPSVSTVQCTHGIKPVVSTQLLLNGSLAEEEVIIRSENITNN
AKNILVQLNTPVQINCTRPNNNTVKSIRIGPGQAFYYTGDIIGDIRQAHC
NVSKATWNETLGKVVKQLRKHFGNNTIIRFAQSSGGDLEVTTHSFNCGGE
FFYCNTSGLFNSTWISNTSVQGSNSTGSNDSITLPCRIKQIINMWQRIGQ
AMYAPPIQGVIRCVSNITGLILTRDGGSTNSTTETFRPGGGDMRDNWRSE
LYKYKVVKIEPLGVAPTRCKRRVVGSHSGSGGSGSGGHAAVGIGAVSLGF
LGAAGSTMGAASMTLTVQARNLLSGIVQQQSNLLRAPEPQQHLLKDTHWG
IKQLQARVLAVEHYLRDQQLLGIWGCSGKLICCTNVPWNSSWSNRNLSEI
WDNMTWLQWDKEISNYTQIIYGLLEESQNQQEKNEQDLLALDGSGGLSER
MLKALNDQLNRELYSAYLYFAMAAYFEDLGLEGFANWMKAQAEEEIGHAL
RFYNYIYDKNGRVELDEIPKPPKEWESPLKAFEAAYEHEKFISKSIYELA
ALAEEEKDYSTRAFLEWFINEQVEEEASVKKILDKLKFAKDSPQILFMLD
KELSARAPKLPGLLMQGGE;
o) BG505 MD39.3 (aka MD39_link14_conoly or BG505_SOSIP_MD39_link14_congly_m);
(SEQ ID NO: 16)
AENLWVTVYYGVPVWKDAETTLFCASDAKAYETEKHNVWATHACVPTDPN
PQEIHLENVTEEFNMWKNNMVEQMHEDIISLWDQSLKPCVKLTPLCVTLQ
CTNVTNNITDDMRGELKNCSFNMTTELRDKKQKVYSLFYRLDVVQINENQ
GNRSNNSNKEYRLINCNTSAITQACPKVSFEPIPIHYCAPAGFAILKCKD
KKFNGTGPCQNVSTVQCTHGIKPVVSTQLLINGSLAEEEVIIRSENITNN
AKNILVQLNTSVQINCTRPNNNTVKSIRIGPGQAFYYTGDIIGDIRQAHC
NVSKATWNETLGKVVKQLRKHFGNNTIIRFAQSSGGDLEVTTHSFNCGGE
FFYCNTSGLFNSTWISNTSVQGSNSTGSNDSITLPCRIKQIINMWQRIGQ
AMYAPPIQGVIRCVSNITGLILTRDGGSTNSTTETFRPGGGDMRDNWRSE
LYKYKVVKIEPLGVAPTRCKRRVVGSHSGSGGSGSGGHAAVGIGAVSLGF
LGAAGSTMGAASMTLTVQARNLLSGIVQQQSNLLRAPEPQQHLLKDTHWG
IKQLQARVLAVEHYLRDQQLLGIWGCSGKLICCTNVPWNSSWSNRNLSEI
WDNMTWLQWDKEISNYTQIIYGLLEESQNQQEKNEQDLLALD;
p) BG505 MD39.3 CD4KO gp151 (aka BG505 MD39.3 CD4KO gn151)
(SEQ ID NO: 17)
AENLWVTVYYGVPVWKDAETTLFCASDAKAYETEKHNVWATHACVPTDPN
PQEIHLENVTEEFNMWKNNMVEQMHEDIISLWDQSLKPCVKLTPLCVTLQ
CTNVTNNITDDMRGELKNCSFNMTTELRDKKQKVYSLFYRLDVVQINENQ
GNRSNNSNKEYRLINCNTSAITQACPKVSFEPIPIHYCAPAGFAILKCKD
KKFNGTGPCQNVSTVQCTHGIKPVVSTQLLLNGSLAEEEVIIRSENITNN
AKNILVQLNTSVQINCTRPNNNTVKSIRIGPGQAFYYTGDIIGDIRQAHC
NVSKATWNETLGKVVKQLRKHFGNNTIIRFAQSSGGDLEVTTHSFNCGGE
FFYCNTSGLFNSTWISNTSVQGSNSTGSNDSITLPCRIKQIINMWQRIGQ
AMYAPPIQGVIRCVSNITGLILTRDGGSTNSTTETFRPGGTDMRDNWRSE
LYKYKVVKIEPLGVAPTRCKRRVVGSHSGSGGSGSGGHAAVGIGAVSLGF
LGAAGSTMGAASMTLTVQARNLLSGIVQQQSNLLRAPEPQQHLLKDTHWG
IKQLQARVLAVEHYLRDQQLLGIWGCSGKLICCTNVPWNSSWSNRNLSEI
WDNMTWLQWDKEISNYTQIIYGLLEESQNQQEKNEQDLLALDKWASLWNW
FDISNWLWYIKIFIMIVGGLIGLRIVFAVLSVIHRVR;
q) BG505 MD39.3 gn151 (aka BG505_MD39_gn151_link14_conoly);
(SEQ ID NO: 18)
AENLWVTVYYGVPVWKDAETTLFCASDAKAYETEKHNVWATHACVPTDPN
PQEIHLENVTEEFNMWKNNMVEQMHEDIISLWDQSLKPCVKLTPLCVTLQ
CTNVTNNITDDMRGELKNCSFNMTTELRDKKQKVYSLFYRLDVVQINENQ
GNRSNNSNKEYRLINCNTSAITQACPKVSFEPIPIHYCAPAGFAILKCKD
KKFNGTGPCQNVSTVQCTHGIKPVVSTQLLLNGSLAEEEVIIRSENITNN
AKNILVQLNTSVQINCTRPNNNTVKSIRIGPGQAFYYTGDIIGDIRQAHC
NVSKATWNETLGKVVKQLRKHFGNNTIIRFAQSSGGDLEVTTHSFNCGGE
FFYCNTSGLFNSTWISNTSVQGSNSTGSNDSITLPCRIKQIINMWQRIGQ
AMYAPPIQGVIRCVSNITGLILTRDGGSTNSTTETFRPGGGDMRDNWRSE
LYKYKVVKIEPLGVAPTRCKRRVVGSHSGSGGSGSGGHAAVGIGAVSLGF
LGAAGSTMGAASMTLTVQARNLLSGIVQQQSNLLRAPEPQQHLLKDTHWG
IKQLQARVLAVEHYLRDQQLLGIWGCSGKLICCTNVPWNSSWSNRNLSEI
WDNMTWLQWDKEISNYTQIIYGLLEESQNQQEKNEQDLLALDKWASLWNW
FDISNWLWYIKIFIMIVGGLIGLRIVFAVLSVIHRVR;
r) BG505_SOSIP_MD39_link_14_m (MD39.2);
(SEQ ID NO: 19)
AENLWVTVYYGVPVWKDAETTLFCASDAKAYETEKHNVWATHACVPTDPN
PQEIHLENVTEEFNMWKNNMVEQMHEDIISLWDQSLKPCVKLTPLCVTLQ
CTNVTNNITDDMRGELKNCSFNMTTELRDKKQKVYSLFYRLDVVQINENQ
GNRSNNSNKEYRLINCNTSAITQACPKVSFEPIPIHYCAPAGFAILKCKD
KKFNGTGPCPSVSTVQCTHGIKPVVSTQLLLNGSLAEEEVIIRSENITNN
AKNILVQLNTPVQINCTRPNNNTVKSIRIGPGQAFYYTGDIIGDIRQAHC
NVSKATWNETLGKVVKQLRKHFGNNTIIRFAQSSGGDLEVTTHSFNCGGE
FFYCNTSGLFNSTWISNTSVQGSNSTGSNDSITLPCRIKQIINMWQRIGQ
AMYAPPIQGVIRCVSNITGLILTRDGGSTNSTTETFRPGGGDMRDNWRSE
LYKYKVVKIEPLGVAPTRCKRRVVGSHSGSGGSGSGGHAAVGIGAVSLGF
LGAAGSTMGAASMTLTVQARNLLSGIVQQQSNLLRAPEPQQHLLKDTHWG
IKQLQARVLAVEHYLRDQQLLGIWGCSGKLICCTNVPWNSSWSNRNLSEI
WDNMTWLQWDKEISNYTQIIYGLLEESQNQQEKNEQDLLALD;
s) BG505.ApexGT2.Gmax:
(SEQ ID NO: 20)
AENLWVTVYYGVPVWKDAETTLFCASDAKAYETEKHNVWATHACVPTDPN
PQEIHLENVTEEFNMWKNNMVEQMHEDIISLWDQSLKPCVKLTPLCVTLN
CTNVTNNITDDMRGELKNCSFNATTELRNKRQKVYSLFYRLDIVPMGENS
TNYRLINCNTSAITQACPKVSFEPIPIHYCAPAGFAILKCKDKKFNGTGP
CQNVSTVQCTHGIKPVVSTQLLLNGSLAEEEVIIRSENITNNAKNILVQL
NTSVQINCTRPNNNTVKSIRIGPGQAFYYTGDIIGDIRQAHCNVSKATWN
ETLGKVVKQLRKHFGNNTIIRFAQSSGGDLEVTTHSFNCGGEFFYCNTSG
LENSTWISNTSVQGSNSTGSNDSITLPCRIKQIINMWQRIGQAMYAPPIQ
GVIRCVSNITGLILTRDGGSTNSTTETFRPGGGDMRDNWRSELYKYKVVK
IEPLGVAPTRCKRRVVGRRRRRRAVGIGAVSLGFLGAAGSTMGAASMTLT
VQARNLLSGIVQQQSNLLRAPEPQQHLLKDTHWGIKQLQARVLAVEHYLR
DQQLLGIWGCSGKLICCTNVPWNSSWSNRNLSEIWDNMTWLQWDKEISNY
TQIIYGLLEESQNQQEKNEQDLLALD;
t) BG505.ApexGT2.2MUT:
(SEQ ID NO: 21)
AENLWVTVYYGVPVWKDAETTLFCASDAKAYETEKHNVWATHACVPTDPN
PQEIHLENVTEEFNMWKNNMVEQMHEDIISLWDQSLKPCVKLTPLCVTLQ
CTNVTNNITDDMRGELKNCSFNATTELRNKRQKVYSLFYRLDIVPMGENS
ANYRLIDCNTSAITQACPKVSFEPIPIHYCAPAGFAILKCKDKKFNGTGP
CPSVSTVQCTHGIKPVVSTQLLINGSLAEEEVIIRSENITNNAKNILVQL
NTPVQINCTRPNNNTVKSIRIGPGQAFYYTGDIIGDIRQAHCNVSKATWN
ETLGKVVKQLRKHFGNNTIIRFAQSSGGDLEVTTHSFNCGGEFFYCNTSG
LFNSTWISNTSVQGSNSTGSNDSITLPCRIKQIINMWQRIGQAMYAPPIQ
GVIRCVSNITGLILTRDGGSTNSTTETFRPGGGDMRDNWRSELYKYKVVK
IEPLGVAPTRCKRRVVGRRRRRRAVGIGAVSLGFLGAAGSTMGAASMTLT
VQARNLLSGIVQQQSNLLRAPEPQQHLLKDTHWGIKQLQARVLAVEHYLR
DQQLLGIWGCSGKLICCTNVPWNSSWSNRNLSEIWDNMTWLQWDKEISNY
TQIIYGLLEESQNQQEKNEQDLLALD;
u) BG505.ApexGT3A:
(SEQ ID NO: 22)
AENLWVTVYYGVPVWKDAETTLFCASDAKAYETEKHNVWATHACVPTDPN
PQEIHLENVTEEFNMWKNNMVEQMHEDIISLWDQSLKPCVKLTPLCVTLQ
CTNVTNNITDDMRGELKNCSFNATTELRNKRVKRYSLFYRLDIVQIDSNR
AKSHYRLINCNTSAITQACPKVSFEPIPIHYCAPAGFAILKCKDKKFNGT
GPCPSVSTVQCTHGIKPVVSTQLLINGSLAEEEVIIRSENITNNAKNILV
QLNTPVQINCTRPNNNTVKSIRIGPGQAFYYTGDIIGDIRQAHCNVSKAT
WNETLGKVVKQLRKHFGNNTIIRFAQSSGGDLEVTTHSFNCGGEFFYCNT
SGLENSTWISNTSVQGSNSTGSNDSITLPCRIKQIINMWQRIGQAMYAPP
IQGVIRCVSNITGLILTRDGGSTNSTTETFRPGGGDMRDNWRSELYKYKV
VKIEPLGVAPTRCKRRVVGRRRRRRAVGIGAVSLGFLGAAGSTMGAASMT
LTVQARNLLSGIVQQQSNLLRAPEPQQHLLKDTHWGIKQLQARVLAVEHY
LRDQQLLGIWGCSGKLICCTNVPWNSSWSNRNLSEIWDNMTWLQWDKEIS
NYTQIIYGLLEESQNQQEKNEQDLLALD;
v) BG505.ApexGT3A.N130:
(SEQ ID NO: 23)
AENLWVTVYYGVPVWKDAETTLFCASDAKAYETEKHNVWATHACVPTDPN
PQEIHLENVTEEFNMWKNNMVEQMHEDIISLWDQSLKPCVKLTPLCVTLN
CTNVTNNITDDMRGELKNCSFNATTELRNKRVKRYSLFYRLDIVQIDSNR
TKSHYRLINCNTSAITQACPKVSFEPIPIHYCAPAGFAILKCKDKKFNGT
GPCPSVSTVQCTHGIKPVVSTQLLINGSLAEEEVIIRSENITNNAKNILV
QLNTPVQINCTRPNNNTVKSIRIGPGQAFYYTGDIIGDIRQAHCNVSKAT
WNETLGKVVKQLRKHFGNNTIIRFAQSSGGDLEVTTHSFNCGGEFFYCNT
SGLFNSTWISNTSVQGSNSTGSNDSITLPCRIKQIINMWQRIGQAMYAPP
IQGVIRCVSNITGLILTRDGGSTNSTTETFRPGGGDMRDNWRSELYKYKV
VKIEPLGVAPTRCKRRVVGRRRRRRAVGIGAVSLGFLGAAGSTMGAASMT
LTVQARNLLSGIVQQQSNLLRAPEPQQHLLKDTHWGIKQLQARVLAVEHY
LRDQQLLGIWGCSGKLICCTNVPWNSSWSNRNLSEIWDNMTWLQWDKEIS
NYTQIIYGLLEESQNQQEKNEQDLLALD;
w) BG505.ApexGT3A.Gmax:
(SEQ ID NO: 24)
AENLWVTVYYGVPVWKDAETTLFCASDAKAYETEKHNVWATHACVPTDPN
PQEIHLENVTEEFNMWKNNMVEQMHEDIISLWDQSLKPCVKLTPLCVTLN
CTNVTNNITDDMRGELKNCSFNATTELRNKRVKRYSLFYRLDIVQIDSNR
TKSHYRLINCNTSAITQACPKVSFEPIPIHYCAPAGFAILKCKDKKFNGT
GPCQNVSTVQCTHGIKPVVSTQLLINGSLAEEEVIIRSENITNNAKNILV
QLNTSVQINCTRPNNNTVKSIRIGPGQAFYYTGDIIGDIRQAHCNVSKAT
WNETLGKVVKQLRKHFGNNTIIRFAQSSGGDLEVTTHSFNCGGEFFYCNT
SGLENSTWISNTSVQGSNSTGSNDSITLPCRIKQIINMWQRIGQAMYAPP
IQGVIRCVSNITGLILTRDGGSTNSTTETFRPGGGDMRDNWRSELYKYKV
VKIEPLGVAPTRCKRRVVGRRRRRRAVGIGAVSLGFLGAAGSTMGAASMT
LTVQARNLLSGIVQQQSNLLRAPEPQQHLLKDTHWGIKQLQARVLAVEHY
LRDQQLLGIWGCSGKLICCTNVPWNSSWSNRNLSEIWDNMTWLQWDKEIS
NYTQIIYGLLEESQNQQEKNEQDLLALD;
x) BG505.ApexGT5:
(SEQ ID NO: 25)
AENLWVTVYYGVPVWKDAETTLFCASDAKAYETEKHNVWATHACVPTDPN
PQEIHLENVTEEFNMWKNNMVEQMHEDIISLWDQSLKPCVKLTPLCVTLQ
CTNVTNNITDDMRGELKNCSFNATTELRNKRQKVYSLFYRLDIVPMVDLW
TNYRLINCNTSAITQACPKVSFEPIPIHYCAPAGFAILKCKDKKFNGTGP
CPSVSTVQCTHGIKPVVSTQLLINGSLAEEEVIIRSENITNNAKNILVQL
NTPVQINCTRPNNNTVKSIRIGPGQAFYYTGDIIGDIRQAHCNVSKATWN
ETLGKVVKQLRKHFGNNTIIRFAQSSGGDLEVTTHSFNCGGEFFYCNTSG
LENSTWISNTSVQGSNSTGSNDSITLPCRIKQIINMWQRIGQAMYAPPIQ
GVIRCVSNITGLILTRDGGSTNSTTETFRPGGGDMRDNWRSELYKYKVVK
IEPLGVAPTRCKRRVVGRRRRRRAVGIGAVSLGFLGAAGSTMGAASMTLT
VQARNLLSGIVQQQSNLLRAPEPQQHLLKDTHWGIKQLQARVLAVEHYLR
DQQLLGIWGCSGKLICCTNVPWNSSWSNRNLSEIWDNMTWLQWDKEISNY
TQIIYGLLEESQNQQEKNEQDLLALDGTKHHHHHH;
y) BG505.ApexGT5.Gmax:
(SEQ ID NO: 26)
AENLWVTVYYGVPVWKDAETTLFCASDAKAYETEKHNVWATHACVPTDPN
PQEIHLENVTEEFNMWKNNMVEQMHEDIISLWDQSLKPCVKLTPLCVTLN
CTNVTNNITDDMRGELKNCSFNATTELRNKRQKVYSLFYRLDIVPMVDLW
TNYRLINCNTSAITQACPKVSFEPIPIHYCAPAGFAILKCKDKKFNGTGP
CQNVSTVQCTHGIKPVVSTQLLINGSLAEEEVIIRSENITNNAKNILVQL
NTSVQINCTRPNNNTVKSIRIGPGQAFYYTGDIIGDIRQAHCNVSKATWN
ETLGKVVKQLRKHFGNNTIIRFAQSSGGDLEVTTHSFNCGGEFFYCNTSG
LFNSTWISNTSVQGSNSTGSNDSITLPCRIKQIINMWQRIGQAMYAPPIQ
GVIRCVSNITGLILTRDGGSTNSTTETFRPGGGDMRDNWRSELYKYKVVK
IEPLGVAPTRCKRRVVGRRRRRRAVGIGAVSLGFLGAAGSTMGAASMTLT
VQARNLLSGIVQQQSNLLRAPEPQQHLLKDTHWGIKQLQARVLAVEHYLR
DQQLLGIWGCSGKLICCTNVPWNSSWSNRNLSEIWDNMTWLQWDKEISNY
TQIIYGLLEESQNQQEKNEQDLLALDGT;
z) MID64_CPG9 (aka BG505_SOSIP_MD39C_CPG9 m): or
(SEQ ID NO: 27)
GGNSSGSLGFLGAAGSTMGAASMTLTVQARNLLSGIVQQQSNLLRAPEPQ
QHLLKLTVWGIKQLQARVLAVEHYLRDQQLLGIWGCSGKLICCTNVPWNS
SWSNRNLSEIWDNMTWLNWSKEISNYTQIIYGLLEESQNQNESNEQDLGG
NGSGGGSGSGGNGSSGLWVTVYYGVPVWKDAETTLFCASDAKAYETEKHN
VWATHECVPTDPNSSEIHLENVTEEFNMWKNNMVEQMHTDIIELWDQSLK
PCVKLTPLCVTLQCTNVTNNITDDMRGELKNCSFNMTTELRDKKQKVYSL
FYRLDVVQINENQGNRSNNSNKEYRLINCNTSAITQACPKVSFEPIPIHY
CAPAGFAILKCKDKKFNGTGPCQNVSTVQCTHGIKPVVSTQLLLNGSLAE
EEVIIRSENITNNAKNILVQLNTSVQINCTRPNNNTVKSIRIGPGQAFYY
TGDIIGDIRQAHCNVSKATWNETLGKVVKQLRKHFGNNTIIRFAQSSGGD
LEVTTHSFNCGGEFFYCNTSGLFNSTWISNTSVQGSNSTGSNDSITLPCR
IKQIINMWQRIGQAMYAPPIQGVIRCVSNITGLILTRDGGSTNSTTETFR
PGGGDMRDNWRSELYKYKVVKIEPLGVAPTRCNRS

2. A protein having at least 90% homology or identity with the sequence of protein of claim 1.

3. A protein having at least 95% homology or identity with the sequence of protein of claim 1.

4. A multimeric protein comprising the protein of claim 1.

5. The protein of claim 1 further comprising an additional cysteine.

6. The protein of claim 4 fused to a multimerization motif.

7. A nucleic acid encoding the protein of claim 1.

8. A nucleic acid having at least 90% homology or identity with the sequence of the nucleic acid of claim 7.

9. A nucleic acid having at least 95% homology or identity with the sequence of the nucleic acid of claim 7.

10. The nucleic acid of claim 8 wherein the nucleic acid is formulated in a lipid nanoparticle (LNP).

11. A method for eliciting an immune response comprising systemically administering to an animal in need thereof an effective amount of the protein of claim 1.

12. The method of claim 11, wherein the animal is a mammal.

13. The method of claim 12, wherein the mammal is a human.

14. A method for eliciting an immune response comprising systemically administering to an animal in need thereof an effective amount of the nucleic acid of claim 8.

15. The method of claim 14, wherein the animal is a mammal.

16. The method of claim 15, wherein the mammal is a human.

17. The method of claim 11, comprising administration of an additional booster dose.

18. The method of claim 17, wherein the booster dose is from a HIV pseudo virus (PSV).


SEQUENCE LISTING (237 sequences)

SEQ ID NO: 1 - protein, 31 aa, synthetic construct
MGILPSPGMPALLSLVSLLSVLLMGCVAETG

SEQ ID NO: 2 - protein, 634 aa, synthetic construct
AENLWVTVYYGVPVWKDAETTLFCASDAKAYETEKHNVWATHACVPTDPNPQEIHLENVTEEFNMWKNNMVEQMHEDIISLWDQSLKPCVKLTPLCVTLQCTNVTNNITDDMRGELKNCSFNATTELRNKRQKVYSLFYRLDIVPMVDLWTNYRLINCNTSAITQACPKVSFEPIPIHYCAPAGFAILKCKDKKFNGTGPCPSVSTVQCTHGIKPVVSTQLLLNGSLAEEEVIIRSENITNNAKNILVQLNTPVQINCTRPNNNTVKSIRIGPGQAFYYTGDIIGDIRQAHCNVSKATWNETLGKVVKQLRKHFGNNTIIRFAQSSGGDLEVTTHSFNCGGEFFYCNTSGLFNSTWISNTSVQGSNSTGSNDSITLPCRIKQIINMWQRIGQAMYAPPIQGVIRCVSNITGLILTRDGGSTNSTTETFRPGGGDMRDNWRSELYKYKVVKIEPLGVAPTRCKRRVVGSHSGSGGSGSGGHAAVGIGAVSLGFLGAAGSTMGAASMTLTVQARNLLSGIVQQQSNLLRAPEPQQHLLKDTHWGIKQLQARVLAVEHYLRDQQLLGIWGCSGKLICCTNVPWNSSWSNRNLSEIWDNMTWLQWDKEISNYTQIIYGLLEESQNQQEKNEQDLLALD

SEQ ID NO: 3 - protein, 634 aa, synthetic construct
AENLWVTVYYGVPVWKDAETTLFCASDAKAYETEKHNVWATHACVSTDPNPQEIHLENVTEEFNMWKNNMVEQMHEDIISLWDQSLKPCVKLTPLCVGLQCTNVTNNITDDMRGELKNCSFNATTELRNKRQKVYSLFYRLDIVPMVDLWTNYRLISCNTSAITQACPKVSFEPIPIHYCAPAGFAILKCKDKKFNGTGPCPSVSTVQCTHGIKPVVSTQLLLNGSLAEEEVIIRSENITNNAKNILVQLNTPVQINCTRPNNNTVKSIRIGPGQAFYYTGDIIGDIRQAHCNVSKATWNETLGKVVKQLRKHFGNNTIIRFAQSSGGDLEVTTHSFNCGGEFFYCNTSGLFNSTWISNTSVQGSNSTGSNDSITLPCRIKQIINMWQRIGQAMYAPPIQGVIRCVSNITGLILTRDGGSTNSTTETFRPGGGDMRDNWRSELYKYKVVKIEPLGVAPTRCKRRVVGSHSGSGGSGSGGHAAVGIGAVSLGFLGAAGSTMGAASMTLTVQARNLLSGIVQQQSNLLRAPEPQQHLLKDTHWGIKQLQARVLAVEHYLRDQQLLGIWGCSGKLICCTNVPWNSSWSNRNLSEIWDNMTWLQWDKEISNYTQIIYGLLEESQNQQEKNEQDLLALD

SEQ ID NO: 4 - protein, 626 aa, synthetic construct
AENLWVTVYYGVPVWKDAETTLFCASDAKAYETEKHNVWATHACVPTDPNPQEIHLENVTEEFNMWKNNMVEQMHEDIISLWDQSLKPCVKLTPLCVTLQCTNVTNNITDDMRGELKNCSFNATTELRNKRQKVYSLFYRLDIVPMGENSTNYRLINCNTSAITQACPKVSFEPIPIHYCAPAGFAILKCKDKKFNGTGPCPSVSTVQCTHGIKPVVSTQLLLNGSLAEEEVIIRSENITNNAKNILVQLNTPVQINCTRPNNNTVKSIRIGPGQAFYYTGDIIGDIRQAHCNVSKATWNETLGKVVKQLRKHFGNNTIIRFAQSSGGDLEVTTHSFNCGGEFFYCNTSGLFNSTWISNTSVQGSNSTGSNDSITLPCRIKQIINMWQRIGQAMYAPPIQGVIRCVSNITGLILTRDGGSTNSTTETFRPGGGDMRDNWRSELYKYKVVKIEPLGVAPTRCKRRVVGRRRRRRAVGIGAVSLGFLGAAGSTMGAASMTLTVQARNLLSGIVQQQSNLLRAPEPQQHLLKDTHWGIKQLQARVLAVEHYLRDQQLLGIWGCSGKLICCTNVPWNSSWSNRNLSEIWDNMTWLQWDKEISNYTQIIYGLLEESQNQQEKNEQDLLALD

SEQ ID NO: 5 - protein, 679 aa, synthetic construct
AENLWVTVYYGVPVWKDAETTLFCASDAKAYETEKHNVWATHACVPTDPNPQEIHLENVTEEFNMWKNNMVEQMHEDIISLWDQSLKPCVKLTPLCVTLQCTNVTNNITDDMRGELKNCSFNATTELRNKRQKVYSLFYRLDIVPMVDLWTNYRLINCNTSAITQACPKVSFEPIPIHYCAPAGFAILKCKDKKFNGTGPCQNVSTVQCTHGIKPVVSTQLLLNGSLAEEEVIIRSENITNNAKNILVQLNTSVQINCTRPNNNTVKSIRIGPGQAFYYTGDIIGDIRQAHCNVSKATWNETLGKVVKQLRKHFGNNTIIRFAQSSGGDLEVTTHSFNCGGEFFYCNTSGLFNSTWISNTSVQGSNSTGSNDSITLPCRIKQIINMWQRIGQAMYAPPIQGVIRCVSNITGLILTRDGGSTNSTTETFRPGGTDMRDNWRSELYKYKVVKIEPLGVAPTRCKRRVVGSHSGSGGSGSGGHAAVGIGAVSLGFLGAAGSTMGAASMTLTVQARNLLSGIVQQQSNLLRAPEPQQHLLKDTHWGIKQLQARVLAVEHYLRDQQLLGIWGCSGKLICCTNVPWNSSWSNRNLSEIWDNMTWLQWDKEISNYTQIIYGLLEESQNQQEKNEQDLLALDKWASLWNWFDISNWLWYIKIFIMIVGGLIGLRIVFAVLSVIHRVR

SEQ ID NO: 6 - protein, 634 aa, synthetic construct
AENLWVTVYYGVPVWKDAETTLFCASDAKAYETEKHNVWATHACVPTDPNPQEIHLENVTEEFNMWKNNMVEQMHEDIISLWDQSLKPCVKLTPLCVTLQCTNVTNNITDDMRGELKNCSFNATTELRNKRQKVYSLFYRLDIVPMVDLWTNYRLINCNTSAITQACPKVSFEPIPIHYCAPAGFAILKCKDKKFNGTGPCQNVSTVQCTHGIKPVVSTQLLLNGSLAEEEVIIRSENITNNAKNILVQLNTSVQINCTRPNNNTVKSIRIGPGQAFYYTGDIIGDIRQAHCNVSKATWNETLGKVVKQLRKHFGNNTIIRFAQSSGGDLEVTTHSFNCGGEFFYCNTSGLFNSTWISNTSVQGSNSTGSNDSITLPCRIKQIINMWQRIGQAMYAPPIQGVIRCVSNITGLILTRDGGSTNSTTETFRPGGTDMRDNWRSELYKYKVVKIEPLGVAPTRCKRRVVGSHSGSGGSGSGGHAAVGIGAVSLGFLGAAGSTMGAASMTLTVQARNLLSGIVQQQSNLLRAPEPQQHLLKDTHWGIKQLQARVLAVEHYLRDQQLLGIWGCSGKLICCTNVPWNSSWSNRNLSEIWDNMTWLQWDKEISNYTQIIYGLLEESQNQQEKNEQDLLALD

SEQ ID NO: 7 - protein, 679 aa, synthetic construct
AENLWVTVYYGVPVWKDAETTLFCASDAKAYETEKHNVWATHACVPTDPNPQEIHLENVTEEFNMWKNNMVEQMHEDIISLWDQSLKPCVKLTPLCVTLQCTNVTNNITDDMRGELKNCSFNATTELRNKRQKVYSLFYRLDIVPMVDLWTNYRLINCNTSAITQACPKVSFEPIPIHYCAPAGFAILKCKDKKFNGTGPCQNVSTVQCTHGIKPVVSTQLLLNGSLAEEEVIIRSENITNNAKNILVQLNTSVQINCTRPNNNTVKSIRIGPGQAFYYTGDIIGDIRQAHCNVSKATWNETLGKVVKQLRKHFGNNTIIRFAQSSGGDLEVTTHSFNCGGEFFYCNTSGLFNSTWISNTSVQGSNSTGSNDSITLPCRIKQIINMWQRIGQAMYAPPIQGVIRCVSNITGLILTRDGGSTNSTTETFRPGGGDMRDNWRSELYKYKVVKIEPLGVAPTRCKRRVVGSHSGSGGSGSGGHAAVGIGAVSLGFLGAAGSTMGAASMTLTVQARNLLSGIVQQQSNLLRAPEPQQHLLKDTHWGIKQLQARVLAVEHYLRDQQLLGIWGCSGKLICCTNVPWNSSWSNRNLSEIWDNMTWLQWDKEISNYTQIIYGLLEESQNQQEKNEQDLLALDKWASLWNWFDISNWLWYIKIFIMIVGGLIGLRIVFAVLSVIHRVR

SEQ ID NO: 8 - protein, 634 aa, synthetic construct
AENLWVTVYYGVPVWKDAETTLFCASDAKAYETEKHNVWATHACVPTDPNPQEIHLENVTEEFNMWKNNMVEQMHEDIISLWDQSLKPCVKLTPLCVTLQCTNVTNNITDDMRGELKNCSFNATTELRNKRQKVYSLFYRLDIVPMVDLWTNYRLINCNTSAITQACPKVSFEPIPIHYCAPAGFAILKCKDKKFNGTGPCQNVSTVQCTHGIKPVVSTQLLLNGSLAEEEVIIRSENITNNAKNILVQLNTSVQINCTRPNNNTVKSIRIGPGQAFYYTGDIIGDIRQAHCNVSKATWNETLGKVVKQLRKHFGNNTIIRFAQSSGGDLEVTTHSFNCGGEFFYCNTSGLFNSTWISNTSVQGSNSTGSNDSITLPCRIKQIINMWQRIGQAMYAPPIQGVIRCVSNITGLILTRDGGSTNSTTETFRPGGGDMRDNWRSELYKYKVVKIEPLGVAPTRCKRRVVGSHSGSGGSGSGGHAAVGIGAVSLGFLGAAGSTMGAASMTLTVQARNLLSGIVQQQSNLLRAPEPQQHLLKDTHWGIKQLQARVLAVEHYLRDQQLLGIWGCSGKLICCTNVPWNSSWSNRNLSEIWDNMTWLQWDKEISNYTQIIYGLLEESQNQQEKNEQDLLALD

SEQ ID NO: 9 - protein, 679 aa, synthetic construct
AENLWVTVYYGVPVWKDAETTLFCASDAKAYETEKHNVWATHACVPTDPNPQEIHLENVTEEFNMWKNNMVEQMHEDIISLWDQSLKPCVKLTPLCVTLQCTNVTNNITDDMRGELKNCSFNATTELRNKRQKVYSLFYRLDIVPMVDLWTNYRLINCNTSAITQACPKVSFEPIPIHYCAPAGFAILKCKDKKFNGTGPCPSVSTVQCTHGIKPVVSTQLLLNGSLAEEEVIIRSENITNNAKNILVQLNTPVQINCTRPNNNTVKSIRIGPGQAFYYTGDIIGDIRQAHCNVSKATWNETLGKVVKQLRKHFGNNTIIRFAQSSGGDLEVTTHSFNCGGEFFYCNTSGLFNSTWISNTSVQGSNSTGSNDSITLPCRIKQIINMWQRIGQAMYAPPIQGVIRCVSNITGLILTRDGGSTNSTTETFRPGGGDMRDNWRSELYKYKVVKIEPLGVAPTRCKRRVVGSHSGSGGSGSGGHAAVGIGAVSLGFLGAAGSTMGAASMTLTVQARNLLSGIVQQQSNLLRAPEPQQHLLKDTHWGIKQLQARVLAVEHYLRDQQLLGIWGCSGKLICCTNVPWNSSWSNRNLSEIWDNMTWLQWDKEISNYTQIIYGLLEESQNQQEKNEQDLLALDKWASLWNWFDISNWLWYIKIFIMIVGGLIGLRIVFAVLSVIHRVR

SEQ ID NO: 10 - protein, 679 aa, synthetic construct
AENLWVTVYYGVPVWKDAETTLFCASDAKAYETEKHNVWATHACVSTDPNPQEIHLENVTEEFNMWKNNMVEQMHEDIISLWDQSLKPCVKLTPLCVGLQCTNVTNNITDDMRGELKNCSFNATTELRNKRQKVYSLFYRLDIVPMVDLWTNYRLISCNTSAITQACPKVSFEPIPIHYCAPAGFAILKCKDKKFNGTGPCPSVSTVQCTHGIKPVVSTQLLLNGSLAEEEVIIRSENITNNAKNILVQLNTPVQINCTRPNNNTVKSIRIGPGQAFYYTGDIIGDIRQAHCNVSKATWNETLGKVVKQLRKHFGNNTIIRFAQSSGGDLEVTTHSFNCGGEFFYCNTSGLFNSTWISNTSVQGSNSTGSNDSITLPCRIKQIINMWQRIGQAMYAPPIQGVIRCVSNITGLILTRDGGSTNSTTETFRPGGGDMRDNWRSELYKYKVVKIEPLGVAPTRCKRRVVGSHSGSGGSGSGGHAAVGIGAVSLGFLGAAGSTMGAASMTLTVQARNLLSGIVQQQSNLLRAPEPQQHLLKDTHWGIKQLQARVLAVEHYLRDQQLLGIWGCSGKLICCTNVPWNSSWSNRNLSEIWDNMTWLQWDKEISNYTQIIYGLLEESQNQQEKNEQDLLALDKWASLWNWFDISNWLWYIKIFIMIVGGLIGLRIVFAVLSVIHRVR

SEQ ID NO: 11 - protein, 634 aa, synthetic construct
AENLWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSAITQACPKVSFEPIPIHYCAPAGFAILKCKDKKFNGTGPCPSVSTVQCTHGIKPVVSTQLLLNGSLAEEEVMIRSENITNNAKNILVQFNTPVQINCTRPNNNTRKSIRIGPGQAFYATGDIIGDIRQAHCNVSKATWNETLGKVVKQLRKHFGNNTIIRFANSSGGDLEVTTHSFNCGGEFFYCNTSGLFNSTWISNTSVQGSNSTGSNDSITLPCRIKQIINMWQRIGQAMYAPPIQGVIRCVSNITGLILTRDGGSTNSTTETFRPGGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALD

SEQ ID NO: 12 - protein, 721 aa, synthetic construct
AENLWVTVYYGVPVWKDAETTLFCASDAKAYETEKHNVWATHACVPTDPNPQEIHLENVTEEFNMWKNNMVEQMHEDIISLWDQSLKPCVKLTPLCVTLQCTNVTNNITDDMRGELKNCSFNMTTELRDKKQKVYSLFYRLDVVQINENQGNRSNNSNKEYRLINCNTSAITQACPKVSFEPIPIHYCAPAGFAILKCKDKKFNGTGPCPSVSTVQCTHGIKPVVSTQLLLNGSLAEEEVIIRSENITNNAKNILVQLNTPVQINCTRPNNNTVKSIRIGPGQAFYYTGDIIGDIRQAHCNVSKATWNETLGKVVKQLRKHFGNNTIIRFAQSSGGDLEVTTHSFNCGGEFFYCNTSGLFNSTWISNTSVQGSNSTGSNDSITLPCRIKQIINMWQRIGQAMYAPPIQGVIRCVSNITGLILTRDGGSTNSTTETFRPGGGDMRDNWRSELYKYKVVKIEPLGVAPTRCKRRVVGSHSGSGGSGSGGHAAVGIGAVSLGFLGAAGSTMGAASMTLTVQARNLLSGIVQQQSNLLRAPEPQQHLLKDTHWGIKQLQARVLAVEHYLRDQQLLGIWGCSGKLICCTNVPWNSSWSNRNLSEIWDNMTWLQWDKEISNYTQIIYGLLEESQNQQEKNEQDLLALDGGGSGGSGGSEQKLISEEDLGGSGGSGGSNAVGQDTQEVIVVPHSLPFKVVVISAILALVVLTIISLIILIMLWQKKPR

SEQ ID NO: 13 - protein, 694 aa, synthetic construct
AENLWVTVYYGVPVWKDAETTLFCASDAKAYETEKHNVWATHACVPTDPNPQEIHLENVTEEFNMWKNNMVEQMHEDIISLWDQSLKPCVKLTPLCVTLQCTNVTNNITDDMRGELKNCSFNMTTELRDKKQKVYSLFYRLDVVQINENQGNRSNNSNKEYRLINCNTSAITQACPKVSFEPIPIHYCAPAGFAILKCKDKKFNGTGPCQNVSTVQCTHGIKPVVSTQLLLNGSLAEEEVIIRSENITNNAKNILVQLNTSVQINCTRPNNNTVKSIRIGPGQAFYYTGDIIGDIRQAHCNVSKATWNETLGKVVKQLRKHFGNNTIIRFAQSSGGDLEVTTHSFNCGGEFFYCNTSGLFNSTWISNTSVQGSNSTGSNDSITLPCRIKQIINMWQRIGQAMYAPPIQGVIRCVSNITGLILTRDGGSTNSTTETFRPGGGDMRDNWRSELYKYKVVKIEPLGVAPTRCKRRVVGSHSGSGGSGSGGHAAVGIGAVSLGFLGAAGSTMGAASMTLTVQARNLLSGIVQQQSNLLRAPEPQQHLLKDTHWGIKQLQARVLAVEHYLRDQQLLGIWGCSGKLICCTNVPWNSSWSNRNLSEIWDNMTWLQWDKEISNYTQIIYGLLEESQNQQEKNEQDLLALDKWASLWNWFDISNWLWYIKIFIMIVGGLIGLRIVFAVLSVIHRVRQGYSPLS

SEQ ID NO: 14 - protein, 694 aa, synthetic construct
AENLWVTVYYGVPVWKDAETTLFCASDAKAYETEKHNVWATHACVPTDPNPQEIHLENVTEEFNMWKNNMVEQMHEDIISLWDQSLKPCVKLTPLCVTLQCTNVTNNITDDMRGELKNCSFNMTTELRDKKQKVYSLFYRLDVVQINENQGNRSNNSNKEYRLINCNTSAITQACPKVSFEPIPIHYCAPAGFAILKCKDKKFNGTGPCPSVSTVQCTHGIKPVVSTQLLLNGSLAEEEVIIRSENITNNAKNILVQLNTPVQINCTRPNNNTVKSIRIGPGQAFYYTGDIIGDIRQAHCNVSKATWNETLGKVVKQLRKHFGNNTIIRFAQSSGGDLEVTTHSFNCGGEFFYCNTSGLFNSTWISNTSVQGSNSTGSNDSITLPCRIKQIINMWQRIGQAMYAPPIQGVIRCVSNITGLILTRDGGSTNSTTETFRPGGGDMRDNWRSELYKYKVVKIEPLGVAPTRCKRRVVGSHSGSGGSGSGGHAAVGIGAVSLGFLGAAGSTMGAASMTLTVQARNLLSGIVQQQSNLLRAPEPQQHLLKDTHWGIKQLQARVLAVEHYLRDQQLLGIWGCSGKLICCTNVPWNSSWSNRNLSEIWDNMTWLQWDKEISNYTQIIYGLLEESQNQQEKNEQDLLALDKWASLWNWFDISNWLWYIKIFIMIVGGLIGLRIVFAVLSVIHRVRQGYSPLS

SEQ ID NO: 15 - protein, 819 aa, synthetic construct
AENLWVTVYYGVPVWKDAETTLFCASDAKAYETEKHNVWATHACVPTDPNPQEIHLENVTEEFNMWKNNMVEQMHEDIISLWDQSLKPCVKLTPLCVTLQCTNVTNNITDDMRGELKNCSFNMTTELRDKKQKVYSLFYRLDVVQINENQGNRSNNSNKEYRLINCNTSAITQACPKVSFEPIPIHYCAPAGFAILKCKDKKFNGTGPCPSVSTVQCTHGIKPVVSTQLLLNGSLAEEEVIIRSENITNNAKNILVQLNTPVQINCTRPNNNTVKSIRIGPGQAFYYTGDIIGDIRQAHCNVSKATWNETLGKVVKQLRKHFGNNTIIRFAQSSGGDLEVTTHSFNCGGEFFYCNTSGLFNSTWISNTSVQGSNSTGSNDSITLPCRIKQIINMWQRIGQAMYAPPIQGVIRCVSNITGLILTRDGGSTNSTTETFRPGGGDMRDNWRSELYKYKVVKIEPLGVAPTRCKRRVVGSHSGSGGSGSGGHAAVGIGAVSLGFLGAAGSTMGAASMTLTVQARNLLSGIVQQQSNLLRAPEPQQHLLKDTHWGIKQLQARVLAVEHYLRDQQLLGIWGCSGKLICCTNVPWNSSWSNRNLSEIWDNMTWLQWDKEISNYTQIIYGLLEESQNQQEKNEQDLLALDGSGGLSERMLKALNDQLNRELYSAYLYFAMAAYFEDLGLEGFANWMKAQAEEEIGHALRFYNYIYDKNGRVELDEIPKPPKEWESPLKAFEAAYEHEKFISKSIYELAALAEEEKDYSTRAFLEWFINEQVEEEASVKKILDKLKFAKDSPQILFMLDKELSARAPKLPGLLMQGGE

SEQ ID NO: 16 - protein, 642 aa, synthetic construct
AENLWVTVYYGVPVWKDAETTLFCASDAKAYETEKHNVWATHACVPTDPNPQEIHLENVTEEFNMWKNNMVEQMHEDIISLWDQSLKPCVKLTPLCVTLQCTNVTNNITDDMRGELKNCSFNMTTELRDKKQKVYSLFYRLDVVQINENQGNRSNNSNKEYRLINCNTSAITQACPKVSFEPIPIHYCAPAGFAILKCKDKKFNGTGPCQNVSTVQCTHGIKPVVSTQLLLNGSLAEEEVIIRSENITNNAKNILVQLNTSVQINCTRPNNNTVKSIRIGPGQAFYYTGDIIGDIRQAHCNVSKATWNETLGKVVKQLRKHFGNNTIIRFAQSSGGDLEVTTHSFNCGGEFFYCNTSGLFNSTWISNTSVQGSNSTGSNDSITLPCRIKQIINMWQRIGQAMYAPPIQGVIRCVSNITGLILTRDGGSTNSTTETFRPGGGDMRDNWRSELYKYKVVKIEPLGVAPTRCKRRVVGSHSGSGGSGSGGHAAVGIGAVSLGFLGAAGSTMGAASMTLTVQARNLLSGIVQQQSNLLRAPEPQQHLLKDTHWGIKQLQARVLAVEHYLRDQQLLGIWGCSGKLICCTNVPWNSSWSNRNLSEIWDNMTWLQWDKEISNYTQIIYGLLEESQNQQEKNEQDLLALD

SEQ ID NO: 17 - protein, 687 aa, synthetic construct
AENLWVTVYYGVPVWKDAETTLFCASDAKAYETEKHNVWATHACVPTDPNPQEIHLENVTEEFNMWKNNMVEQMHEDIISLWDQSLKPCVKLTPLCVTLQCTNVTNNITDDMRGELKNCSFNMTTELRDKKQKVYSLFYRLDVVQINENQGNRSNNSNKEYRLINCNTSAITQACPKVSFEPIPIHYCAPAGFAILKCKDKKFNGTGPCQNVSTVQCTHGIKPVVSTQLLLNGSLAEEEVIIRSENITNNAKNILVQLNTSVQINCTRPNNNTVKSIRIGPGQAFYYTGDIIGDIRQAHCNVSKATWNETLGKVVKQLRKHFGNNTIIRFAQSSGGDLEVTTHSFNCGGEFFYCNTSGLFNSTWISNTSVQGSNSTGSNDSITLPCRIKQIINMWQRIGQAMYAPPIQGVIRCVSNITGLILTRDGGSTNSTTETFRPGGTDMRDNWRSELYKYKVVKIEPLGVAPTRCKRRVVGSHSGSGGSGSGGHAAVGIGAVSLGFLGAAGSTMGAASMTLTVQARNLLSGIVQQQSNLLRAPEPQQHLLKDTHWGIKQLQARVLAVEHYLRDQQLLGIWGCSGKLICCTNVPWNSSWSNRNLSEIWDNMTWLQWDKEISNYTQIIYGLLEESQNQQEKNEQDLLALDKWASLWNWFDISNWLWYIKIFIMIVGGLIGLRIVFAVLSVIHRVR

SEQ ID NO: 18 - protein, 687 aa, synthetic construct
AENLWVTVYYGVPVWKDAETTLFCASDAKAYETEKHNVWATHACVPTDPNPQEIHLENVTEEFNMWKNNMVEQMHEDIISLWDQSLKPCVKLTPLCVTLQCTNVTNNITDDMRGELKNCSFNMTTELRDKKQKVYSLFYRLDVVQINENQGNRSNNSNKEYRLINCNTSAITQACPKVSFEPIPIHYCAPAGFAILKCKDKKFNGTGPCQNVSTVQCTHGIKPVVSTQLLLNGSLAEEEVIIRSENITNNAKNILVQLNTSVQINCTRPNNNTVKSIRIGPGQAFYYTGDIIGDIRQAHCNVSKATWNETLGKVVKQLRKHFGNNTIIRFAQSSGGDLEVTTHSFNCGGEFFYCNTSGLFNSTWISNTSVQGSNSTGSNDSITLPCRIKQIINMWQRIGQAMYAPPIQGVIRCVSNITGLILTRDGGSTNSTTETFRPGGGDMRDNWRSELYKYKVVKIEPLGVAPTRCKRRVVGSHSGSGGSGSGGHAAVGIGAVSLGFLGAAGSTMGAASMTLTVQARNLLSGIVQQQSNLLRAPEPQQHLLKDTHWGIKQLQARVLAVEHYLRDQQLLGIWGCSGKLICCTNVPWNSSWSNRNLSEIWDNMTWLQWDKEISNYTQIIYGLLEESQNQQEKNEQDLLALDKWASLWNWFDISNWLWYIKIFIMIVGGLIGLRIVFAVLSVIHRVR

SEQ ID NO: 19 - protein, 642 aa, synthetic construct
AENLWVTVYYGVPVWKDAETTLFCASDAKAYETEKHNVWATHACVPTDPNPQEIHLENVTEEFNMWKNNMVEQMHEDIISLWDQSLKPCVKLTPLCVTLQCTNVTNNITDDMRGELKNCSFNMTTELRDKKQKVYSLFYRLDVVQINENQGNRSNNSNKEYRLINCNTSAITQACPKVSFEPIPIHYCAPAGFAILKCKDKKFNGTGPCPSVSTVQCTHGIKPVVSTQLLLNGSLAEEEVIIRSENITNNAKNILVQLNTPVQINCTRPNNNTVKSIRIGPGQAFYYTGDIIGDIRQAHCNVSKATWNETLGKVVKQLRKHFGNNTIIRFAQSSGGDLEVTTHSFNCGGEFFYCNTSGLFNSTWISNTSVQGSNSTGSNDSITLPCRIKQIINMWQRIGQAMYAPPIQGVIRCVSNITGLILTRDGGSTNSTTETFRPGGGDMRDNWRSELYKYKVVKIEPLGVAPTRCKRRVVGSHSGSGGSGSGGHAAVGIGAVSLGFLGAAGSTMGAASMTLTVQARNLLSGIVQQQSNLLRAPEPQQHLLKDTHWGIKQLQARVLAVEHYLRDQQLLGIWGCSGKLICCTNVPWNSSWSNRNLSEIWDNMTWLQWDKEISNYTQIIYGLLEESQNQQEKNEQDLLALD

SEQ ID NO: 20 - protein, 626 aa, synthetic construct
AENLWVTVYYGVPVWKDAETTLFCASDAKAYETEKHNVWATHACVPTDPNPQEIHLENVTEEFNMWKNNMVEQMHEDIISLWDQSLKPCVKLTPLCVTLNCTNVTNNITDDMRGELKNCSFNATTELRNKRQKVYSLFYRLDIVPMGENSTNYRLINCNTSAITQACPKVSFEPIPIHYCAPAGFAILKCKDKKFNGTGPCQNVSTVQCTHGIKPVVSTQLLLNGSLAEEEVIIRSENITNNAKNILVQLNTSVQINCTRPNNNTVKSIRIGPGQAFYYTGDIIGDIRQAHCNVSKATWNETLGKVVKQLRKHFGNNTIIRFAQSSGGDLEVTTHSFNCGGEFFYCNTSGLFNSTWISNTSVQGSNSTGSNDSITLPCRIKQIINMWQRIGQAMYAPPIQGVIRCVSNITGLILTRDGGSTNSTTETFRPGGGDMRDNWRSELYKYKVVKIEPLGVAPTRCKRRVVGRRRRRRAVGIGAVSLGFLGAAGSTMGAASMTLTVQARNLLSGIVQQQSNLLRAPEPQQHLLKDTHWGIKQLQARVLAVEHYLRDQQLLGIWGCSGKLICCTNVPWNSSWSNRNLSEIWDNMTWLQWDKEISNYTQIIYGLLEESQNQQEKNEQDLLALD

SEQ ID NO: 21 - protein, 626 aa, synthetic construct
AENLWVTVYYGVPVWKDAETTLFCASDAKAYETEKHNVWATHACVPTDPNPQEIHLENVTEEFNMWKNNMVEQMHEDIISLWDQSLKPCVKLTPLCVTLQCTNVTNNITDDMRGELKNCSFNATTELRNKRQKVYSLFYRLDIVPMGENSANYRLIDCNTSAITQACPKVSFEPIPIHYCAPAGFAILKCKDKKFNGTGPCPSVSTVQCTHGIKPVVSTQLLLNGSLAEEEVIIRSENITNNAKNILVQLNTPVQINCTRPNNNTVKSIRIGPGQAFYYTGDIIGDIRQAHCNVSKATWNETLGKVVKQLRKHFGNNTIIRFAQSSGGDLEVTTHSFNCGGEFFYCNTSGLFNSTWISNTSVQGSNSTGSNDSITLPCRIKQIINMWQRIGQAMYAPPIQGVIRCVSNITGLILTRDGGSTNSTTETFRPGGGDMRDNWRSELYKYKVVKIEPLGVAPTRCKRRVVGRRRRRRAVGIGAVSLGFLGAAGSTMGAASMTLTVQARNLLSGIVQQQSNLLRAPEPQQHLLKDTHWGIKQLQARVLAVEHYLRDQQLLGIWGCSGKLICCTNVPWNSSWSNRNLSEIWDNMTWLQWDKEISNYTQIIYGLLEESQNQQEKNEQDLLALD

SEQ ID NO: 22 - protein, 628 aa, synthetic construct
AENLWVTVYYGVPVWKDAETTLFCASDAKAYETEKHNVWATHACVPTDPNPQEIHLENVTEEFNMWKNNMVEQMHEDIISLWDQSLKPCVKLTPLCVTLQCTNVTNNITDDMRGELKNCSFNATTELRNKRVKRYSLFYRLDIVQIDSNRAKSHYRLINCNTSAITQACPKVSFEPIPIHYCAPAGFAILKCKDKKFNGTGPCPSVSTVQCTHGIKPVVSTQLLLNGSLAEEEVIIRSENITNNAKNILVQLNTPVQINCTRPNNNTVKSIRIGPGQAFYYTGDIIGDIRQAHCNVSKATWNETLGKVVKQLRKHFGNNTIIRFAQSSGGDLEVTTHSFNCGGEFFYCNTSGLFNSTWISNTSVQGSNSTGSNDSITLPCRIKQIINMWQRIGQAMYAPPIQGVIRCVSNITGLILTRDGGSTNSTTETFRPGGGDMRDNWRSELYKYKVVKIEPLGVAPTRCKRRVVGRRRRRRAVGIGAVSLGFLGAAGSTMGAASMTLTVQARNLLSGIVQQQSNLLRAPEPQQHLLKDTHWGIKQLQARVLAVEHYLRDQQLLGIWGCSGKLICCTNVPWNSSWSNRNLSEIWDNMTWLQWDKEISNYTQIIYGLLEESQNQQEKNEQDLLALD

SEQ ID NO: 23 - protein, 628 aa, synthetic construct
AENLWVTVYYGVPVWKDAETTLFCASDAKAYETEKHNVWATHACVPTDPNPQEIHLENVTEEFNMWKNNMVEQMHEDIISLWDQSLKPCVKLTPLCVTLNCTNVTNNITDDMRGELKNCSFNATTELRNKRVKRYSLFYRLDIVQIDSNRTKSHYRLINCNTSAITQACPKVSFEPIPIHYCAPAGFAILKCKDKKFNGTGPCPSVSTVQCTHGIKPVVSTQLLLNGSLAEEEVIIRSENITNNAKNILVQLNTPVQINCTRPNNNTVKSIRIGPGQAFYYTGDIIGDIRQAHCNVSKATWNETLGKVVKQLRKHFGNNTIIRFAQSSGGDLEVTTHSFNCGGEFFYCNTSGLFNSTWISNTSVQGSNSTGSNDSITLPCRIKQIINMWQRIGQAMYAPPIQGVIRCVSNITGLILTRDGGSTNSTTETFRPGGGDMRDNWRSELYKYKVVKIEPLGVAPTRCKRRVVGRRRRRRAVGIGAVSLGFLGAAGSTMGAASMTLTVQARNLLSGIVQQQSNLLRAPEPQQHLLKDTHWGIKQLQARVLAVEHYLRDQQLLGIWGCSGKLICCTNVPWNSSWSNRNLSEIWDNMTWLQWDKEISNYTQIIYGLLEESQNQQEKNEQDLLALD

SEQ ID NO: 24 - protein, 628 aa, synthetic construct
AENLWVTVYYGVPVWKDAETTLFCASDAKAYETEKHNVWATHACVPTDPNPQEIHLENVTEEFNMWKNNMVEQMHEDIISLWDQSLKPCVKLTPLCVTLNCTNVTNNITDDMRGELKNCSFNATTELRNKRVKRYSLFYRLDIVQIDSNRTKSHYRLINCNTSAITQACPKVSFEPIPIHYCAPAGFAILKCKDKKFNGTGPCQNVSTVQCTHGIKPVVSTQLLLNGSLAEEEVIIRSENITNNAKNILVQLNTSVQINCTRPNNNTVKSIRIGPGQAFYYTGDIIGDIRQAHCNVSKATWNETLGKVVKQLRKHFGNNTIIRFAQSSGGDLEVTTHSFNCGGEFFYCNTSGLFNSTWISNTSVQGSNSTGSNDSITLPCRIKQIINMWQRIGQAMYAPPIQGVIRCVSNITGLILTRDGGSTNSTTETFRPGGGDMRDNWRSELYKYKVVKIEPLGVAPTRCKRRVVGRRRRRRAVGIGAVSLGFLGAAGSTMGAASMTLTVQARNLLSGIVQQQSNLLRAPEPQQHLLKDTHWGIKQLQARVLAVEHYLRDQQLLGIWGCSGKLICCTNVPWNSSWSNRNLSEIWDNMTWLQWDKEISNYTQIIYGLLEESQNQQEKNEQDLLALD

SEQ ID NO: 25 - protein, 635 aa, synthetic construct
AENLWVTVYYGVPVWKDAETTLFCASDAKAYETEKHNVWATHACVPTDPNPQEIHLENVTEEFNMWKNNMVEQMHEDIISLWDQSLKPCVKLTPLCVTLQCTNVTNNITDDMRGELKNCSFNATTELRNKRQKVYSLFYRLDIVPMVDLWTNYRLINCNTSAITQACPKVSFEPIPIHYCAPAGFAILKCKDKKFNGTGPCPSVSTVQCTHGIKPVVSTQLLLNGSLAEEEVIIRSENITNNAKNILVQLNTPVQINCTRPNNNTVKSIRIGPGQAFYYTGDIIGDIRQAHCNVSKATWNETLGKVVKQLRKHFGNNTIIRFAQSSGGDLEVTTHSFNCGGEFFYCNTSGLFNSTWISNTSVQGSNSTGSNDSITLPCRIKQIINMWQRIGQAMYAPPIQGVIRCVSNITGLILTRDGGSTNSTTETFRPGGGDMRDNWRSELYKYKVVKIEPLGVAPTRCKRRVVGRRRRRRAVGIGAVSLGFLGAAGSTMGAASMTLTVQARNLLSGIVQQQSNLLRAPEPQQHLLKDTHWGIKQLQARVLAVEHYLRDQQLLGIWGCSGKLICCTNVPWNSSWSNRNLSEIWDNMTWLQWDKEISNYTQIIYGLLEESQNQQEKNEQDLLALDGTKHHHHHH

SEQ ID NO: 26 - protein, 628 aa, synthetic construct
AENLWVTVYYGVPVWKDAETTLFCASDAKAYETEKHNVWATHACVPTDPNPQEIHLENVTEEFNMWKNNMVEQMHEDIISLWDQSLKPCVKLTPLCVTLNCTNVTNNITDDMRGELKNCSFNATTELRNKRQKVYSLFYRLDIVPMVDLWTNYRLINCNTSAITQACPKVSFEPIPIHYCAPAGFAILKCKDKKFNGTGPCQNVSTVQCTHGIKPVVSTQLLLNGSLAEEEVIIRSENITNNAKNILVQLNTSVQINCTRPNNNTVKSIRIGPGQAFYYTGDIIGDIRQAHCNVSKATWNETLGKVVKQLRKHFGNNTIIRFAQSSGGDLEVTTHSFNCGGEFFYCNTSGLFNSTWISNTSVQGSNSTGSNDSITLPCRIKQIINMWQRIGQAMYAPPIQGVIRCVSNITGLILTRDGGSTNSTTETFRPGGGDMRDNWRSELYKYKVVKIEPLGVAPTRCKRRVVGRRRRRRAVGIGAVSLGFLGAAGSTMGAASMTLTVQARNLLSGIVQQQSNLLRAPEPQQHLLKDTHWGIKQLQARVLAVEHYLRDQQLLGIWGCSGKLICCTNVPWNSSWSNRNLSEIWDNMTWLQWDKEISNYTQIIYGLLEESQNQQEKNEQDLLALDGT

SEQ ID NO: 27 - protein, 635 aa, synthetic construct
GGNSSGSLGFLGAAGSTMGAASMTLTVQARNLLSGIVQQQSNLLRAPEPQQHLLKLTVWGIKQLQARVLAVEHYLRDQQLLGIWGCSGKLICCTNVPWNSSWSNRNLSEIWDNMTWLNWSKEISNYTQIIYGLLEESQNQNESNEQDLGGNGSGGGSGSGGNGSSGLWVTVYYGVPVWKDAETTLFCASDAKAYETEKHNVWATHECVPTDPNSSEIHLENVTEEFNMWKNNMVEQMHTDIIELWDQSLKPCVKLTPLCVTLQCTNVTNNITDDMRGELKNCSFNMTTELRDKKQKVYSLFYRLDVVQINENQGNRSNNSNKEYRLINCNTSAITQACPKVSFEPIPIHYCAPAGFAILKCKDKKFNGTGPCQNVSTVQCTHGIKPVVSTQLLLNGSLAEEEVIIRSENITNNAKNILVQLNTSVQINCTRPNNNTVKSIRIGPGQAFYYTGDIIGDIRQAHCNVSKATWNETLGKVVKQLRKHFGNNTIIRFAQSSGGDLEVTTHSFNCGGEFFYCNTSGLFNSTWISNTSVQGSNSTGSNDSITLPCRIKQIINMWQRIGQAMYAPPIQGVIRCVSNITGLILTRDGGSTNSTTETFRPGGGDMRDNWRSELYKYKVVKIEPLGVAPTRCNRS

SEQ ID NO: 28 - protein, 14 aa, synthetic construct
SHSGSGGSGSGGHA

SEQ ID NO: 29 - protein, 6 aa, synthetic construct
VIHRVR

SEQ ID NO: 30 - protein, 10 aa, synthetic construct
RVRQGYSPLS

SEQ ID NO: 31 - protein, 17 aa, synthetic construct
YYDFWYSDGYYYYYMDV

SEQ ID NO: 32 - protein, 9 aa, synthetic construct
GTKHHHHHH

SEQ ID NO: 33 - DNA, 22 bp, synthetic construct
  source 1..22: mol_type = other DNA
gctcagggaartagcccttgac

SEQ ID NO: 34 - DNA, 19 bp, synthetic construct
  source 1..19: mol_type = other DNA
tgggaagatggatacagtt

SEQ ID NO: 35 - protein, 15 aa, synthetic construct
TTYDFWSGYYYYYDV

SEQ ID NO: 36 - protein, 20 aa, synthetic construct
TTYYDFWSGYYYYYYYYMDV

SEQ ID NO: 37 - protein, 26 aa, synthetic construct
ARGTDYTIDDQGIRYQGSGTFWYFDV

SEQ ID NO: 38 - protein, 13 aa, synthetic construct
ARYYYGSGWYFDL

SEQ ID NO: 39 - protein, 28 aa, synthetic construct
VREAGGPDYRNGYNYYDFGYYNYHYMDV

SEQ ID NO: 40 - protein, 18 aa, synthetic construct
ARYYDFWSGYYYYYYMDV

SEQ ID NO: 41 - protein, 32 aa, synthetic construct
LTGSKHRLRDYFLYNEYGPNYEEWDYLATLDV

SEQ ID NO: 42 - protein, 15 aa, synthetic construct
ARDYGDYDYGDYMDV

SEQ ID NO: 43 - protein, 37 aa, synthetic construct
AKDLGESENEEWATDYYDFSIGYPGQDPRGVVGAFDI

SEQ ID NO: 44 - protein, 14 aa, synthetic construct
ARYYDFWSGYYFDI

SEQ ID NO: 45 - protein, 31 aa, synthetic construct
VREAGGPDYRNGYNYYDFYDGYYYNYHYMDV

SEQ ID NO: 46 - protein, 10 aa, synthetic construct
YYDFWSGYYT

SEQ ID NO: 47 - protein, 9 aa, synthetic construct
YYYYYYMDV

SEQ ID NO: 48 - protein, 30 aa, synthetic construct
AREAGGPDYRNGYNYYDFWSGYYYYYYMDV

SEQ ID NO: 49 - protein, 15 aa, synthetic construct
MGVEAGDESQHNTLI

SEQ ID NO: 50 - protein, 10 aa, synthetic construct
GVETDDHDFR

SEQ ID NO: 51 - protein, 7 aa, synthetic construct
GVETDDH

SEQ ID NO: 52 - protein, 5 aa, synthetic construct
GVEDH

SEQ ID NO: 53 - protein, 17 aa, synthetic construct
VEAGGPDYRNGYNYDNH

SEQ ID NO: 54 - protein, 13 aa, synthetic construct
EAGGPDYRNGYNN

SEQ ID NO: 55 - protein, 13 aa, synthetic construct
EAGGPDYRNGYNT

SEQ ID NO: 56 - protein, 12 aa, synthetic construct
EAGGPDYRNGYN

SEQ ID NO: 57 - protein, 20 aa, synthetic construct
ACDEFGHIKLMNPQRSTVWY

SEQ ID NO: 58 - protein, 137 aa, synthetic construct
QVQLVESGGGVVQPGSSLRLSCAASGFDFSRQGMHWVRQAPGQGLEWVAFIKYDGSEKYHADSVWGRLSISRDNSKDTLYLQMNSLRVEDTATYFCVREAGGPDYRNGYNYYDFYDGYYNYHYMDVWGKGTTVTVSS

SEQ ID NO: 59 - protein, 20 aa, synthetic construct
YYYYYYMDVWGKGTTVTVSS

SEQ ID NO: 60 - protein, 137 aa, synthetic construct
QVQLVESGGGVVQPGSSLRLSCAASGFDFSRQGMHWVRQAPGQGLEWVAFIKYDGSEKYHADSVWGRLSISRDNSKDTLYLQMNSLRVEDTATYFCVREAGGPDYRNGYNYWSFYDGYYYYYYMDVWGKGTTVTVSS

SEQ ID NO: 61 - protein, 137 aa, synthetic construct
QVQLVESGGGVVQPGSSLRLSCAASGFDFSRQGMHWVRQAPGQGLEWVAFIKYDGSEKYHADSVWGRLSISRDNSKDTLYLQMNSLRVEDTATYFCVREAGGPDYRNGYNYWSFYDGYYNYYYMDVWGKGTTVTVSS

SEQ ID NO: 62 - protein, 137 aa, synthetic construct
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCAREAGGPDYRNGYNYYDFYDGYYNYHYMDVWGKGTTVTVSS

SEQ ID NO: 63 - protein, 137 aa, synthetic construct
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCAREAGGPDYRNGYNYYDFWSGYYNYYYMDVWGKGTTVTVSS

SEQ ID NO: 64 - protein, 137 aa, synthetic construct
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCAREAGGPDYRNGYNYYDFWSGYYTYYYMDVWGKGTTVTVSS

SEQ ID NO: 65 - protein, 137 aa, synthetic construct
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCAREAGGPDYRNGYNYYDFWSGYYYYYYMDVWGKGTTVTVSS

SEQ ID NO: 66 - protein, 137 aa, synthetic construct
QVQLVESGGGVVQPGGSLRLSCLASGFTFHKYGMHWVRQAPGKGLEWVALISDDGMRKYHSDSMWGRVTISRDNSKNTLYLQFSSLKVEDTAMFFCAREAGGPIWHDDVKYYDFNDGYYNYHYMDVWGKGTTVTVSS

SEQ ID NO: 67 - protein, 137 aa, synthetic construct
QVQLVESGGGVVQPGGSLRLSCLASGFTFHKYGMHWVRQAPGKGLEWVALISDDGMRKYHSDSMWGRVTISRDNSKNTLYLQFSSLKVEDTAMFFCAREAGGPIWHDDVKYYDFWSGYYYYYYMDVWGKGTTVTVSS

SEQ ID NO: 68 - protein, 137 aa, synthetic construct
QVQLVESGGGVVQPGRSLRLSCAASGFTFSSYGMHWVRQAPGKGLEWVAVISYDGSNKYYADSVKGRFTISRDNSKNTLYLQMNSLRAEGTAVYYCAREAGGPIWHDDVKYYDFNDGYYNYHYMDVWGKGTTVTVSS

SEQ ID NO: 69 - protein, 137 aa, synthetic construct
QVQLVESGGGVVQPGRSLRLSCAASGFTFSSYGMHWVRQAPGKGLEWVAVISYDGSNKYYADSVKGRFTISRDNSKNTLYLQMNSLRAEGTAVYYCAREAGGPIWHDDVKYYDFWSGYYNYYYMDVWGKGTTVTVSS

SEQ ID NO: 70 - protein, 137 aa, synthetic construct
QVQLVESGGGVVQPGRSLRLSCAASGFTFSSYGMHWVRQAPGKGLEWVAVISYDGSNKYYADSVKGRFTISRDNSKNTLYLQMNSLRAEGTAVYYCAREAGGPIWHDDVKYYDFWSGYYYYYYMDVWGKGTTVTVSS

SEQ ID NO: 71 - protein, 134 aa, synthetic construct
EVQLVESGGGLVKPGGSLRLACVGSEFTFSEAWMTWVRQAPGKGLEWVGHMRPTTEGGAKDYAAAVRGRFTIARDDSKSTLYLQMNSLKIEDTGVYYCMTGVERGDFWSDDYSQHYNTYLIDVWGKGTTVTVSS

SEQ ID NO: 72 - protein, 134 aa, synthetic construct
EVQLVESGGGLVKPGGSLRLSCAASGFTFTNAWLDWVRQAPGKGLEWVGRIKSKTDGGTTDHAAPVKGRFTISRDDSKNTVYLQMNSLKIEDTAVYYCTTGVETYDFWSGYDDHYYDYYFKDVWGKGTTVTVSS

SEQ ID NO: 73 - protein, 134 aa, synthetic construct
EVQLVESGGGLVKPGGSLRLSCAASGFTFSNAWMSWVRQAPGKGLEWVGRIKSKTDGGTTDYAAPVKGRFTISRDDSKNTLYLQMNSLKTEDTAVYYCMTGVETYDFWSGYDDHYYDYYFRDVWGKGTTVTVSS

SEQ ID NO: 74 - protein, 134 aa, synthetic construct
EVQLVESGGGLVKPGGSLRLSCAASGFTFSNAWMSWVRQAPGKGLEWVGRIKSKTDGGTTDYAAPVKGRFTISRDDSKNTLYLQMNSLKTEDTAVYYCMTGVETYDFWSGYDDHYYDYYYMDVWGKGTTVTVSS

SEQ ID NO: 75 - protein, 134 aa, synthetic construct
EVQLVESGGGLVKPGGSLRLSCAASGFTFSNAWMSWVRQAPGKGLEWVGRIKSKTDGGTTDYAAPVKGRFTISRDDSKNTLYLQMNSLKTEDTAVYYCMTGVETYDFWSGYYDHYYDYYYMDVWGKGTTVTVSS

SEQ ID NO: 76 - protein, 81 aa, synthetic construct
LCVTLQCTNVTNNITDDMRGELKNCSFNMTTELRDKKQKVYSLFYRLDVVQINENQGNRSNNSNKEYRLINCNTSAITQAC

SEQ ID NO: 77 - protein, 81 aa, synthetic construct
LCVTLQCTNVTNNITDDMRGELKNCSFNMTTELRDKRVRRYSLFYRLDVVQINENQGNRSNNSNKEYRLINCNTSAITQAC

SEQ ID NO: 78 - protein, 75 aa, synthetic construct
LCVTLQCTNVTNNITDDMRGELKNCSFNMTTELRDKKQKVYSLFYRLDIVQIDSNRAKSHYRLINCNTSAITQAC

SEQ ID NO: 79 - protein, 81 aa, synthetic construct
LCVTLQCTNVTNNITDDMRGELKNCSFNMTTELRDKRVKRYSLFYRLDVVQINENQGNRSDNSNKEYRLINCNTSAITQAC

SEQ ID NO: 80 - protein, 73 aa, synthetic construct
LCVTLQCTNVTNNITDDMRGELKNCSFNATTELRNKRQKVYSLFYRLDIVPMGENSTNYRLINCNTSAITQAC

SEQ ID NO: 81 - protein, 73 aa, synthetic construct
LCVTLQCTNVTNNITDDMRGELKNCSFNATTELRNKRQKVYSLFYRLDIVPMGENSANYRLIDCNTSAITQAC

SEQ ID NO: 82 - protein, 81 aa, synthetic construct
LCVTLQCTNVTNNITDDMRGELKNCSFNATTELRNKRVKRYSLFYRLDIVQIDENQGNRSNRAKSHYRLINCNTSAITQAC

SEQ ID NO: 83 - protein, 81 aa, synthetic construct
LCVTLNCTNVTNNITDDMRGELKNCSFNATTELRNKRVKRYSLFYRLDIVQIDENQGNRSNRTKSHYRLINCNTSAITQAC

SEQ ID NO: 84 - protein, 81 aa, synthetic construct
LCVTLQCTNVTNNITDDMRGELKNCSFNATTELRNKRQKVYSLFYRLDIVPMVENQGNRSNDLWTNYRLINCNTSAITQAC

SEQ ID NO: 85 - protein, 81 aa, synthetic construct
LCVTLNCTNVTNNITDDMRGELKNCSFNATTELRNKRQKVYSLFYRLDIVPMVENQGNRSNDLWTNYRLINCNTSAITQAC

SEQ ID NO: 86 - protein, 25 aa, synthetic construct
TTGVETYDFWSGYYDHYYYYYYMDV

SEQ ID NO: 87 - protein, 25 aa, synthetic construct
TTGVETYDFWSGYDDHYYDYYFRDV

SEQ ID NO: 88 - protein, 25 aa, synthetic construct
TTGVETYDFWSGYDDHYYDYYFMDV

SEQ ID NO: 89 - protein, 25 aa, synthetic construct
TAGVETYDFWSGYDDHYYDYYYMDV

SEQ ID NO: 90 - protein, 25 aa, synthetic construct
TTGVETYDFWSGYDDHYYDYYFKDV

SEQ ID NO: 91 - protein, 25 aa, synthetic construct
TTGVETYDFWSAYRDDYYDYYYMDV

SEQ ID NO: 92 - protein, 25 aa, synthetic construct
MTGVETRDFWSGYYDDYYDHYFMDV

SEQ ID NO: 93 - protein, 25 aa, synthetic construct
MTGVETYDFWSAYDDHYYDYYFTDV

SEQ ID NO: 94 - protein, 25 aa, synthetic construct
MTGVETKDFWSGYYDDYYDHYFIDV

SEQ ID NO: 95 - protein, 25 aa, synthetic construct
MTGVETRDFWSGYYDDYYDHYFIDV

SEQ ID NO: 96 - protein, 25 aa, synthetic construct
MTGVETYSFWHDDRDDYYDYYYMDV

SEQ ID NO: 97 - protein, 25 aa, synthetic construct
MTGVETKDFWGDDYGDYYDHYFIDV

SEQ ID NO: 98 - protein, 25 aa, synthetic construct
MTGVETGDFWSDDYANYYDPYLIDV

SEQ ID NO: 99 - protein, 25 aa, synthetic construct
MTGVETGDFWSDDYGNYYDPYLIDV

SEQ ID NO: 100 - protein, 25 aa, synthetic construct
MTGVETGDFWSDYYAQYYDPYLIDV

SEQ ID NO: 101 - protein, 25 aa, synthetic construct
NTGVESYDFWSDDYSQYQYKYFWDV

SEQ ID NO: 102 - protein, 25 aa, synthetic construct
NTGVESYDFWSDDYAQYEYKYFRDV

SEQ ID NO: 103 - protein, 25 aa, synthetic construct
NTGVETYDFWSAYDDSYYDKYFTDV

SEQ ID NO: 104 - protein, 25 aa, synthetic construct
NTGVESYDFWSDDYSQYDYTYLSDV

SEQ ID NO: 105 - protein, 25 aa, synthetic construct
MTGVETGDFWSDEYSQHYDPYLIDV

SEQ ID NO: 106 - protein, 25 aa, synthetic construct
MTGVETGDFWSDDYSQHYNSYLIDV

SEQ ID NO: 107 - protein, 26 aa, synthetic construct
MTGVETKYDFWSGYYDDYYDHYFIDV

SEQ ID NO: 108 - protein, 25 aa, synthetic construct
MTGVERGGFWSDEYSQHWDPYLIDV

SEQ ID NO: 109 - protein, 25 aa, synthetic construct
MTGVEWYDFWSDEYSQEETKYFKDV

SEQ ID NO: 110 - protein, 25 aa, synthetic construct
MTGVEAGDFWSDEYSQHYNTYLIDV

SEQ ID NO: 111 - protein, 25 aa, synthetic construct
MTGVEKGDFWSDDYSQHYNTYLIDV

SEQ ID NO: 112 - protein, 25 aa, synthetic construct
MTGVERGDFWSDDYSQHYNTYLIDV

SEQ ID NO: 113 - protein, 15 aa, synthetic construct
ARYYYGSGSYWYFDL

SEQ ID NO: 114 - protein, 26 aa, synthetic construct
ARGTDYTIDDAGIHYQGSGTFWYFDL

SEQ ID NO: 115 - protein, 26 aa, synthetic construct
ARGTDYTIDDQGRFYQGSGTFWYFDF

SEQ ID NO: 116 - protein, 26 aa, synthetic construct
ARGTDYTIDDQGIFYKGSGTFWYFDL

SEQ ID NO: 117 - protein, 30 aa, synthetic construct
VREAGGPDYRNGYNYYDFYDGYYNYHYMDV

SEQ ID NO: 118 - protein, 30 aa, synthetic construct
AREAGGPIWHDDVKYYDFNDGYYNYHYMDV

SEQ ID NO: 119 - protein, 34 aa, synthetic construct
TRGSKHRLRDYVLYDDYGLINYQEWNDYLEFLDV

SEQ ID NO: 120 - protein, 34 aa, synthetic construct
TGGSKHRLRDYVLYDDYGLINQQEWNDYLEFLDV

SEQ ID NO: 121 - protein, 33 aa, synthetic construct
LTGSKHRLRDYFLYNEYGPNYEEWGDYLATLDV

SEQ ID NO: 122 - protein, 34 aa, synthetic construct
AKGSKHRLRDYALYDDDGALNWAVDVDYLSNLEF

SEQ ID NO: 123 - protein, 34 aa, synthetic construct
ARGSKHRLRDYVMYDDYGALQWAVYVDYLSNLDV

SEQ ID NO: 124 - protein, 34 aa, synthetic construct
AKGSKHRLRDYALYDDIGALQWAVDVDYLSTLEF

SEQ ID NO: 125 - protein, 33 aa, synthetic construct
VKGSKFRLREWADYNEWGLVSAQHGDYVTQLGI

SEQ ID NO: 126 - protein, 34 aa, synthetic construct
VRGAKFRLRHDATYDYWNDLLWADDRDYVTQLDL

SEQ ID NO: 127 - protein, 34 aa, synthetic construct
VRGAKFRLRHDATYDYYNDLLWADDRDYVTQLDL

SEQ ID NO: 128 - protein, 33 aa, synthetic construct
VKGQKFRLTEWADYNEFGLVAAQKGDYVTQLDV

SEQ ID NO: 129 - protein, 33 aa, synthetic construct
VKGQKFRLTEWADYNEFGLVAAEKGDYVTQLDV

SEQ ID NO: 130 - protein, 34 aa, synthetic construct
VRGSKFRLRNDAIYDYWNDLLWADDGDYVTKLDL

SEQ ID NO: 131 - protein, 33 aa, synthetic construct
ARRTEKQLRAEYVLDQEDGFYREEAIYITVLDV

SEQ ID NO: 132 - protein, 33 aa, synthetic construct
VKGLKFRLREWSDYNEFGLVAAQHGDYVTQMEV

SEQ ID NO: 133 - protein, 37 aa, synthetic construct
AKDVGDYKSDEWGTEYYDISISYPIQDPRAMVGAFDL

SEQ ID NO: 134 - protein, 37 aa, synthetic construct
AKDIREYECEYWTSDYYDFGRPQPCIDSRGVVGTFDV

SEQ ID NO: 135 - protein, 37 aa, synthetic construct
AKDLREDECEEWWSDYYDFGKQLPCRKSRGVAGIFDG

SEQ ID NO: 136 - protein, 37 aa, synthetic construct
AKDLREDECEEWWSDYYDFGKQLPCRKSRGVAGIFDK

SEQ ID NO: 137 - protein, 37 aa, synthetic construct
ARDQRYYECEEWASDYYDFGREQPCLDPRGVVGIFDL

SEQ ID NO: 138 - protein, 38 aa, synthetic construct
ARDLRELECEEWTLYNYYDFGSRGPCVDPRGVAGSFDV

SEQ ID NO: 139 - protein, 37 aa, synthetic construct
AKDLREDECEEWWSDYYDFGKKLPCRKSRGVAGVFDK

SEQ ID NO: 140 - protein, 39 aa, synthetic construct
VRDQREDECEEWWSDYYDFGRELPCRKFRGLGLAGIFDI

SEQ ID NO: 141 - protein, 39 aa, synthetic construct
VKDQREDECEEWWSDYYDFGRELPCRKSRGLGLAGIFDM

SEQ ID NO: 142 - protein, 37 aa, synthetic construct
AKDMREYECEYWTSDYYDFGRPQPCIDRRGVVGIFDM

SEQ ID NO: 143 - protein, 37 aa, synthetic construct
VKDMRELECEEWASDYYDFGKPQPCLDRRGVSGISAW

SEQ ID NO: 144 - protein, 37 aa, synthetic construct
ARDLRESECEEWESDYYDFGKKGPCVKPRGVAGGLDL

SEQ ID NO: 145 - protein, 37 aa, synthetic construct
ARDVREMECEEWASDYYDFGRGGPCRDFRGVVGILDI

SEQ ID NO: 146 - protein, 37 aa, synthetic construct
ARDLREYECELWASDYYDFGKPQPCEDPRGVVGTSDI

SEQ ID NO: 147 - protein, 37 aa, synthetic construct
ARDMREMECEEWASDYYDFGRPGPCRDLRGVVGTFDI

SEQ ID NO: 148 - protein, 37 aa, synthetic construct
ARDMREMECEEWASDYYDFGRSGPCRDFRGVVGVFDI

SEQ ID NO: 149 - protein, 37 aa, synthetic construct
ARDSREYECELWTSDYYDFGKPQPCIDTRDVGGLFDM

SEQ ID NO: 150 - protein, 39 aa, synthetic construct
VRDQREDECEERWSDYYHFGRVLPCRKYRGLGLAGVFDI

SEQ ID NO: 151 - protein, 39 aa, synthetic construct
VRDRQEDECEEWWSDYYAFGRGGPCRKYHGQGLAGIFDI

SEQ ID NO: 152 - protein, 37 aa, synthetic construct
AKDLREYECEEWRSDYYDFGRDQPCIDSQGVVGILDI

SEQ ID NO: 153 - protein, 37 aa, synthetic construct
AKDLGGIKNDEWGTDYYDISVSYPVQDPRAVAGIFDV

SEQ ID NO: 154 - protein, 38 aa, synthetic construct
AKDLREDECEEWWSDYYDFGKQLPCAKSRGGLVGIADN

SEQ ID NO: 155 - protein, 39 aa, synthetic construct
VRDQREDECEEWWSDYYDFGKELPCRKFRGLGLAGIFDV

SEQ ID NO: 156 - protein, 39 aa, synthetic construct
VRDQREDECEEWWSDYYDFGKELPCRKFRGLGLAGIFDI

SEQ ID NO: 157 - protein, 39 aa, synthetic construct
VRDRQEDECVEWWSDYYNFGRELPCSKFRGLGLAGIFDI

SEQ ID NO: 158 - protein, 39 aa, synthetic construct
VRDRQEDECEEWWSDYYNFGRELPCRKFRGQGLAGIFDI

SEQ ID NO: 159 - protein, 39 aa, synthetic construct
VRDRQEDECEEWWSDYYNFGRELPCRKFRGPGLAGIFDI

SEQ ID NO: 160 - protein, 37 aa, synthetic construct
VKDLRELECEEWTSDYYDFGRPQPCIDPRGVSGISAM

SEQ ID NO: 161 - protein, 37 aa, synthetic construct
ARDQRYYECEEWASDYYDFGREQPCQDPRGVVGIFDI

SEQ ID NO: 162 - protein, 10 aa, synthetic construct
YRKGDSGFWS

SEQ ID NO: 163 - protein, 7 aa, synthetic construct
YYQKGSG

SEQ ID NO: 164 - protein, 7 aa, synthetic construct
YGNDDEY

SEQ ID NO: 165 - protein, 5 aa, synthetic construct
YDFIL

SEQ ID NO: 166 - protein, 21 aa, synthetic construct
TTYYDFWSGYYTYYYYYYMDV

SEQ ID NO: 167 - protein, 25 aa, synthetic construct
TTGVEYYDFWSGYYDHYYYYYYMDV

SEQ ID NO: 168 - protein, 25 aa, synthetic construct
TTGVEYYDFWSGYYTHYYYYYYMDV

SEQ ID NO: 169 - protein, 25 aa, synthetic construct
TTGVETYDFWSGYDDHYYYYYYMDV

SEQ ID NO: 170 - protein, 25 aa, synthetic construct
ARVKYYYDFWSGYYTPGYYYYGMDV

SEQ ID NO: 171 - protein, 25 aa, synthetic construct
AKKSVHYDFWSGYESELGYYYGMDV

SEQ ID NO: 172 - protein, 25 aa, synthetic construct
ARFSDHYDFWSGYPAGKYYYYYMDV

SEQ ID NO: 173 - protein, 25 aa, synthetic construct
AKDLPNYDFWSGYYTGLYYYYGMDV

SEQ ID NO: 174 - protein, 25 aa, synthetic construct
AKGVDSYDFWSGYYEDYYYYYGMDV

SEQ ID NO: 175 - protein, 25 aa, synthetic construct
TTDIEDYDFWSGYYPDYYYYYYMDV

SEQ ID NO: 176 - protein, 25 aa, synthetic construct
ARDRRSYDFWSGYYTSKVDYYYMDV

SEQ ID NO: 177 - protein, 25 aa, synthetic construct
ARDVNTYDFWSGYYVDYYYYYGMDV

SEQ ID NO: 178 - protein, 25 aa, synthetic construct
TTDSTYYDFWSGYYDRSYYYYYMDV

SEQ ID NO: 179 - protein, 25 aa, synthetic construct
ARDSTYYDFWSGYYRGRFYYYGMDV

SEQ ID NO: 180 - protein, 25 aa, synthetic construct
AKDRVWGDFWSGYYTSYYYYYYMDV

SEQ ID NO: 181 - protein, 25 aa, synthetic construct
ARDAMYYDFWSGYYSPTYYYYYMDV

SEQ ID NO: 182 - protein, 25 aa, synthetic construct
AKDIKAGSFWSGYYPDFYYYYGMDV

SEQ ID NO: 183 - protein, 25 aa, synthetic construct
ARDPKYYDFWSGYYTSGLYYYGMDV

SEQ ID NO: 184 - protein, 25 aa, synthetic construct
TTGGDYYDFWSGYYTGPYYYYYMDV

SEQ ID NO: 185 - protein, 25 aa, synthetic construct
ARSFTYYDFWSGYSWDHSAFGVMDV

SEQ ID NO: 186 - protein, 25 aa, synthetic construct
TTDPRYYDFWSGYYGSYYYYYYMDV

SEQ ID NO: 187 - protein, 25 aa, synthetic construct
ARVVTYYDFWSGYYTGKHPESGMDV

SEQ ID NO: 188 - protein, 25 aa, synthetic construct
ARDNEHYDFWSGYYNPESDYYYMDV

SEQ ID NO: 189 - protein, 25 aa, synthetic construct
ARVGSYYDFWSGYYPGGSDYYYMDV

SEQ ID NO: 190 - protein, 25 aa, synthetic construct
ARALYAGDFWSGYYNAYYYYYGMDV

SEQ ID NO: 191 - protein, 25 aa, synthetic construct
ARVLKSYDFWSGYYEEAYYYYGMDV

SEQ ID NO: 192 - protein, 25 aa, synthetic construct
AKDGRYYDFWSGYYDPYYYYYGMDV

SEQ ID NO: 193 - protein, 25 aa, synthetic construct
AKDYSHYDFWSGYYTYKYYYYYMDV

SEQ ID NO: 194 - protein, 25 aa, synthetic construct
TREGSYYDFWSGDYSAFYYYYYMDV

SEQ ID NO: 195 - protein, 25 aa, synthetic construct
AKVGSNYDFWSGYFNDYYYYYYMDV

SEQ ID NO: 196 - protein, 25 aa, synthetic construct
ARDQKAYDFWSGYYSNSLYYYGMDV

SEQ ID NO: 197 - protein, 25 aa, synthetic construct
AREGRGYDFWSGYYSLDYYYYYMDV

SEQ ID NO: 198 - protein, 30 aa, synthetic construct
AREAGGPIWHDDVKYYDFWSGYYYYYYMDV

SEQ ID NO: 199 - protein, 30 aa, synthetic construct
AKDASPSSLPLGVGDYDFWSGPLSGYYMDV

SEQ ID NO: 200 - protein, 30 aa, synthetic construct
AREFVQYCSSTSCYLYDFWSGYQNYYGMDV

SEQ ID NO: 201 - protein, 30 aa, synthetic construct
ARAGYCSSTSCYPSGYDFSESYYYYYGMDV

SEQ ID NO: 202 - protein, 30 aa, synthetic construct
ARGPGPIDHRQTPRYYDFWSGYPYYYGMDV

SEQ ID NO: 203 - protein, 30 aa, synthetic construct
AGYCSSTSCYPEITYYDFWSGYSTQDGMDV

SEQ ID NO: 204 - protein, 30 aa, synthetic construct
ARDRAPPTGPEQPRYYDFWSGYYRTYGMDV

SEQ ID NO: 205 - protein, 30 aa, synthetic construct
AKESSGSFLNNVSRYYDFWSGYHYYYGMDV

SEQ ID NO: 206 - protein, 30 aa, synthetic construct
ARAPEVPAAIRWGAYYDFWSGYLEGYGMDV

SEQ ID NO: 207 - protein, 30 aa, synthetic construct
ARDGIVVVPAAIRPYYDFWSGYPDPYYMDV

SEQ ID NO: 208 - protein, 30 aa, synthetic construct
ARGPYYCSSTSCYAEYDFWSGSDYYYYMDV

SEQ ID NO: 209 - protein, 30 aa, synthetic construct
TTDWAVRDPGPVGAYYDFWSETEYYYGMDV

SEQ ID NO: 210 - protein, 30 aa, synthetic construct
AKDLSGSSWFIPPKTYDFWSGYPPDYGMDV

SEQ ID NO: 211 - protein, 30 aa, synthetic construct
AKDLFGGLGHPGAPDYDFWSGYFKPYYMDV

SEQ ID NO: 212 - protein, 30 aa, synthetic construct
ARDMTSRSSSRGHPSYDFWSGYSYYYYMDV

SEQ ID NO: 213 - protein, 30 aa, synthetic construct
AKDQGPRPIGSDKGYYDFWSGSYYYYGMDV

SEQ ID NO: 214 - protein, 30 aa, synthetic construct
TTDFFYRLSEGKVQYYDFWSGYYYYYGMDV

SEQ ID NO: 215 - protein, 30 aa, synthetic construct
ARRGEVVVPAAIVPGYDFWSGPHTDYYMDV

SEQ ID NO: 216 - protein, 30 aa, synthetic construct
ARDMSRVSERDGGENYDFWSGRPYYYYMDV

SEQ ID NO: 217 - protein, 26 aa, synthetic construct
ARGTDYTIDDQGIYYYGSGSYWYFDL

SEQ ID NO: 218 - protein, 26 aa, synthetic construct
ARDVKGRQQARALYYYGSGENWYFDL

SEQ ID NO: 219 - protein, 26 aa, synthetic construct
AKDSGGSTEVIRDYYYGSGSPRYFDL

SEQ ID NO: 220 - protein, 16 aa, synthetic construct
ARDYGDYYYYYYGMDV

SEQ ID NO: 221 - protein, 33 aa, synthetic construct
ARGSKHRLRDYFDYGDYGPNYEEWYYYYYGMDV

SEQ ID NO: 222 - protein, 33 aa, synthetic construct
ANSIWMTRKGGSDYGDYRRPDGLKNYYYYYMDV

SEQ ID NO: 223 - protein, 33 aa, synthetic construct
ATVGTRRGGVANDYGDYVSMAVPVDIHYYPMDV

SEQ ID NO: 224 - protein, 33 aa, synthetic construct
ARVGFTGSLQPPDYGDYGFRDPKDYYYYYGMDV

SEQ ID NO: 225 - protein, 33 aa, synthetic construct
ARVGLTGSLQPLDYGDYGIRDSKDYYYYYGMDV

SEQ ID NO: 226 - protein, 17 aa, synthetic construct
ARYYDFWSGYYTDAFDI

SEQ ID NO: 227 - protein, 38 aa, synthetic construct
TTDSGASPASRLCPSPNYDFWSGYYTRECGRYYDAFDI

SEQ ID NO: 228 - protein, 24 aa, synthetic construct
TGVETYDFWSGYDDHYYYYYYMDV

SEQ ID NO: 229 - protein, 24 aa, synthetic construct
TGVETYDFWSGYDDHYYDYYFRDV

SEQ ID NO: 230 - protein, 19 aa, synthetic construct
TYYDFWSGYYYYYYYYMDV

SEQ ID NO: 231 - protein, 134 aa, synthetic construct
EVQLVESGGGLVKPGGSLRLSCAASGFTFSNAWVSWVRQAPGKGKEWVGRIKSKTDGGTTDYAAPVKGRFTISRDDSKNTLYLQMNSLKTEDTAVYYCTTGVETYDFWSGYDDHYYDYYFRDVWGKGTTVTVSS

SEQ ID NO: 232 - protein, 27 aa, synthetic construct
CTTGVETYDFWSGYDDHYYDYYFRDVW

SEQ ID NO: 233 - protein, 27 aa, synthetic construct
CTTGVETYDFWSGYDDHYYYYYYMDVW

SEQ ID NO: 234 - protein, 27 aa, synthetic construct
CTTGVETYDFWSXYXDXYYXYXYXDXW

SEQ ID NO: 235 - protein, 5 aa, synthetic construct
YDFWS

SEQ ID NO: 236 - protein, 4 aa, synthetic construct
YYGS

SEQ ID NO: 237 - protein, 137 aa, synthetic construct
QVQLVESGGGVVQPGGSLRLSCLASGFTFHKYGMHWVRQAPGKGLEWVALISDDGMRKYHSDSMWGRVTISRDNSKNTLYLQFSSLKVEDTAMFFCAREAGGPIWHDDVKYYWSNDGYYNYHYMDVWGKGTTVTVSS